(19) United States
(12) Patent Application Publication
(10) Pub. No.: US 2018/0064662 A1
(43) Pub. Date: Mar. 8, 2018

(54) NOVEL COMPOSITIONS AND USES OF METFORMIN AGENTS

(71) Applicants: THE GENERAL HOSPITAL CORPORATION, Boston, MA (US); XTUIT PHARMACEUTICALS, INC., Waltham, MA (US)

(72) Inventors: Dai Fukumura, Newton, MA (US); Rakesh K. Jain, Wellesley, MA (US); Joao Incio, Boston, MA (US); Peter Blume-Jensen, Waltham, MA (US)

(73) Assignees: THE GENERAL HOSPITAL CORPORATION, Boston, MA (US); XTUIT PHARMACEUTICALS, INC., Waltham, MA (US)

(21) Appl. No.: 15/555,756

(22) PCT Filed: Nov. 25, 2015
(86) PCT No.: PCT/US15/62710

§ 371 Date: Sep. 5, 2017

Related U.S. Application Data
(60) Provisional application No. 62/128,759, filed on Mar. 5, 2015.
(60) Provisional application No. 62/252,926, filed on Nov. 9, 2015.

(51) Int. Cl. A61K 31/155; A61K 45/06; A61B 5/00; C07K 16/28; C07K 16/22; A61K 9/50

(52) U.S. Cl. CPC A61K 31/155; A61K 45/06; A61B 5/00; A61K 2300/00; C07K 16/22; A61K 9/5084; C07K 16/2863

(57) ABSTRACT

Methods and compositions for improving the delivery and/or efficacy of a therapy (e.g., an anti-cancer, anti-fibrotic or anti-inflammatory therapy) are disclosed. The invention is based, at least in part, on the discovery that metformin, a widely prescribed anti-diabetic drug, can affect the tumor microenvironment (e.g., directly, i.e., independent of its effects on cancer cells themselves or tumor metabolism). In embodiments described herein, metformin has been shown to reduce the amount of extracellular matrix, including collagen 1 and hyaluronan, in the fibro-inflammatory tumor microenvironment in a subject (e.g., a subject with a desmoplastic tumor).

### RELATED APPLICATIONS

This Application claims benefit under 35 U.S.C. §119(e) of the U.S. Provisional Application No. 62/128,759 filed Mar. 5, 2015 and U.S. Provisional Application No. 62/252,926, filed Nov. 9, 2015, the contents of each of which are incorporated herein by reference in their entirety.

### GOVERNMENT SUPPORT

This invention was made with Government support under Grant Nos. CA080124, CA085140, CA96915, CA115767, CA126642 awarded by NIH and Contract No. CDMRP BC095991 awarded by the DOD.

### BACKGROUND

The prognosis for patients with pancreatic cancer is poor. Patients with pancreatic cancer typically have an overall five-year rate survival of about 7% (American Cancer Society Facts and Figures 2015). Obesity and type-2 diabetes mellitus (DM2) have become a pandemic worldwide (McCarthy M. Bmj. 2014; 348:g3962; and Smyth S, Heron A. Nat Med. 2006;12(1): 75-80). Recent studies have demonstrated that these metabolic abnormalities are associated with the increased incidence, progression and poor prognosis of pancreatic ductal adenocarcinoma (PDAC) (Genkinger J M et al., International Journal of Cancer 2011;129(7): 1708-17; Kasenda B et al. BMC Cancer 2014;14: 728; Hori M et al. Clinical and Translational Gastroenterology. 2014;5:e53; Hursting et al. Annals of the New York Academy of Sciences 2012;1271: 82-7; Pischon T et al., Proc Nutr Soc. 2008;67(2): 128-45; and Calle E E et al., Oncogene 2004;23(38): 6365-78). DM2 and obesity may promote PDAC through pro-tumorigenic insulin and insulin-like growth factor-1 (IGF-1), as well as chronic inflammation.

Metformin is a widely prescribed anti-diabetic generic drug. Metformin is also administered to diabetic PDAC patients (Evans J M, Donnelly L A, Emslie-Smith A M, Alessi D R, Morris A D. Metformin and Reduced Risk of Cancer in Diabetic Patients. BMJ 2005;330(7503): 1304-5). A number of preventative early-stage clinical trials are ongoing, or about to be initiated, investigating metformin's anti-cancer potential. However, these studies have yielded inconsistent results, in which some but not all patients showed therapeutic benefit after metformin treatment. One reason for these results is the limited understanding of the biological role(s) of metformin in cancer. Insights into the mechanisms by which metformin exerts its anti-cancer effects can provide a basis to enhance its therapeutic efficacy either as a single agent or in combination with other agents to treat cancer. A need also exists to identify those patients that are likely benefit from a therapeutic regimen that includes metformin.

### SUMMARY

The invention is based, at least in part, on the discovery that metformin, a widely prescribed anti-diabetic drug, can affect the tumor microenvironment (e.g., directly, i.e., independent of its effects on cancer cells themselves or tumor metabolism). In embodiments described herein, metformin has been shown to reduce the amount of extracellular matrix, including collagen 1 and hyaluronan, in the fibro-inflammatory tumor microenvironment in a subject (e.g., a subject with a desmoplastic tumor). This effect co-incides with a reduction of angiotensin 2 type 1 receptor expression, which has previously been shown to both reduce solid stress, and hence allow for vessel decompression and reperfusion, as well as reduce TGF-beta signaling. Accordingly, metformin can be used to improve the delivery and/or efficacy of therapeutics, e.g., cancer and/or anti-fibrotic therapeutics. For example, metformin can be used to potentiate the action of other therapies that affect the microenvironment, e.g., tumor or liver microenvironment, including but not limited to, anti-hypertensive and/or collagen-modifying therapies, anti-fibrotic therapies, anti-inflammatory therapies, immune-checkpoint inhibitor therapies, and/or other microenviroments modulators (e.g., anti-angiogenic therapies). In some embodiments, metformin, alone or in combination with the agents described herein, can be used in a subject receiving chemotherapy (e.g., a patient having decreased chemotherapy efficacy e.g., due to decreased delivery and/or efficacy due to cancer fibrosis and desmoplastic microenvironment). In other embodiments, metformin can be used in a subject in combination with a treatment targeting fibrosis (e.g., anti-hypertensive and/or collagen modifying therapies), inflammation and/or immunosuppression (e.g., anti-cytokines, immune checkpoint inhibitors) or hypoxia (e.g., anti-angiogenic therapy). Thus, new compositions and uses of metformin in a subject, e.g., a patient having a cancer or a fibrotic disorder, alone or in combination with therapeutics targeting disease processes including fibrosis, inflammation, immunosuppression, and hypoxia, are disclosed.

In some embodiments, the inventors have discovered, inter alia, that metformin treatment reduced desmoplasia in fibrotic cancers. The appended examples show, in part, that obesity aggravated desmoplasia, which can lead to a reduced delivery and/or response to a chemotherapy. For example, metformin treatment reduced tumor levels of hyaluronan, and collagen-I in a preclinical obese/diabetic mouse model of syngeneic pancreatic ductal adenocarcinoma (PDAC). Without being bound by theory, the alleviation of desmoplasia can occur, at least in part, through a direct effect on hyaluronan and collagen-I production by pancreatic stellate cells. This effect was demonstrated to be associated with a reduction of AT1/PDGF-β expression and TGF-β/SMAD-2 signaling, consistent with a role of AT1 signaling in collagen-I and hyaluronan synthesis. Furthermore, metformin reduced the recruitment of tumor-associated macrophages and their expression of M2 markers in vivo at clinically relevant doses. Thus, metformin, alone or in combination with anti-hypertensive and/or collagen modifying therapies, anti-fibrotic therapies, anti-inflammatory therapies, immune-checkpoint inhibitor therapies, and/or other microenviroments modulators (e.g., anti-angiogenic therapies) can alleviate desmoplasia and tumor progression, e.g., in overweight or obese subjects.

In related embodiments, the effects of metformin on desmoplasia is primarily detected in subjects with a body mass index (BMI) higher than 25 (e.g., overweight and obese subjects). Said subjects may have increased levels of one or more extracellular matrix components and/or deregulated desmoplastic signaling activity in tumors. The appended examples show, in part, that obesity aggravated desmoplasia leads to a reduced delivery and/or response to chemotherapy. The effects of metformin on overweight and obese subjects suggest that a weight/metabolic-related parameter, e.g., BMI, can be used as a biomarker for improved effectiveness of a therapy, e.g., a metformin therapy and/or an AHCM therapy. With approximately 200 trials ongoing to address the effect of metformin on diabetic and non-diabetic cancer patients, understanding the effects of metformin can provide novel biomarkers of response and define strategies of patient stratification. For example as demonstrated herein, BMI can be used as a biomarker for evaluating, e.g., predicting, the response to metformin in a patient with a desmoplastic cancer, e.g., a pancreatic or breast cancer. This finding provides an advantage to current metformin therapies since treatment with metformin has shown inconsistent results in cancer patients. Stratification of cancer patients (e.g., cancer patients with desmoplastic tumors, e.g., pancreatic or breast cancer) using the weight/metabolic-related parameters described herein provides an improved metformin therapy. Thus, methods to evaluate, e.g., identify and/or stratify, a cancer patient for a therapy, e.g., metformin therapy and/or an AHCM therapy, utilizing a weight/metabolic-related biomarker are disclosed.

Accordingly, in one aspect, the invention features a method of improving the delivery and/or efficacy of a therapy (e.g., a cancer therapy, or an anti-fibrotic or anti-inflammatory therapy), or treating or preventing a disorder, e.g., a cancer, or a fibrotic or an inflammatory disorder, in a subject. The method includes administering a metformin agent to the subject, wherein the metformin agent is administered, alone or in combination with, one, two, three or more of:

(i) an anti-hypertensive and/or a collagen modifying agent (referred to herein as “AHCM” or “AHCM agent”) (e.g., an angiotensin receptor blocker (ARB));

(ii) a microenvironment modulator (e.g., an anti-angiogenic inhibitor, e.g., a low-dose anti-angiogenic inhibitor) and/or other stromal modulators;

(iii) an anti-inflammatory agent (e.g., a cytokine inhibitor); or

(iv) an inhibitor of an immune checkpoint molecule; and

optionally, administering the therapy (e.g., the cancer therapy, or the anti-fibrotic or anti-inflammatory therapy), under conditions sufficient to treat or prevent the disorder (e.g., the cancer, or the fibrotic or inflammatory disorder), in the subject, or to improve the delivery and/or efficacy of the therapy (e.g., the cancer therapy, or the anti-fibrotic or anti-inflammatory therapy) provided to the subject.

In a related aspect, the invention features a method of improving the delivery and/or efficacy of a therapy (e.g., a cancer therapy, or an anti-fibrotic or anti-inflammatory therapy), or treating or preventing a disorder, e.g., a cancer, or a fibrotic or an inflammatory disorder, in a subject. The method includes administering a metformin agent to the subject, in combination with an AHCM (e.g., an ARB), and

optionally, administering the therapy (e.g., the cancer therapy, or the anti-fibrotic or anti-inflammatory therapy), under conditions sufficient to treat or prevent the disorder (e.g., the cancer, or the fibrotic or inflammatory disorder), in the subject, or to improve the delivery and/or efficacy of the therapy (e.g., the cancer therapy, or the anti-fibrotic or anti-inflammatory therapy) provided to the subject.

In a related aspect, the invention features a method of improving the delivery and/or efficacy of a therapy (e.g., a cancer therapy, or an anti-fibrotic or anti-inflammatory therapy), or treating or preventing a disorder, e.g., a cancer, or a fibrotic or inflammatory disorder, in a subject. The method includes administering a metformin agent to the subject, in combination with a microenvironment modulator (e.g., an anti-angiogenic inhibitor, e.g., a low-dose anti-angiogenic inhibitor) and/or other stromal modulators, and

optionally, administering the therapy (e.g., the cancer therapy, or the anti-fibrotic or anti-inflammatory therapy), under conditions sufficient to treat or prevent the disorder (e.g., the cancer, or the fibrotic or inflammatory disorder), in the subject, or to improve the delivery and/or efficacy of the therapy (e.g., the cancer therapy, or the anti-fibrotic or anti-inflammatory therapy) provided to the subject.

In another aspect, the invention features a method for treating or preventing a liver disorder or condition in a subject. The method includes administering to the subject one or both of a metformin agent or an AHCM, and a vascular/stromal normalizing dose (e.g., a sub-anti-angiogenic dose) of a second agent chosen from one or more of: anti-angiogenic agent, sorafenib or an inhibitor of the angiopoietin-Tie-2 pathway (e.g., an Ang-1 or an Ang-2 inhibitor), thereby treating or preventing the liver disorder or condition.

In a related aspect, the invention features a method of improving the delivery and/or efficacy of a therapy (e.g., a cancer therapy, or an anti-fibrotic or anti-inflammatory therapy), or treating or preventing a disorder, e.g., a cancer, or a fibrotic or an inflammatory disorder, in a subject. The method includes administering a metformin agent to the subject, in combination with, an anti-inflammatory agent (e.g., a cytokine inhibitor), and

optionally, administering the therapy (e.g., the cancer therapy, or the anti-fibrotic or anti-inflammatory therapy), under conditions sufficient to treat or prevent the disorder (e.g., the cancer, or the fibrotic or inflammatory disorder), in the subject, or to improve the delivery and/or efficacy of the therapy (e.g., the cancer therapy, or the anti-fibrotic or anti-inflammatory therapy) provided to the subject.

In a related aspect, the invention features a method of improving the delivery and/or efficacy of a therapy (e.g., a cancer therapy, or an anti-fibrotic or anti-inflammatory therapy), or treating or preventing a disorder, e.g., a cancer, or a fibrotic or inflammatory disorder, in a subject. The method includes administering a metformin agent to the subject, in combination with, an inhibitor of an immune checkpoint molecule, and

optionally, administering the therapy (e.g., the cancer therapy, or the anti-fibrotic or anti-inflammatory therapy), under conditions sufficient to treat or prevent the disorder (e.g., the cancer, or the fibrotic or inflammatory disorder), in the subject, or to improve the delivery and/or efficacy of the therapy (e.g., the cancer therapy, or the anti-fibrotic or anti-inflammatory therapy) provided to the subject.

In any of the methods described herein, the method further includes identifying the subject as being in need of improved delivery and/or efficacy of the therapy (e.g., the cancer therapy, or the anti-fibrotic or anti-inflammatory therapy). In some embodiments, the method includes identifying the subject as having a desmoplastic disorder (e.g., a cancer, or a fibrotic or inflammatory disorder). In some embodiments, the method includes identifying the subject as being overweight or obese, e.g., as having a BMI greater than 25. In yet other embodiments, the method further includes identifying the subject as having a metabolic disorder, e.g., a systemic metabolic disorder. In embodiments, responsive to said identification, administering the metformin agent, alone or in combination with, one, two, three or more of: (i) an AHCM (e.g., an ARB); (ii) a microenvironment modulator (e.g., an anti-angiogenic inhibitor) and/or other stromal modulators; (iii) an anti-inflammatory agent (e.g., a cytokine inhibitor); or (iv) an inhibitor of an immune checkpoint molecule.

In another aspect, the invention features a method of improving the delivery and/or efficacy of a therapy (e.g., a cancer therapy, an anti-fibrotic therapy or an anti-inflammatory therapy), or treating or preventing a disorder (e.g., a cancer, a fibrotic disorder, or an inflammatory disorder), in an overweight or obese subject. The method includes:

administering to the subject, one or both of:

(i) an anti-hypertensive and/or a collagen modifying agent (AHCM) (e.g., an angiotensin receptor blocker (ARB));

(ii) a microenvironment modulator (e.g., an anti-angiogenic inhibitor, e.g., a low-dose anti-angiogenic inhibitor) and/or other stromal modulator;

optionally, administering the therapy, under conditions sufficient to treat or prevent the disorder, in the subject, or to improve the delivery and/or efficacy of the therapy provided to the subject.

In one embodiment, the subject is identified as being overweight or obese e.g., is identified as having a BMI greater than 25.

In another embodiment, the method further comprises identifying the subject as being overweight or obese, e.g., as having a BMI greater than 25, and responsive to said determination administering the AHCM, the microenvironment modulator, and/or the other stromal modulator.

In any of the methods described herein, the method further includes evaluating, e.g., acquiring a value for, a weight/metabolic-related parameter (e.g., BMI) for the subject. In some embodiments, responsive to a determination of the weight/metabolic-related parameter indicative of overweight or obesity (e.g., BMI value greater than 25), administering the metformin agent, alone or in combination with, one, two, three or more of: (i) an AHCM (e.g., an ARB); (ii) a microenvironment modulator (e.g., an anti-angiogenic inhibitor) and/or other stromal modulators; (iii) an anti-inflammatory agent (e.g., a cytokine inhibitor); or (iv) an inhibitor of an immune checkpoint molecule. In other embodiments, responsive to a determination of the weight/metabolic-related parameter indicative of normal or underweight in the subject, discontinuing or not administering the metformin agent, alone or in combination with, one, two, three or more of: (i) an AHCM (e.g., an ARB); (ii) a microenvironment modulator (e.g., an anti-angiogenic inhibitor, e.g., a low-dose anti-angiogenic inhibitor) and/or other stromal modulators; (iii) an anti-inflammatory agent (e.g., a cytokine inhibitor); or (iv) an inhibitor of an immune checkpoint molecule.

In another aspect, the invention features a combination or composition (e.g., one or more compositions or dosage forms), that includes a metformin agent in combination with one, two, three or more of:

(i) an AHCM (e.g., an ARB);

(ii) a microenvironment modulator (e.g., an anti-angiogenic inhibitor, e.g., a low-dose anti-angiogenic inhibitor) and/or other stromal modulators;

(iii) an anti-inflammatory agent (e.g., a cytokine inhibitor); or

(iv) an inhibitor of an immune checkpoint molecule; and

optionally, a therapy (e.g., a cancer therapy, or an anti-fibrotic or anti-inflammatory therapy).

In yet another aspect, the invention features a combination or composition (e.g., one or more compositions or dosage forms as described herein), for use in treating a disorder, e.g., a cancer, or a fibrotic or inflammatory disorder, or improving the delivery and/or efficacy of a therapy (e.g., a cancer therapy, or an anti-fibrotic or anti-inflammatory therapy). In embodiments, the composition for use includes a metformin agent, alone or in combination with one, two, three or more of:

(i) an AHCM (e.g., an ARB);

(ii) a microenvironment modulator (e.g., an anti-angiogenic inhibitor, e.g., a low-dose anti-angiogenic inhibitor) and/or other stromal modulators;

(iii) an anti-inflammatory agent (e.g., a cytokine inhibitor); or

(iv) an inhibitor of an immune checkpoint molecule; and

optionally, a therapy (e.g., a cancer therapy, or an anti-fibrotic or anti-inflammatory therapy).

Formulations, e.g., dosage formulations, and kits, e.g., therapeutic or diagnostic kits, that include a metformin agent, alone or in combination with one, two, three or more of:

(i) an AHCM (e.g., an ARB);

(ii) a microenvironment modulator (e.g., an anti-angiogenic inhibitor, e.g., a low-dose anti-angiogenic inhibitor) and/or other stromal modulators;

(iii) an anti-inflammatory agent (e.g., a cytokine inhibitor); or

(iv) an inhibitor of an immune checkpoint molecule; and

optionally, a therapy (e.g., a cancer therapy, or an anti-fibrotic or anti-inflammatory therapy), and (optionally) instructions for use, are also disclosed.

In another aspect, the invention features a method of evaluating, e.g., identifying and/or stratifying, responsiveness of a subject, e.g., a cancer patient, for a therapy, e.g., metformin therapy and/or an AHCM therapy. The method includes e.g., acquiring a value for, a weight/metabolic-related parameter (e.g., BMI) for the subject. In some embodiments, responsive to a determination of a weight/metabolic-related parameter indicative of overweight or obesity (e.g., BMI value greater than 25), performing one, two, three or more of:

(i) identifying the subject as being likely to respond to the therapy, e.g., the metformin therapy and/or the AHCM therapy;

(ii) stratifying the subject, or a patient populations (e.g., stratifying the subject) as being likely to respond (e.g., responders vs. non-responders) to the therapy, e.g., the metformin therapy and/or the AHCM therapy;

(iii) more effectively monitor the therapy, e.g., the metformin therapy and/or the AHCM therapy; or

(iv) administering the metformin agent, alone or in combination with, one, two, three or more of: (i) an AHCM (e.g., an ARB); (ii) a microenvironment modulator (e.g., an anti-angiogenic inhibitor, e.g., a low-dose anti-angiogenic inhibitor) and/or other stromal modulators; (iii) an anti-inflammatory agent (e.g., a cytokine inhibitor); or (iv) an inhibitor of an immune checkpoint molecule.

In some embodiments, responsive to a determination of a weight/metabolic-related parameter indicative of normal or underweight (e.g., BMI value less than 25), performing one, two, three or more of:

(i) identifying the subject as being less likely to respond to the therapy, e.g., the metformin therapy and/or the AHCM therapy;

(ii) stratifying the subject, or a patient populations (e.g., stratifying the subject) as being less likely to respond (e.g., responders vs. non-responders) to the therapy, e.g., the metformin therapy and/or the AHCM therapy;

(iii) more effectively monitor the therapy, e.g., the metformin therapy and/or the AHCM therapy; or

(iv) discontinuing or not administering the metformin agent, alone or in combination with, one, two, three or more of: (i) an AHCM (e.g., an ARB); (ii) a microenvironment modulator (e.g., an anti-angiogenic inhibitor, e.g., a low-dose anti-angiogenic inhibitor) and/or other stromal modulators; (iii) an anti-inflammatory agent (e.g., a cytokine inhibitor); or (iv) an inhibitor of an immune checkpoint molecule.

Additional features or embodiments of any of the methods, combinations, compositions, dosage formulations, and kits described herein include one or more of the following:

### Metformin Agents

In certain embodiments, the metformin agent is a biguanide. In some embodiments, the metformin agent includes two linked guanidine moieties. Exemplary biguanides include, but are not limited to, metformin, phenformin, buformin, and biguanide, or any functional analog, derivative, or salt of any of the aforesaid compounds.

In some embodiments, the metformin agent is described as a compound of Formula (I):

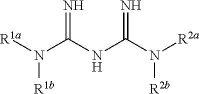

or a pharmaceutically acceptable salt thereof, wherein each of R^{1a}, R^{1b}, R^{2a}, and R^{2b} is independently hydrogen, C₁-C₆ alkyl, cycloalkylalkyl, or arylalkyl.

In some embodiments, each of R^{1a} and R^{1b} is hydrogen. In some embodiments, each of R^{2a} and R^{2b} is independently hydrogen or C₁-C₆ alkyl. In some embodiments, each of R^{2a} and R^{2b} is independently C₁-C₄ alkyl. In some embodiments, each of R^{2a} and R^{2b} is independently C₁-C₂ alkyl. In some embodiments, each of R^{2a} and R^{2b} is independently methyl. In some embodiments, each of R^{1a} and R^{1b} is hydrogen, and each of R^{2a} and R^{2b} is methyl. In some embodiments, the compound of Formula (I) is metformin, e.g., 3-(diaminomethylidene)-1,1-dimethylguanidine. In some embodiments, the compound of Formula (I) is metformin, e.g., as depicted in FIG. 32.

In some embodiments, the metformin agent is administered to the subject as a particle (e.g., a nanoparticle) or as a free agent, as described herein. In some embodiments, the combinations, compositions, dosage formulations include the metformin agent as a particle (e.g., a nanoparticle) or as a free agent, as described herein.

### AHCM

In some embodiments, the AHCM is chosen from one or more of: an angiotensin II receptor type 1 blocker (AT₁ blocker or ARB), an renin antagonist, an angiotensin converting enzyme (ACE) inhibitor, an antagonist of renin angiotensin aldosterone system (“RAAS antagonist”), a thrombospondin 1 (TSP-1) inhibitor, a transforming growth factor beta 1 (TGF-β1) inhibitor, a connective tissue growth factor (CTGF) inhibitor, a stromal cell-derived growth factor 1 alpha (SDF-1a) inhibitor, e.g., a CXCR-4 antagonist, e.g., AMD3100 or MSX-122, an AT₂ receptor agonist (e.g., CGP 42112A); an endothelin receptor antagonist (ERA), e.g., bosentan; an angiotensin II receptor type 2 agonist (AT₂ agonist), e.g. C21; a vitamin D receptor (VDR) agonist, e.g., vitamin D analog, e.g., calcipotriol); or a combination of two or more of the above.

In some embodiments, the AHCM is an AT₁ inhibitor. In an embodiment, the AT₁ blocker is chosen from one or more of: losartan (COZAAR®), valsartan (DIOVAN®), candesartan (ATACAND®), azilsartan, eprosartan (e.g., eprosartan mesylate (TEVETEN®)), EXP 3174, irbesartan (AVAPRO®), L158,809, olmesartan (BENICAR®), saralasin, telmisartan (MICARDIS®), or a derivative or metabolite thereof.

In one embodiment, the AHCM is a RAAS antagonist. In an embodiment, the RAAS antagonist is chosen from one or more of: aliskiren (TEKTURNA®, RASILEZ®), remikiren (Ro 42-5892), enalkiren (A-64662), SPP635, or a derivative thereof.

In yet another embodiment, the AHCM is an ACE inhibitor. In an embodiment, the ACE inhibitor is chosen from one or more of: benazepril (LOTENSIN®), captopril (CAPOTEN®), enalapril (VASOTEC®), fosinopril (MONOPRIL®), lisinopril (PRINIVIL®, ZESTRIL®), moexipril (UNIVASC®), perindopril (ACEON®), quinapril (ACCUPRIL®), ramipril (ALTACE®), trandolapril (MAVIK®), or a derivative thereof.

In yet another embodiment, the AHCM is a TSP-1 inhibitor. In an embodiment, the TSP-1 inhibitor is chosen from one or more of: ABT-510, CVX-045, LSKL, or a derivative thereof.

In one embodiment, the AHCM is a TGF-β1 inhibitor, e.g., an anti-TGF-β1 antibody, a TGF-β1 peptide inhibitor. In certain embodiment, the TGF-β1 inhibitor is chosen from one or more of: CAT-192, fresolimumab (GC1008), LY 2157299, Peptide 144 (P144), SB-431542, SD-208, compounds described in U.S. Pat. No. 7,846,908 and U.S. Patent Application Publication No. 2011/0008364, or a derivative thereof.

In yet another embodiment, the AHCM is a CTGF inhibitor. In certain embodiment, the CTGF inhibitor is chosen from one or more of: DN-9693, FG-3019, and compounds described in European Patent Application Publication No. 1839655, U.S. Pat. No. 7,622,454, or a derivative thereof.

In yet another embodiment, the AHCM is an inhibitor of stromal cell-derived growth factor 1 alpha (SDF-1a/CXCL12a). In certain embodiments, the SDF-1a inhibitor is an anti-SDF1a antibody or fragment thereof. In other embodiments, the SDF-1a inhibitor is an inhibitor of an SDF-1a receptor (e.g., a CXCR4 inhibitor), for example Plerixafor (AMD-3100).

Unless the context describes otherwise, the term “AHCM” may refer to one or more agents as described herein. The exemplary AHCMs are described herein are not limiting, e.g, derivatives or metabolites of AHCMs described herein can be used in the methods, combinations, compositions, dosage formulations, and kits disclosed herein. Additional description of the AHCMs is provided throughout, including the sections below entitled “AHCMs.” Any of the AHCMs disclosed herein, including those listed in the section entitled “AHCMs” can be used in the methods, combinations, compositions, dosage formulations, and kits disclosed herein. In some embodiments, the AHCM is administered to the subject as a particle (e.g., a nanoparticle) or as a free agent, as described herein. In some embodiments, the combinations, compositions, dosage formulations include the AHCM as a particle (e.g., a nanoparticle) or as a free agent, as described herein.

### Microenvironment Modulators

In some embodiments, the microenvironment modulator is chosen from one or more of an anti-angiogenic therapy, an agent that decreases the level or production of hyaluronic acid, an inhibitor of the hedgehog pathway, an agent that improves drug penetration in tumors (e.g., a disulfide-based cyclic RGD peptide peptide (iRGD) or an analogue thereof), a taxane therapy, an agent that modulates (e.g, inhibits) a hypoxia inducible factor (HIF) (e.g., HIF-1a and HIF-2a), an agent that decreases the level or production of collagen or procollagen, an agent that modulates the crosslinking of matrix molecules, an agent that depletes or changes the differentiation state of fibroblasts or stellate cells, an anti-fibrotic agent (e.g., a pirfenidone (PFD, 5-methyl-1-phenyl-2-(1H)-pyridone); or a combination of two or more of the above.

In one embodiment, the microenvironment modulator is an anti-angiogenic agent. In one embodiment, the anti-angiogenic agent is chosen from a VEGF-inhibitor, an inhibitor of the angiopoietin-Tie-2 pathway (e.g., an Ang-1 or an Ang-2 inhibitor), or sorafenib. Examples of anti-angiopoietin/Tie-2 pathway agents (or inhibitors of the angiopoietin-Tie-2 pathway) include, but are not limited to, AMG 386, CVX-060, CVX-241, MEDI-3617, REGN910, AMG-780, CEP-1198, ARRY-614, MGCD265, Regorafenib, and combinations thereof. In one embodiment, the anti-angiogenic agent is an inhibitor of tyrosine or Serine/Threonin kinases, such as VEGFR, PDGFR, c-kit receptors, b-Raf, or combinations thereof. Additional examples of anti-angiogenic agents include, but are not limited to, agents that inhibit oncogene activation (e.g., anti-EGFR such as gefitinib; anti-HER2 such as Trastuzumab; anti-P13K-AKT-mTOR such as NVPBEZ235, P1-103, Palomid-529, Nelfinavir; anti-Ras such as FTIs); agents that target androgens (e.g., Castration or endocrine therapy); agents that inhibits inflammatory cytokine-induced VEGF activation; anti-P1GF agents; anti-integrin agents (e.g., Cilengitide); agents that target PHD2/HIF pathway; anti-Rgs5 agents; Ang-1 agonistic agents; SEMA3A/NRP-1 agonistic agents; PDGF-B agonistic agents; eNOS agonistic agents; PDGF-C agonistic agents; PDGF-D agonistic agents, IFN-β agonistic agents; TSP-1 agonistic agents; anti-TNFα/TNFR agents; anti-TGFβ/TGFR agents; anti-VE-PTP agents; anti-MMP agents (e.g., anti-MMP-2; anti-MMP-9; anti-MMP-14); WNT agonistic agents; extracellular matrix-inducing agents (e.g., fibronectin; laminin; netrin-1; thrombospondin 1, etc.); Notch1 agonistic agents; Frizzled agonistic agents; and a combination of two or more thereof. Agents for anti-angiogenic/vascular normalization strategies as described in Goel et al. (2011) Physiol Rev. 91: 1071-1121, and Jain (2014) Cancer Cell 26(5): 605-622, the contents of which are incorporated herein by reference, can also be used as an anti-angiogentic agent for the compositions and methods described herein.

In one embodiment, the anti-angiogenic agent, e.g., sorafenib is administered to a subject at a vascular/stromal normalizing dose (e.g., a sub-anti-angiogenic dose, also referred to herein as a “low dose”) as a particle or a free agent, e.g., as described herein in the context of treatment of fibrotic conditions or disorders (e.g., liver diseases or disorders, kidney fibrosis, cardiovascular diseases, or idiopathic pulmonary fibrosis).

In another embodiment, the other stromal modulator is chosen from an inhibitor of a receptor for a VEGF ligand (e.g., a Flt-1-2, and/or -3 receptor), an inhibitor of an FGF receptor, a c-Met/HGF receptor inhibitor, a TNFR inhibitor, a cytokine/cytokine receptor inhibitor, a JAK/STAT3 inhibitor, an Osteopontin (SPP1) modulator, a Bone morphogenic protein (BMPs) inhibitor, an inhibitor of FAK, a CSF-1R inhibitor, a c-Kit inhibitor, DDR1 inhibitor, a metabolic inhibitor, and/or a mitochondrial inhibitor.

Additional description of the microenvironment modulators and/or other stromal modulators is provided throughout, including the section below entitled “Microenvironment Modulators.” Any of the microenvironment modulators and/or other stromal modulators disclosed herein, including those listed in the section entitled “Microenvironment Modulators” can be used in the methods, combinations, compositions, dosage formulations, and kits disclosed herein. In some embodiments, the microenvironment modulator and/or other stromal modulator is administered to the subject as a particle (e.g., a nanoparticle) or as a free agent, as described herein. In some embodiments, the combinations, compositions, dosage formulations include the microenvironment modulator and/or other stromal modulator as a particle (e.g., a nanoparticle) or as a free agent, as described herein.

### Anti-Inflammatory Agents

In one embodiment, the anti-inflammatory agent is an agent that blocks, inhibits, or reduces inflammation or signaling from an inflammatory signaling pathway. In one embodiment, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23, interferons (IFNs), e.g., IFNα, IFNβ, IFNγ, IFN-γ inducing factor (IGIF), transforming growth factor-β (TGF-β), transforming growth factor-α (TGF-α), tumor necrosis factors TNF-α, TNF-β, TNF-RI, TNF-RII, CD23, CD30, CD40L, EGF, G-CSF, GDNF, PDGF-BB, RANTES/CCL5, IKK, NF-κB, TLR2, TLR3, TLR4, TL5, TLR6, TLR7, TLR8, TLR8, TLR9, and/or any cognate receptors thereof.

In one embodiment, the anti-inflammatory agent is an IL-1 or IL-1 receptor antagonist, such as anakinra (KINERET®), rilonacept, or canakinumab.

In one embodiment, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061.

In one embodiment, the anti-inflammatory agent is a TNF-α antagonist, e.g., an anti-TNFα antibody, such as infliximab (REMICADE®), golimumab (SIMPONI®), adalimumab (HUMIRA®), certolizumab pegol (CIMZIA®) or etanercept.

In one embodiment, the anti-inflammatory agent is a corticosteroid. Exemplary corticosteroids include, but are not limited to, cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PREZONE®), and prednisone (DELTASONE®, LIQUID PRED®, METICORTEN®, ORASONE®)), and bisphosphonates (e.g., pamidronate (AREDIA®), and zoledronic acid (ZOMETAC®).

Additional description of the anti-inflammatory agents is provided throughout, including the section below entitled “Anti-inflammatory Agents.” Any of the anti-inflammatory agents disclosed herein, including those listed in the section entitled “Anti-inflammatory Agents” can be used in the methods, combinations, compositions, dosage formulations, and kits disclosed herein. In some embodiments, the anti-inflammatory agent is administered to the subject as a particle (e.g., a nanoparticle) or as a free agent, as described herein. In some embodiments, the combinations, compositions, dosage formulations include the anti-inflammatory agent as a particle (e.g., a nanoparticle) or as a free agent, as described herein.

### Immune Checkpoint Inhibitors

Immune checkpoint inhibitors, as described herein, refer to molecules that block, inhibit, or reduce activity of one or more immune checkpoint molecules. The inhibitors can be an antibody, an antigen binding fragment thereof, an immunoadhesin, a fusion protein, or an oligopeptide. In embodiments, the immune checkpoint inhibitor is an inhibitor of one or more of any of the following immune checkpoint molecules: PD-1, PD-L1, PD-L2, CTLA4, B7-H3, B7-H4, HVEM, BTLA, a killer-cell immunoglobulin-like receptor (KIR), LAG3, TIM3, CEACAM-1, CEACAM-3, CEACAM-5, GAL9, VISTA, TIGIT, LAIR1, CD160, 2B4, TGFRbeta, and A2aR.

In one embodiment, the immune checkpoint inhibitor is a PD-1 inhibitor. In one embodiment, the PD-1 inhibitor is chosen from one or more of: nivolumab (MDX-1106 or BMS-936558), pembrolizumab (MK03475), pidilizumab (CT-011), or AMP-224.

In one embodiment, the immune checkpoint inhibitor is a PD-L1 inhibitor. In one embodiment, the PD-L1 inhibitor is chosen from one or more of: YW243.55.570, MPDL3280A, MEDI-4736, MSB-0010718C, or MDX-1105 (BMS-936559).

In one embodiment, the immune checkpoint inhibitor is a TIM inhibitor. In one embodiment, the TIM inhibitor is chosen from one or more of: anti-TIM3 antibody RMT3-23 and clone 8B.2C12, or a bispecific anti-TIM3 and anti-PD-1 antibody.

In one embodiment, the immune checkpoint inhibitor is a LAG3 inhibitor. In one embodiment, the LAG3 inhibitor is chosen from one or more of: BMS-986016 (Bristol-Myers Squib), IMP701 (Immutep), IMP731 (Immutep and GlaxoSmithKline), IMP321 (Immutep), and antibodies disclosed in WO2010/019570.

In one embodiment, the immune checkpoint inhibitor is a CEACAM inhibitor, e.g., a CEACAM-1 inhibitor, a CEACAM-3 inhibitor, and/or a CEACAM-5 inhibitor.

Additional description of the immune checkpoint inhibitor is provided throughout, including the section below entitled “Immune Checkpoint Inhibitor.” Any of the immune checkpoint inhibitors disclosed herein, including those listed in the section entitled “Immune Checkpoint Inhibitor” can be used in the methods, combinations, compositions, dosage formulations, and kits disclosed herein. In some embodiments, the immune checkpoint inhibitor is administered to the subject as a particle (e.g., a nanoparticle) or as a free agent, as described herein. In some embodiments, the combinations, compositions, dosage formulations include the immune checkpoint inhibitor as a particle (e.g., a nanoparticle) or as a free agent, as described herein.

In other embodiments, the metformin agent can be used in combination with an activator of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

### Therapies

In some embodiments, the therapy administered in combination with the metformin agent and/or one or more of (i) an AHCM (e.g., an ARB); (ii) a microenvironment modulator (e.g., an anti-angiogenic inhibitor, e.g., a low-dose anti-angiogenic inhibitor) and/or other stromal modulators; (iii) an anti-inflammatory agent (e.g., a cytokine inhibitor); or (iv) an inhibitor of an immune checkpoint molecule, is a cancer therapy or an anti-fibrotic therapy.

In some embodiments, the cancer therapy is or includes an anti-cancer agent, including but not limited to, a small molecule, a kinase inhibitor, an alkylating agent, a vascular disrupting agent, a microtubule targeting agent, a mitotic inhibitor, a topoisomerase inhibitor, an anti-angiogenic agent, or an anti-metabolite. In one embodiment, the agent, e.g., the therapeutic agent, is a taxane (e.g., paclitaxel, docetaxel, larotaxel or cabazitaxel). In some embodiments, the anti-cancer agent is an anthracycline (e.g., doxorubicin). In some embodiments, the anti-cancer agent is a platinum-based agent (e.g., cisplatin or oxaliplatin). In some embodiments, the anti-cancer agent is a pyrimidine analog (e.g., gemcitabine). In some embodiments, the anti-cancer agent is chosen from camptothecin, irinotecan, rapamycin, FK506, 5-FU, leucovorin, or a combination thereof. In other embodiments, the anti-cancer agent is a protein biologic (e.g., an antibody molecule), or a nucleic acid therapy (e.g., an antisense or inhibitory double stranded RNA molecule).

In some embodiments, the cancer therapy is chosen from one or more of:

(i) a cancer therapeutic chosen from a viral cancer therapeutic agent, a lipid nanoparticle of an anti-cancer therapeutic agent, a polymeric nanoparticle of an anti-cancer therapeutic agent, an antibody against a cancer target, a dsRNA agent, an antisense RNA agent, or a chemotherapeutic agent;

(ii) an immunotherapy (e.g., an immune-cell therapy or adoptive immunotherapy);

(iii) radiation;

(iv) surgery;

(v) a photodynamic therapy; or

(vi) any combination of (i)-(v).

In some embodiments, the lipid nanoparticle is chosen from pegylated liposomal doxorubicin (DOXIL®) or liposomal paclitaxel (e.g., Abraxane®).

In other embodiments, the chemotherapeutic agent is a cytotoxic or a cytostatic agent. In some embodiments, the chemotherapeutic agent is an antimicrotubule agent, a topoisomerase inhibitor, a taxane, an antimetabolite, a mitotic inhibitor, an alkylating agent, or an intercalating agent. In some embodiments, the chemotherapeutic agent is chosen from gemcitabine, cisplatin, epirubicin, 5-fluorouracil, paclitaxel, oxaliplatin, or leucovorin.

In some embodiments, the antibody against the cancer target is chosen from an antibody against HER-2/neu, HER3, VEGF, or EGFR.

In some embodiments, the cancer therapy is a tyrosine kinase inhibitor chosen from sunitinib, erlotinib, gefitinib, sorafenib, icotinib, lapatinib, neratinib, vandetanib, BIBW 2992 or XL-647, or an anti-EGFR antibody chosen from cetuximab, panitumumab, zalutumumab, nimotuzumab necitumumab or matuzumab.

In other embodiments, the cancer therapy is chosen from one of more of: an anti-angiogenic agent, or a vascular targeting agent or a vascular disrupting agent.

Additional examples of anti-cancer agents are disclosed herein. Any of the anti-cancer agents disclosed herein, including those listed in the Detailed Description, can be used in free form or as a particle, or other composition disclosed herein.

In other embodiments, the therapy administered in combination with the metformin agent and/or one or more of (i) an AHCM (e.g., an ARB); (ii) a microenvironment modulator (e.g., an anti-angiogenic inhibitor, e.g., a low-dose anti-angiogenic inhibitor) and/or other stromal modulators; (iii) an anti-inflammatory agent (e.g., a cytokine inhibitor); or (iv) an inhibitor of an immune checkpoint molecule, is an anti-fibrotic therapy. In some embodiments, the anti-fibrotic therapy includes an anti-fibrotic agent (e.g., a pirfenidone (PFD, 5-methyl-1-phenyl-2-(1H)-pyridone).

### Administration

In some embodiments, administration of the metformin agent, alone or in combination with one or more of: i) AHCM, ii) microenvironment modulator, iii) anti-inflammatory agent, and/or iv) immune checkpoint inhibitor results in (e.g., is administered at a dose that causes), one or more of: decreases the level or production of an extracellular matrix component, such as a fiber (e.g., collagen, procollagen), and/or a polysaccharide (e.g., a glycosaminoglycan such as hyaluronan or hyaluronic acid); decreases the level or production of collagen or procollagen; decreases the level or production of hyaluronic acid; decreases tumor fibrosis; increases interstitial tumor transport; improves tumor perfusion; increases tumor oxygenation; decreases tumor hypoxia; decreases tumor acidosis; enables immune cell infiltration; decreases immunosuppression; increases antitumor immunity; decreases the production of cancer stem cells (also referred to herein as tumor-initiating cells); or enhances the efficacy (e.g., penetration or diffusion), of the therapy, e.g., the cancer therapy (e.g., radiation, photodynamic therapy, chemotherapeutics and immunotherapies) in a tumor or tumor vasculature, in the subject.

In some embodiments, the metformin agent and i) the AHCM agent, ii) the microenvironment modulator or stromal modulator, iii) the anti-inflammatory agent, and/or iv) the immune-checkpoint agent, as described herein, are administered in combination, e.g., concurrently or sequentially. Administration of the agents can occur in any order, via the same or different route, or in the same or different composition, e.g., pharmaceutical composition. In some embodiments, one, two, three or more of the metformin agent and i) the AHCM agent, ii) the microenvironment modulator or stromal modulator, iii) the anti-inflammatory agent, and/or iv) the immune-checkpoint agent, as described herein, are administered a free agent, or as a pharmaceutical composition or forumulation (e.g., a particle, e.g., a nanoparticle as described herein).

In certain embodiments, the metformin agent and i) the AHCM agent, ii) the microenvironment modulator or stromal modulator, iii) the anti-inflammatory agent, and/or iv) the immune-checkpoint agent, as described herein, are administered concurrently (e.g., administration of the two or more agents at the same time or day, or within the same treatment regimen) and/or sequentially (e.g., administration of one agent over a period of time followed by administration of another agent for a second period of time, or within different treatment regimens). In other embodiments, administration of two or more agents occur in overlapping treatment regimens (e.g., administration of one agent is initiated before the completion of the treatment regimen of another agent, or the administration of one agent is completed before the termination of the treatment regimen of another agent).

In one embodiment, the metformin agent is administered prior to i) the AHCM, ii) the microenvironment modulator, iii) the anti-inflammatory agent, and/or iv) the immune checkpoint inhibitor. In other embodiments, the metformin agent is administered prior to i) the AHCM, ii) the microenvironment modulator, iii) the anti-inflammatory agent, and/or iv) the immune checkpoint inhibitor, and followed by concurrent administration of the metformin agent and i) the AHCM, ii) the microenvironment modulator, iii) the anti-inflammatory agent, and/or iv) the immune checkpoint inhibitor.

In certain embodiments, the metformin agent and i) the AHCM, ii) the microenvironment modulator, iii) the anti-inflammatory agent, and/or iv) the immune checkpoint inhibitor are administered concurrently. For example, in certain embodiments, the metformin agent and i) the AHCM, ii) the microenvironment modulator, iii) the anti-inflammatory agent, and/or iv) the immune checkpoint inhibitor are administered at the same time, on the same day, or within the same treatment regimen. In certain embodiments, the metformin agent is administered before i) the AHCM, ii) the microenvironment modulator, iii) the anti-inflammatory agent, and/or iv) the immune checkpoint inhibitor on the same day or within the same treatment regimen.

In certain embodiments, the metformin agent is concurrently administered with i) the AHCM, ii) the microenvironment modulator, iii) the anti-inflammatory agent, and/or iv) the immune checkpoint inhibitor for a period of time, after which point treatment with i) the AHCM, ii) the microenvironment modulator, iii) the anti-inflammatory agent, and/or iv) the immune checkpoint inhibitor is stopped and treatment with the metformin agent continues.

In other embodiments, the metformin agent is concurrently administered with i) the AHCM, ii) the microenvironment modulator, iii) the anti-inflammatory agent, and/or iv) the immune checkpoint inhibitor for a period of time, after which point treatment with the metformin agent is stopped and treatment with i) the AHCM, ii) the microenvironment modulator, iii) the anti-inflammatory agent, and/or iv) the immune checkpoint inhibitor continues.

In certain embodiments, the metformin agent and i) the AHCM, ii) the microenvironment modulator, iii) the anti-inflammatory agent, and/or iv) the immune checkpoint inhibitor are administered sequentially. For example, in certain embodiments, the metformin agent is administered after the treatment regimen of i) the AHCM, ii) the microenvironment modulator, iii) the anti-inflammatory agent, and/or iv) the immune checkpoint inhibitor has ceased. In certain embodiments, i) the AHCM, ii) the microenvironment modulator, iii) the anti-inflammatory agent, and/or iv) the immune checkpoint inhibitor is administered after the treatment regimen of the metformin agent has ceased.

In some embodiments, the metformin agent and i) the AHCM, ii) the microenvironment modulator, iii) the anti-inflammatory agent, and/or iv) the immune checkpoint inhibitor can be administered in a pulse administration. In other embodiments, they can be administered as a pulse-chase administration, e.g., where the metformin agent is administered for a brief period of time (pulse), followed by administration of i) the AHCM, ii) the microenvironment modulator, iii) the anti-inflammatory agent, and/or iv) the immune checkpoint inhibitor for a longer period of time (e.g., chase), or vice versa.

In yet other embodiments, the method includes administering the metformin agent and i) the AHCM, ii) the microenvironment modulator, iii) the anti-inflammatory agent, and/or iv) the immune checkpoint inhibitor prior to, concurrently with, or sequentially to a second, third or more additional therapy (e.g., cancer therapy or anti-fibrotic therapy as described herein).

The combination therapies described herein can be administered to the subject systemically (e.g., orally, parenterally, subcutaneously, intravenously, rectally, intramuscularly, intraperitoneally, intranasally, transdermally, or by inhalation or intracavitary installation).

In other embodiments, the metformin agent and i) the AHCM, ii) the microenvironment modulator, iii) the anti-inflammatory agent, and/or iv) the immune checkpoint inhibitor are administered as separate compositions, e.g., pharmaceutical compositions. In other embodiments, the metformin agent and i) the AHCM, ii) the microenvironment modulator, iii) the anti-inflammatory agent, and/or iv) the immune checkpoint inhibitor are administered separately, but via the same route (e.g., orally or intravenously). In other embodiments, the metformin agent and i) the AHCM, ii) the microenvironment modulator, iii) the anti-inflammatory agent, and/or iv) the immune checkpoint inhibitor are administered by different routes (e.g., metformin agent is administered orally; i) the AHCM, ii) the microenvironment modulator, iii) the anti-inflammatory agent, and/or iv) the immune checkpoint inhibitor is administered subcutaneously or intravenously). In still other instances, the metformin agent and i) the AHCM, ii) the microenvironment modulator, iii) the anti-inflammatory agent, and/or iv) the immune checkpoint inhibitor are administered in the same composition, e.g., pharmaceutical composition.

In some embodiments, at least one, two, three or all of the AHCM, the metformin agent, the microenvironment modulator, the other stromal modulator, the anti-inflammatory agent, or a therapy is administered as a particle (e.g., a nanoparticle as disclosed herein). Any particle disclosed herein can be used in these methods and compositions including a polymeric particle or a lipid particle, e.g., as described herein.

In one embodiment, the AHCM alone, or in combination with the microenvironment modulator, can be administered (as a particle or free agent) at any time before, during or after the therapy.

In some embodiments, the metformin agent is administered orally. In certain embodiments, the metformin agent and/or i) the AHCM, ii) the microenvironment modulator, iii) the anti-inflammatory agent, and/or iv) the immune checkpoint inhibitor are administered locally or intratumorally (e.g., via an oncolytic virus).

In some embodiments, the metformin agent is administered as a pharmaceutical composition comprising one or more i) the AHCM, ii) the microenvironment modulator, iii) the anti-inflammatory agent, and/or iv) the immune checkpoint inhibitor, and a pharmaceutically acceptable excipient.

### Particles

In one embodiment, the particle, e.g., a particle used in the methods and compositions described herein, includes the agent (e.g., the metformin agent, the AHCM, the microenvironment modulator, the other stromal modular, the anti-cancer and/or the anti-fibrotic or anti-inflammatory therapy) has a size to include a small molecule therapeutic or a protein, e.g., an antibody.

In one embodiment, the particle is substantially or completely size-excluded from reaching arteriole smooth muscle, which is protected by non-leaky vessels. In other embodiments, the particle selectively penetrates a leaky vessel, e.g., a leaky vessel of a tumor or liver.

In some embodiments, the particle, e.g., a particle as described herein, has hydrodynamic diameter of greater than about 1, 5, 10, 15, 20, 25, 30, 35, 45, 50, 75, 100, 150, 200 nm, but less than 300 nm, e.g., as a nanoparticle. In one embodiment, the particle, e.g., a particle as described herein, has a hydrodynamic diameter of less than 100 nm, less than 90 nm, less than 80 nm, less than 70 nm, less than 60 nm, less than 50 nm, less than 40 nm, less than 30 nm, less than 20 nm, less than 15 nm, less than 14 nm, less than 13 nm, less than 12 nm, less than 11 nm, less than 10 nm, less than 5 nm, or less than 1 nm. In other embodiments, the particle, e.g., a particle described herein, has a hydrodynamic diameter between about 5 to 50 nm, 10 to 40 nm, 10 to 30 nm, or 10 to 20 nm.

In an embodiment, the AHCM, the microenvironment modulator and/or the other stromal modulator: is a small molecule therapeutic; is a protein, e.g., an antibody or an antibody fragment thereof or conjugate thereof (e.g., an antibody drug conjugate); or is provided in a particle. In one embodiment, the AHCM is chosen from one or more of: an angiotensin II receptor blocker (AT₁ blocker or ARB), an antagonist of RAAS antagonist, an ACE inhibitor, a TSP-1 inhibitor, a TGF-β1 inhibitor, a CTGF inhibitor, an SDF-1a inhibitor; an ERA; an AT₂ agonist; a VDR agonist; or a combination of two, three or more of the above.

In an embodiment, an AHCM, microenvironment modulator and/or other stromal modulator is administered as an entity having a hydrodynamic diameter of greater than about 1 nm (e.g., greater than about 1, 5, 10, 20, 50, 75, 100, 150, 200, or 500) and a second therapeutic agent (e.g., an anti-cancer and/or an anti-fibrotic or anti-inflammatory therapy) is administered as an entity having a hydrodynamic diameter of about 1 nm or less. In one embodiment, the AHCM is present in the entity without a second therapeutic agent (e.g., a chemotherapeutic agent). The AHCM can be formulated for extended release, e.g., in an extended release formulation for substantially continuous release for hours, days, weeks, months or years.

In an embodiment, an AHCM, microenvironment modulator and/or other stromal modulator is administered as an entity having a hydrodynamic diameter of about 1 nm, or less, and a second therapeutic agent (e.g., an anti-cancer and/or an anti-fibrotic or anti-inflammatory therapy) is administered as an entity having a hydrodynamic diameter of about 1 nm or greater (e.g., greater than about 1, 5, 10, 20, 50, 75, 100, 150, 200, 500, or 1,000 nm).

In an embodiment, an AHCM, microenvironment modulator and/or other stromal modulator is administered as an entity having a hydrodynamic diameter of less than, or equal to, about 1 nm and a second therapeutic agent (e.g., an anti-cancer and/or an anti-fibrotic or anti-inflammatory therapy) is administered as an entity having a hydrodynamic diameter of less than about 1 nm.

In an embodiment, an AHCM, microenvironment modulator and/or other stromal modulator is administered as an entity having a hydrodynamic diameter of greater than about 1 nm (e.g., greater than about 1, 5, 10, 20, 50, 75, 100, 150, 200, 500, or 1,000 nm), and a second therapeutic agent (e.g., an anti-cancer and/or an anti-fibrotic or anti-inflammatory therapy) is administered as an entity having a hydrodynamic diameter of greater than about 1 nm (e.g., greater than about 1, 5, 10, 20, 50, 75, 100, 150, 200, 500, or 1,000 nm).

The AHCM, microenvironment modulator and/or other stromal modulator and the second therapeutic agent (e.g., the anti-cancer and/or the anti-fibrotic or anti-inflammatory therapy) can be in separate or the same entity. For example, if provided as separate entities the AHCM can be provided as a first particle and the second therapeutic agent (e.g., the anti-cancer and/or the anti-fibrotic or anti-inflammatory therapy) provided as a second particle (e.g., where the second particle has a structural property (e.g., size or composition) or a functional property (e.g., release kinetics or a pharmacodynamic property) that differs from the first particle). Alternatively, an AHCM, microenvironment modulator and/or other stromal modulator and the second therapeutic agent (e.g., the anti-cancer and/or the anti-fibrotic or anti-inflammatory therapy) can be provided on the same entity, e.g., in the same nanoparticle.

In an embodiment, the AHCM, microenvironment modulator and/or other stromal modulator is selected from a therapeutic entity having a hydrodynamic diameter: equal to or less than 1 or 2 nm; between 2- 20, 10-25, 20-40, 40, 50-150 nm; between 10, 15, 20, 25, 35, 40, 45, 50-100 nm; between 10, 15, 20, 25, 35, 40, 45, 50-200 nm; between 10, 15, 20, 25, 35, 40, 45, 50, 75, 100, 150, 200, 300-500 nm; and between 10, 15, 20, 25, 35, 40, 45, 50, 75, 100, 150, 200, 300, 1000 nm; or 10, 15, 20,25, 35, 45, 50, 75, 100, 150 or 200 nm.

In an embodiment, the therapy (e.g., a second therapeutic agent as described herein, e.g., the anti-cancer and/or the anti-fibrotic or anti-inflammatory therapy): is a small molecule therapeutic with a hydrodynamic diameter of 1 nm or less; is a protein, e.g., an antibody or an antibody fragment thereof or conjugate thereof (e.g., an antibody drug conjugate); or is provided in a particle.

In an embodiment, the therapy (e.g., a second therapeutic agent as described herein, e.g., the anti-cancer and/or the anti-fibrotic or anti-inflammatory therapy) is administered as an entity having a hydrodynamic diameter of greater than about 1, 5, 10, 20, 30, 40, 50, 60, 70, 80, 90, 100, 150, 200, or 500 nm. For example, the second therapeutic agent (e.g., the anti-cancer and/or the anti-fibrotic or anti-inflammatory therapy) can be a protein, e.g., an antibody or an antibody fragment or conjugate thereof (e.g., an antibody drug conjugate). The second therapeutic agent (e.g., the anti-cancer and/or the anti-fibrotic or anti-inflammatory therapy) can also be administered as a particle, e.g., a polymeric nanoparticle or a liposome, that includes the agent as a small molecule therapeutic (i.e., a molecule having a hydrodynamic diameter of about 1 nm or less) or a protein, e.g., an antibody.

In an embodiment, the second agent, e.g., the anti-cancer and/or the anti-fibrotic or anti-inflammatory therapy, is selected from a therapeutic entity having a hydrodynamic diameter: equal to or less than 1 or 2 nm; between 2- 20, 10-25, 20-40, 40, 50-150 nm; between 10, 15, 20, 25, 35, 40, 45, 50-100 nm; between 10, 15, 20, 25, 35, 40, 45, 50-200 nm; between 10, 15, 20, 25, 35, 40, 45, 50, 75, 100, 150, 200, 300-500 nm; and between 10, 15, 20, 25, 35, 40, 45, 50, 75, 100, 150, 200, 300-1000 nm; or 10, 15, 20,25, 35, 45, 50, 75, 100, 150 or 200 nm.

In an embodiment, the AHCM, microenvironment modulator and/or other stromal modulator, the second agent, e.g., the anti-cancer and/or the anti-fibrotic or anti-inflammatory therapy), each independently, can be provided as an entity having the following size ranges (in nm): a hydrodynamic diameter of less than or equal to 1, or between 0.1 and 1.0 nm, e.g., that of a typical small molecule; a hydrodynamic diameter of between 5 and 20, or 5 and 15 nm, e.g., that of a protein, e.g., an antibody; or a hydrodynamic diameter of 10-5,000, 20-1, 000, 10-500, 10-200, 10-150, or 10-100, 10-25, 20-40, 40, 50-150 nm; between 10, 15, 20, 25, 35, 40, 45, 50-100 nm; between 10, 15, 20, 25, 35, 40, 45, 50-200 nm; between 10, 15, 20, 25, 35, 40, 45, 50, 75, 100, 150, 200, 300-500 nm; and between 10, 15, 20, 25, 35, 40, 45, 50, 75, 100, 150, 200, 300-1000 nm; or 10, 15, 20,25, 35, 45, 50, 75, 100, 150 or 200 nm, e.g., a range of typical nanoparticles.

### Subjects

The compositions and methods described herein can be used to treat subjects having characteristics or needs defined herein. In some embodiments a subject, or a treatment for a subject, is selected on the basis of a characteristic described herein. In one embodiment, the methods described herein allow optimized selection of patients and therapies. In some embodiments, subjects can be selected or identified prior to subjecting them to any aspects of the methods described herein.

In one embodiment, the subject is selected, or is identified, as being in need of receiving the metformin, the AHCM and/or the microenvironment modulator on the basis of optimizing a therapy, e.g., the need for improved delivery and/or efficacy of the therapy (e.g., the cancer therapy, or an anti-fibrotic or anti-inflammatory therapy).

In one embodiment, the subject does not have hypertension, or is not being treated for hypertension, at the time of initiation of the AHCM treatment, or at the time of selection of the patient for AHCM administration. In an embodiment, the subject treated is not a hypertensive patient, e.g., does not have a medical history of high blood pressure, or has not been treated with an anti-hypertensive agent. In one embodiment, the subject treated has normal or low mean arterial blood pressure. In other embodiments, the subject treated has not undergone, or is not being treated with anti-hypertensive therapy.

In an embodiment, the subject, e.g., patient, has not been administered a dose of an AHCM, e.g., an AHCM named herein, or any AHCM, within 5, 10, 30, 60 or 100 days of, the diagnosis of the disorder, e.g., the cancer, or the fibrotic or inflammatory disorder, or the initiation of the AHCM dosing.

In an embodiment, the subject, e.g., a subject with normal or low blood pressure, is selected or is identified on the basis of being in need of a metformin agent, an AHCM and/or the microenvironment modulator, e.g., is selected or is identified as being in need of receiving the metformin agent, the AHCM and/or the microenvironment modulator on the basis of optimizing a therapy, e.g., the need for improved delivery and/or efficacy of the therapy (e.g., the cancer or fibrotic therapy).

In some embodiments, a subject who is in need of receiving the metformin, the AHCM and/or the microenvironment modulator on the basis of the need for improved delivery or efficacy of the cancer or fibrotic therapy, or optimizing the therapy, are the subjects who partially respond or do not respond to the therapy alone.

In an embodiment, the metformin agent, the AHCM and/or the microenvironment modulator is selected for treating a subject, on the basis of its ability to optimize a treatment, e.g., a cancer therapy, or an anti-fibrotic or anti-inflammatory therapy, e.g., improving delivery and/or efficacy of the therapy, e.g., the cancer therapy, or the anti-fibrotic or anti-inflammatory therapy.

In some embodiments, the subject has, or is identified as having, a metabolic disorder (e.g., diabetes, e.g., type II diabetes).

In some embodiments, the subject does not have, or is not identified as having, a metabolic disorder (e.g., diabetes, e.g., type II diabetes).

In one embodiment, the subject is in need of cancer therapy, or an anti-fibrotic or anti-inflammatory therapy. In another embodiment, the subject is in need of, or being considered for, a cancer therapy, or an anti-fibrotic or anti-inflammatory therapy (e.g., treatment described herein). In certain embodiments, the method includes the step of determining if the subject has a cancer, or a fibrotic or inflammatory disorder, and, responsive to said determination, administering the metformin, the AHCM and/or the microenvironment modulator, and the agent (e.g., a cancer therapy, or an anti-fibrotic or anti-inflammatory therapy).

In other embodiments, the subject is at risk of developing, or having a recurrence of, a cancer, e.g., a subject with pre-neoplasia or a genetic pre-disposition for cancer (e.g., a subject having a BRCA1 mutation; or a breast cancer patient treated with in an adjuvant setting (e.g., with tamoxifen).

In other embodiments, the subject has early-cancer, or more progressive (e.g., moderate), or metastatic cancer.

In one embodiment, the subject has a solid, fibrotic tumor chosen from one or more of pancreatic (e.g., pancreatic adenocarcinoma or pancreatic ductal adenocarcinoma), breast, colorectal, colon, lung (e.g., small or non-small cell lung cancer), skin, ovarian, prostate, cervix, gastrointestinal (e.g., carcinoid or stromal), stomach, head and neck, kidney, or liver cancer, or a metastatic lesion thereof. Additional examples of cancers treated are described herein below.

In one embodiment, the subject has a fibrotic or desmoplastic solid tumor, e.g., a tumor having one or more of: limited tumor perfusion, compressed blood vessels, high interstitial fluid pressure (IFPs), increased hypoxia, or fibrotic tumor interstitium. In certain embodiments, the subject has a tumor having (e.g., elevated levels of) extracellular matrix components, such as fibers (e.g., collagen, procollagen) and/or polysaccharides (e.g., glycosaminoglycans such as hyaluronan or hyaluronic acid). The levels of the extracellular matrix components in the tumor can vary depending on the particular cancer type, the stage of malignancy, and/or in response to cancer therapy. For example, certain tumors may show elevated levels of extracellular matrix components in response to chemotherapy and/or radiation. In such cancers, the AHCM alone or in combination with the microenvironment modulator can be administered at any time before, during or after the cancer therapy. In an embodiment, the fibrotic or desmoplastic solid tumor is PDAC.

In other embodiments, the subject has a hyperproliferative cancerous condition (e.g., a benign, pre-malignant or malignant condition). The subject can be one at risk of having the disorder, e.g., a subject having a relative afflicted with the disorder, or a subject having a genetic trait associated with risk for the disorder. In one embodiment, the subject can be symptomatic or asymptomatic. In an embodiment, the subject harbors an alteration in an oncogenic gene or gene product. In an embodiment, the subject is a patient who is undergoing cancer therapy (e.g., the same or other anti-cancer agents, surgery and/or radiation). In an embodiment, the subject is a patient who has undergone cancer therapy (e.g., other anti-cancer agents, surgery and/or radiation). In one embodiment, the subject has not been treated with the cancer therapy.

In one embodiment, the subject is a patient with a metastatic cancer, e.g., a metastatic form of a cancer disclosed herein (one or more of pancreatic (e.g., pancreatic adenocarcinoma), breast, colorectal, lung (e.g., small or non-small cell lung cancer), skin, ovarian, or liver cancer.

In one embodiment, the subject is a patient having treatment-resistant cancer or hyperproliferative disorder.

In one embodiment, the subject is, or is identified as being, overweight or obese. Assessment of overweight and obesity can be determined by the classification of body mass index (BMI) as defined by “Clinical Guidelines on the Identification, Evaluation, and Treatment of Overweight and Obesity in Adults” from the National Institutes of Health. Body mass index is obtained by dividing a subject's weight, e.g., in kilograms (kg) by the square of the subject's height, e.g., in meters (m). Subjects with a BMI 18.5 to 24.9 are typically classified as normal weight, while subjects with a BMI 25.0 to 29.9 are classified as overweight. Subjects with a BMI 30.0 or greater are classified as obese, and can be subdivided into three classes: Class I (BMI=30.0 to 34.9; Class II (BMI=35.0 to 39.9); and Class III (BMI is greater or equal to 40).

In one embodiment, the subject is overweight, e.g., the subject has a BMI of greater than or equal to 25.0 but less than or equal to 29.9.

In another embodiment, the subject is, or is identified as being, obese, e.g., the subject has a BMI of greater than or equal to 30, e.g., greater than 30, greater than 35, greater than 40, greater than 45, or greater than 50.

Obesity can also be associated with one or more of: desmoplasia, e.g., in adipose tissues and the pancreas; dysfunctional adipocytes, e.g., hypertrophied adipocytes; increased hypoxia; fibrosis; accumulation of fat, e.g., steatosis; increased angiotensin II (AngII) type-1 receptor (AT1) signaling; and/or increased expression, production, and/or secretion of pro-inflammatory cytokines, e.g., interleukin-lbeta (IL-1 beta).

In an embodiment, the subject is, or is identified as being, overweight or obese, and has a fibrotic or a hyperproliferative cancerous condition described herein. In an embodiment, the subject is, or is identified as being, overweight or obese and has a fibrotic disorder described herein. In an embodiment, the subject is, or is identified as being, overweight or obese and has a liver disorder or condition described herein.

In one embodiment, the subject is, or is identified as being, overweight or obese, and has a fibrotic or desmoplastic tumor, e.g., a tumor having one or more of: limited tumor perfusion, compressed blood vessels, high interstitial fluid pressure (IFPs), increased hypoxia, or fibrotic tumor interstitium. In certain embodiments, the subject is overweight or obese, and has a tumor having (e.g., elevated levels of) extracellular matrix components, such as fibers (e.g., collagen, procollagen), fibroblasts (e.g., elevated levels of cancer associated fibroblasts (CAFs) or increased activity of CAFs) and/or polysaccharides (e.g., glycosaminoglycans such as hyaluronan or hyaluronic acid).

In one embodiment, the subject is overweight or obese, and has pancreatic ductal adenocarcinoma (PDAC).

In other embodiments, the subject is, or is identified as being, overweight or obese, has a fibrotic or a hyperproliferative cancerous condition described herein, and exhibits one, two, three, four or more of: increased angiogenesis; increased inflammatory cell infiltration, e.g., in adipose tissues; shows enhanced tumor progression and/or metastatis; shows increased recruitment of tumor-associated macrophages (TAM); or shows increased activation of an angiogenic pathway, e.g., VEGFR-1 pathway.

In one embodiment, the subject is overweight or obese, and has breast cancer.

In certain embodiments, the subject (e.g., an overweight or obese subject having a cancer or a fibrotic condition as described herein (e.g., a breast or pancreatic cancer, a desmoplastic tumor)) is treated with an anti-angiogenic agent, e.g., a VEGF/VEGFR inhibitor. The VEGF/VEGFR inhibitor can be administered alone or in combination with an anti-diabetic therapy, e.g., a metformin agent as described herein. Accordingly, a method for treating an overweight or obese subject having a cancer or a fibrotic condition as described herein (e.g., a breast or pancreatic cancer, a desmoplastic tumor)). The method includes: administering to the subject an anti-angiogenic agent, e.g., a VEGF/VEGFR inhibitor (e.g., an inhibitor as disclosed herein) alone or in combination an anti-diabetic therapy, e.g., a metformin agent as described herein, in an amount sufficient to treat the cancer or the fibrotic conditions. In one embodiment, the administration reduces one, two, three, four or more of: angiogenesis; inflammatory cell infiltration, e.g., in adipose tissues; tumor progression and/or metastatis; recruitment of tumor-associated macrophages (TAM); or activation of an angiogenic pathway, e.g., VEGFR-1 pathway. The method can further comprise administering to the subject an AHCM (e.g., a composition comprising an AHCM as described herein), e.g., prior to, concurrently with, or after the anti-angiogenic agent and/or the metformin agent.

In other embodiments, the subject (e.g., an overweight or obese subject having a cancer or a fibrotic condition described herein (e.g., a breast or pancreatic cancer, desmoplastic tumor)) is treated with an AHCM (e.g., a composition comprising an AHCM as described herein) in combination with an anti-angiogenic agent, e.g., a VEGF/VEGFR inhibitor, a metformin agent, or a combination of the anti-angiogenic agent and the metformin agent.

In such embodiments where the subject is, or is identified as being, overweight or obese, and has a hyperproliferative cancerous condition as described herein, e.g., a fibrotic or desmoplastic tumor, the AHCM is administered in combination with an anti-cancer therapy, e.g., a chemotherapeutic. In other embodiments, the AHCM is administered in combination with an anti-angiogenic agent, e.g., a VEGF/VEGFR inhibitor, a metformin agent, or a combination of both. In an embodiment, administration of the AHCM is initiated prior to the initiation of administration of the anti-cancer, anti-angiogenic, or anti-diabetic therapy (one or more of which are referred to herein as “the therapy”). In an embodiment, administration of the AHCM is concurrent with the administration of the therapy. In an embodiment, therapy with the AHCM continues during the entire therapy schedule. In yet other embodiments, administration of the AHCM is discontinued prior to cessation of the therapy. In other embodiments, administration of the AHCM is continued after cessation of the therapy. Administration of an AHCM with other therapies is further described herein in the section entitled “Additional Combination Therapies.”

In other embodiments where the subject is, or is identified as being, overweight or obese, and has a fibrotic condition, or a hyperproliferative cancerous condition as described herein, e.g., a fibrotic or desmoplastic tumor, any of the AHCM, the anti-angiogenic therapy, the anti-diabetic therapy, the anti-cancer therapy, or a combination thereof, can be administered as a particle as described herein. The particles can include a single agent or combination of agents. In an embodiment, the particle or agent comprises an agent (e.g., an ARB, a chemotherapeutic, an anti-diabetic drug, and/or an inhibitor of the VEGF pathway). In an embodiment, administration of the ARB-containing particle is concurrent with the administration of an anti-diabetic drug, e.g., the metformin agent, a chemotherapeutic, and/or an inhibitor of the VEGF pathway.

In any of the aforesaid embodiments, the AHCM can be administered as a free agent or as a composition (e.g., as a particle as described herein) comprising the AHCM). In certain embodiments, at least one, two or all of the AHCM, the anti-angiogenic agent, the anti-cancer agent, or the anti-diabetic therapy is administered as a particle.

In one embodiment, the subject treated is a mammal, e.g., a primate, typically a human (e.g., a patient having, or at risk of, a cancer or a fibrotic disorder as described herein).

In certain embodiments, the subject treated has a disorder chosen from one or more of a hyperproliferative disorder, a cancer, a fibrotic disorder, an inflammatory disorder or an autoimmune disorder.

In one embodiment, the subject treated has a hyperproliferative disorder, e.g., a hyperproliferative connective tissue disorder (e.g., a hyperproliferative fibrotic disease). In one embodiment, the hyperproliferative fibrotic disease is multisystemic or organ-specific. Exemplary hyperproliferative fibrotic diseases include, but are not limited to, multisystemic (e.g., systemic sclerosis, multifocal fibrosclerosis, sclerodermatous graft-versus-host disease in bone marrow transplant recipients, nephrogenic systemic fibrosis, scleroderma), and organ-specific disorders (e.g., fibrosis of the lung, liver, heart, kidney, pancreas, skin and other organs).

In other embodiment, the subject treated has a hyperproliferative genetic disorder, e.g., a hyperproliferative genetic disorder chosen from Marfan's syndrome or Loeys-Dietz syndrome.

In other embodiments, the hyperproliferative disorder (e.g., the hyperproliferative fibrotic disorder) is chosen from one or more of chronic obstructive pulmonary disease, asthma, aortic aneurysm, radiation-induced fibrosis, skeletal-muscle myopathy, diabetic nephropathy, and/or arthritis.

### Exemplary Disorders and Conditions

In certain embodiments, the disorder treated with the compositions and methods disclosed herein is chosen from one or more of a hyperproliferative disorder, a cancer (e.g., a solid or fibrotic cancer), a fibrotic disorder or condition, an inflammatory disorder or condition, or an autoimmune disorder.

In one embodiment, the disorder, e.g., a cancer, treated is an epithelial, a mesenchymal or a hematologic malignancy. In an embodiment, the cancer treated is a solid tumor (e.g., carcinoid, carcinoma or sarcoma), a soft tissue tumor (e.g., a heme malignancy), and a metastatic lesion, e.g., a metastatic lesion of any of the cancers disclosed herein.

In one embodiment, the cancer treated is a fibrotic or desmoplastic solid tumor, e.g., a tumor having one or more of: limited tumor perfusion, compressed blood vessels, high interstitial fluid pressure (IFPs), or fibrotic tumor interstitium.

In one embodiment, the solid tumor is chosen from one or more of pancreatic (e.g., pancreatic adenocarcinoma (e.g., pancreatic ductal adenocarcinoma (PDA or PDAC)), breast, gastric, colorectal, lung (e.g., small or non-small cell lung cancer), skin, ovarian, prostate, or liver cancer. Additional examples of cancers treated are described herein below.

In certain embodiments, the cancer treated contains (e.g., has elevated levels of) extracellular matrix components, such as fibers (e.g., collagen, procollagen) and/or polysaccharides (e.g., glycosaminoglycans such as hyaluronan or hyaluronic acid). The levels of the extracellular matrix components in the cancer can vary depending on the particular cancer type, the stage of malignancy, and/or in response to cancer therapy. For example, certain cancer may show elevated levels of extracellular matrix components in response to chemotherapy and/or radiation. In such cancers, the metformin agent,

In one embodiment, the cancer or tumor treated is a solid, fibrotic tumor chosen from one or more of pancreatic (e.g., pancreatic adenocarcinoma or pancreatic ductal adenocarcinoma), breast, colorectal, colon, lung (e.g., small or non-small cell lung cancer), skin, ovarian, prostate, cervix, gastrointestinal (e.g., carcinoid or stromal), stomach, head and neck, kidney, brain cancer or liver cancer (e.g. HCC), or a metastatic lesion thereof. Additional examples of cancers treated are described herein below.

In one embodiment, the disorder is fibrotic or desmoplastic solid tumor, e.g., a tumor having one or more of: limited tumor perfusion, compressed blood vessels, high interstitial fluid pressure (IFPs), or fibrotic tumor interstitium. In certain embodiments, the subject has a tumor having (e.g., elevated levels of) extracellular matrix components, such as fibers (e.g., collagen, procollagen) and/or polysaccharides (e.g., glycosaminoglycans such as hyaluronan or hyaluronic acid). The levels of the extracellular matrix components in the tumor can vary depending on the particular cancer type, the stage of malignancy, and/or in response to cancer therapy. For example, certain tumors may show elevated levels of extracellular matrix components in response to chemotherapy and/or radiation. In such cancers, the metformin agent and/or the AHCM alone or in combination with the microenvironment modulator can be administered at any time before, during or after the cancer therapy.

In certain embodiments, the disorder is chosen from one or more of a hyperproliferative disorder, a cancer, a fibrotic disorder or condition, an inflammatory disorder or condition, or an autoimmune disorder.

In one embodiment, the disorder is a hyperproliferative disorder, e.g., a hyperproliferative connective tissue disorder (e.g., a hyperproliferative fibrotic disease). In one embodiment, the fibrotic (e.g., hyperproliferative fibrotic) disease is multisystemic or organ-specific. Exemplary fibrotic diseases include, but are not limited to, multisystemic (e.g., systemic sclerosis, multifocal fibrosclerosis, sclerodermatous graft-versus-host disease in bone marrow transplant recipients, nephrogenic systemic fibrosis, scleroderma), and organ-specific disorders (e.g., fibrosis of the lung, liver, heart, kidney, pancreas, skin and other organs). In other embodiments, the fibrotic disease is chosen from liver fibrosis (e.g., liver cirrhosis, NASH, and other conditions described herein), pulmonary fibrosis, renal fibrosis, fibrosis of the bone marrow (e.g., myelofibrosis), and the like.

In other embodiment, the disorder is a hyperproliferative genetic disorder, e.g., a hyperproliferative genetic disorder chosen from Marfan's syndrome or Loeys-Dietz syndrome.

In other embodiments, the hyperproliferative disorder (e.g., the hyperproliferative fibrotic disorder) is chosen from one or more of chronic obstructive pulmonary disease, asthma, aortic aneurysm, radiation-induced fibrosis, skeletal-muscle myopathy, diabetic nephropathy, and/or arthritis.

In one embodiment, disorder is an inflammatory condition or disorder, e.g., as described herein. In one embodiment, the inflammatory disorder is osteomyelitis, e.g., chronic osteomyelitis.

In other embodiments, the disorder or condition treated using the methods and compositions disclosed herein is a fibrotic or liver disorder or condition. In one embodiment, the fibrotic disorder is a liver disorder. In one embodiment, the metformin agent, alone or in combination with, one, two, three or more of: (i) an AHCM (e.g., an ARB); (ii) a microenvironment modulator (e.g., an anti-angiogenic inhibitor, e.g., a low-dose anti-angiogenic inhibitor) and/or other stromal modulators; (iii) an anti-inflammatory agent (e.g., a cytokine inhibitor); or (iv) an inhibitor of an immune checkpoint molecule, and/or a fibrotic or liver disorder therapy (e.g., as described herein) are administered to a subject.

Additional examples of disorders, therapies and combination therapies that can be used in the compositions and methods of the invention are provided in the Detailed Description.

Headings or numbered or lettered elements, e.g., (a), (b), (i) etc, are presented merely for ease of reading. The use of headings or numbered or lettered elements in this document does not require the steps or elements be performed in alphabetical order or that the steps or elements are necessarily discrete from one another.

All publications, patent applications, patents, and other references mentioned herein are incorporated by reference in their entirety.

Other features, objects, and advantages of the invention will be apparent from the description and drawings, and from the claims.

### BRIEF DESCRIPTION OF THE DRAWINGS

FIGS. 1A and 1B show that metformin treatment is associated with reduced hyaluronan levels in human pancreatic cancers in overweight/obese patients. FIG. 1A is representative histology images showing the effect of metformin on tumor hyaluronan levels in normal weight or overweight/obese patients (n=22 controls, 7 metformin). FIG. 1B is a graphical quantification of immunohistochemical analysis of total tumor hyaluronan levels. Metformin decreases the hyaluronan-positive area fraction (%) in patients with body mass index (BMI)>25. Data are presented as the mean±standard error. * p<0.05 vs. control in patients with BMI>25.

FIGS. 2A, 2B, 2C, 2D, 2E, 2F, 2G, and 2H show that metformin reduces ECM and AT-1 expression in AK4.4 pancreatic cancer model in overweight/obese mice. After 10 weeks of high-fat diet, AK4.4 tumors were orthotopically implanted in obese FVB mice Animals were randomly assigned to metformin in drinking water (300 mg/kg) or no treatment at day 7 until day 21 when tumors were collected. FIG. 2A is representative immunohistochemistry images showing the effect of metformin on tumor hyaluronan and collagen-I levels in AK4.4 tumors (n=3-4). FIG. 2B is quantification of hyaluronan expression in AK4.4 tumors. Metformin decreases the hyaluronan-positive area fraction (%). FIG. 2C is a graphical quantification of collagen-I expression in AK4.4 tumors. Metformin decreases the collagen-I-positive area fraction (%). FIG. 2D is representative immunohistochemistry images showing the effect of metformin on expression (co-localization) of hyaluronan and collagen-I levels in activated (αSMA-positive) pancreatic stellate cells (PSCs) in AK4.4 tumors (n=3-4). FIG. 2E is quantification of hyaluronan expression in PSCs. Metformin decreases the percentage of activated PSCs expressing hyaluronan. FIG. 2F is quantification of collagen-I expression in PSCs. Metformin decreases the percentage of activated PSCs expressing collagen-I. FIG. 2G is representative Western blots for angiotensin receptor-1 (AT-1) expression in AK4.4 tumors. β-actin is used as a control for protein loading. FIG. 2H is densitometric analysis of AT-1 expression normalized to β-actin. Metformin decreases the expression of AT-1. Data are presented as the mean±standard error. * p<0.05 vs. control.

FIGS. 3A, 3B, 3C, and 3D show that metformin reduces activation and collagen-I/hyaluronan production by pancreatic stellate cells. PSCs were incubated in vitro with metformin (1 mM) for 48 h. FIG. 3A is representative immunocytochemistry images showing the effect of metformin on tumor hyaluronan and collagen-I levels in human pancreatic stellate cells (PSCs) in vitro (n=2). FIG. 3B is the quantification of hyaluronan expression in PSCs. Metformin decreases the expression of hyaluronan on PSCs. FIG. 3C is the quantification of the expression of collagen-I in PSCs. Metformin decreases the expression of collagen-I on PSCs. αSMA denotes activated PSCs. FIG. 3D is representative Western blots for the expression of fibrosis-related markers and signaling proteins in pancreatic stellate cells (PSCs) treated with metformin at 0, 0.1, 1 and 10 mM. Metformin decreases the expression of fibrosis-related markers and signaling proteins on PSCs. β-actin is used as a control for protein loading. Densitometric analysis of protein expression normalized to β-actin or total protein (in the case of phosphorylated protein) is depicted as numbers below the representative bands. Data are presented as the mean±standard error. *p<0.05 vs. control.

FIGS. 4A, 4B, 4C, 4D, 4E, and 4F show that metformin reduces cytokine production by macrophages and inflammation in tumors a PDAC mouse model. FIG. 4A shows the effect of metformin (300 mg/kg in drinking water, please see full experimental procedure on FIG. 2 legend in Example 1) on the protein expression of major cytokines in AK4.4 tumors (n=4-5) using multiplex protein array. Metformin treatment associated with reduced IL-1β and CXCL-1 expression in tumors. FIG. 4B is representative immunocytochemistry images showing the effect of metformin metformin on the expression of F4/80 (immunofluorescence) in Ak4.4 tumors (percentage of viable tumor area) (n=4). FIG. 4C shows metformin-treated tumors had significantly reduced levels of F4/80-positive tumor-associated macrophages (TAMs). FIG. 4D shows the effect of metformin (0-0.2 mM) on the gene expression (qPCR) of M1 and M2 markers in RAW 264.7 macrophages in vitro. Clinically relevant dose (0.05) metformin treatment reduces expression of M2 markers in macrophages in vitro, including Arg-1 and IL-10. FIG. 4E shows the effect of metformin on the gene expression (qPCR) of M1 and M2 markers in TAMs isolated from PAN02 tumors in vivo (n=3). Metformin treatment reduced expression of the M2 markers Arg-1 and IL-10 in TAMs in vivo. FIG. 4F is representative Western blots for the expression of signaling proteins in RAW 264.7 (mouse leukaemic monocyte-macrophages) cells treated with metformin at 0, 0.05, 0.1, 0.2 and 0.4mM. Metformin decreases the activation of signaling pathways and increased activation of AMPKα on RAW cells. β-actin is used as a control for protein loading. Densitometric analysis of protein expression normalized to β-actin or total protein (in the case of phosphorylated protein) is depicted as numbers below the representative bands. Data are presented as mean±standard error in A, B and D. * p<0.05, ** p<0.01 vs. control.

FIGS. 5A, 5B, 5C, 5D, 5E, and 5F show that metformin reduces expression of MMP-9, markers of epithelial-to-mesenchymal transition and metastasis in a PDAC mouse model. FIG. 5A shows expression of genes associated with ECM remodeling, EMT and inflammation in AK4.4 tumors from control and metformin-treated mice. Data normalized to control group. 3-4 samples per group pooled in one single PCR array plate. Metformin reduces the expression of pro-tumor genes and increases the expression of anti-tumor genes. FIG. 5B is representative Western blots showing the effect of metformin (300 mg/Kg) on MMPs and epithelial-to-mesenchymal transition (EMT) markers in AK4.4 tumors. β-actin is used as a control for protein loading. FIG. 5C is densitometric analysis of protein expression normalized to β-actin. Metformin decreases the expression of MMP-9 and vimentin and increases the expression of e-cadherin in Ak4.4 tumors. FIG. 5D shows MMP activity in AK4.4 tumors from control and metformin-treated mice. Metformin decreases the activity of MMPs. FIG. 5E shows the effect of metformin on the percentage of mice affected (incidence) with mesenteric (peritoneal) and abdominal wall (retroperitoneal) metastasis in AK4.4 and PAN02 models (n=3-8). Metformin reduced the percentage of mice that develop wall metastasis in the more metastatic model (PAN02 model) and induced a tendency for reduced wall as well as mesenteric metastasis in the less metastatic AK4.4 model. FIG. 5F shows the effect of metformin on the number (average) of mesenteric (peritoneal) and abdominal wall (retroperitoneal) metastasis per mouse in the Ak4.4 and PAN02 models (n=3-8). Metformin reduced the number of wall metastasis in the PAN02 model. There were also trends for fewer mesenteric metastasis in AK4.4 and PAN02 tumors. Data in A and C are presented as the mean±standard error. *p<0.05 vs. control.

FIG. 6 is a schematic showing that metformin inactivates pancreatic stellate cells (PSCS) and tumor-associated macrophages (TAMs), alleviates the fibro-inflammatory tumor microenvironment and reduces metastasis. Metformin treatment reduces collagen-I and HA production by PSCs, leading to decreased fibrosis in PDACs. Metformin treatment also reduces cytokine production, infiltration and M2 polarization of TAMs, leading to decreased inflammation. These lead to improved desmoplasia and reduced ECM remodeling, EMT, and metastasis.

FIG. 7 shows that metformin treatment does not reduce collagen-I levels in human pancreatic cancers. Effect of metformin on tumor collagen-I levels in normal weight or overweight/obese patients. Quantification was performed on immunohistochemical analysis of total tumor collagen-I levels. Metformin treatment does not alter tumor collagen-I-positive area fraction (%) (n=23 controls, 6 metformin). Data are presented as the mean±standard error.

FIGS. 8A, 8B, 8C, 8D, 8E and 8F shows that metformin reduces density of ECM producing PSCs in PAN02 tumors. FIG. 8A is representative immunohistochemistry images showing the effects of metformin on α-SMA, hyaluronan and collagen-I levels in PAN02 tumors. FIGS. 8B and 8C are quantification of immunohistochemical analysis of hyaluronan and collagen-I expression. FIG. 8D is immunohistochemical analysis of total α-SMA/hyaluronan double positive cells. Metformin induced a tendency for decreased area fraction (%) of hyaluronan-positive activated PSCs (n=5-6). FIG. 8E is immunohistochemical analysis of total α-SMA/collagen-I double positive cells. Metformin decreases the area fraction (%) of collagen-I-positive activated PSCs (n=5-6). Data are presented as the mean±standard error. *p<0.05 vs. control.

FIG. 9 shows that high doses of metformin reduce the viability of PSCs in vitro. Effect of metformin on human PSC viability. PSCs were incubated with metformin at increasing doses for 48 h, and MTT viability assay was performed. At high doses (>7.5mM), metformin significantly reduces cell viability. Values are the mean±standard error. *p<0.05, **p<0.01, ***p<0.001 vs. untreated control.

FIGS. 10A, 10B, and 10C shows that metformin reduces pSTAT3 expression in PDACs. FIG. 10A shows representative blots showing the effect of metformin on phospho and total p38, STAT3, JNK and NFKB expression in AK4.4 and PAN02 tumors. β-actin is used as a control for protein loading. FIGS. 10B and 10C shows the densitometric analysis of protein expression normalized to total levels of the protein of interest. Metformin decreases the expression of p-STAT3 in both tumor models.

FIGS. 11A, 11B, and 11C show metformin reduces inflammation in a second PDAC mouse model (PAN02). FIG. 10A shows the effect of metformin on the protein expression of major cytokines in the PAN02 tumor model. FIG. 10B shows the effect of metformin on the levels of CD45(+)CD11b(+)F4/80(+) TAMs in PAN02 tumors (percentage of total viable cells as assessed by flow cytometry) (n=4-7). A tendency for reduced numbers of TAMs with metformin was observed. Data are presented as the mean±standard error. *p<0.05 vs. control. FIGS. 10C and 10D show that metformin treatment associates with reduced IL-1β expression in tumors (FIG. 10C) and plasma (FIG. 10D) (n=6-7).

FIG. 12 shows that metformin reduces the viability of macrophages in vitro. Effect of metformin on macrophage viability in vitro. Macrophages were incubated with metformin at increasing doses for 48 h, and MTT viability assay was performed. At doses>0.2 mM metformin significantly reduces cell viability. Data are presented as the mean±standard error. *p<0.05, **p<0.01 vs. untreated control.

FIG. 13 is representative Western blots for the expression of additional metabolic markers (AMPK-β and ACC) on RAW 264.7 (mouse leukaemic monocyte-macrophages) cells treated with metformin at 0, 0.05, 0.1, 0.2 and 0.4mM. Metformin increased the activation of ACC when cells were in serum media. Densitometric analysis of protein expression normalized to total protein is depicted as numbers below the representative bands. Data are presented as the mean±standard error. *p<0.05 vs. control.

FIGS. 14A, 14B, 14C, and 14D show the effects of metformin on ECM remodeling and EMT in a second PDAC mouse model (PAN02). FIG. 14A shows the expression of genes associated with ECM remodeling, EMT and inflammation in PAN02 tumors from control and metformin-treated mice. Data normalized to control group. 3-4 samples per group pooled in one single PCR array plate. Metformin reduces the expression of MMPs and increases the expression of Timp-4. FIG. 14B shows representative Western blots for the expression of MMP-9 on PSCs treated with metformin at 0, 0.1, 1 and 10 mM. Metformin decreased expression of MMP-9. Densitometric analysis of protein expression normalized to total protein is depicted as numbers below the representative bands. FIG. 14C is representative blots showing the effect of metformin on MMPs and epithelial-to-mesenchymal transition (EMT) markers in PAN02 tumors. β-actin is used as a control for protein loading. FIG. 14D is the densitometric analysis of protein expression normalized to β-actin. Metformin induced a tendency for decreased MMP-2 and vimentin in PAN02 tumors. Data are presented as the mean±standard error.

FIGS. 15A, 15B, and 15C show the metabolic effects of metformin are not present in both PDAC mouse model. FIG. 15A shows the effects of metformin on systemic levels of glucose, insulin, and insulin-like growth factor-I (IGF-I) in PAN02 tumor bearing mice (n=7-8). Metformin reduces levels of glucose, insulin and induced a trend for reduced levels of IGF-I in circulation. FIG. 15B shows the effect of metformin on insulin in AK4.4 tumor bearing mice. FIG. 15C is representative blots showing the effect of metformin on metabolic markers in PAN02 and AK4.4 tumors. β-actin is used as a control for protein loading. Also shown is densitometric analysis of protein expression normalized to total protein or β-actin in the case of LC3B. Metformin decreases the expression of p-IRS-1 and increased the expression of p-AMPK-β and p-ACC in PAN02 but not in Ak4.4 tumors. Data are presented as the mean±standard error. *p<0.05, **p<0.01 vs control.

FIG. 16 shows that metformin does not affect body weight in tumor-bearing animals. Effect of metformin on body weight after tumor implantation (n=4-10). Metformin does not affect body weight in tumor-bearing mice. Data are presented as the mean±standard error.

FIGS. 17A, 17B, 17C, 17D, 17E, and 17F show that obesity promotes tumor initiation and progression. FIG. 17A shows that high-fat (60%) versus low-fat (10%) diets generated a difference in body weight (BW) in C57BL/6, FVB and the spontaneous PDAC (KPC and iKRAS) models. Diet started at six weeks of age, continued for ten weeks (C57BL/6 and FVB), at which time tumors were implanted, and then continued until the end of experiments. In the spontaneous tumor models, diets were administered until tumor collection. Mice genetically deficient for leptin (ob/ob) on a standard chow for seven weeks gained weight compared to agematched WT mice (n=8-10/group for C57B1/6, FVB and ob/ob, 4-10/group for KPC, 7-21/group for iKRAS). Left bars represent mice fed a low-fat diet, right bars represent mice fed a high fat diet for all groups except C57/B6 on the far right of the graph, in which the right bar represents the ob/ob mice. FIG. 17B shows the time to develop tumors of about 1 g in iKRAS mice fed low (left bars) or high-fat diet (right bars). FIG. 17C shows the effect of obesity on tumor growth. PAN02 and AK4.4 syngeneic tumors were orthotopically implanted in C57BL/6 and FVB mice respectively at ten weeks of diet (lean diet, left bars; and obese diet or ob/ob mice, right bars); ob/ob mice were implanted with PAN02 tumors at seven weeks of age. Tumors were collected 21 days later. Obese animals presented with higher tumor weights than lean counterparts in all models (n=8-10/group). FIG. 17D is representative images of mesenteric peritoneal dissemination in lean and obese mice implanted with PAN02 tumors. Mesenteries collected at the same time as tumors in FIG. 17C. FIG. 17E is a graph showing the quantification of mesenteric peritoneal metastasis in the PAN02 model. FIG. 17F is a graph showing the quantification of retro-peritoneal metastasis in the AK4.4 model. Data are shown as mean±standard error of the mean (SEM). P values were determined by the Student t-test. *P<0.05; **P<0.01; ***P<0.001.

FIGS. 18A, 18B, 18C, 18D, 18E, 18F, 18G, 18H, 18J, 18I, 18J, and 18K show that obesity aggravates tumor desmoplasia. FIG. 18A is images showing adipocyte enlargement and fibrosis in visceral adipose tissue and tumors from obese mice. Masson's Trichrome staining denotes fibrosis in blue. Arrows: Adipocytes. Scale bars: 200 μm. Quantification of adipocyte count (FIG. 18B) and size (FIG. 18C) in PAN02 and AK4.4 tumors indicates an enrichment for enlarged adipocytes in the tumor microenvironment in lean (left bars) and obese mice (right bars) (n=3 tumors/group, 8 ROIs/tumor). FIG. 18D shows representative pictures of the adipose tissue-tumor interaction, revealing increased expression of fibrosis where tumors invade the adjacent adipose tissue. On the far right, tumor epithelium is observed in close proximity to fibrotic adipose tissue and normal pancreas. Tumor sections were stained for Masson's Trichrome. Scale bars: 100 μm (PAN02, left panel), 200 μm (AK4.4, middle panel), 500 μm (Ak4.4 right panel). FIG. 18E is representative pictures of collagen-I staining (immunofluorescence) in tumors. Scale bars: 1 mm. FIG. 18F is representative pictures of fibrillar collagen in tumors using second harmonic generation (SHG). Scale bars: 100 μm. FIG. 18G is a graph showing the quantification of collagen expression normalized to lean animals. Tumors from obese mice presented with increased collagen-I expression in three different tumor models. FIG. 18H is a graph showing the quantification of fibrillary collagen normalized to lean animals. Tumors from obese mice presented with increased expression of fibrillar collagen in PAN02 and AK4.4 orthotopic PDACs. (n=3-6/group). In FIGS. 18G and 18H, left bars represent lean mice, right bars represent obese mice. FIG. 18I is representative pictures of αSMA expression in AK4.4, PAN02 and KPC tumors by immunofluorescence. FIG. 18J is a graph showing the quantification of αSMA expression by immunofluorescence was performed as a % of αSMA expression in DAPI+ viable tumor area (FIG. 18J), as well as a % of double positive αSMA/Col-I expression in DAPI+ viable tumor area (FIG. 18K) (n=3-6/group). In FIGS. 18J and 18K, left bars represent lean mice, right bars represent obese mice. Representative pictures of αSMA/Col-I double staining in PAN02 and AK4.4 tumors are in FIGS. 26D and 26E. Data are shown as mean±SEM. P values were determined by the Student t-test. *P<0.05; **P<0.01.

FIGS. 19A, 19B, 19C, 19D, and 19E shows that obesity-aggravated desmoplasia impairs drug delivery. FIG. 19A is representative pictures of CD31(+) vessels and lectin in PAN02 tumors. Scale bars: 200 μm FIG. 19B is a graph showing the quantification of the total (CD31+) and lectin-positive (CD31/lectin+) vessel area in PAN02 tumors. Obese mice (right bars) presented with decreased perfusion (n=3-6 tumors/group) compared to lean mice (left bars). FIG. 19C shows the protein expression of hypoxia markers in PAN02 tumors. Obese mice presented with increased hypoxia in tumors. FIG. 19D shows the effect of obesity on the delivery of chemotherapy to tumors. 5-FU quantified via high performance liquid chromatography (HPLC). Obesity decreased delivery of the chemotherapeutic agent (n=4 tumors/group). FIG. 19E shows the effect of obesity on response to chemotherapy. PAN02 tumors were orthotopically implanted at ten weeks of diet, treatments were initiated at day seven post-implantation and tumors resected at day 19. 5-FU was less effective in preventing PAN02 tumor growth in obese animals than in lean (two-way ANOVA, n=8-10/group). Data are shown as mean±SEM. P values were determined by the Student t-test unless otherwise stated. *P<0.05; **P<0.01

FIGS. 20A, 20B, 20C, 20D, 20E, 20F, 20G, 20H, 20I, 20J, 20K, and 20L shows that blockade of AT1 reverses the obesity-aggravated desmoplasia and improves response to chemotherapy. FIGS. 20A and 20B shows the effect of obesity on target genes of AT1 signaling. Expression of genes associated with AT-1 pathway activation and fibrosis/desmoplasia is increased in PAN02 (FIG. 20A) and AK4.4 (FIG. 20B) tumors from obese mice (right bars) in comparison to lean mice (left bars). Depicted genes where a 2-fold change in mRNA expression was observed in either tumor model. Data normalized to lean group. 3-4 samples per group pooled in one single PCR array fibrosis gene set plate. FIG. 20C shows that losartan reduced tumor αSMA protein expression more dramatically in the obese setting in AK4.4 tumors. FIG. 20D shows the quantification of protein expression was normalized to tubulin. FIG. 20E shows that losartan reduced tumor fibrillar collagen (top panels) as well as collagen-1 expression (bottom panels) in AK4.4 tumors from obese mice. Scale bars: 100 μm (SHG) and 1 mm (Col-1) FIGS. 20F-H shows the quantification of collagen was performed as a % of a region of interest (ROI) for SHG (n=4 tumors/group, 8 ROIs per tumor) (FIG. 20F) and as a % of viable tumor area in the whole tumor for collagen-1 immunofluorescence (n=4-6 tumors/group) (AK4.4 in FIG. 20G and PAN02 in FIG. 20H). FIG. 20I shows the protein expression by western blotting of AK4.4 tumors revealed that losartan normalized the obesity-augmented expression of several AT1 signaling and desmoplasia-related markers, i.e. AT1, TGFB, SMAD2, vimentin, snail, MMP9, and phospho-p38. Of note, similar to αSMA, the changes of AT1, as well as other desmoplasia related markers, were relatively mild in the lean setting. FIG. 20J shows the quantification of protein expression was normalized to tubulin (far left bars represent lean mice; middle left bars represent obese mice; middle right bars represent lean mice treated with losartan; and far right bars represent obese mice treated with losartan.) (Depicted are significant differences between control and losartan treatment). FIG. 20K shows that in the PAN02 model, losartan and AT1 genetic deficiency (Agtr1a-/-mice) improved response to chemotherapy in obese, but not in lean animals. FIG. 20L shows that in the AK4.4 model, losartan improved response to chemotherapy in both lean and obese settings but with higher magnitude in obese setting (FIGS. 20K and 20L: Two-way ANOVA with Bonferroni correction for multiple comparisons, n=4-8 tumors/group. Depicted are significant differences of treatment groups compared to control or 5-FU groups). Data are shown as mean±SEM with the exception of FIGS. 20A and 20B. P values were determined by the Student t-test unless otherwise stated. *P<0.05; **P<0.01, ***p<0.001.

FIGS. 21A, 21B, 21C, 21D, 21E, 21F, 21G, 21H, 21I, and 21J shows that tumor-associated neutrophils mediate obesity-induced tumor progression and aggravated desmoplasia. FIG. 21A shows the effect of obesity on immune cell infiltration in PDACs. Obesity promoted infiltration of myeloid Gr-1(+)F4/80(−) cell population in PAN02 tumors in obese mice. FIG. 21B is a quantification normalized by total viable cells (i) or total CD45 leucocytes (ii) (n=4-6 tumors/group). FIG. 21C is representative FACS scatter plots of CD45(+)CD11b(+)Ly6G(+) tumor associated neutrophils (TANs), CD8(+) cytotoxic lymphocytes and CD4(+)CD25(+) regulatory T cells in PAN02 tumors in lean and obese setting. FIGS. 21D and 21E shows quantification normalized by total viable cells (FIG. 21D) or total CD4 cells (FIG. 21E) (n=3-6 tumors/group). Obese promoted an increase in TANs and a decrease in CD8 cells in PAN02 tumors. A strong tendency for increased Tregs was also observed. FIG. 21F shows the effect of TAN depletion (TAN-D) on PDAC growth in obese mice. TAN depletion from day 1 using anti-Ly6G specific pharmacological inhibitory antibody in obese mice significantly reverted the obesity-increased tumor weight in PAN02 and AK4.4 models (n=4-6 tumors/group). FIG. 21G shows the preferential accumulation of TANs in areas with activated PSCs. Scale bars: 1 mm (whole tumors) and 100 m (caption). FIG. 21I shows TAN depletion reduced AT1 expression, collagen production and MMP9 expression in PAN02 tumors in obese animals. FIG. 21J shows TAN depletion led to increasing in perfusion in PAN02 tumors in obese animals. % of CD31(+), lectin(+) or double positive vessel density in the viable area of whole tumors. (n=4-6 tumors/group). For bar graphs in FIGS. 21A, 21B, 21D, and 21E, left bars represent lean mice, and right bars represent obese mice. For the bar graph in FIG. 21J, left bars represent obese mice, and right bars represent obese mice with TAN depletion. Data are shown as mean±SEM. P values were determined by the Student t-test, or one-way ANOVA for panels C and E. *P<0.05; **P<0.01, ***p<0.001.

FIGS. 22A, 22B, 22C, 22D, 22E, 22F, 22G, 22H, 22I, 22J, and 22K shows the adipose microenvironment promotes TAN infiltration and fibrosis via IL-1β. FIG. 22A shows an effect of obesity on cytokine expression in PAN02 tumors. Multiplex protein revealed that PAN02 tumors from obese mice (right bars) had increased expression of IL-1β (n=4-6 tumors/group) in comparison to lean mice (left bars). FIG. 22B shows that IL-1β was abundantly expressed by adipocytes and PSCs in the adipocyte-rich areas where PSCs predominate in PAN02 tumors. Scale bars: 200 μm (upper panels), 30 μm (lower panels). FIG. 22C shows the effect of IL-1β blockade on immune cell profile. An anti-IL1β neutralizing antibody decreased CD45(+)CD11b(+)Ly6G(+) TAN infiltration while recovering CD4+ and CD8+ Tcells (FIGS. 22C and 22D) and decreasing Tregs (FIG. 22E) (one-way ANOVA, n=3-6 tumors/group). In FIG. 22D, left bars represent lean mice, middle bars represent obese mice, and right bars represent obese mice with IL-1B inhibition. FIG. 22E shows that IL-1β blockade normalized obesity-aggravated tumor growth. (one-way ANOVA, n=3-6 tumors/group). FIG. 22G is a western blot showing that IL-1β inhibition decreased obesity-induced αSMA and AT1 expression (bands are part of a larger WB. Lean control group is depicted in FIG. 3E). FIG. 22H shows IL-1β expression in TANs. Immunofluorescence for PAN02 tumor sections denoting co-localization. Scale bar: 30 μm. FIG. 22I shows TAN depletion using Ly6G specific antibody abolished obesity-induce IL1-β expression in PAN02 tumors. (one-way ANOVA, n=4-6 tumors/group). FIGS. 22J and 22H shows the effect of AT1 blockade on immune cell profile. In tumors implanted in AT1-KO (PAN02), TANs were decreased. This was associated with increased CD8 cells and reduced Tregs (n=3-6 tumors/group). In FIGS. 22J and 22K, the far left bars represent lean mice; the middle left bars represent obese mice; the middle right bars represent lean AT1-KO mice; and the far right bars represent obese AT1-KO mice. Data in FIGS. 22C, 22D, 22E, 22J and 22K were parts of the same experiment. Data are shown as mean±SEM. P values were determined by the Student t-test unless otherwise stated. *P<0.05; **P<0.01.

FIGS. 23A, 23B, 23C, 23D, and 23E shows that PDACs from obese patients recapitulate the findings in preclinical models. FIG. 23A is representative pictures of adipocytes in human PDAC from patients with normal weight [Body mass index (BMI)<25] and obesity (BMI>30). Scale bars: 100 μm. FIG. 23B shows the quantification of adipocyte size in human PDACs. Tumors from obese patients presented with hypertrophied adipocytes (n=8 tumors/group). FIG. 23C is representative pictures of Collagen-I and HA in human PDAC from patients with normal weight (BMI<25) and obesity (BMI>30). Scale bars: 1 mm. FIG. 23D shows the quantification of Collagen-I and HA in human PDACs (n=8 tumors/group); left bars represent patients with BMI<25 and right bars represent patients with BMI>30. Data are shown as mean±SEM. P values were determined by the Student t-test. *P<0.05; **P<0.01. FIG. 23E is a graphical summary of the key findings in this study. PDACs in obese hosts present with increased fatty stroma, inflammation, and desmoplasia. The amplified crosstalk between CAAs, TANs and PSCs that occurs in obesity leads to an aggravation of desmoplasia, increased tumor progression and reduced response to chemotherapy.

FIG. 24 shows the effect of obesity on KPC tumor initiation. Time to develop tumors of about 1 g in KPC mice fed low (lean mice, left bars) or high-fat diet (obese mice, right bars). Data are shown as mean±SEM. P value was determined by the Student t-test.

FIGS. 25A and 25B shows the adipose tissue - tumor interaction. FIG. 25A is representative pictures of PAN02, AK4.4 and iKRAS tumors invading visceral adipose tissue in obese mice. FIG. 25B is additional pictures depicting an association of fibrosis with adipocytes in AK4.4 tumors from obese mice. Masson's trichrome staining in tumors revealed a predominance of fibrosis content in areas rich in adipocytes or adjacent to adipose tissue. Scale bars: 500 μm (upper panels, lower left panel), 250 μm (lower right panel).

FIGS. 26A, 26B, 26C, 26D, 26E, and 26F shows the co-expression of collagen-I and hyaluronan in PSCs, and impact of obesity on tumor hyaluronan levels. FIG. 26A is representative pictures and FIG. 26B shows the quantification of hyaluronan (HA) binding protein (HABP, which detects HA) in AK4.4 tumors from lean and obese mice. Scale bars: 1 mm (n=3-6/group). FIG. 26C shows the quantification of HA (ELISA) in PAN02 tumors from lean and obese mice (n=3-6/group). FIG. 26D shows immunofluorescence demonstrating that αSMA-expressing PSCs associate with collagen-1 expression in PAN02 tumors. Whole tumor staining depicted on the left picture; caption of an area where the two markers overlap in the center picture; amplification of the center figure on the right. Scale bars: 1 mm (far left panel), 200 μm (center panel), 50 μm (caption). FIG. 26E is a representative picture of co-expression of αSMA with collagen-I and hyaluronan in AK4.4 tumors. Scale bar: 200 μm. FIG. 26E is a western blot denoting the effect of obesity on PSC marker αSMA expression in PAN02 tumors. Data in FIGS. 26B and 26C are shown as mean±SEM. P values were determined by the Student t-test.

FIGS. 27A, 27B, 27C, and 27D show that obesity-aggravated desmoplasia reduces perfusion and efficacy of chemotherapy in AK4.4 tumors. FIG. 27A shows the effect of obesity on AK4.4 tumor perfusion. Quantification of total and lectin-positive vessel area in AK4.4 tumors. Obese mice (right bars) presented with decreased perfusion (n=5-12 tumors/group) compared to lean mice (left bars). FIG. 27B shows the effect of obesity on protein expression of hypoxia markers in AK4.4 tumors. Obese mice presented with increased expression of the hypoxia marker Hif-1a in tumors. FIG. 27C shows the effect of obesity on the delivery of doxorubicin to PAN02 tumors. Doxorubicin quantified via immunofluorescence (n=4 tumors/group). FIG. 27D shows the effect of obesity on response to chemotherapy. AK4.4 syngeneic tumors were orthotopically implanted at ten weeks of diet; treatments were initiated at day 7 post-implantation and tumors resected at day 19. 5-FU was less effective in preventing tumor growth in obese animals (two-way ANOVA, n=6-8/group). Data in FIGS. 27A, 27C and 27D, are shown as mean±SEM. P values were determined by the Student t-test unless otherwise stated. *P<0.05.

FIGS. 28A, 28B, 28C, 28D, 28E, 28F, and 28G shows the additional effects of AT-1 inhibition on obesity-aggravated desmoplasia, perfusion and drug delivery. FIG. 28A shows the double immunofluorescence for αSMA and AT1 receptor in two orthotopic PDACs. ˜70% of activated PSCs expressed AT1 receptor in PAN02 and ˜35% in AK4.4. Scale bar: 30 μm (left panels), 500 μm (right panel). FIG. 28B shows the protein expression of the signaling molecules downstream of AT1 inPAN02 and AK4.4 tumors, revealing increased activity in obese mice. FIG. 28C shows the effect of losartan on the expression of fibrosis/desmoplasia-related markers in AK4.4 tumors. mRNA expression of markers of tumor fibrosis/desmoplasia was increased in tumors in obese mice and was reverted by losartan. Losartan did not alter these markers in lean mice (3-4 samples per group were pooled for the PCR array analysis). FIG. 28D shows the effect of losartan on αSMA expression in PAN02 tumors. Losartan induced a tendency for reduced tumor αSMA protein expression in obese but not lean setting (Two-way ANOVA, n=3-6/group). FIG. 28E is a western blot showing a decrease in αSMA expression in PAN02 tumors implanted in obese AT1 KO mice compared with obese WT mice (bands are part of a larger WB. Lean control group is depicted in FIG. 26F). FIGS. 26F and 26G shows that losartan tended to improve perfusion (FIG. 26F) and increase chemotherapy delivery (FIG. 26G) in PAN02 (left panels) and AK4.4 (right panels) tumors from obese but not lean mice (Two-way ANOVA with Bonferroni correction for multiple comparisons, n=4-8 tumors per group). Data in FIGS. 26D, 26F and 26G, are shown as mean±SEM. In FIGS. 28C and 28F, far left bars represent lean mice, middle left bars represent lean mice treated with losartan, middle right bars represent obese mice, and far left bars represent obese mice treated with losartan.

FIGS. 29A and 29B shows the effect of obesity on immune cell infiltration and cytokine profile in AK4.4 tumors. FIG. 29A shows that obesity promoted infiltration of myeloid Gr-1(+)F4/80(−) cell population in AK4.4 tumors in obese mice. Quantification normalized by total CD45 leucocytes (n=4 tumors/group). FIG. 29B shows that obesity associated with increased levels of IL-1β in AK4.4 tumors in obese mice (n=4 tumors/group). Data are shown as mean±SEM. P values were determined by the Student t-test. *P<0.05. In FIGS. 29A and 29B, left bars represent lean mice, and right bars represent obese mice.

FIGS. 30A, 30B, 30C, and 30D shows the effects of TAN depletion on vessel perfusion and cytokine expression in obese mice. FIG. 30A is a representative FACS scatter plots of CD45(+)CD11b(+)Ly6G(+) tumor-associated neutrophils (TANs) in control and TAN-depleted obese mice. Ly6G specific inhibition led to a significant reduction (˜90%) of the Ly6G(+) cell population in PAN02 tumors from obese mice. FIG. 30B shows the effect of TAN depletion on vessel perfusion in AK4.4 tumors in obese animals. % of CD31(+), lectin(+) or double positive vessel density in the viable area of whole AK4.4 tumors. FIG. 30C shows the % of CD31 expression that co-stains with lectin in PAN02 and AK4.4 tumors (n=4-6 tumors/group). FIG. 30D shows that TAN depletion reduced the expression of CXCL-1 (IL-8, KC), and tended to reduce the expression of TNFα and IL-12 in PAN02 tumors from obese mice. Data in FIGS. 30B, 30C, and 30D are shown as mean±SEM. P values were determined by the Student t-test. *P<0.05. In FIGS. 30B, 30C, and 30D, left bars represent obese mice and right bars represent TAN-depleted obese mice.

FIGS. 31A, 31B, and 31C shows the effect of losartan on the immune tumor microenvironment. FIG. 31A shows the effect of losartan on cytokine expression in PAN02 and AK4.4 tumors in obese mice. Multiplex protein revealed that losartan reduced the expression of multiple cytokines including IL-1β in PDACs (n=4-7 tumors/group). FIGS. 31B and 31C show that within the CD45 population, losartan treatment tended to decrease the enrichment for GR(+)F480(−) cells (FIG. 31B), and within CD4 cells, the enrichment for T regulatory cells (FIG. 31C) (n=4 tumors/group). Error bars represent standard error of the mean. P values were determined by the Student t-test in A. *P<0.05, **P<0.01, ***P<0.001.

FIG. 32 is a representative picture of AT1 expression in cancer-associated adipocytes in PAN02 tumors. Cancer-associated adipocytes (arrows), similar to normal adipocytes, express AT1. Scale bar: 100 μm.

FIGS. 33A, 33B, and 33C show that diet-induced obesity attenuates the effect of ant-VEGF therapy on BC progression. EO771 (top panels) and MCA-IV (bottom panels) tumors grown in obese and lean mice were treated with anti-VEGF antibody (B20) or control IgG. FIG. 33A shows tumor growth. FIG. 33B shows tumor volume at day 9. FIG. 33C shows Kaplan Meyer survival curves. Anti-VEGF is less effective in reducing tumor growth in obese mice.

FIGS. 34A, 34B, and 34C show lung metastasis collected from EO771 tumor-bearing lean and obese animals treated with B20. Obesity associates with increased metastatic burden in mice treated with anti-VEGF.

FIGS. 35A, 35B, 35C, and 35D show that obesity attenuates the effect of anti-VEGF therapy on VEGF signaling, vessel density and cell proliferation. FIG. 35A shows that tumors in obese setting are hypovascular, and vessel density decrease by B20 is attenuated. FIG. 35B shows that tumors from obese mice denote an increased baseline expression for signaling pathways downstream of VEGF. In obese mice, the signaling activity remains elevated despite B20, whereas it is decreased in lean mice. FIGS. 35C and 35D show that consequently, in B20-treated mice, mitosis and cell proliferation are higher in obese animals.

FIGS. 36A, 36B, 36C, 36D, and 36E show that obesity-promoted infiltration of cancer associated adipocytes protects tumor cells from anti-VEGF therapy. FIGS. 36A-36C show an abundance of mitotic cells, increased expression of Ki-67 marker and phospho-AKT and phospho-pS6 in tumor adipocyte-rich areas. FIG. 36D shows adipocyte-rich areas in tumors, located close to the local mammary fat pad, associated with viable tumor after anti-VEGF therapy. FIG. 36E shows tumors in obese mice denote enrichment of adipocytes. Representative picture of Red Oil-O staining on the right lower panel, confirming the presence of adipocytes in tumors.

FIGS. 37A, 37B, and 37C show that the adipocyte-rich regions in obese tumors are hypo-vascular and hypoxic. FIGS. 37A and 37B show adipose-rich areas associate with reduced vessel density and increased hypoxia. FIG. 37C shows that consistant with the small effect on tumor vasculature in obese mice, anti-VEGF therapy did not affect oxygen levels in the already hypoxic tumors in these mice.

FIGS. 38A, 38B, 38C, 38D, 38E, 38F, 38G, 38H, and 38I show that adipocytes and infiltrated myeloid cells in adipocyte-rich regions overexpress IL-6. FIGS. 38A and 38B show mRNA levels and protein expression of pro-inflammatory cytokines and pro-angiogenic growth factors in EO771 tumors from animals treated with B20 denotes increased IK-6 expression in obese mice. FIGS. 38C and 38D show IL-6 originates from hypoxic adipocyte-rich areas (arrows). FIGS. 38E and 38F show IL-6 production from adipocytes and immune cells of monocyte-macrophage (CD11b) lineage infiltrating hypoxic adipocyte-rich areas. FIG. 38G shows that in animals treated with anti-VEGF therapy, obesity associated with increased levels of IL-6 downstream signaling. FIGS. 38H and 38I show P-STAT3 was particularly expressed in IL-6R positive tumor cells adjacent to macrophages infiltrating adipocyte-rich areas.

FIGS. 39A, 39B, 39C, 39D, 39E, 39F, 39G, 39H, and 39I show that IL-6 inhibition overcomes resistance to anti-VEGF therapy in obese mice. FIGS. 39A and 39B show that specific IL-6 blockade sensitizes tumors to anti-VEGF therapy in the obese setting, reducing the tumor growth rate (39A) and metastasis (39B) to the level of lean animals treated with anti-VEGF. FIGS. 39C-39E show that IL-6 inhibition in combination with anti-VEGF therapy decreases the obesity-induced upregulation of IL-6 signaling (P-STAT3, P-JNK, P-p38) (39C), tumor cell proliferation (39D), and mitotic count (39E). FIG. 39F shows that IL-6 inhibition does not increase B20-induced necrosis in tumors. FIG. 39G shows that IL-6 inhibition decreases tumor vessls density (i), increases pericyte coverage (ii) and perfusion (iii and iv), and reduces hypoxia (v-vi). FIG. 39H shows that after anti-VEGF therapy, infiltration of myeloid cells (CD11b+) increases in tumors, and IL-6-inhibition (genetically and pharmacologically) reduces these cells in the obese setting, but not lean. Similar changes occur with CD8, CD4, and CD4+CD25 regulatory cells (Tregs). FIG. 39I shows that anti-VEGF prolongs survival more dramatically in lean animals treated with doxorubicin than in obese. IL-6 inhibition in combination with anti-VEGF therapy plus doxorubicin is ineffective in lean animals when compared with anti-VEGF plus doxorubicin, but prolongs survival in obese animals by 28%.

FIGS. 40A, 40B, 40C, 40D, 40E, 40F, 40G, 40H, 40I, 40J, and 40K show that in the absence of obesity-promoted IL-6 upregulation, FGF-2 mediates resistance to anti-VEGF therapy in obesity. FIG. 40A shows that levels of IL-6 and FGF-2 vary among breast cancer patients. FIGS. 40B and 40C show that in a second BC model (MCA-IV), FGF-2 levels in tumors are increased in obese versus lean animals treated with anti-VEGF. FIG. 40D shows that FGF-2 is expressed by adipocytes (upper panel) and by fibroblasts (SMA+) (lower panel). FIG. 40E shows that similar to E0771, anti-VEGF therapy is ineffective on vessel density in MCA-IV tumors from obese mice, but FIG. 40F shows that specific inhibition of FGF-2 improves tumor growth response to anti-VEF in the obese setting. FIGS. 40G-40J show that in addition, metformin decreases FGF-2 gene (40G) and protein (40H) expression, downstream signaling (40I) and vessel density (40H) in obese animals treated with anti-VEGF. FIG. 40K shows that consequently, metformin improved tumor growth response to anti-VEGF in the obese setting.

FIGS. 41A, 41B, 41C, and 41D show that obesity associates with reduced efficacy of anti-VEGF therapy and increased circulating levels of IL-6 and FGF-2 in BC patients. IL-6 associates with worse prognosis. FIG. 41A shows that Spearman's correlation tests show that at baseline (pre-treatment with bevacizumab in the neao-adjuvant setting), Visceral Fat Area (VFA) or Subcutaneous Fat Area (SFA) associates negatively with vessel count, whereas this difference is not observed 14 days after bevacizumab initiation (i). FIG. 41A also shows that bevacizumab reduces tumor vessels in normal weight but not in obese patients (ii). FIG. 41B shows that baseline VFA associates positively with hypoxia, whereas this difference is not observed 14 days after bevacizumab initiation (i). FIG. 41B also shows that bevacizumab acutely increases tumor hypoxia in normal weight but not in obese patients (ii). FIG. 41C is a table denoting a positive (green) and negative (red) association between circulating markers and parameters of obesity (BMI, VFA, SFA). FIG. 41D associates IL-6 with worse prognosis in obese patients.

FIG. 42 shows that obesity promotes resistance to anti-VEGF therapy in mouse models of breast cancer by promoting a pro-inflammatory trumor environment with an abundance of in loco protumorigenic factors, in particular IL-6 and FGF-2. Obese BC patients present with increased levels of circulating IL-6 and FGF-2, and decreased vascular response to anti-VEGF. In addition, IL-6 associates with worse prognosis in obese patients.

FIG. 43 shows the effect of obesity on response to anti-VEGF treatment.

FIGS. 44A and 44B show that obesity promotes breast cancer progression.

FIGS. 45A, 45B, 45C, 45D, 45E, and 45F show that obesity promotes pancreatic cancer progession. FIG. 45A shows no tumor growth acceleration in obese resistant mice. FIG. 45B shows lean and obese phenotype in C57/B6 animals obtained by feeding a low (10%) or high (60%) fat diet after 10 weeks, at which time PAN02 tumors were implanted. FIGS. 45C-45F show obesity promoted tumor cell proliferation, reduced apoptosis, tumor growth and metastasis (tumors grown for 3 weeks).

FIGS. 46A and 46B show that obesity promotes TAMs infiltration and immunosupression in pancreatic cancer. PAN02 tumors from obese mice presented with increased tumor-associated macrophages (TAMs) and increased pro-tumor/M2 cytokines.

FIGS. 47A, 47B, 47C, 47D, 47E, and 47F show that stromal VEGFR-1 mediates the obesity-induced pancreatic cancer progression. FIG. 47A shows that stromal VEGFR-1 inhibition prevents weight gain. FIGS. 47B-47D show that stromal VEGFR-1 inhibition (Flt^{TK-/-} mice) prevented the obesity-promoted increased tumor growth and metastasis, without affecting lean mice. FIG. 47E shows the improved metastatic burden in obese Flt1^{TK-/-} mice associated with decreased body weight loss from implantation until tumor extraction compared to obese WT mice. FIG. 47F shows that VEGFR-1 inhibition decreased ki67 expression in tumors from obese mice.

FIGS. 48A, 48B, 48C, 48D, 48E, 48F, 48G, and 48H show that Stromal VEGF-1 does not affect vasculature and TAM infiltration but skews tumor immunity in obesity to an M2 phenotype. FIGS. 48A and 48B show that despite an abundance of VEGR-1 expression in TAMs in PAN02 tumors, stromal VEGFR-1 inhibition (Flt^{TK-/-} mice) did not affect infiltration of this and other myeloid or lymphoid cells. FIGS. 48C-48E show that however, VEGFR-1 inhibition reduced expression of M2 and increased expression of M1 markers in tumors, which associated with polarization of macrophages to M1 phenotype. FIGS. 48F and 48G show that expression of phospho-p38, abundantly expressed in macrophages, was reduced by VEGFR-1 inhibition in tumors from obese mice to the levels seen in lean. FIG. 48H shows that VEGFR-1 inhibition did not affect tumor vessel density.

FIGS. 49A, 49B, and 49C show that stromal VEGFR-1 mediates obesity-induced cancer progression in a second tumor type (breast cancer). Stromal VEGFR-1 inhibition (Flt^{TK-/-} mice) prevented the obesity-promoted metastasis in a breast cancer model (EO771) and improved the metastatic burden (decreased body weight loss from implantation until tumor extraction compared to obese WT mice).

FIGS. 50A, 50B, 50C, and 50D show that Stromal VEGFR-1 inhibition prevents weigh gain but promotes hyperinsulinemia in obese mice. FIGS. 50A and 50B show that stromal VEGFR-1 inhibition prevented weigh gain in HFD fed mice, without affecting lean mice. FIGS. 50C and 50D show that however, VEGFR-1 inhibition augmented glucose intolerance in obese mice and increased plasma insulin levels in tumor bearing obese mice.

FIGS. 51A-51L shows effect of VEGFR1 signaling inhibition on obesity-induced tumor progression in PAN02 pancreatic cancer model: (FIG. 51A) Body weight of C57/B6 animals fed a low (10%) or high (60%) fat diet after 10 weeks, at which time PAN02 tumors were implanted. (FIG. 51B) Tumor weights of PAN02 tumors collected 3 weeks after tumor implantation in lean and obese mice (n=8 mice per group). (FIG. 51C) i: Average number of mesenteric metastasis per mouse. Mesenteries collected at the same time as tumors from panel A.

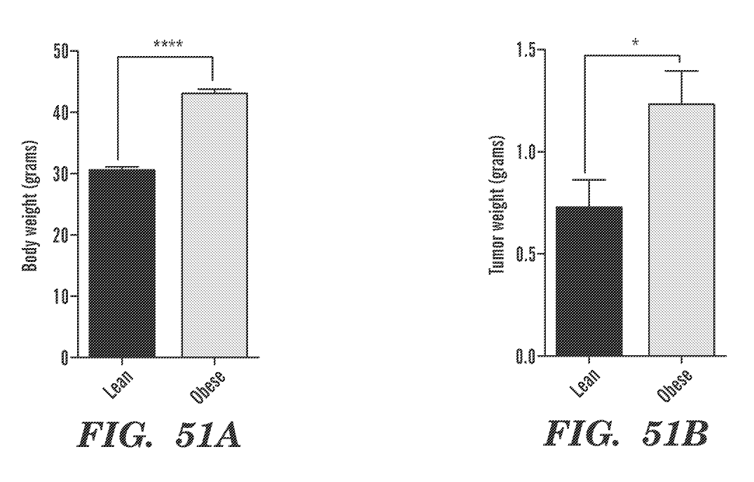

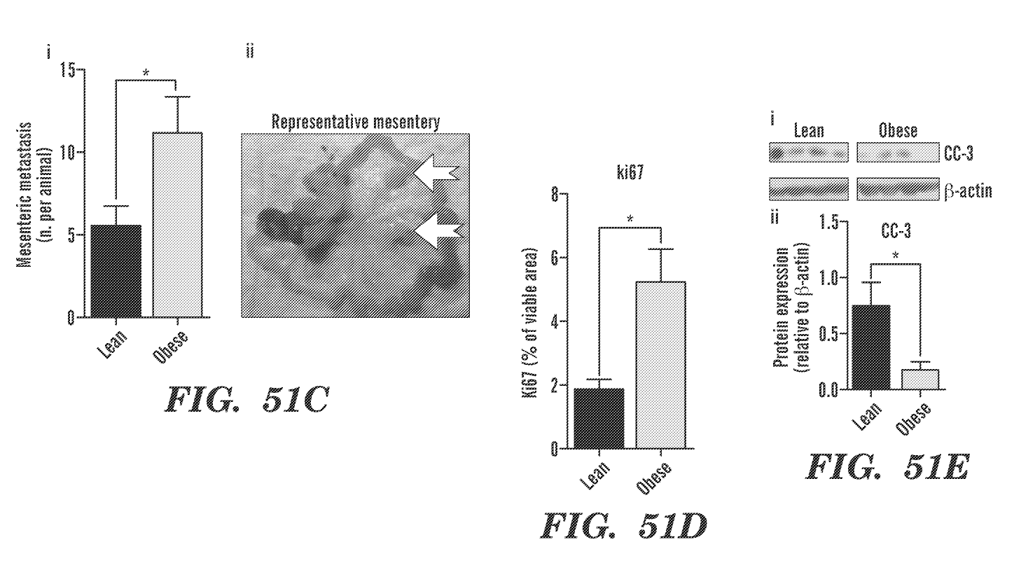

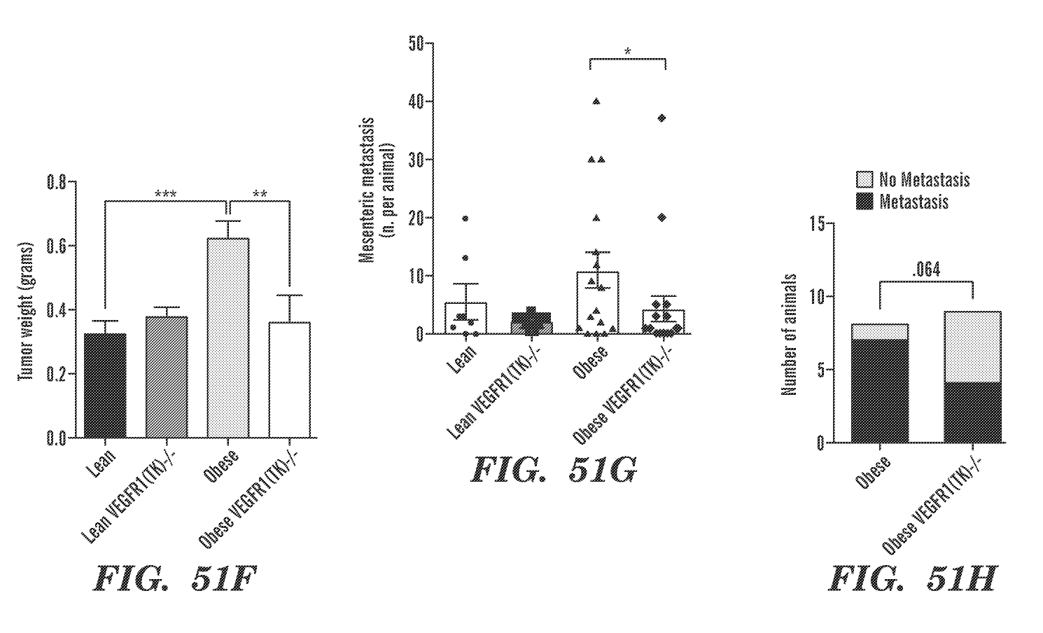

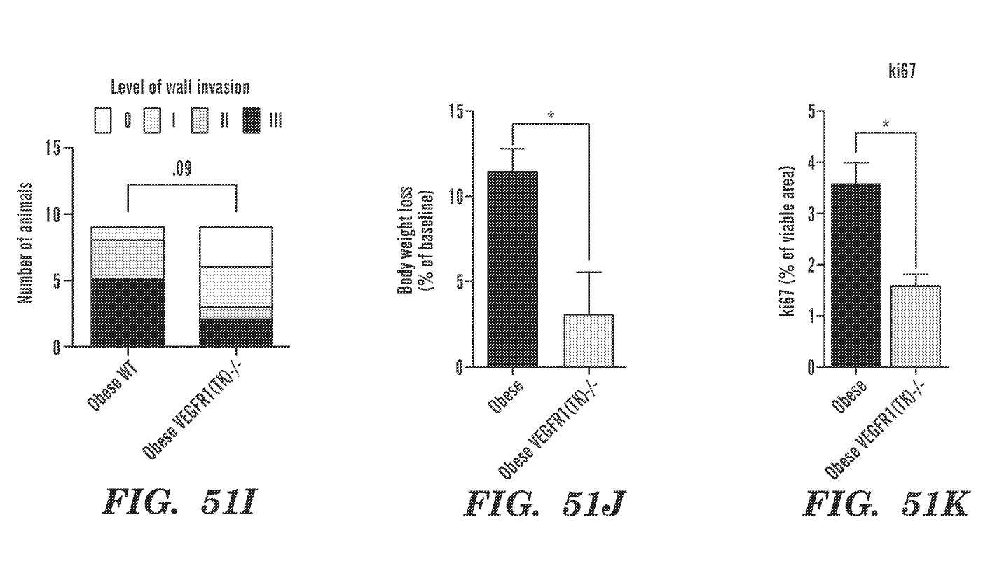

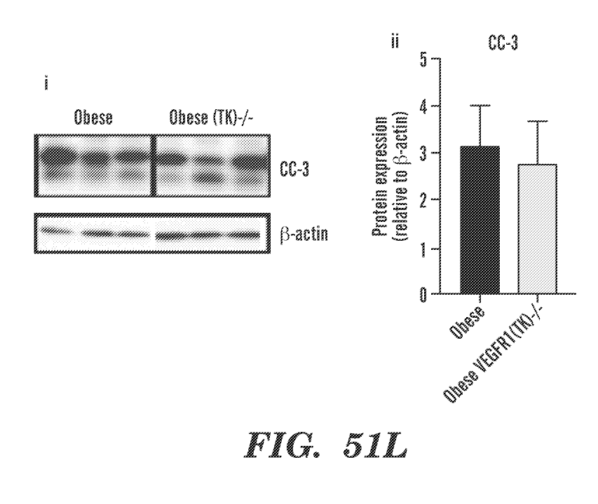

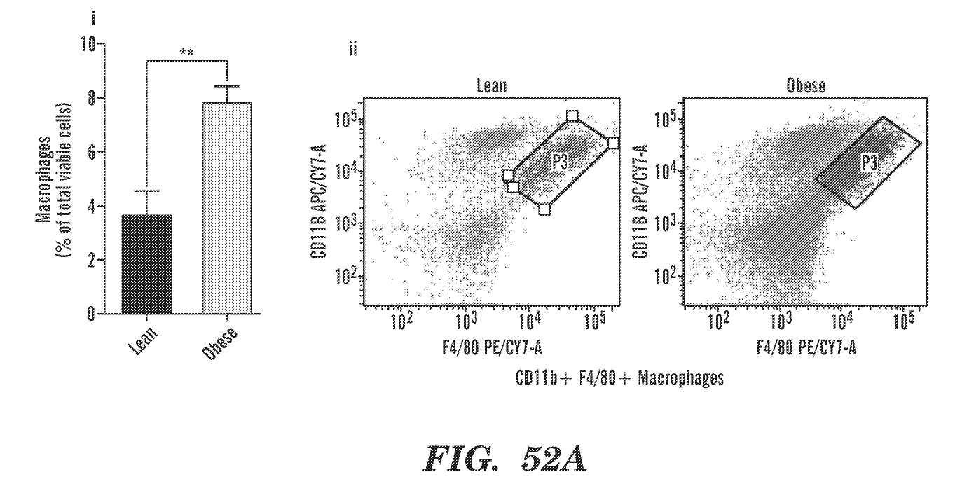

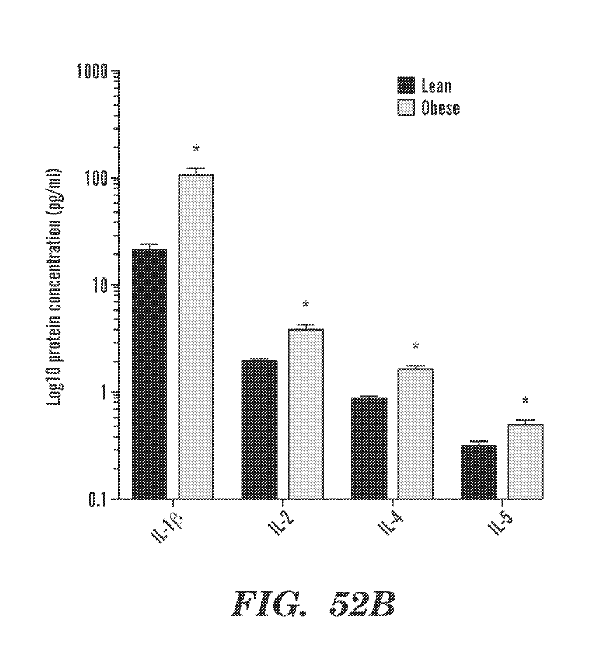

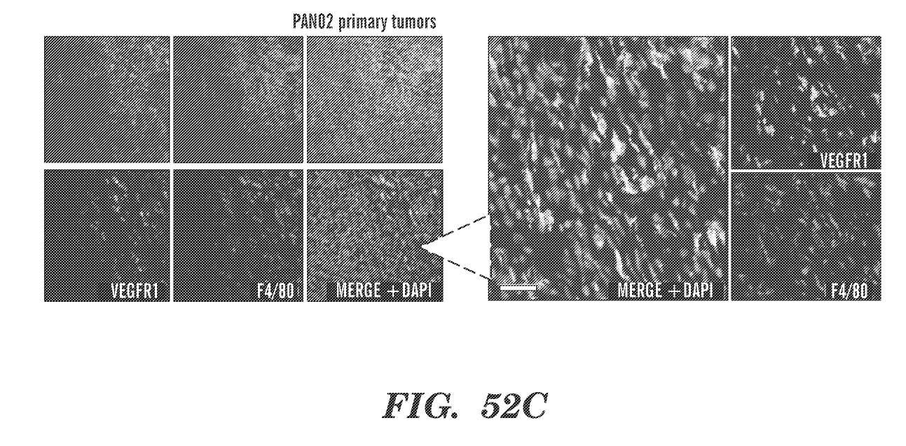

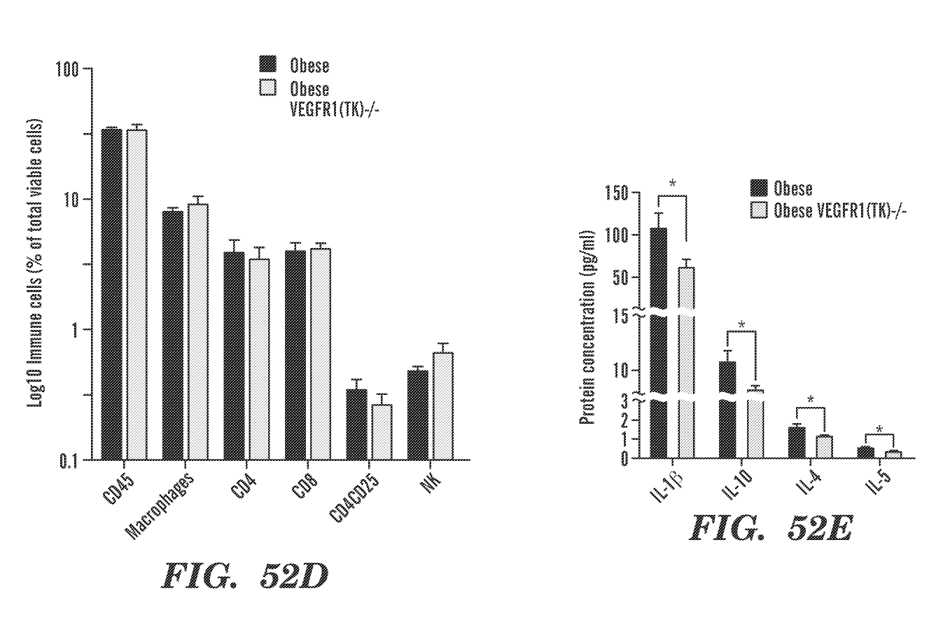

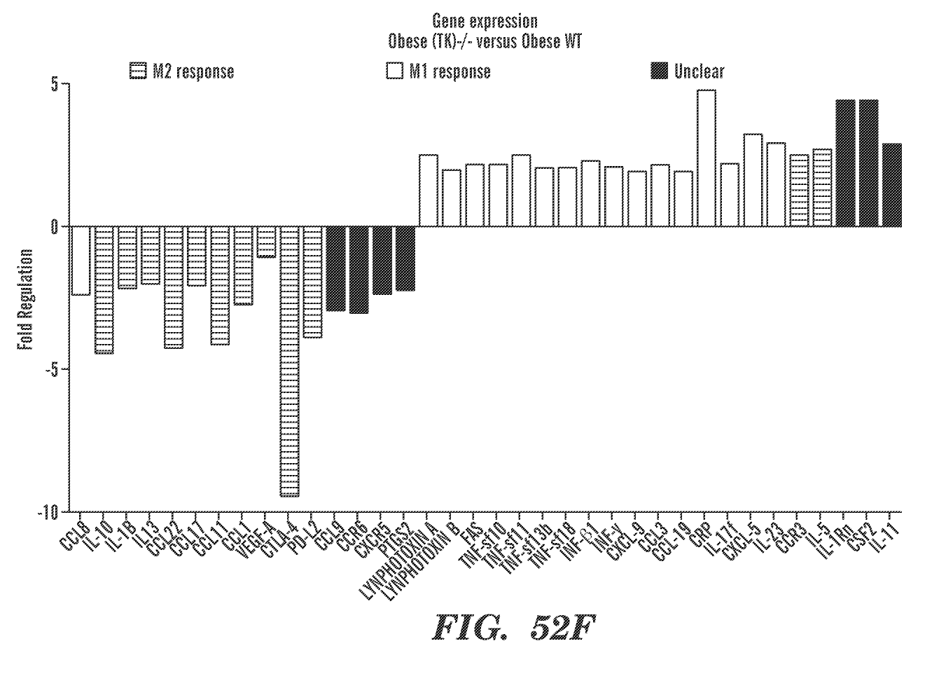

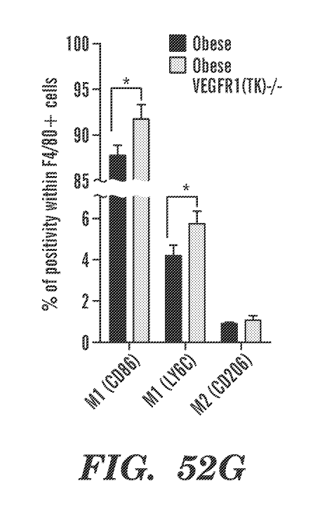

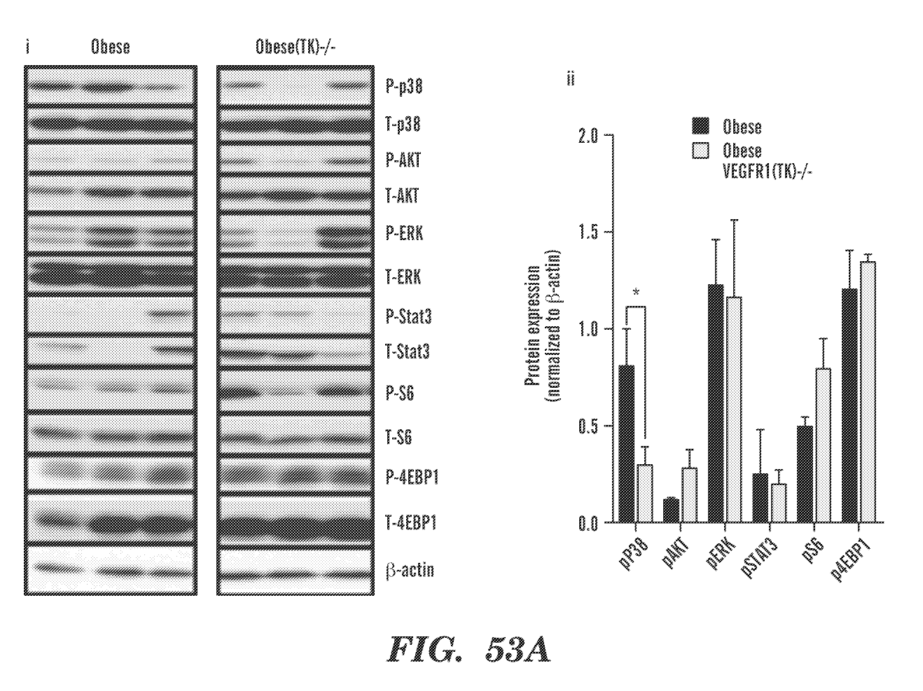

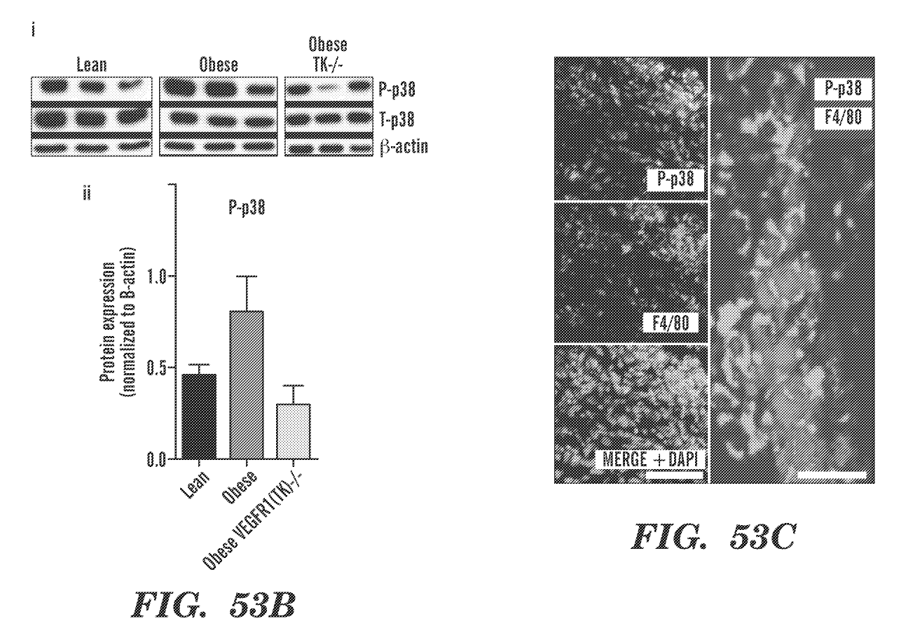

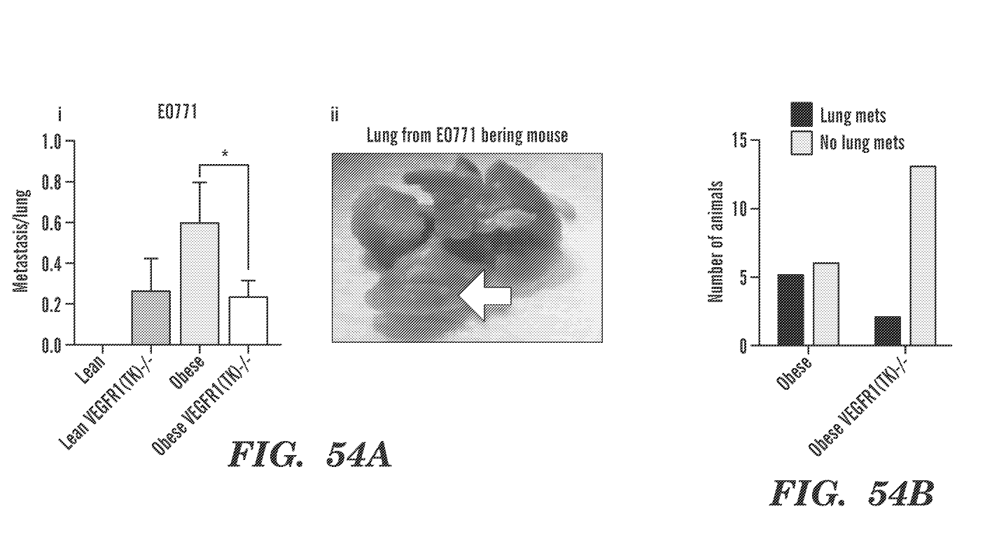

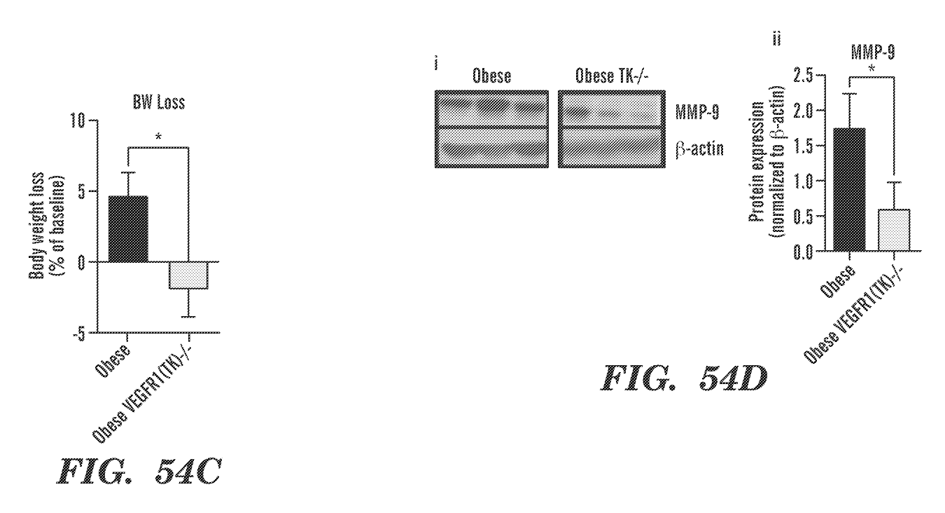

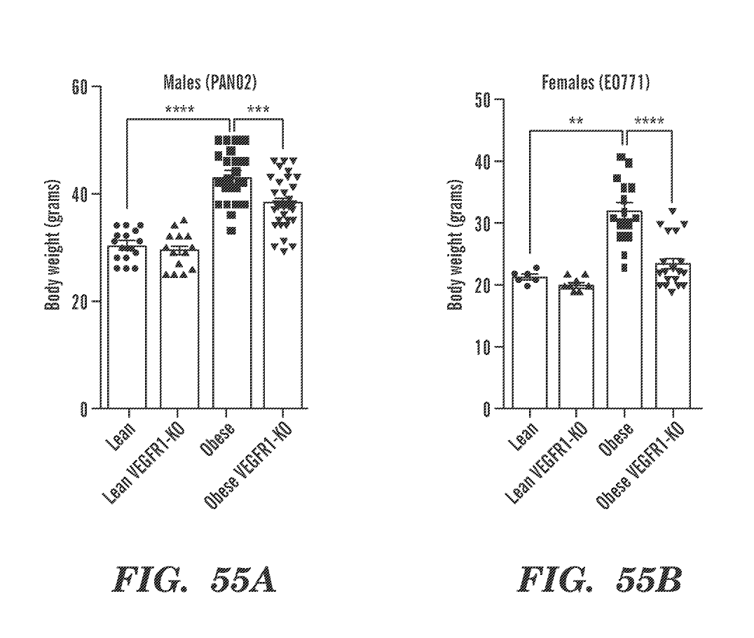

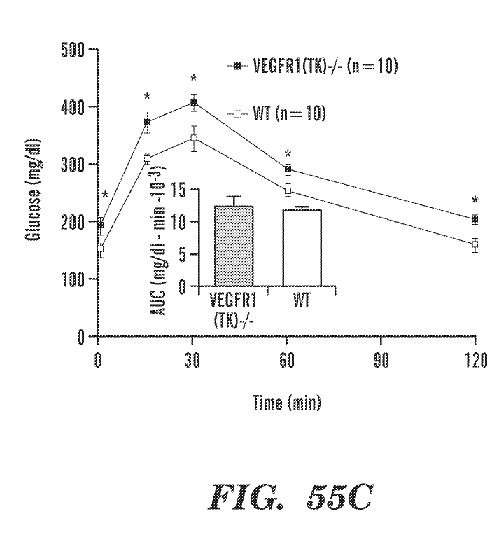

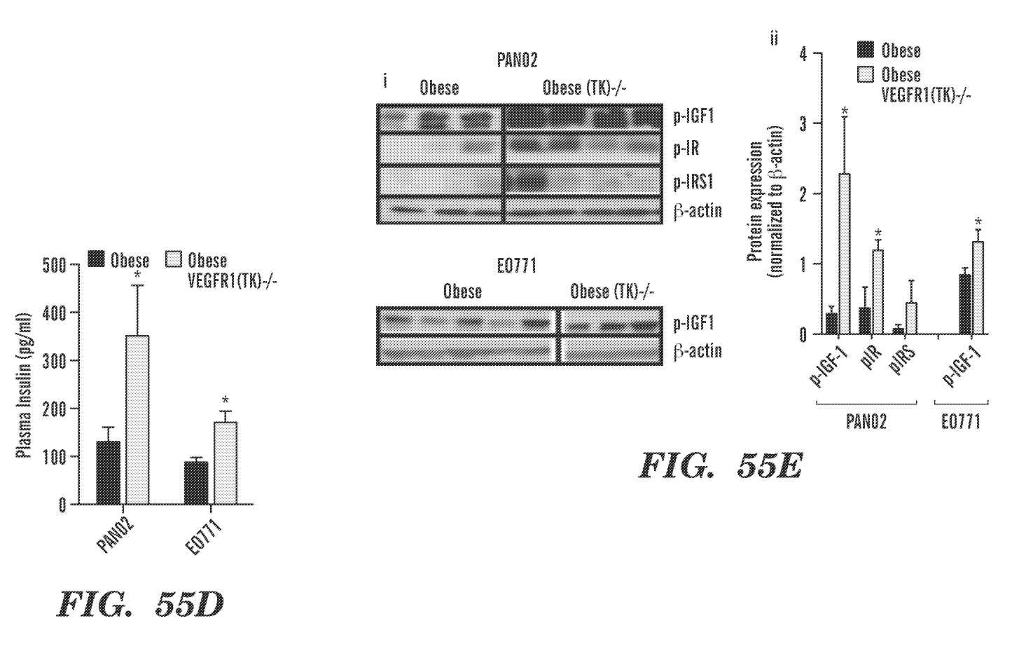

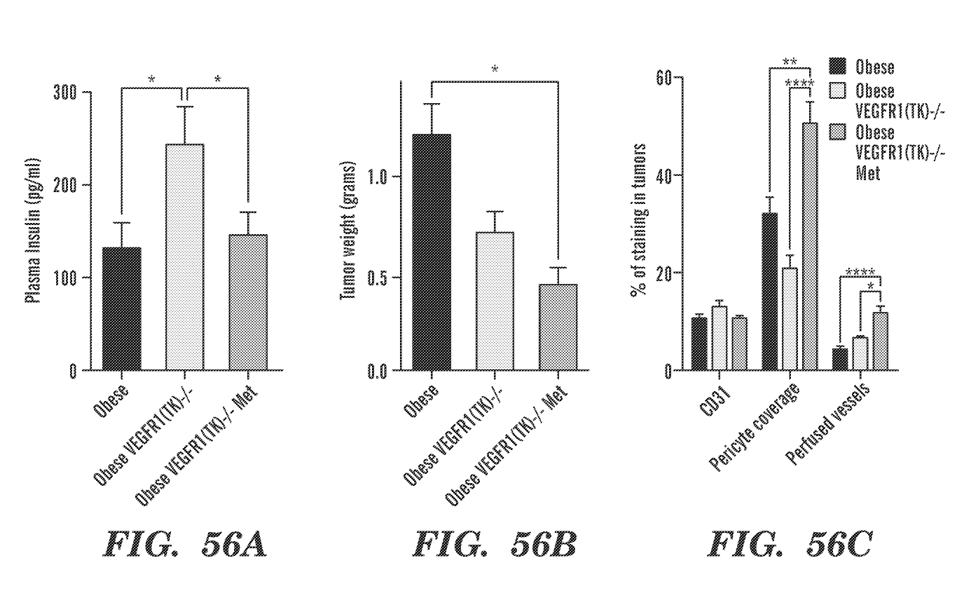

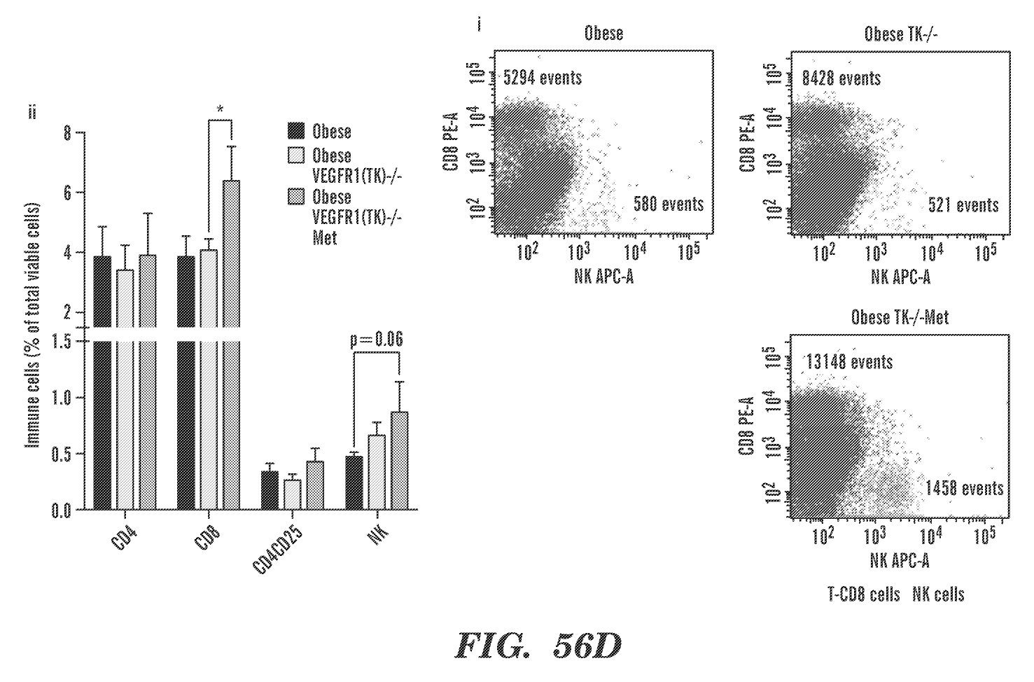

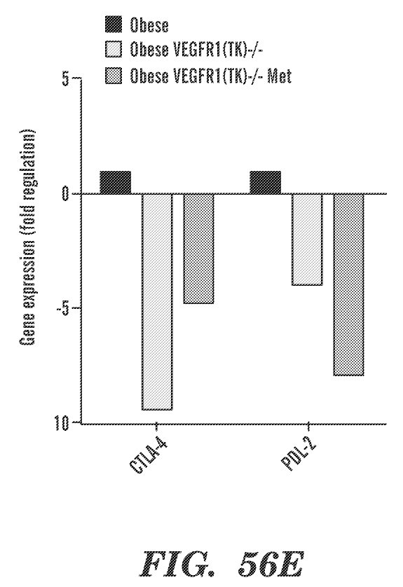

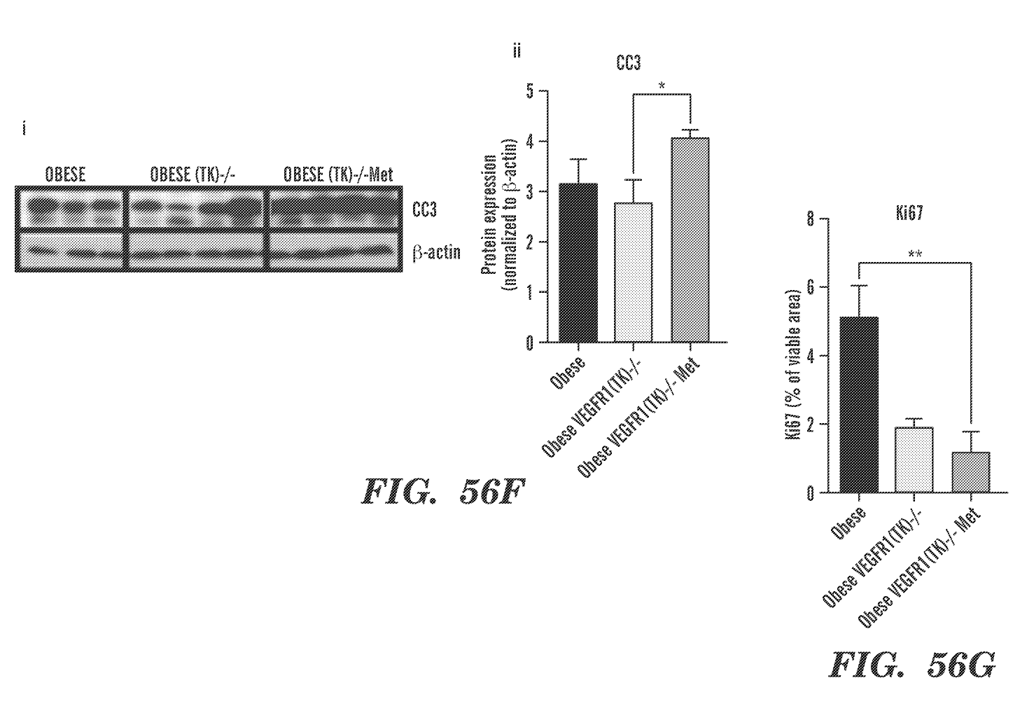

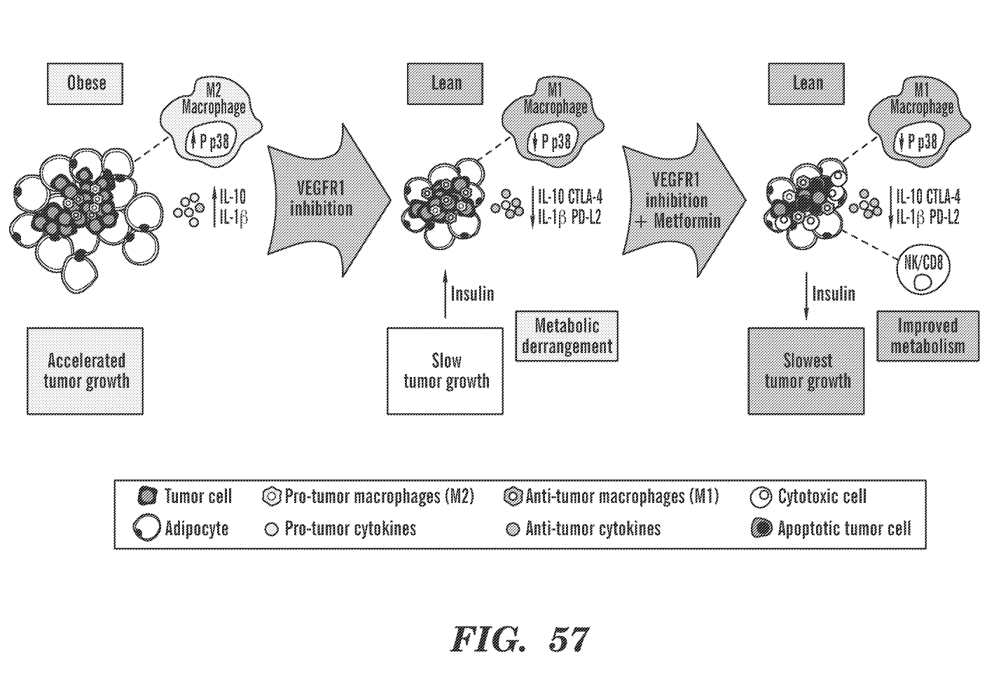

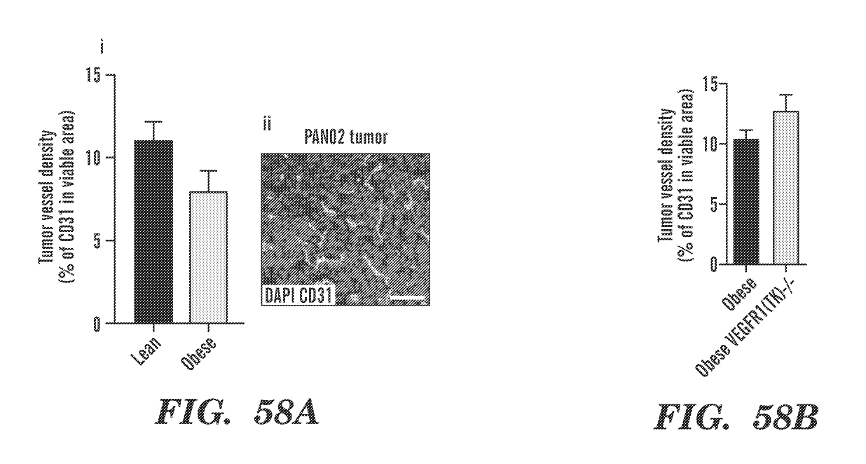

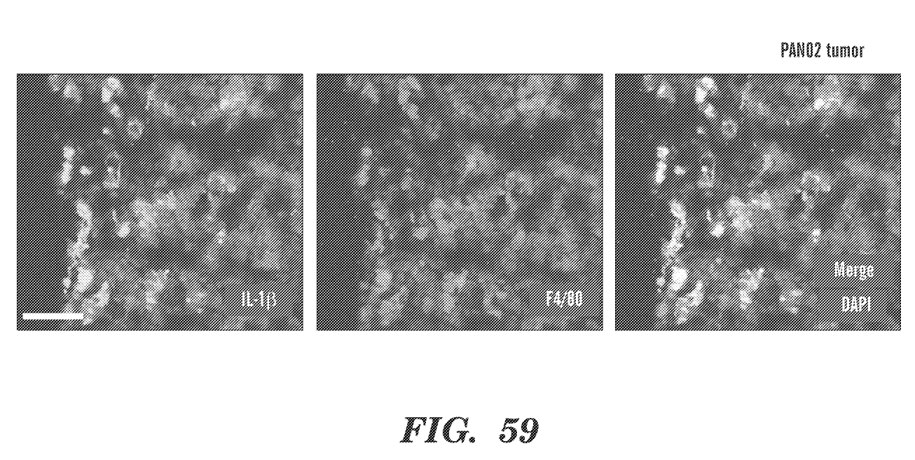

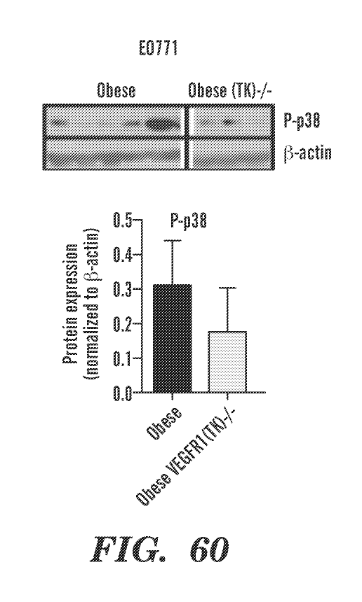

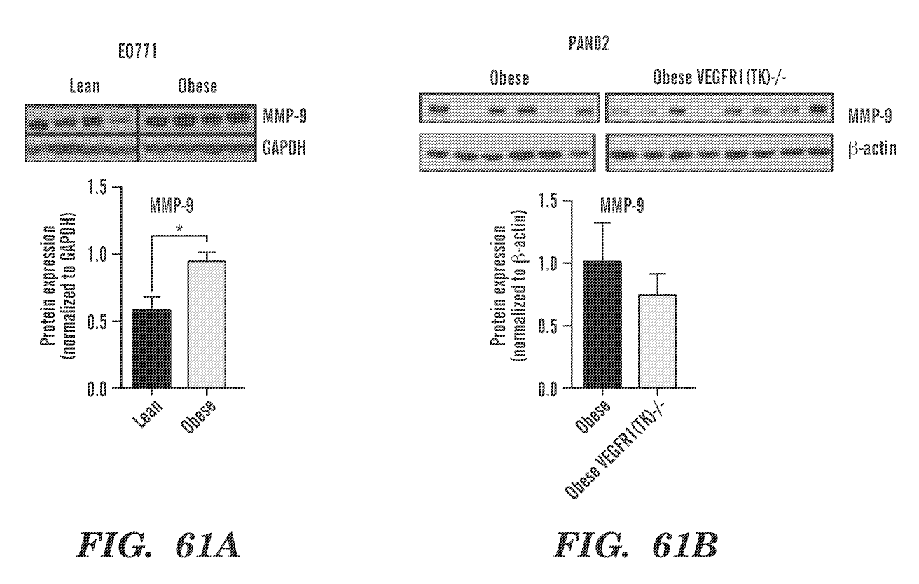

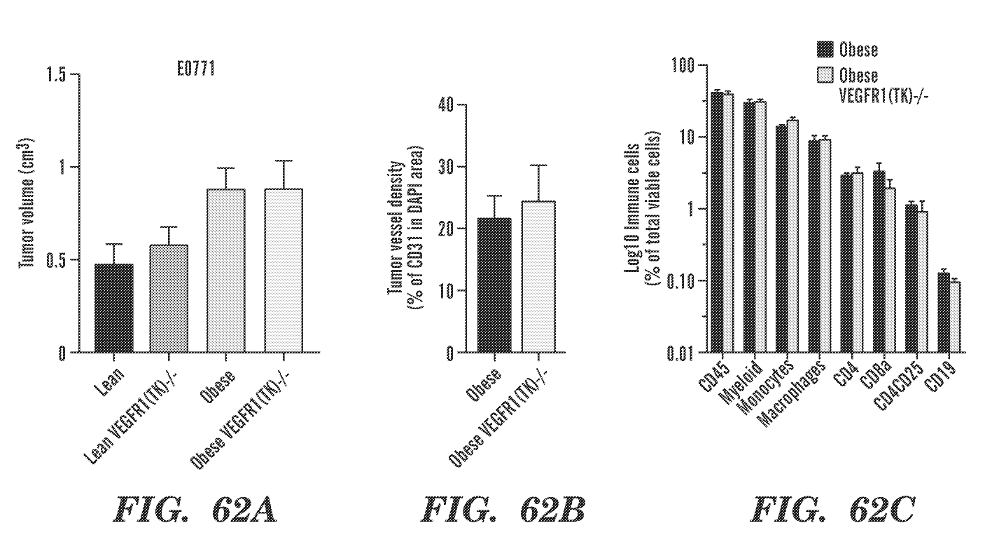

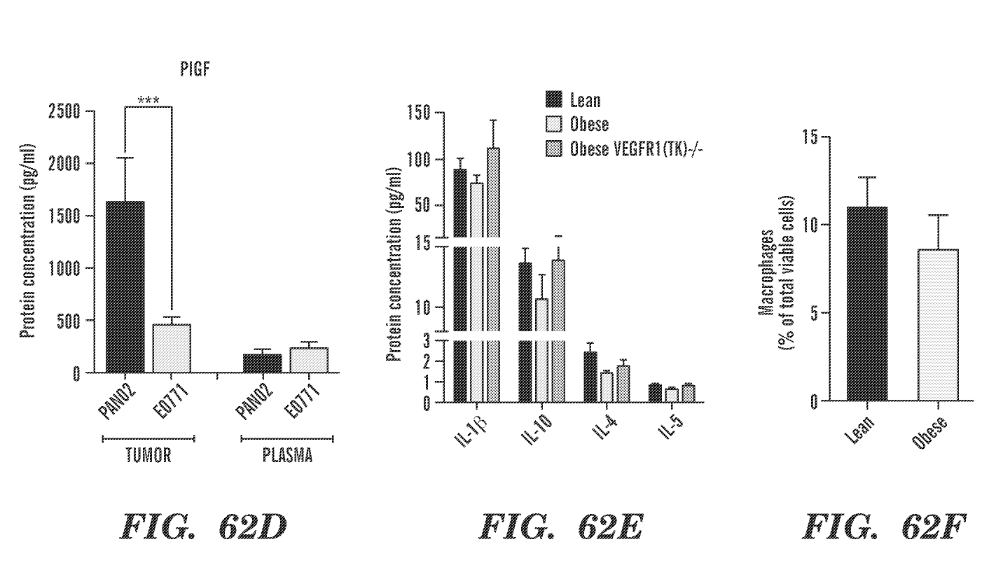

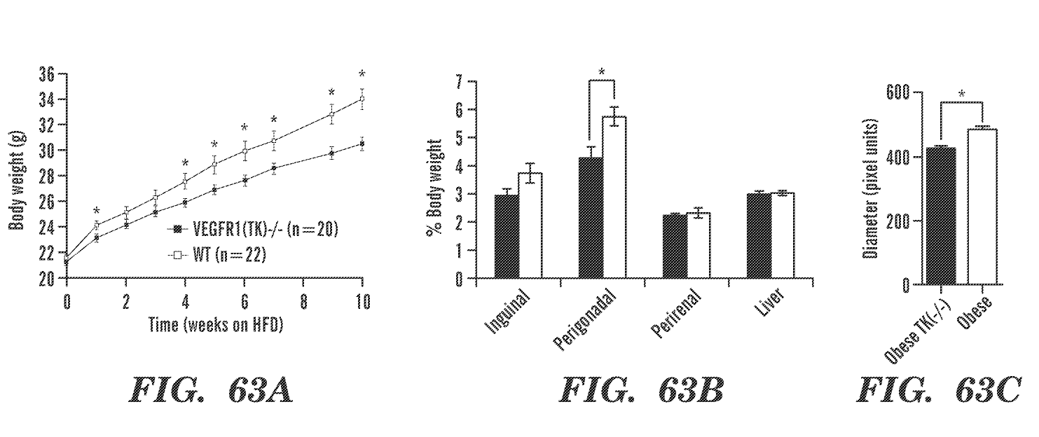

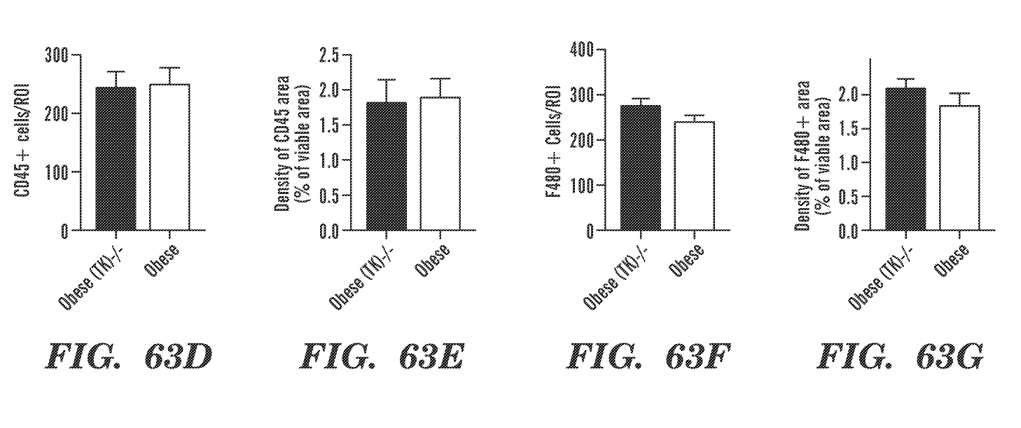

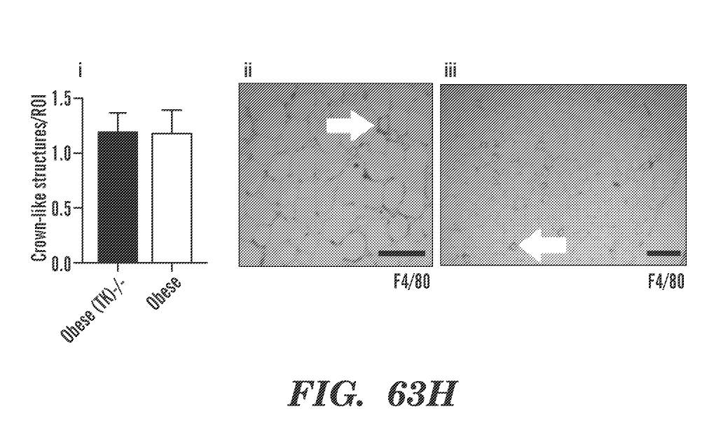

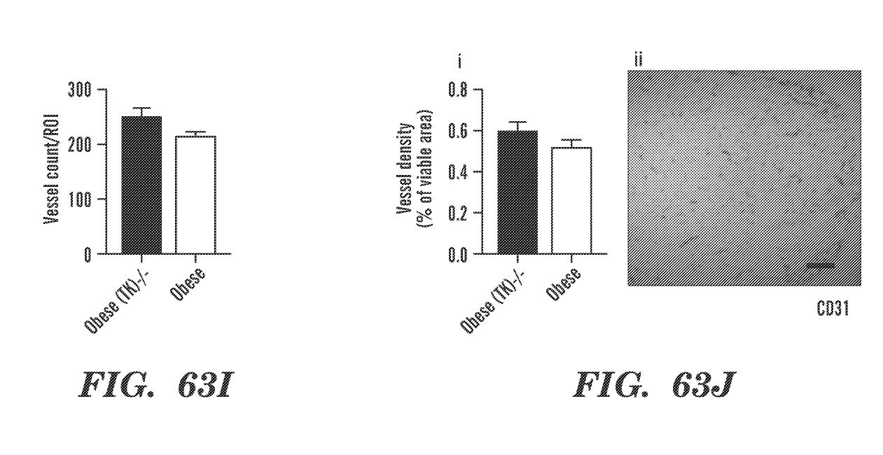

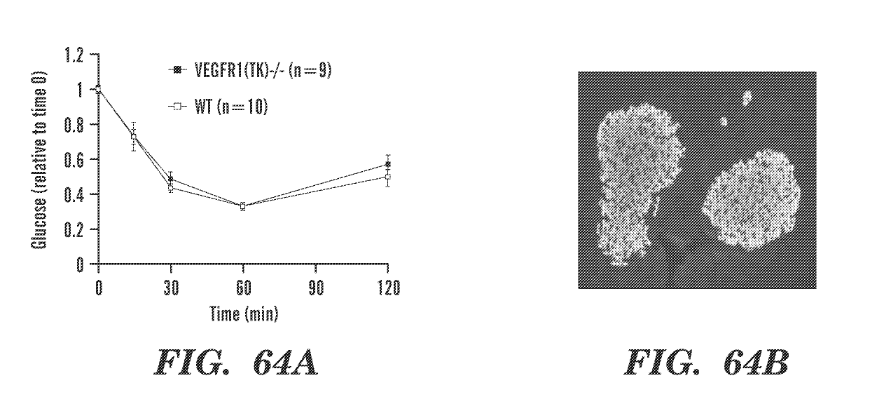

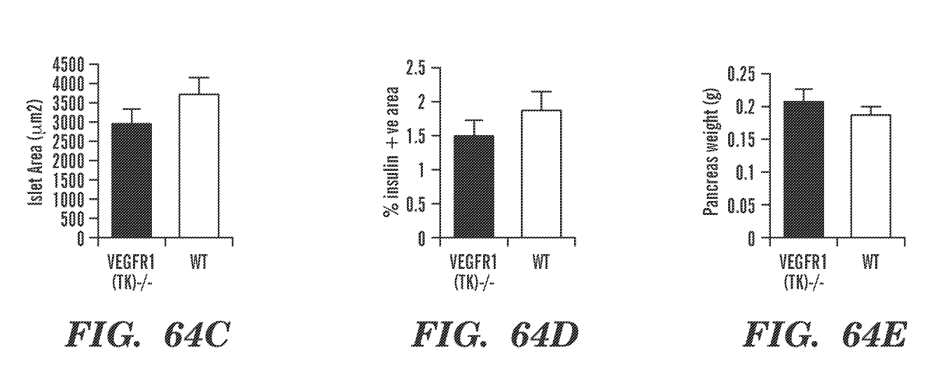

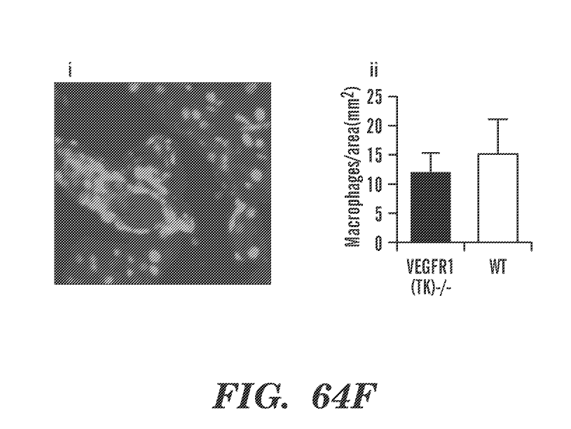

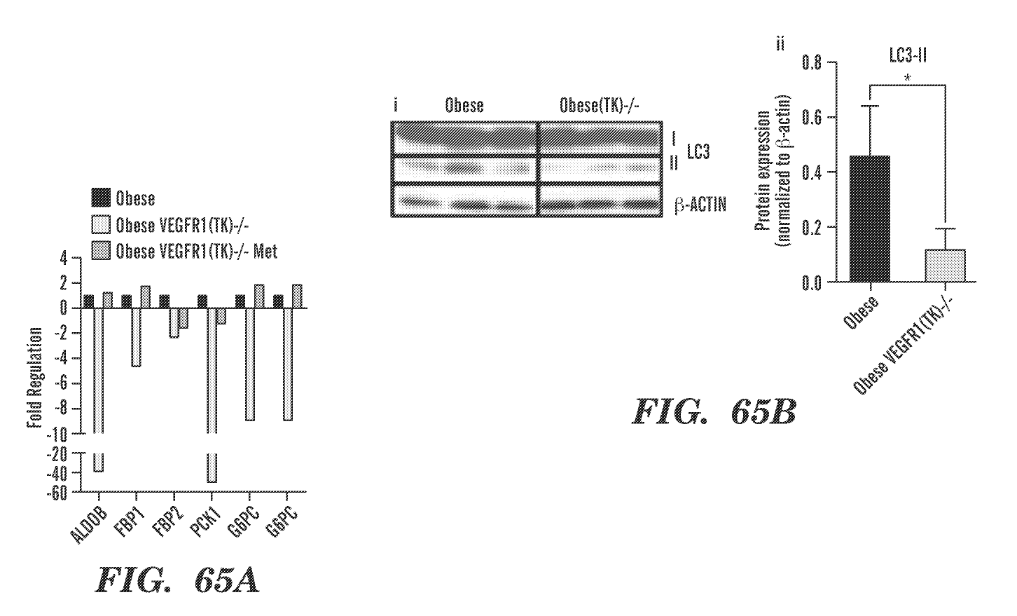

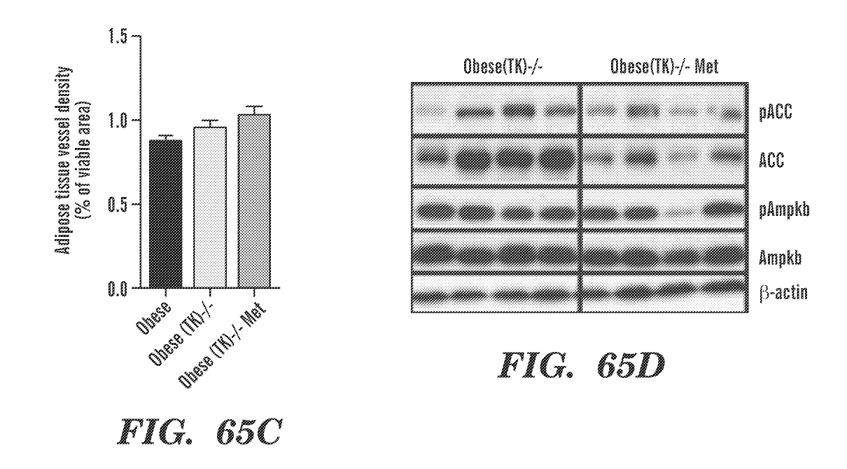

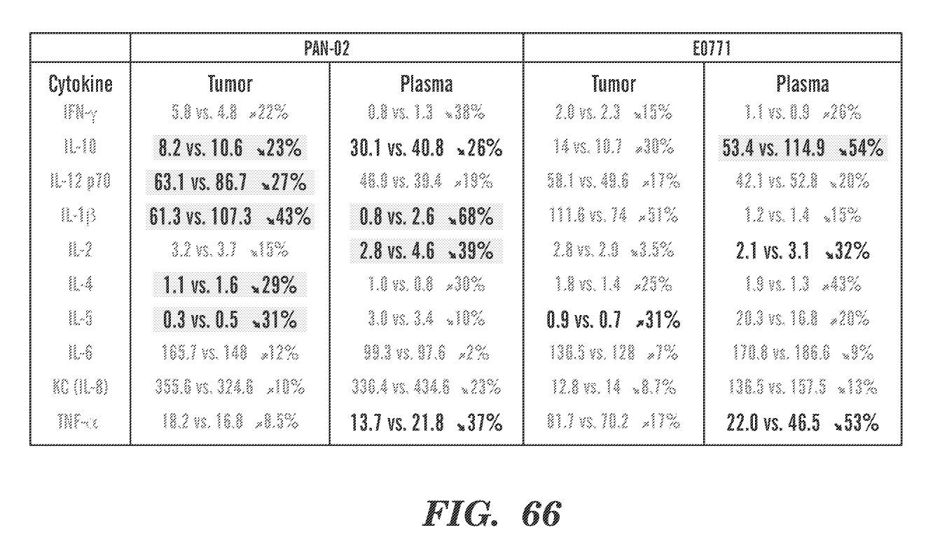

ii: Representative image of mesenteric metastases (arrows). (FIG. 51D) Quantification of the expression of Ki67 proliferation marker in tumors from lean and obese mice, determined by immunofluorescence (n=3-6). (FIG. 51E) i: Protein expression of cleaved caspase-3 (CC-3) apoptotic marker in tumors from lean and obese mice, determined by western blotting. ii: Protein quantification normalized to β-actin. (FIG. 51F) Tumor weights of PAN02 tumors collected 3 weeks after tumor source implantation in obese or lean WT or Flt1TK−/− mice (n=8-15 mice per group). (FIG. 51G) Average number of mesentery metastasis. Mesenteries collected at the time of tumor removal in panel E. (FIG. 51H) Number of mice affected with mesentery metastasis in obese WT or Flt1^{TK-/-} mice. (FIG. 51I) Number of mice that presented with different levels of retroperitoneal wall invasion in obese WT or Flt1TK−/− mice Animals were of given a score 0 to 3 based on the extent of invasion: 0= no invasion, 1= less than 3 metastasis, 2= less than 6 metastasis, 3= more than 6 metastasis. Fisher's exact test was used in both panels. P values are depicted. (FIG. 51J) Body weight loss from implantation until tumor extraction in obese WT or Flt1TK−/− mice. (FIG. 51K) Quantification of ki67 in tumors from obese WT and Flt1TK−/− mice (n=3-6). (FIG. 51L) i: Protein expression of cleaved caspase-3 (CC-3) apoptotic marker. ii: Protein quantification normalized to β-actin. *<0.05, **P<0.01, ***P<0.005, ****P<0.0001 using two-tailed t test for FIGS. 51A-51B, Mann-Whitney test for FIGS. 51C-51E and 51J-51L, two-way analysis of variance for FIGS. 51F-51G, and Fisher's exact test for FIGS. 51H-51I. Error bars in panels represent standard error of the mean.

FIGS. 52A-52G show effect of VEGFR-1 blockade on obesity-altered tumor microenvironment: (FIG. 52A) i: Flow cytometry was used to determine macrophage infiltration in PAN02 tumors from lean and obese mice (left, n=3-6 per group). ii: Representative figures of CD11b (myeloid) and F4/80+ double positive cells indicating macrophages. (FIG. 52B) Elisa was used to determine the cytokine expression in PAN02 tumors from lean and obese mice (n=3-6 per group). (FIG. 52C) Left panels: Expression of VEGFR-1 and F4/80 in two representative PAN02 tumors obtained by immunofluorescence (left). Right panels: Amplification of a region of interest (right). Scale bar: 100 μm (FIG. 52D) Flow cytometry was used to determine immune cell infiltration in PAN02 tumors from obese WT and Flt1^{TK-/-} mice (n=6 per group). (FIG. 52E) ELISA was used to determine the cytokine expression in PAN02 tumors from obese WT and Flt1^{TK-/-} mice (n=6 per group). (FIG. 52F) Gene expression of M1/M2 markers comparing tumors from obese Flt1^{TK-/-} to obese WT. Four samples were pooled into each PCR array plate. (FIG. 52G) Flow cytometry performed to determine the expression of the M1 markers CD86 and LY6C and M2 marker CD186 in the macrophage population in tumors from obese WT and Flt1TK−/− mice (n=6 per group). *P<0.05, **P<0.01. Statistical significances were calculated using Mann-Whitney test for all FIGS. 52A-52G (except FIGS. 52F). Error bars in panels represent standard error of the mean.

FIGS. 53A-53C show effects of VEGFR1 inhibition on signaling pathways in tumors from obese mice: (FIG. 53A) i: Western blot of total and phosphorylated p38, AKT, ERK, STAT3, pS6 and 4EBP1 from PAN02 tumor lysates from obese and obese Flt1^{TK-/-} mice. ii: Quantification of phospho-protein levels relative to β-actin on the right. (FIG. 53B) i: Western blot of total and phosphorylated p38 from PAN02 tumor lysates from lean, obese and obese Flt1^{TK-/-} mice. ii: Quantification of phospho-protein levels of p38 relative to β-actin in the lower panel. (FIG. 53C) Expression of phosphorylated-p38 (P-p38) and F4/80 in PAN02 tumors. Scale bars: 100 μm (left), 30 μm (right). *P<0.05. Statistical significances were calculated by Mann-Whitney test for FIG. 53A and Kruskal-Wallis for FIG. 53B. Error bars in panels represent standard error of the mean.

FIGS. 54A-54D show effect of VEGFR-1 blockade on obesity-induced breast tumor progression. (FIG. 54A) i: Average number of lung metastasis per mice: Lungs collected 3 weeks after implantation of E0771 tumors in lean and obese WT or Flt1TK−/− mice. ii: Representative image of metastasis in the lungs (blue arrow, right panel) (n=6-11 mice per group). (FIG. 54B) Incidence (number of mice affected with lungs metastasis) in obese WT or Flt1^{TK-/-} mice. (FIG. 54C) Body weight loss from implantation until tumor extraction in obese WT or Flt1^{TK-/-} mice. (FIG. 54D) i: Western blot analysis of MMP-9 from E0771 primary tumor lysates from obese WT and obese Flt1TK−/− mice. ii Quantification of protein levels of MMP-9 relative to β-actin. *P<0.05. Statistical significances were calculated by two-way ANOVA for FIG. 54A, Fisher's exact test for FIG. 54B and Mann-whiney tests for FIGS. 54C-54D. Error bars in panels represent standard error of the mean.

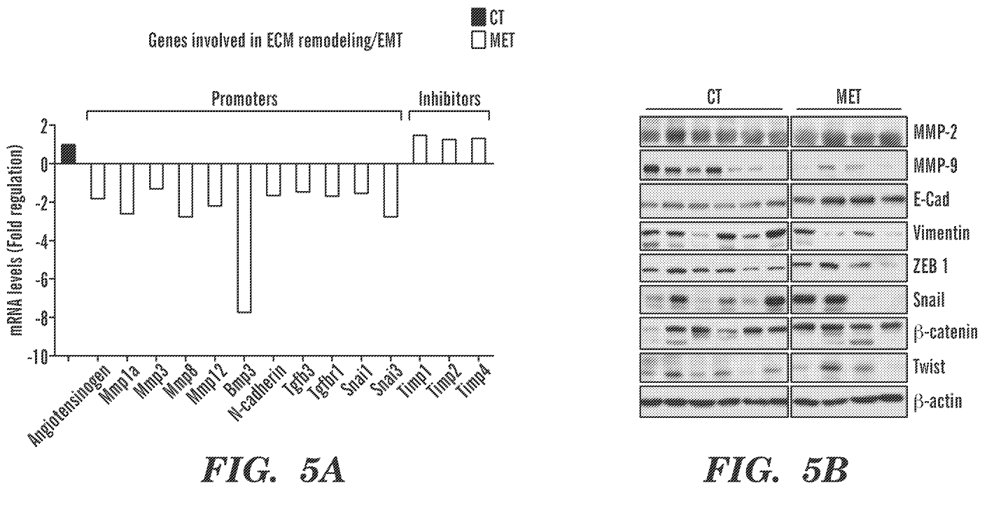

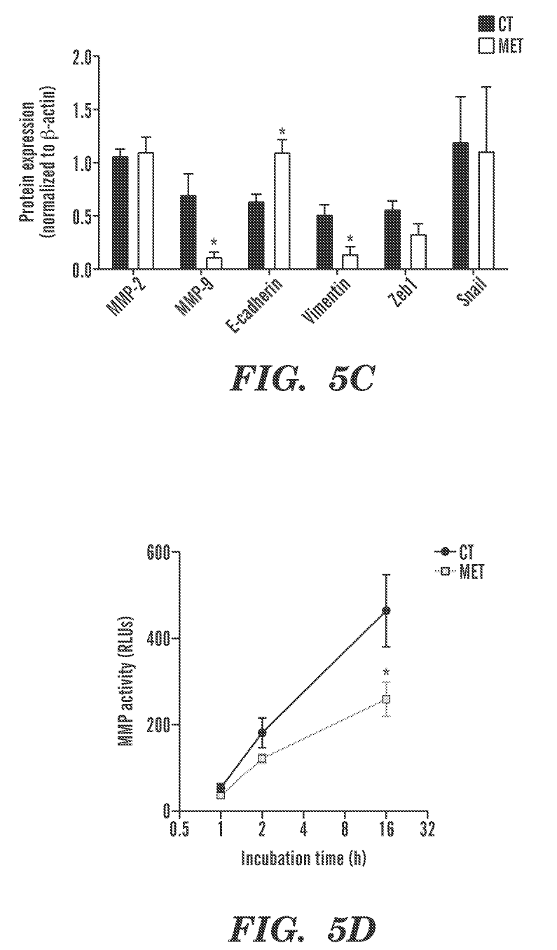

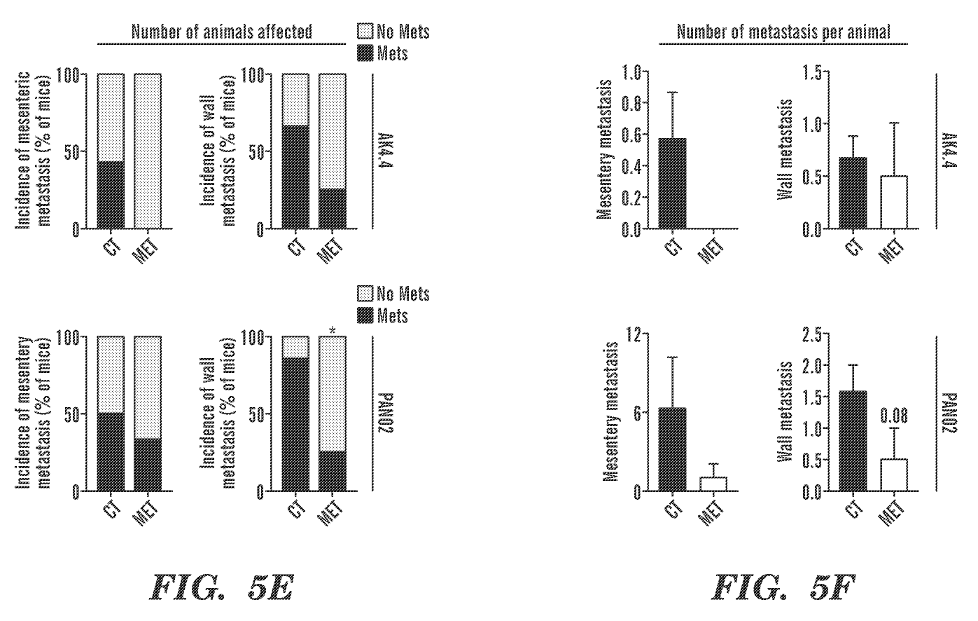

FIGS. 55A-55E show metabolic effects of VEGFR1 inhibition: (FIGS. 55A-55B) Body weight of male (FIG. 55A) and female (FIG. 55B) mice after 10 weeks of high fat or low fat diet in WT or Flt1^{TK-/-} group. Number of animals depicted as dots on each column (FIG. 5C) Glucose tolerance test in Flt1^{TK-/-} vs. WT obese mice. Number of mice per group depicted in the figure. (FIG. 55D) Levels of plasma Insulin in WT vs Flt1^{TK-/-} obese mice 3 weeks after implantation of PAN02 or E0771 tumors (n=4-8). (FIG. 55E) i: Western blot analysis of phosphorylated IGF-1R, IR, and IRS-1 in PANO2 tumor lysates and phosphorylated IGF-1R in E0771 tumor lysates from obese WT and obese Flt1^{TK-/-} mice. ii: Quantification of phospho-protein levels relative to β-actin on the right panel. *P<0.05, **P<0.01, ***P<0.005, ****P<0.001 using two way analysis of variance for FIGS. 55A-55B, two tailed t-test for FIG. 55C, and Mann-Whitney test for FIGS. 55D-55E. Error bars in panels represent standard error of the mean.

FIGS. 56A-56G show effects of VEGFR1 inhibition in combination with metformin on systemic metabolism and tumor growth in obese mice: (FIG. 56A) Levels of plasma insulin in obese mice WT, Flt1^{TK-/-} and Flt1^{TK-/-} treated with metformin, 3 weeks after implantation of PAN02 (n=8-10). (FIG. 56B) Tumor weights of PAN02 tumors 3 weeks after chunk implantation in obese mice WT, Flt1^{TK-/-} and Flt1^{TK-/-} treated with metformin (n=8-10). (FIG. 56C) Percentage of CD31+ expression in total DAPI+ viable area, pericyte coverage (percentage of CD31+ staining that also colocalizes with SMA+ staining) and perfused vessels (% of CD31/Lectin double positive vessels in total DAPI+ viable area) in tumors from panel B (n=4). (FIG. 56D) i: Representative figures of CD8 (purple) and NK (orange) cells in tumors from panel B. ii: Quantification of immune cells (% of total viable cells) (n=4-6). (FIG. 56E) Gene expression of CTLA-4 and PD-L2 comparing tumors from obese Flt1TK−/− to obese WT. Data extracted from a PCR array assay, four samples were pooled into each PCR array plate. (FIG. 56F) i: Western blot analysis of cleaved caspase-3 (CC3) from PAN02 tumors lysates from panel B. ii: Quantification relative to β-actin in the right panel. (FIG. 56G) Quantification of Ki67 expression in tumors from panel B, obtained using immunofluorescence (n=3-6). *P<0.05 using two-tailed t-test for FIGS. 56A-56C. *P<0.05 **P<0.01, ****P<0.005 using one way analysis of variance with Turkey's multiple comparisons test for panels FIGS. 56A-56D and 56F, and Kruskal-Wallis for FIG. 56E. Error bars in panels represent standard error of the mean.

FIG. 57 is a schematic diagram showing VEGFR1 as a “dual” target in the obesity-cancer connection: On the one hand preventing expansion of adipose tissue in obesity, and on the other, directly affecting tumor progression by promoting M1 macrophage polarization and production of pro-tumor cytokines. VEGFR1 inhibition aggravates the systemic metabolism, however this can be normalized using metformin, which also provides additional benefits at the tumor site by improving vessel perfusion and infiltration of cytotoxic cells.

FIGS. 58A-58B show effects of obesity and VEGFR-1 inhibition on PAN02 tumor vasculature: (FIGS. 58A-58B) Percentage of CD31+ expression in total DAPI+ viable area in PAN02 tumors (n=4) in lean versus obese mice (FIG. 58A) or in obese versus obese Flt1^{TK-/-} mice (FIG. 58B). Representative image of tumor vessel staining (FIG. 58A right). Scale bar: 100 μm. Mann-Whitney test was used to assess differences between groups. Error bars in panels represent standard error of the mean.

FIG. 59 is a set of fluorescent images showing production of IL-1β by macrophages in PAN02 tumors: Co-expression of IL-1β and F4/80+ by immunofluorescence in PAN02 tumors. Scale bar: 50 μm.

FIG. 60 shows effect of VEGFR-1 blockade on P38 expression in E0771 tumors from obese mice: Western blot analysis of total and phosphorylated P38 from E0771 tumor lysates from obese and obese Flt1^{TK-/-} mice (upper panel). Quantification of phospho-protein levels of P38 relative to β-actin (lower panel). Mann-Whitney test was used to assess differences between groups. Error bars represent standard error of the mean.

FIGS. 61A-61B show effect of obesity on MMP9 expression in E0771 tumors from obese mice. (FIG. 61A) Western blot analysis of MMP-9 from E0771 primary tumor lysates from lean and obese obese mice (upper panel). Quantification of MMP-9 levels relative to GAPDH (lower panel). (FIG. 61B) Western blot analysis of MMP-9 from PAN02 primary tumor lysates from obese and obese Flt1TK−/− mice (upper panel). Quantification of MMP-9 levels relative to β-actin (lower panel). Mann-Whitney test was used to assess differences between groups. Error bars represent standard error of the mean.

FIGS. 62A-62F show effects of VEGFR-1 blockade on obesity-induced breast tumor progression. (FIG. 62A) Tumor volumes of E0771 tumors 3 weeks after cell implantation in obese or lean WT or Flt1TK−/− mice (n=6-11 mice per group). (FIG. 62B) Percentage of CD31+ expression in total DAPI+ viable area in E0771 tumors (n=4) (FIG. 62C) Flow cytometry was used to determine immune cell infiltration in E0771 tumors from obese WT and Flt1TK−/− mice (n=3-4 per group). (FIG. 62D) Elisa was used to determine the P1GF expression in plasma and tumors from PAN02 and E0711 tumor bearing obese mice (n=3-6 per group). (FIG. 62E) ELISA was used to determine the cytokine expression in E0711 tumors obese WT or Flt1TK−/− mice (n=3-6 per group). (FIG. 62F) Flow cytometry was used to determine macrophage infiltration in E0771 tumors from lean and obese mice (n=3-4 per group). Two way ANOVA was used to assess a difference between groups in FIG. 62A, and Mann-Whitney test in FIGS. 62B-62F. ***P<0.01; error bars in panels represent standard error of the mean.

FIGS. 63A-63J show effects of VEGFR1 inhibition on body weight gain, immune cell infiltration and vasculature in adipose tissues during obesity. (FIG. 63A) Body weight gain in WT and Flt1^{TK-/-} mice fed high-fat diet. Number of animals is depicted in the figure. (FIG. 63B) Weights of inguinal, perigonadal, and perirenal fat pads, as well as the liver, in proportion to total body weight. Number of animals is depicted in the figure. (FIG. 63C) Adipocyte diameter measured in visceral adipose tissue in Flt1^{TK-/- vs. WT obese mice. Averages were taken from at least} 8 randomly selected 10X fields for each adipose tissue, 7-9 adipose tissues for each genotype. (FIGS. 63D-63H) Count (number) and area density of CD45 positive cells (FIGS. 63D-63E), F4/80 positive cells (FIGS. 63F-63G) or crown-like (F4/80+ staining around adipocytes) structures (FIG. 63H) in visceral adipose tissue from Flt1^{TK-/-} vs. WT obese mice. i. quantitative graph of crown-like structures. ii-iii, representative images of macrophage (F4/80, brown) staining as well as crown-like structures (arrows), in 4 and 10 X micrographs. (FIGS. 63I-63J) Count (number) of CD31+ cells (FIG. 63I), as well as density of CD31+ staining per viable area (FIG. 63J) in visceral adipose tissue from Flt1^{TK-/-} vs. WT obese mice. i. quantitative graph of vessel density. ii. representative image of vessel (CD31+) staining. Averages taken from at least 8 randomly selected 10X fields for each adipose tissue, 7-9 adipose tissues for each genotype. Scale bars: 1000 μm. *P<0.05 using t-test for FIGS. 63A-63C, and Mann-Whitney test for FIGS. 63D-63J. Error bars in panels represent standard error of the mean.

FIGS. 64A-64F show effects of VEGFR1 inhibition on insulin tolerance and insulin production by the pancreas during obesity. (FIG. 64A) Insulin tolerance in WT or Flt1^{TK-/-} obese mice. (FIGS. 64B-64D) Representative picture of pancreatic islet showing positive insulin staining (FIG. 64B). Mean pancreatic islet area (FIG. 64C) and insulin+staining area (FIG. 64D) as a percentage of total pancreas cross-sectional area in Flt1^{TK-/-} and WT obese mice. Images were taken from over 290 individual islets and from over 11 animals for each genotype. (FIG. 64E) Pancreas tissue mass in Flt1^{TK-/-} and WT obese mice (n=6 for each genotype). (FIG. 64F) i. representative image of pancreatic macrophage showing positive staining for ER-MP23. Nuclei stained with DAPI. ii. pancreatic macrophage content. Averages taken from at least 5 randomly selected 10x fields for each pancreas, 4 pancreases for each genotype. T-test was used to assess a difference between groups. Error bars in panels represent standard error of the mean.

FIGS. 65A-65D show effects of VEGFR1 inhibition and metformin on tumor metabolism and vessel density in obese mice. (FIG. 65A) Results from a PCR metabolism gene array, selection of gluconeogenic genes in PAN02 tumors from WT, Flt1^{TK-/-} and Flt1^{TK-/-} with metformin obese mice (n=4 mice per group composite). (FIG. 65B) Western blot analysis of the autophagic marker LC3 from PAN02 tumor lysates obese WT and obese Flt1TK−/− mice (i). Quantification of LC3-II relative to β-actin (ii). (FIG. 65C) Percentage of CD31+ staining per viable area in visceral adipose tissue from PAN02 tumor bearing obese mice WT, Flt1^{TK-/-} and Flt1^{TK-/-} treated with metformin. Averages taken from at least 8 randomly selected 10X fields for each adipose tissue, 7-9 adipose tissues for each genotype. (FIG. 65D) Western blot analysis of phosho and total Acetyl-CoA Carboxylase (ACC) and 5′-AMP-activated protein kinase Beta (AMPK-β) from PANO2 tumors lysates from obese mice Flt1^{TK-/-} and Flt1^{TK-/-} treated with metformin. *P<0.05 using Mann-Whitney test for FIG. 65B, and one-way ANOVA for FIG. 65C. Error bars in FIGS. 65B-65C represent standard error of the mean.

FIG. 66 is a table showing tumor and plasma cytokine quantification in Flt1^{TK-/-} vs. WT obese mice. Values represent the protein concentration in Flt1^{TK-/-} vs. WT in PAN02 or E0771 bearing obese mice, followed by the percentage of change. Bold and highlighted represent significant differences (P<0.05), bold represents trends. Significant differences calculated using two-tailed t-test comparing absolute values of cytokine levels between WT vs. Flt1^{TK-/-} groups.

### DETAILED DESCRIPTION OF THE INVENTION

The invention is based, at least in part, on the discovery that metformin reduces the fibro-inflammatory microenvironment in a cancer patient. Without being bound by theory, the effect of metformin in the microenvironment is believed to be extrinsically directed, independent of its effects on cancer cells or tumor metabolism, so-called tumor-intrinsic mechanisms.

Metformin is a glucose-lowering drug that is often prescribed for diabetic cancer patients. Metformin treatment has shown benefit in some, but not all studies; these inconsistent results suggest that a subset of tumors or cancer patients may not respond to metformin. The mechanism of action of metformin in cancer is not well understood. Thus, gaining a better understanding of how metformin acts in the tumor microenvironment and at the site of the tumor will inform how to achieve greater therapeutic benefit and/or identify those patients that would benefit the most from metformin treatment.

Without being bound by theory, it is believed that metformin can increase the efficacy or potentiate the action of other therapeutic agents, e.g., cancer therapies described herein. It does so by reducing desmoplasia, extracellular matrix component (ECM) production, and inflammation. In overweight patients and obese patients—who appear to have increased levels of extracellular matrix components and deregulated, desmoplastic signaling activity in tumors, metformin treatment reduced tumor levels of hyaluronan. The robust affect of on hyaluronan and to a lesser extent collagen-I was confirmed in preclinical obese/diabetic mouse models of syngeneic pancreatic ductal adenocarcinoma. Furthermore, the alleviation of desmoplasia occurred, at least in part, through a direct effect on hyaluronan and collagen-I production by pancreatic stellate cells. This was associated with a reduction of AT1/PDGF-β expression and TGF-β/SMAD-2 signaling, consistent with the role of AT1 signaling in collagen-I and hyaluronan synthesis. Furthermore, metformin reduced the recruitment of tumor-associated macrophages and their expression of M2 markers in vivo at clinically relevant doses.

In certain embodiments disclosed herein, metformin treatment alleviates desmoplasia in pancreatic tumors in overweight and obese patients with a body mass index (BMI) greater than 25, but not in normal weight patients. This demonstrates that BMI can be used as a biomarker for predicting the efficacy of metformin treatment and identifying or selecting patients that will respond to metformin treatment. Thus, methods are provided herein for assessing or selecting patients that would respond to, e.g., derive therapeutic benefit from, metformin treatment or a combination therapy comprising metformin and another therapeutic agent.

Thus, methods and compositions for improving the efficacy of therapeutics, e.g., cancer therapeutics, liver disorder therapies, and fibrotic disorder therapies, are disclosed. Methods and compositions for treating or preventing a cancer (e.g., a solid tumor such as a desmoplastic tumor) by administering to a subject a metformin agent in combination with one or more anti-hypertensive and/or collagen modifying (AHCM) agent, anti-angiogenic agent, anti-inflammatory agent, and/or immune checkpoint inhibitor are disclosed. Methods for identifying or selecting patients to receive the combination therapies comprising administration of a metformin agent and one or more AHCM agent, anti-angiogenic agent, anti-inflammatory agent, and/or immune checkpoint inhibitor, are also disclosed.

Certain terms are first defined.

“About” and “approximately” shall generally mean an acceptable degree of error for the quantity measured given the nature or precision of the measurements. Exemplary degrees of error are within 20 percent (%), typically, within 10%, and more typically, within 5%, 4%, 3%, 2% or 1% of a given value or range of values.

“Delivery,” as used herein in the context of delivery of an agent(s) to a tumor, refers to the placement of the agent(s) in sufficient proximity to one or more (or all) of: the tumor vasculature, the tumor interstitial matrix, or tumor cells or tumor-associated cells (e.g., fibroblasts), to have a desired effect. The agent(s) can be, e.g., a cancer therapy (e.g., a cancer therapeutic agent(s) as described herein), or a diagnostic or imaging agent(s). Unless noted otherwise, the term “agent” or “agent(s)” as used generically herein can include one, two or more agents.

In one embodiment, the cancer therapeutic agent includes, e.g., one or more of a small molecule, a protein or a nucleic acid drug, an oncolytic virus, a vaccine, an antibody or a fragment thereof, or a combination thereof. The cancer therapeutic agent can be “free” or packaged or formulated into a delivery vehicle, e.g., a particle, e.g., a nanoparticle (e.g., a lipid nanoparticle, a polymeric nanoparticle, or a viral particle). Delivery of a therapeutic agent is characterized by placement of the therapeutic agent in sufficient proximity to the cell to alter an activity of the cell, e.g., to modulate the microenvironment around the cell, kill the cell, and/or reduce its ability to divide.

In embodiments, increased (or improved) delivery (as compared with a delivery which is the same or similar except that it is carried out in the absence of a metformin agent) can include one or more of:

increased delivery to, or amount or concentration in, the tumor vasculature, of the agent;

increased delivery to, or amount or concentration in, the tumor, e.g., the tumor vasculature interstitial matrix, of the agent;

increased delivery to, or amount or concentration in, in the tumor cells or tumor-associated cells (e.g., fibroblasts), of the agent;

increased flow rate, e.g., of the agent, in the tumor vasculature;

improved (or normalized) vasculature morphology (e.g., less tumor-like);

decompression of tumor vasculature;

increased pore size, or rate of diffusion of the agent, in the tumor, e.g., in the interstitial matrix;

increased perfusion of the agent, in the tumor, e.g., in the interstitial matrix;

broader and/or more homogeneous distribution of the agent throughout the tumor;

broader and/or more homogeneous distribution of the agent throughout the tumor interstitial matrix;

increased proportion of the agent in the tumor, e.g., the tumor interstitial matrix, as opposed to non-tumor tissue, e.g., peripheral blood;

inhibition of the TGF-beta pathway in the tumor, e.g., in the tumor vasculature interstitial matrix;

inhibition of the CTGF pathway in the tumor, e.g., in the tumor vasculature interstitial matrix;

inhibition of activity of the angiotension-II type-1 receptor;

decrease in fibrosis, in the tumor, e.g., the tumor vasculature interstitial matrix;

decrease in the level or production of an extracellular matrix component, such as a fiber (e.g., collagen, procollagen), and/or a polysaccharide (e.g., a glycosaminoglycan such as hyaluronan or hyaluronic acid);

decrease in collagen or collagen deposition, in the tumor, e.g., the tumor vasculature interstitial matrix; or

decrease hyaluronan levels in the tumor, e.g., the tumor vasculature interstitial or stromal matrix.

In some embodiments, increased (or improved) delivery (as compared with a delivery which is the same or similar except that it is carried out in the absence of a metformin agent) can also include increased amount of the agent distributed to at least a portion of the tumor. In some embodiments, the increased amount of the agent delivered to the tumor in the presence of a metformin agent can be distributed homogenously or heterogeneously throughout the tumor.

“Efficacy” as used herein in the context of therapy, e.g., cancer therapy, can be characterizes as the extent to which a therapy has a desired effect, including but not limited to, alleviation of a symptom, diminishment of extent of disease, stabilized state of disease, delay or slowing of disease progression, amelioration or palliation of the disease state, and remission (whether partial or total), whether detectable or undetectable.

Improved efficacy, in the context of efficacy of cancer therapy, can be characterized by one or more of the following: an increase in an anti-tumor effect, of the cancer therapy, and/or a lessening of unwanted side effects (e.g., toxicity), of the cancer therapy, as compared with a treatment which is the same or similar except that it is carried out in the absence of treatment with a metformin agent. In one embodiment, the increase in the anti-tumor effect of the cancer therapy includes one or more of: inhibiting primary or metastatic tumor growth; reducing primary or metastatic tumor mass or volume; reducing size or number of metastatic lesions; inhibiting the development of new metastatic lesions; reducing one or more of non-invasive tumor volume or metabolism; providing prolonged survival; providing prolonged progression-free survival; providing prolonged time to progression; and/or enhanced quality of life.

In some embodiments, the term “improved efficacy” as used herein, with respect to a cancer therapy in combination with a metformin agent, can refer to an increase in reduction of primary or metastatic tumor growth by at least about 5%, at least about 10%, at least about 20%, at least about 30%, at least about 40%, at least about 50%, at least about 60%, at least about 70%, at least about 80%, at least about 90%, at least about 95%, up to and including 100%, as compared to the reduction of primary or metastatic tumor growth during a cancer therapy alone (i.e., in the absence of a metformin agent). In some embodiments, the administration of a metformin agent in combination with a cancer therapy can increase the reduction of primary or metastatic tumor growth by at least about 1-fold, at least about 2-fold, at least about 3-fold, at least about 5-fold, at least about 6-fold, at least about 7-fold, or higher, as compared to the reduction of primary or metastatic tumor growth during a cancer therapy alone (i.e., in the absence of a metformin agent). Methods for monitoring tumor growth in vivo are well known in the art, e.g., but not limited to, X-ray, CT scan, MRI and other art-recognized medical imaging methods.

In some embodiments, the term “improved efficacy” as used herein, with respect to a cancer therapy in combination with a metformin agent, can refer to an increase in perfusion of an anti-cancer agent (e.g., low molecular weight therapeutics or nanotherapeutics such as DOXIL® or immune cells) into a tumor, e.g., by at least about 5%, at least about 10%, at least about 20%, at least about 30%, at least about 40%, at least about 50%, at least about 60%, at least about 70%, at least about 80%, at least about 90%, at least about 95%, up to and including 100%, as compared to perfusion of an anti-cancer agent alone (i.e., in the absence of a metformin agent). In some embodiments, the administration of a metformin agent in combination with a cancer therapy can increase perfusion of an anti-cancer agent (e.g., low molecular weight therapeutics or nanotherapeutics such as DOXIL®) into a tumor, by at least about 1-fold, at least about 2-fold, at least about 3-fold, at least about 5-fold, at least about 6-fold, at least about 7-fold, or higher, as compared to the perfusion efficiency of an anti-cancer agent alone (i.e., in the absence of a metformin agent). Methods to measure tumor perfusion in vivo are well established in the art, including, but not limited to, positron emission tomography (PET), and ultrasound or contrast-enhanced ultrasound.

In some embodiments, the term “improved efficacy” as used herein, with respect to a cancer therapy in combination with a metformin agent, can refer to an increase in reduction in expression level of at least one biomarker, e.g., at least one cancer biomarker (e.g., in a biological sample such as a blood sample, a serum sample, a plasma sample or a tissue biopsy), e.g., by at least about 5%, at least about 10%, at least about 20%, at least about 30%, at least about 40%, at least about 50%, at least about 60%, at least about 70%, at least about 80%, at least about 90%, at least about 95%, up to and including 100%, as compared to the reduction in expression level of the at least one cancer biomarker when administered with a cancer therapy, alone (i.e., in the absence of a metformin agent). In some embodiments, the administration of a metformin agent in combination with a cancer therapy can increase the reduction in expression level of at least one biomarker (e.g., in a biological sample such as a blood sample, a serum sample, a plasma sample or a tissue biopsy) by at least about 1-fold, at least about 2-fold, at least about 3-fold, at least about 5-fold, at least about 6-fold, at least about 7-fold, or higher, as compared to the reduction in expression level of the at least one cancer biomarker when administered with a cancer therapy alone (i.e., in the absence of a metformin agent). Examples of a biomarker in the serum, plasma or tissue can include, but are not limited to, TGF-beta 1, TGF-beta 2, CTGF, TSP-1, hyaluronic acid (HA), collagen I, collagen II, collagen III, or collagen IV. Expression levels of biomarkers can be measured on a transcript level and/or a protein level, using any art-recognized analytical methods, e.g., PCR, western blot, ELISA, and/or immunostaining.

“Blood pressure” is usually classified based on the systolic and diastolic blood pressures. “Systolic blood pressure” or Psys refers to the blood pressure in vessels during a heart beat. “Diastolic blood pressure” or Pdias refers to the pressure between heartbeats. A systolic or the diastolic blood pressure measurement higher than the accepted normal values for the age of the individual is classified as prehypertension or hypertension. A systolic or the diastolic blood pressure measurement lower than the accepted normal values for the age of the individual is classified as hypotension. A “normal” systolic pressure for an adult is typically in the range of 90-120 mmHg; a “normal” diastolic pressure is usually in the range of 60-80 mmHg. In the population, the average blood pressure (Psys/Pdias ratio) can range from 110/65 to 140/90 mmHg for an adult; 95/65 mmHg for a 1 year infant, and 100/65 mmHg for a 6-9 year old.

Hypertension has several subclassifications including, prehypertension (120/80 to 139/89 mmHg); hypertension stage I (140/90 to 159 to 99 mmHg), hypertension stage II (greater or equal to 160/100 mmHg, and isolated systolic hypertension (greater or equal to 140/90 mmHg). Isolated systolic hypertension refers to elevated systolic pressure with normal diastolic pressure and is common in the elderly. These classifications are made after averaging a patient's resting blood pressure readings taken on two or more office visits.

Hypertension is generally diagnosed on the basis of a persistently high blood pressure.

Usually this requires three separate sphygmomanometer measurements at least one week apart. Often, this entails three separate visits to the physician's office. Initial assessment of the hypertensive patient should include a complete medical history and physical examination.

As used herein, “hypertension” or “high blood pressure,” refers to a prehypertensive or a hypertensive stage having a systolic pressure of 120 or greater (typically, 140 or greater) and a diastolic pressure of 80 or greater (all blood pressures herein are expressed as mmHg).

As used herein, the term “mean arterial pressure” (MAP) is art recognized and refers to the average over a cardiac cycle and is determined by the cardiac output (CO), systemic vascular resistance (SVR), and central venous pressure (CVP), MAP=(CO×SVR)+CVP. MAP can be approximately determined from measurements of the systolic pressure (Psys) and the diastolic pressure (Pdias), while there is a normal resting heart rate, MAP is approximately Pdias+1/3 (Psys-Pdias).

“Anti-hypertensive agent,” as used herein refers to an agent (e.g., a compound, a protein) that when administered at a selected dose (referred to herein as “an anti-hypertensive dose”) reduces blood pressure, typically in a patient (e.g., a hypertensive patient). Anti-hypertensive agents are routinely used clinically to treat patients with high blood pressure at doses known in the art. Exemplary anti-hypertensive agents, include but are not limited to, renin angiotensin aldosterone system antagonists (“RAAS antagonists”), angiotensin converting enzyme (ACE) inhibitors, and angiotensin II receptor blockers (AT₁ blockers). Exemplary anti-hypertensive doses of some of these agents are also disclosed herein.

“Sub-anti-hypertensive dose,” as used herein, refers to a dose of an anti-hypertension agent that is typically less than the lowest dose that would be used to treat a patient for high blood pressure. In an embodiment, a sub-anti-hypertensive dose has one or more of the following properties:

it does not substantially lower blood pressure, e.g., the mean arterial blood pressure, of the subject, e.g., a hypertensive subject;

it reduces mean arterial blood pressure in the subject by less than 1%, 5%, 10%, 15%, 20%, 25%, 30%, 35%, 40%, 45%, 50%:

it reduces blood pressure by less than 1%, 5%, 10%, 15%, 20%, 25%, 30%, 35%, 40%, 45%, 50%, 60%, 70%, 80%, 90%, or less of the reduction caused by a standard of care anti-hypertensive dose for that AHCM;

it is less than 1%, 5%, 10%, 15%, 20%, 25%, 30%, 35%, 40%, 45%, 50%, 60%, 70%, 80%, 90% of the dose of that AHCM that would bring the subject's blood pressure into the normal range, e.g, 120 systolic and 80 diastolic, or a dose that would bring the subjects blood pressure into the range of to 120+/−5 systolic and 80+/−5 diastolic; or

it is less than a standard of care anti-hypertensive dose.

In certain embodiments, the ability of a dose to meet one or more of these standards can be made as measured after a preselected number of dosages, e.g., 1, 2, 5, or 10, or after sufficient dosages that a steady state level, e.g., plasma level, is attained.

An “AHCM,” as used herein, can be an agent having one or more of the following properties:

it is an antagonist of renin angiotensin aldosterone system (“RAAS antagonist”),

it is an angiotensin converting enzyme (ACE) inhibitor,

it is an angiotensin II receptor blocker (AT₁ blocker),

it is a thrombospondin 1 (TSP-1) inhibitor,

it is a transforming growth factor beta 1 (TGF-131) inhibitor,

it is an inhibitor of SDF-1a; or

it is a connective tissue growth factor (CTGF) inhibitor.

“Treating” a tumor, as used herein, typically refers to one or more of the following:

inhibiting primary or metastatic tumor growth;

reducing primary or metastatic tumor mass or volume;

reducing size or number of metastatic lesions;

inhibiting the development of new metastatic lesions;

reducing one or more of non-invasive tumor volume or metabolism;

providing prolonged survival;

providing prolonged progression-free survival;

providing prolonged time to progression; and/or enhanced quality of life.

Various aspects of the invention are described in further detail below. Additional definitions are set out throughout the specification.

### Metformin Agents

In certain embodiments, the metformin agent is a biguanide. In some embodments, the metformin agent is characterized by two linked guanidine moieties. Exemplary biguanides include, but are not limited to, metformin, phenformin, buformin, and biguanide, or any functional analog, derivative, or a salt of any of the aforesaid compounds.

In some embodiments, the metformin agent is described by a compound of Formula (I):

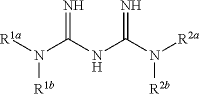

or a pharmaceutically acceptable salt thereof, wherein each of R^{1a} R^{1b}and R^{2b} is independently hydrogen, C₁-C₆ alkyl, cycloalkylalkyl, or arylalkyl.

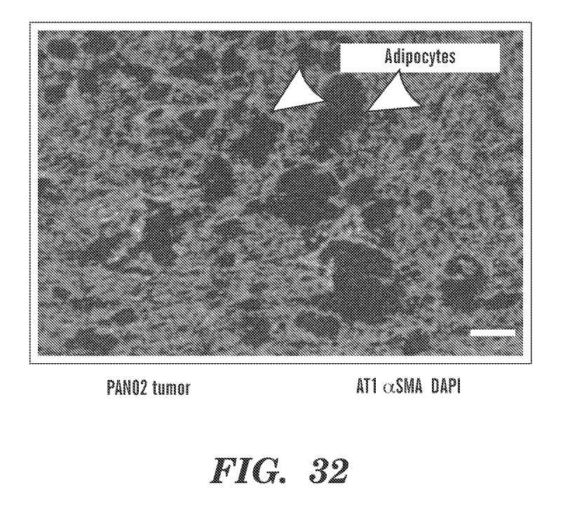

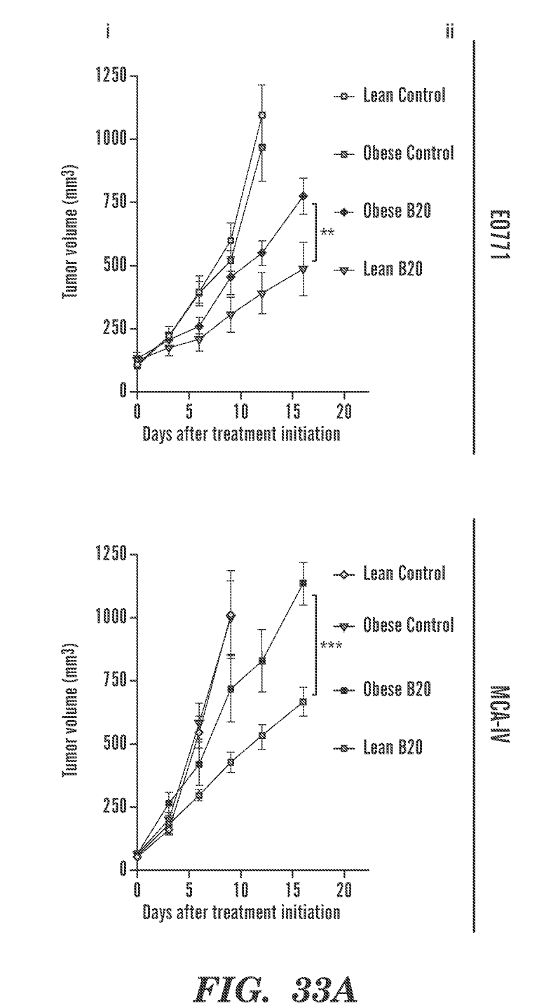

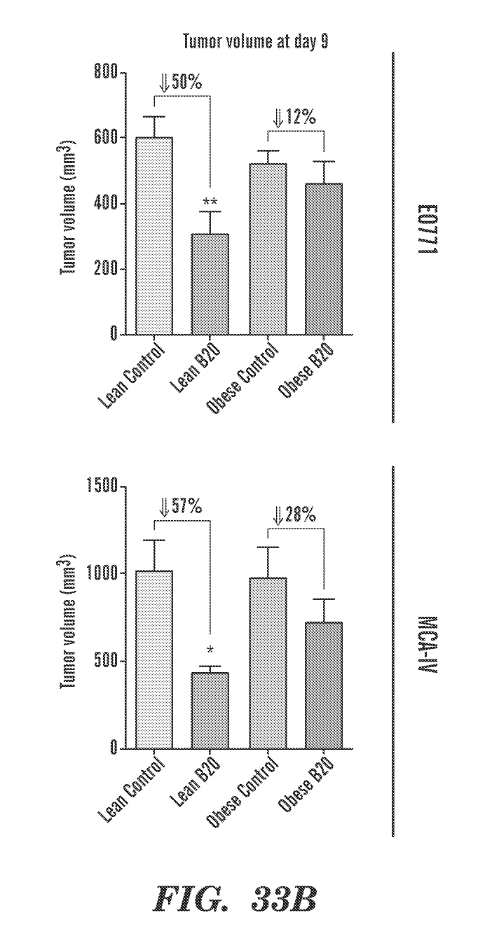

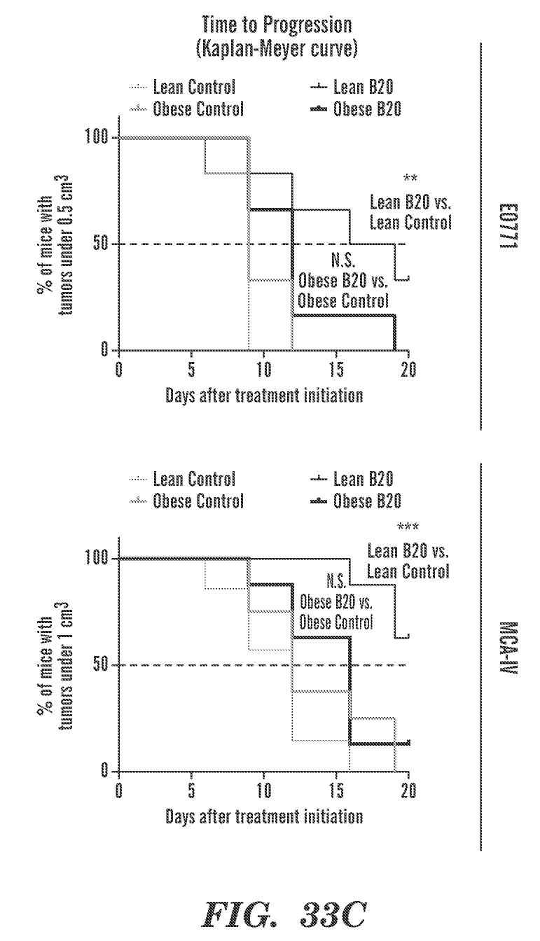

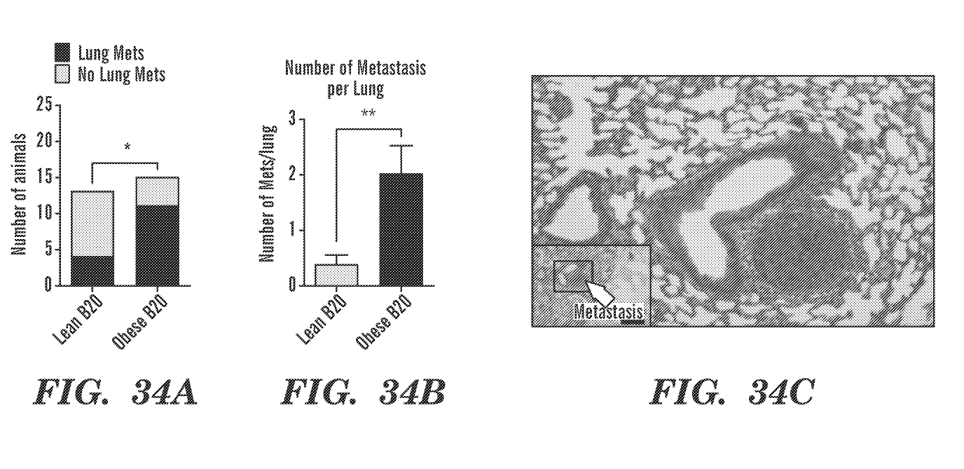

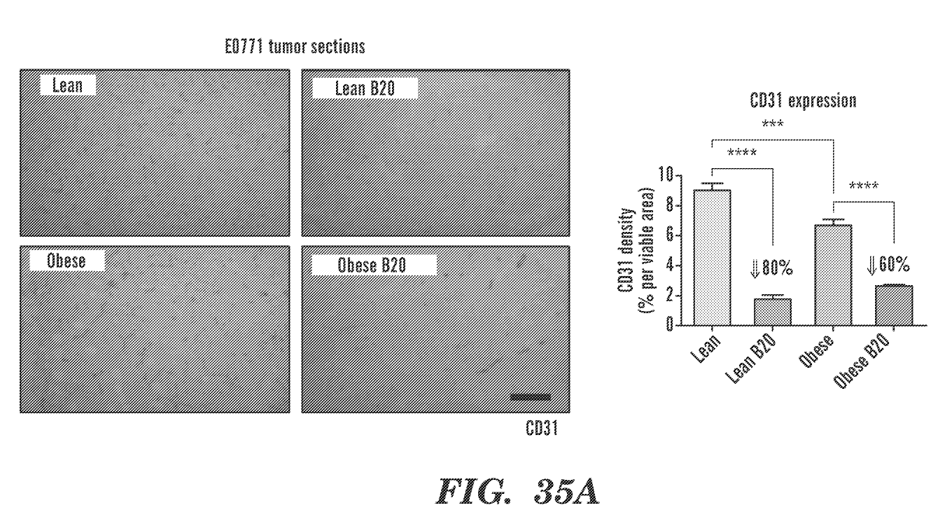

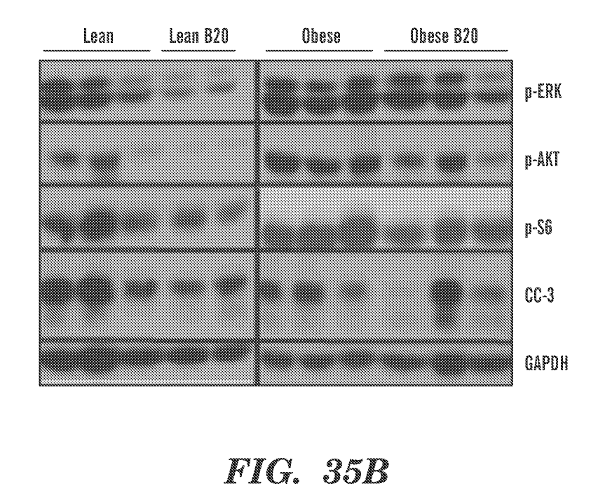

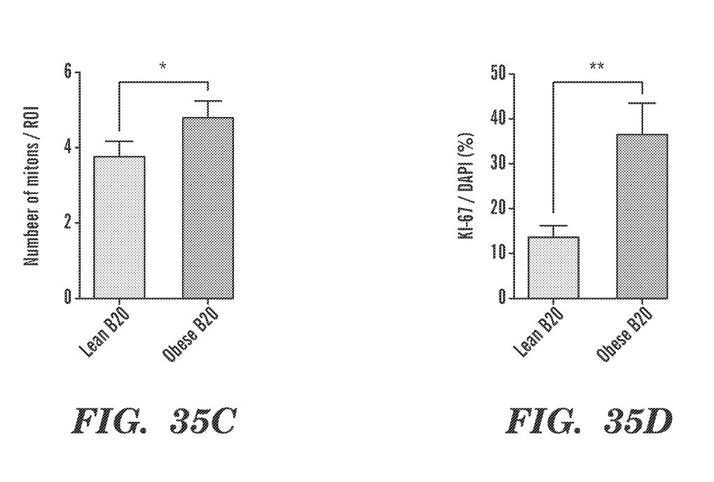

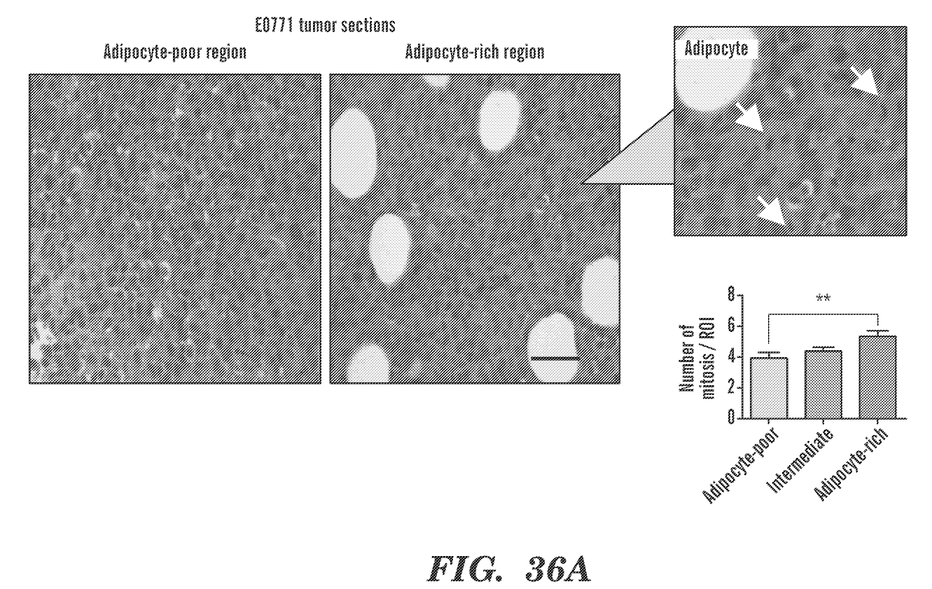

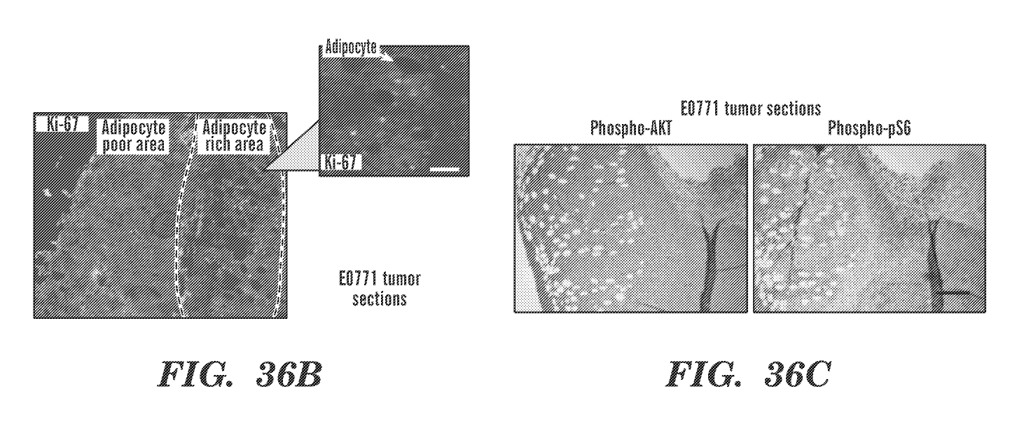

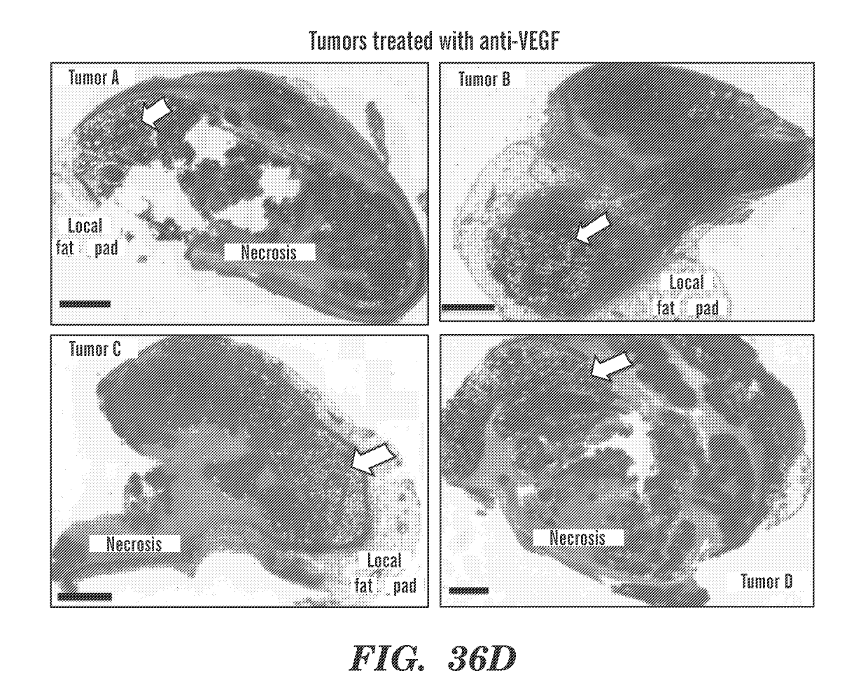

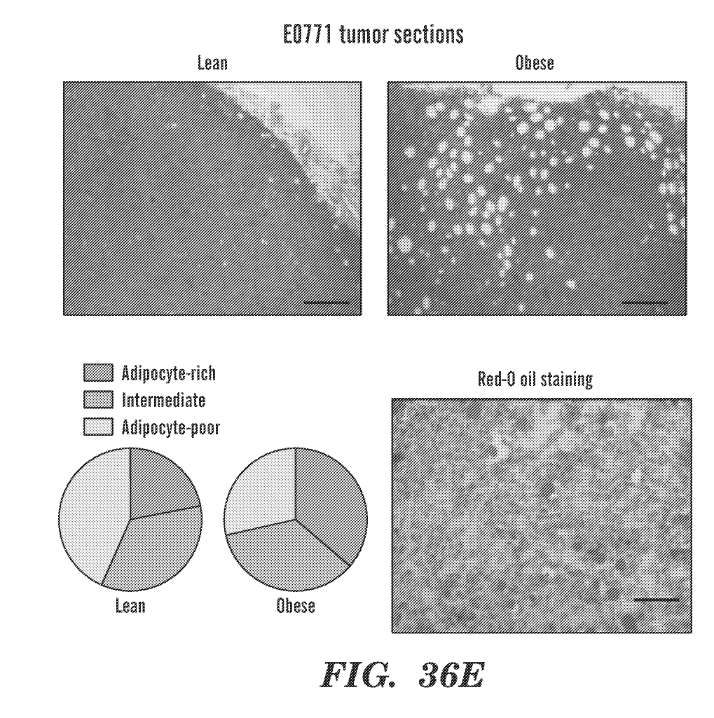

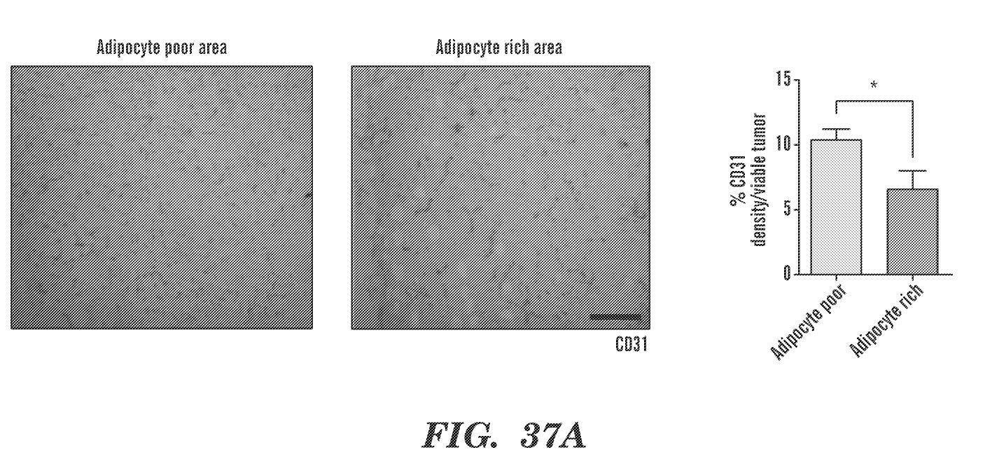

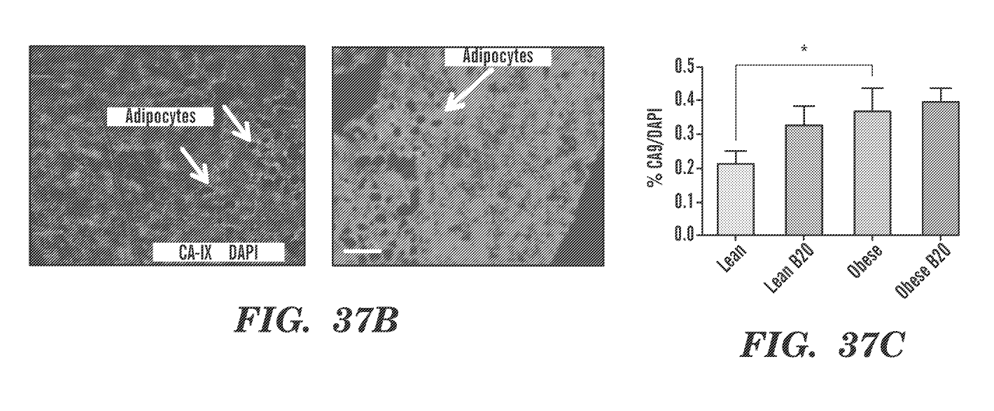

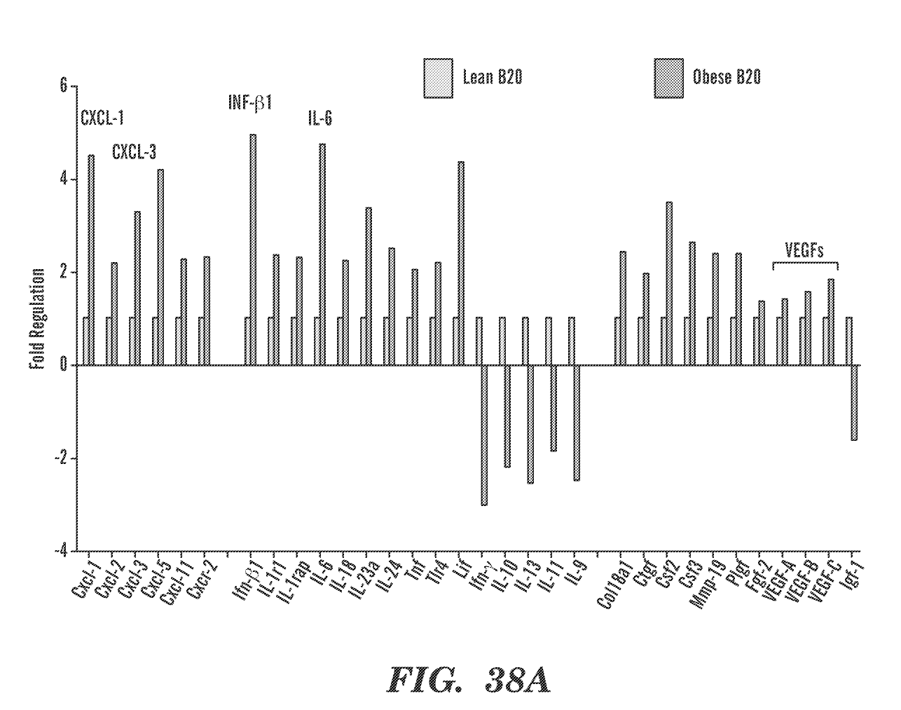

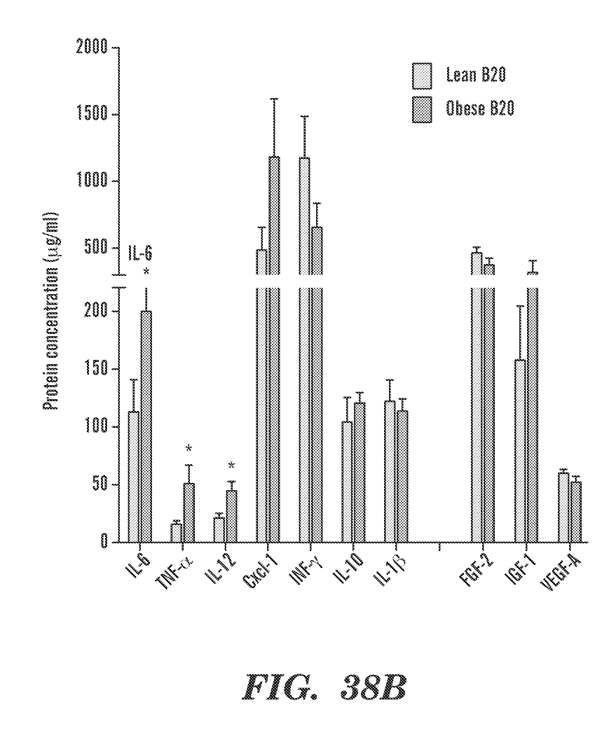

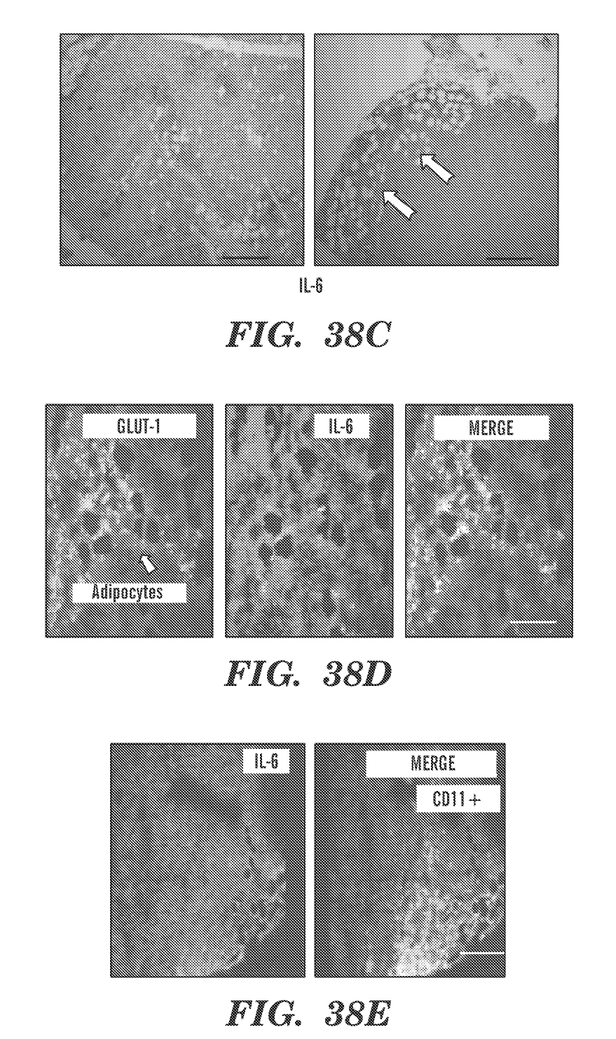

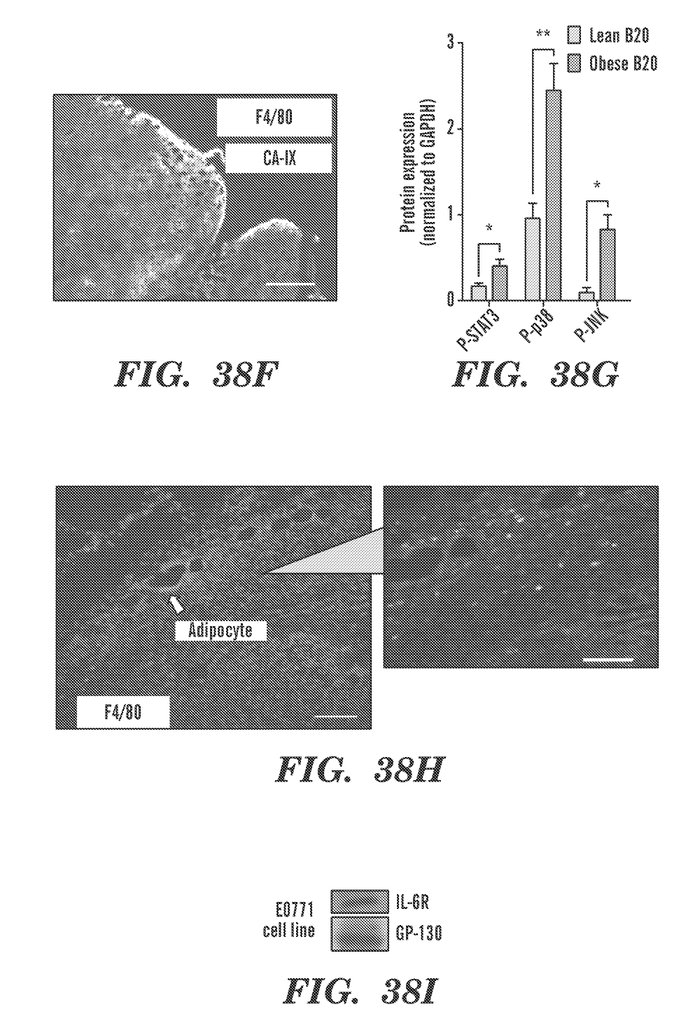

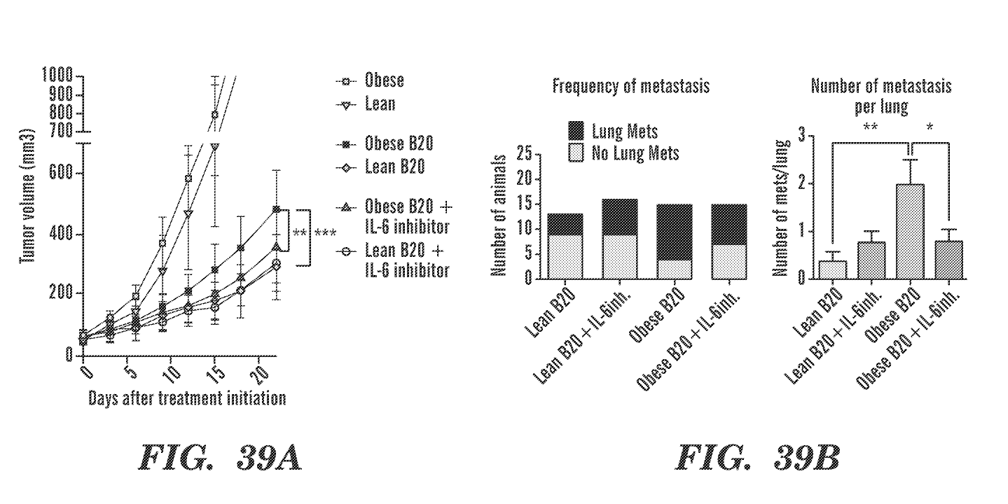

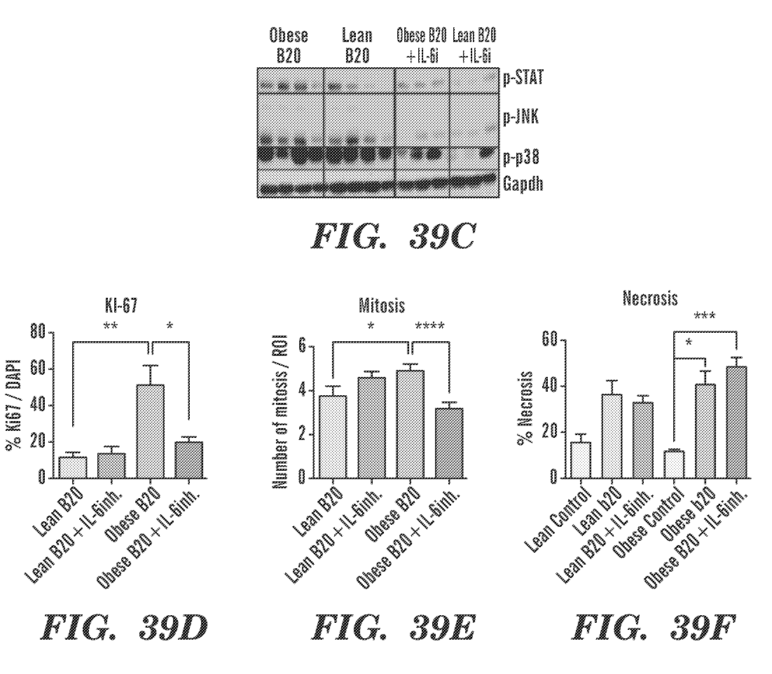

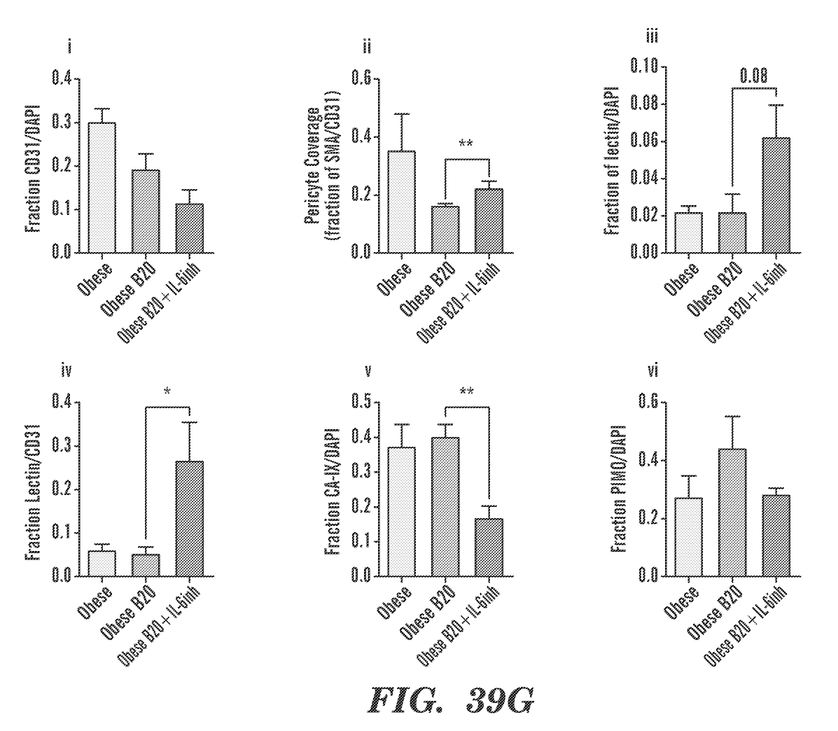

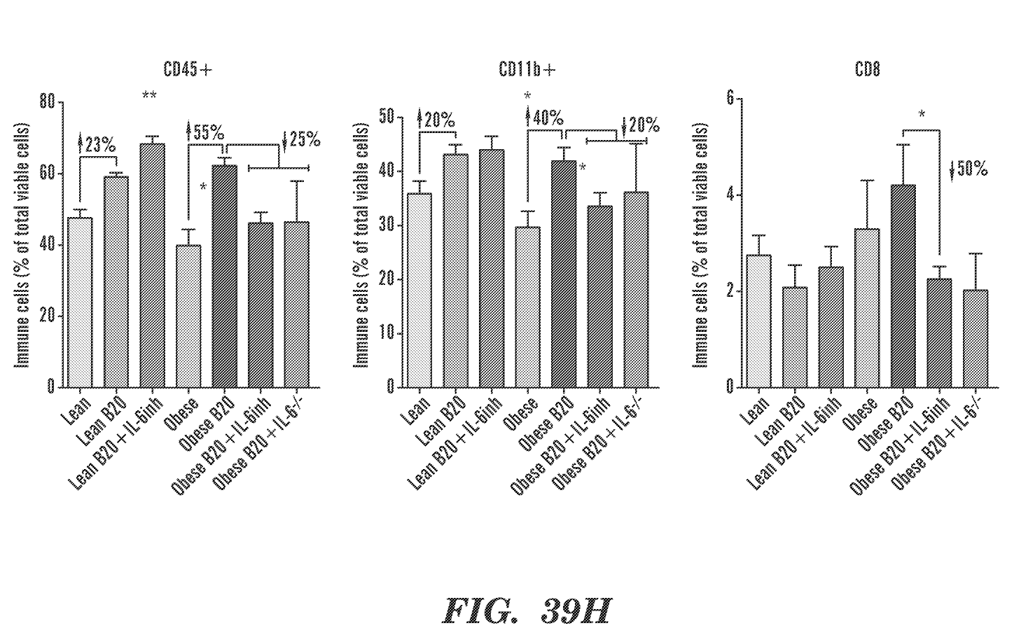

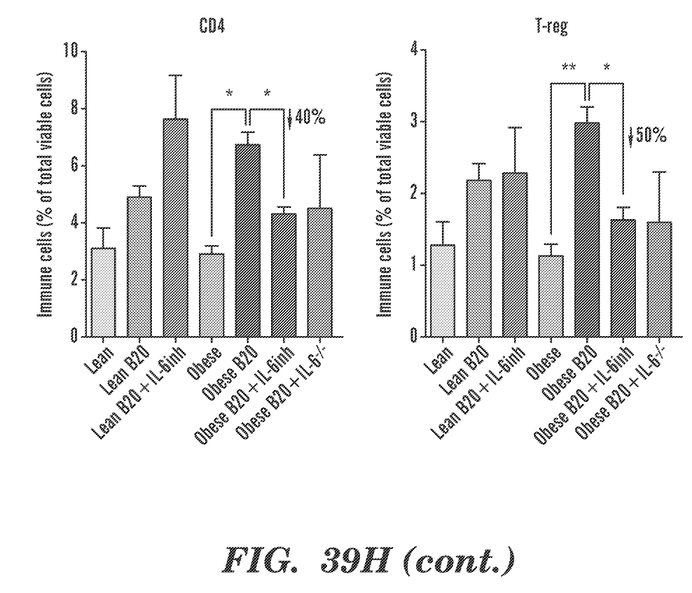

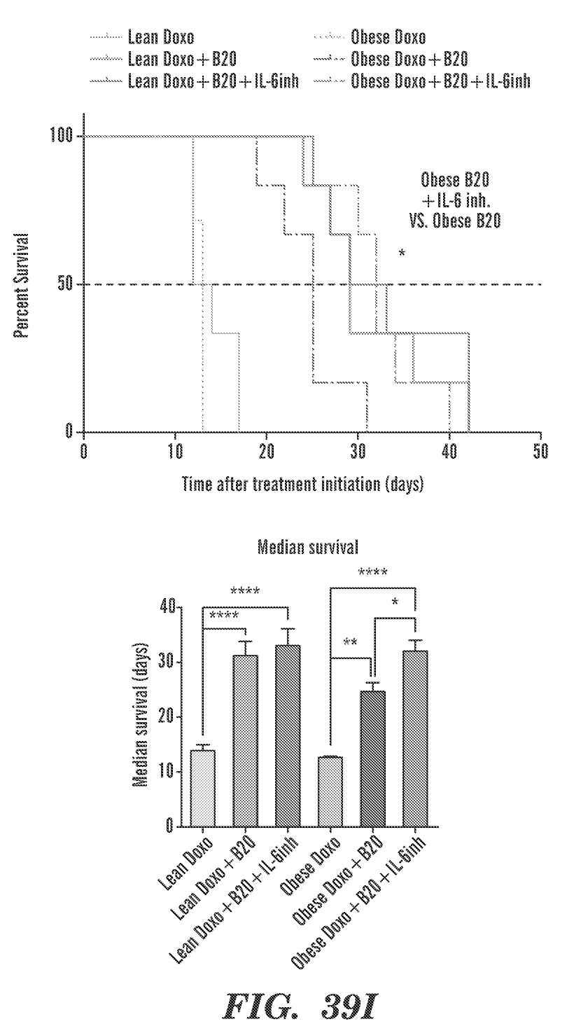

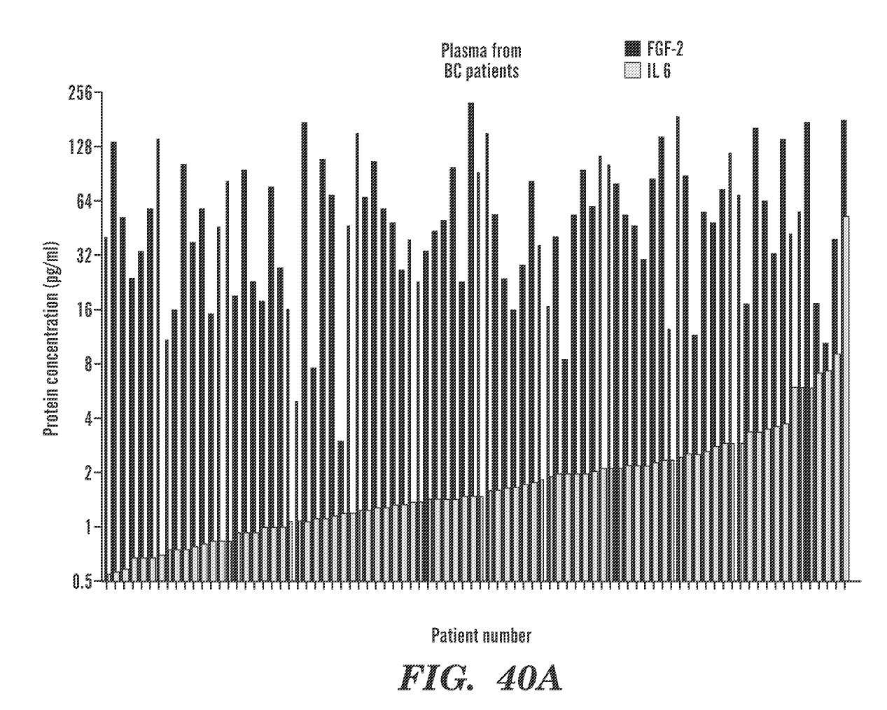

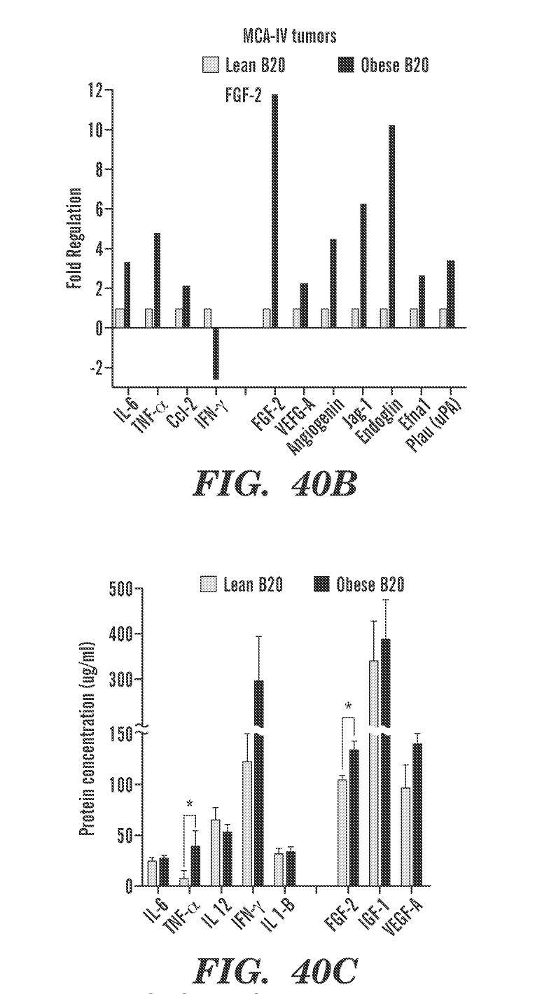

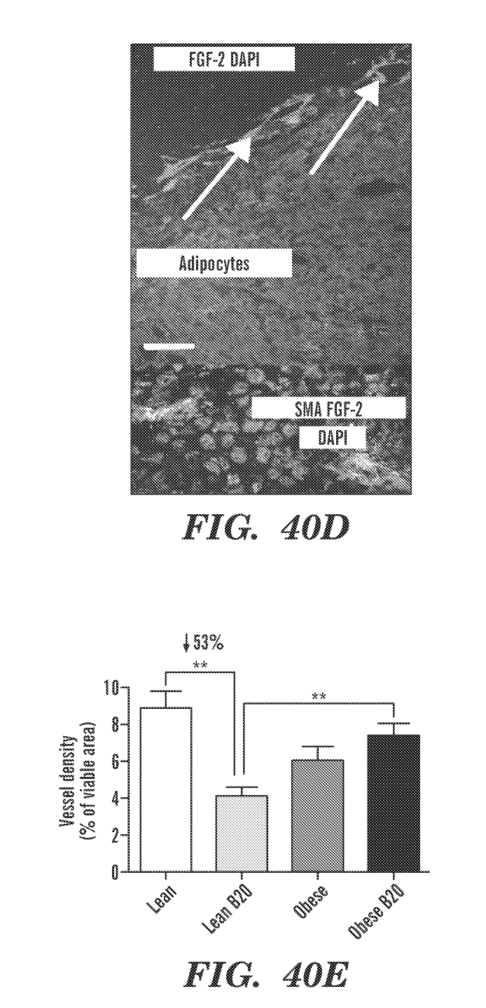

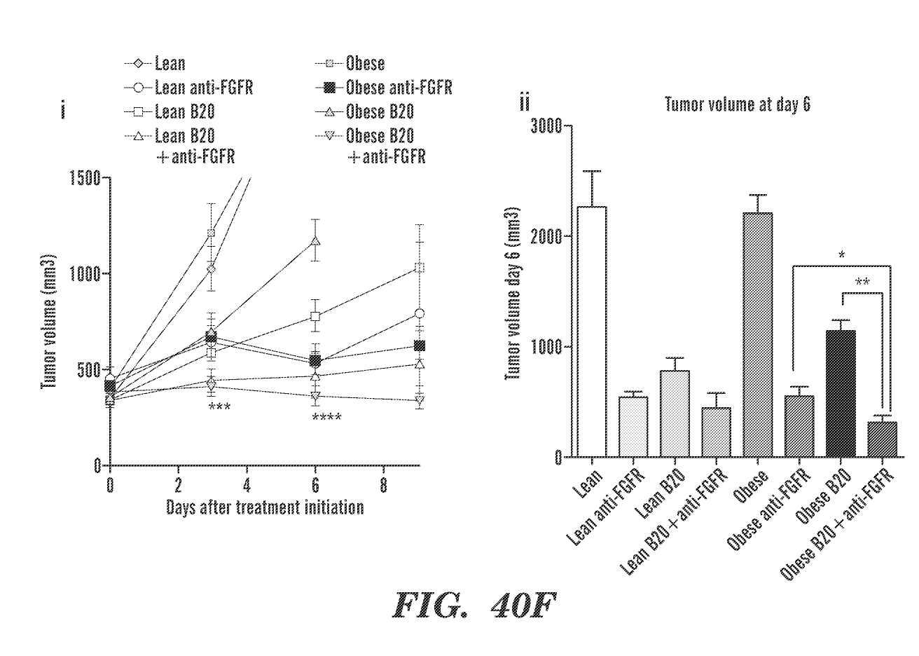

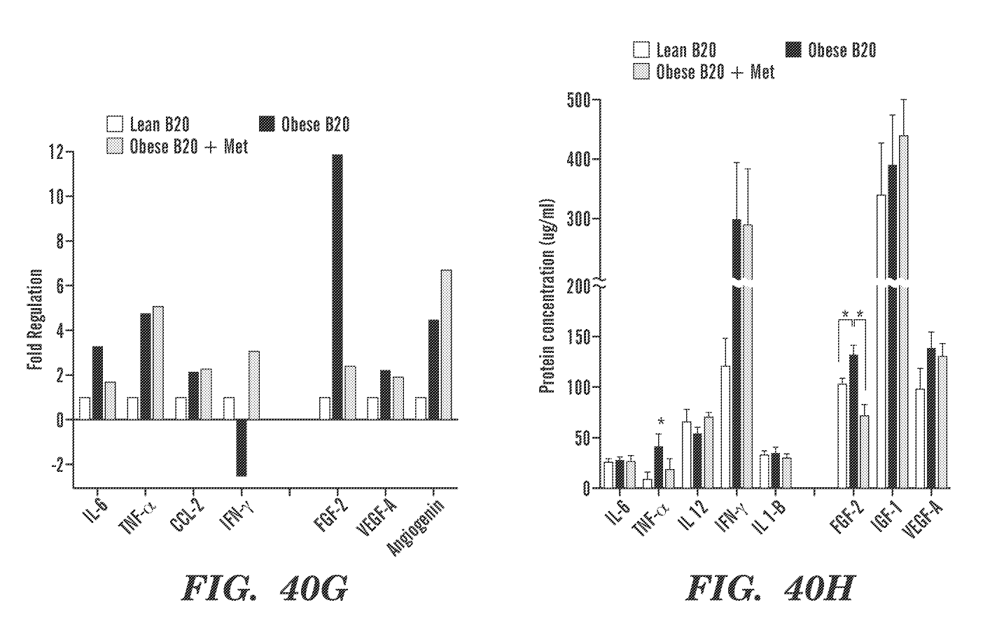

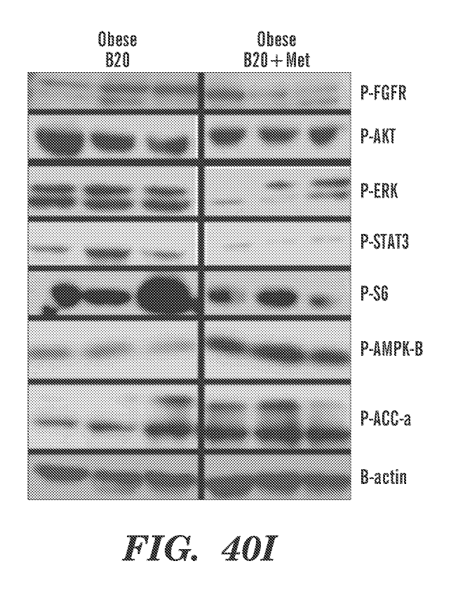

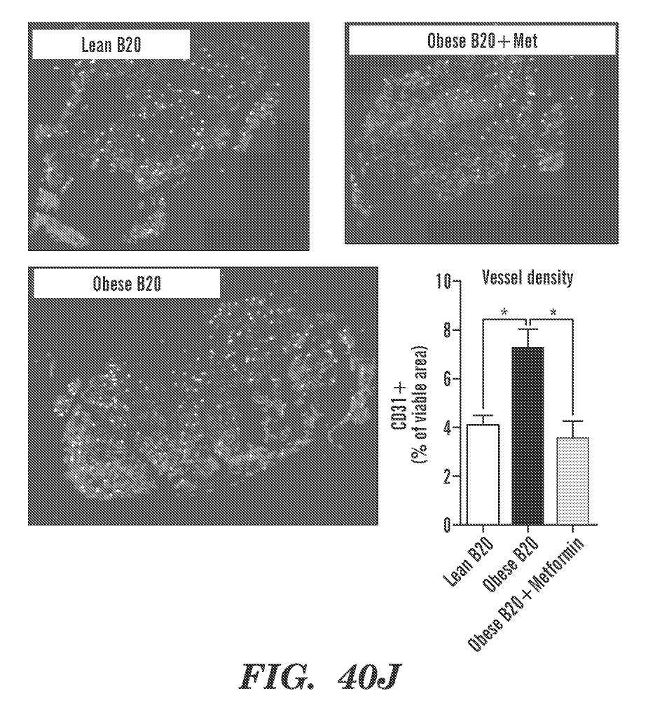

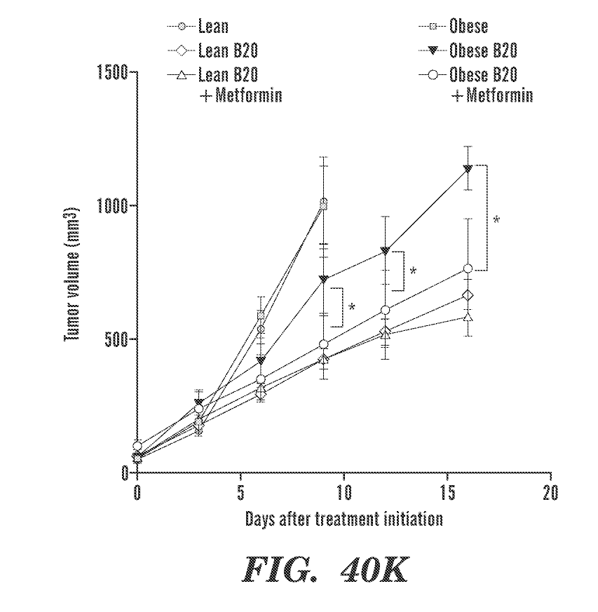

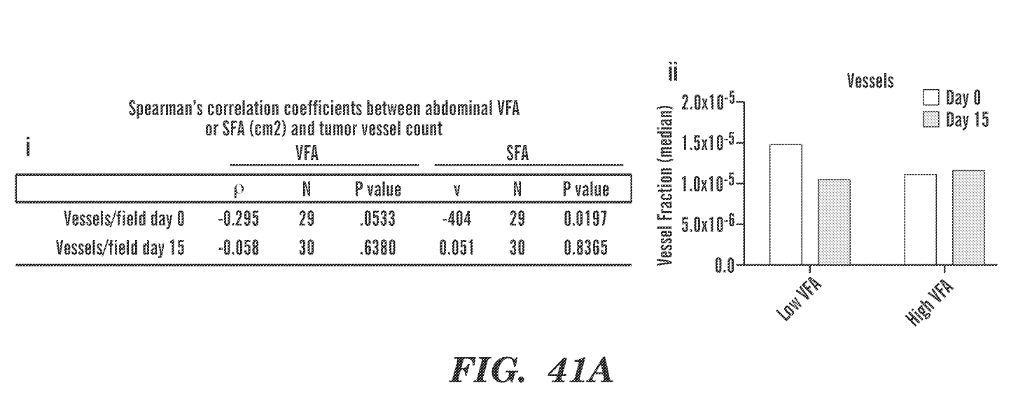

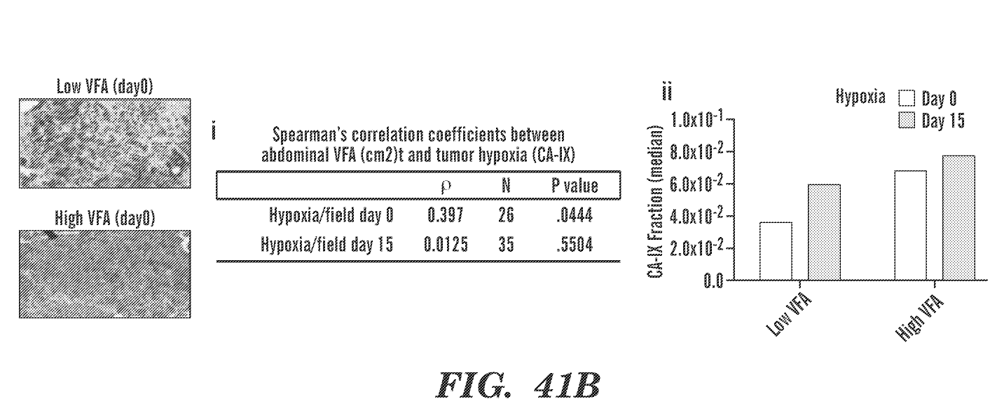

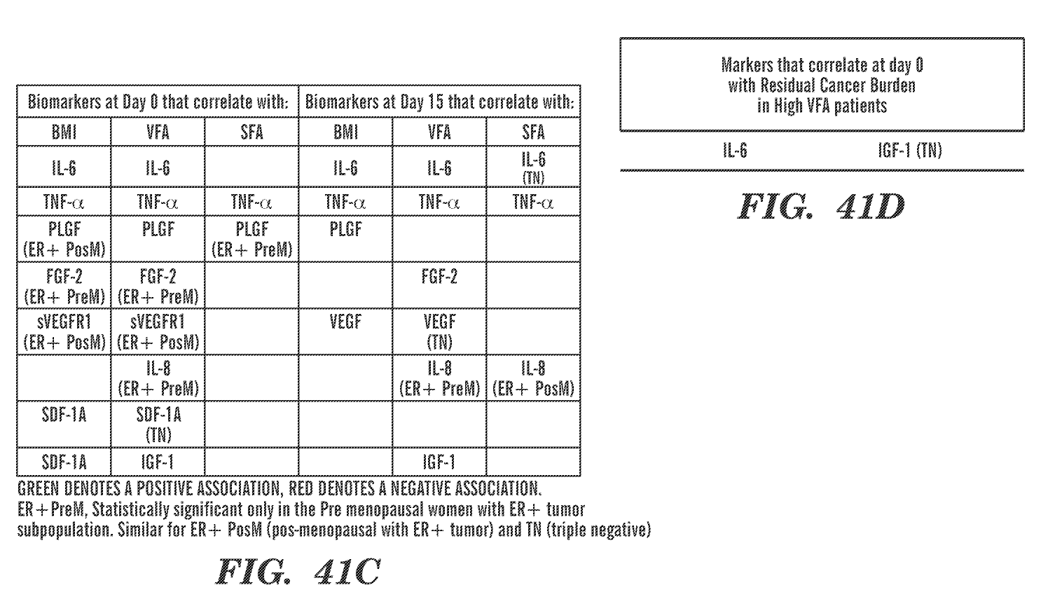

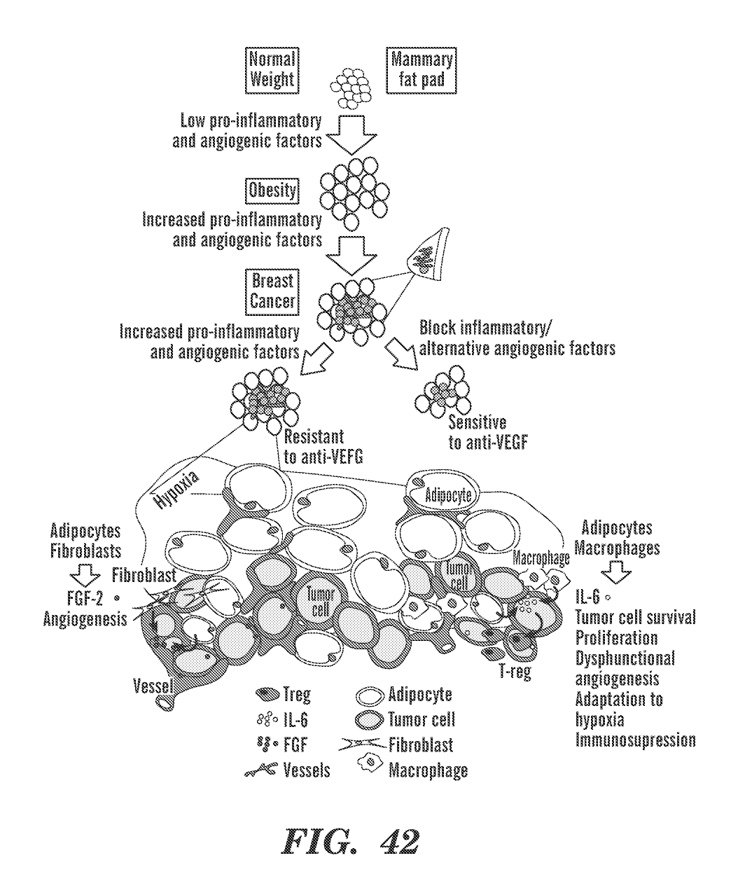

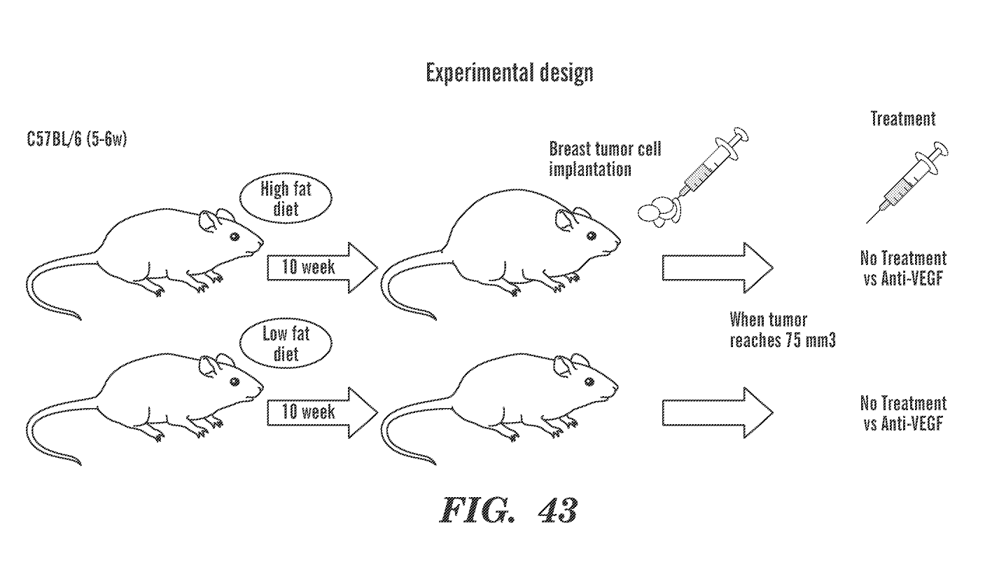

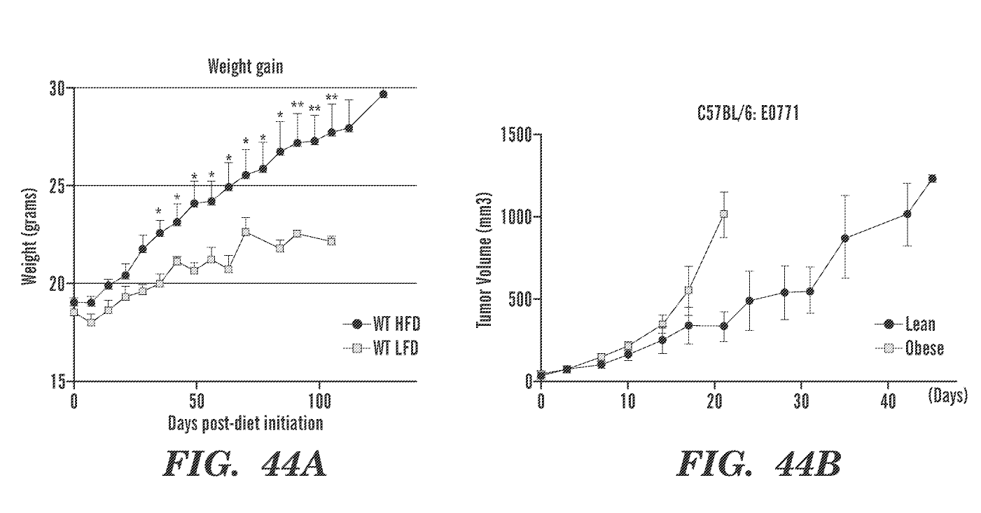

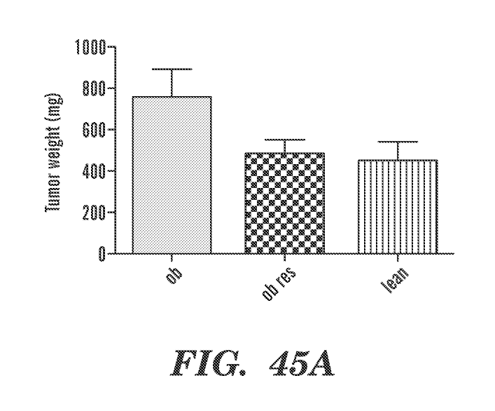

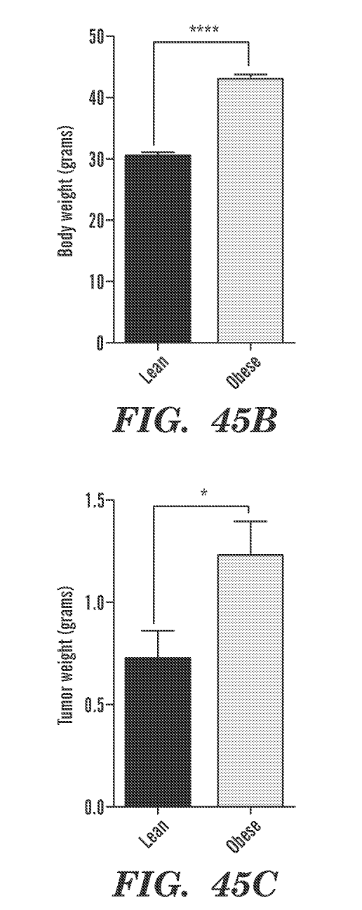

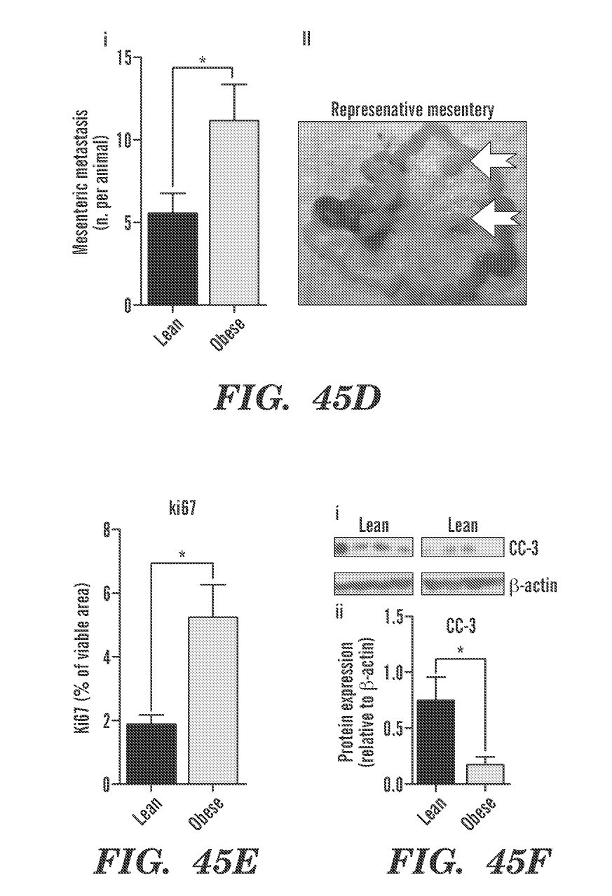

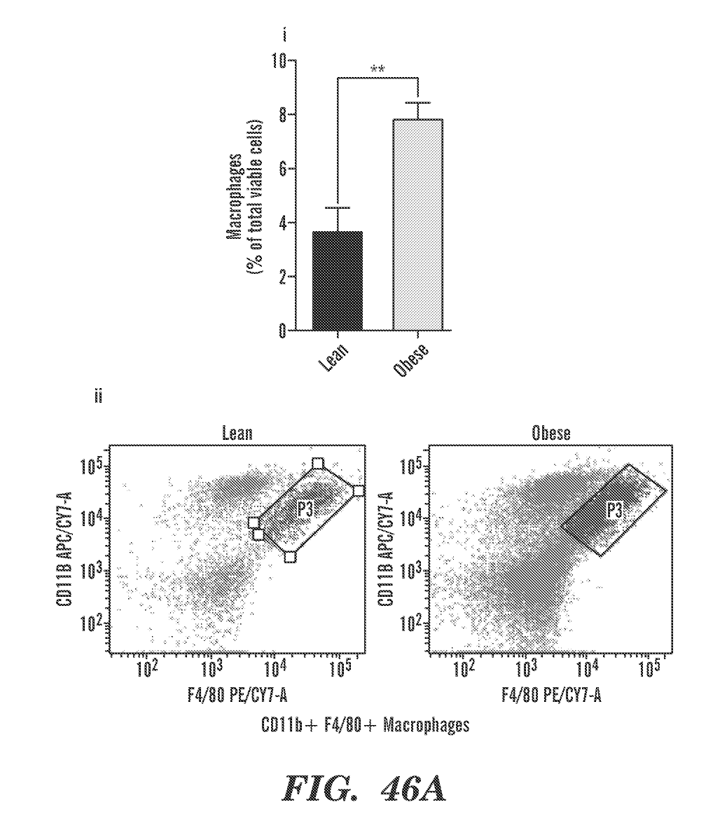

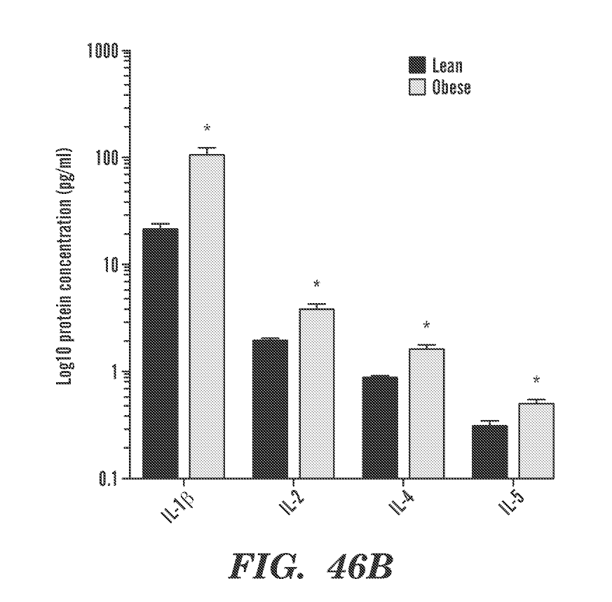

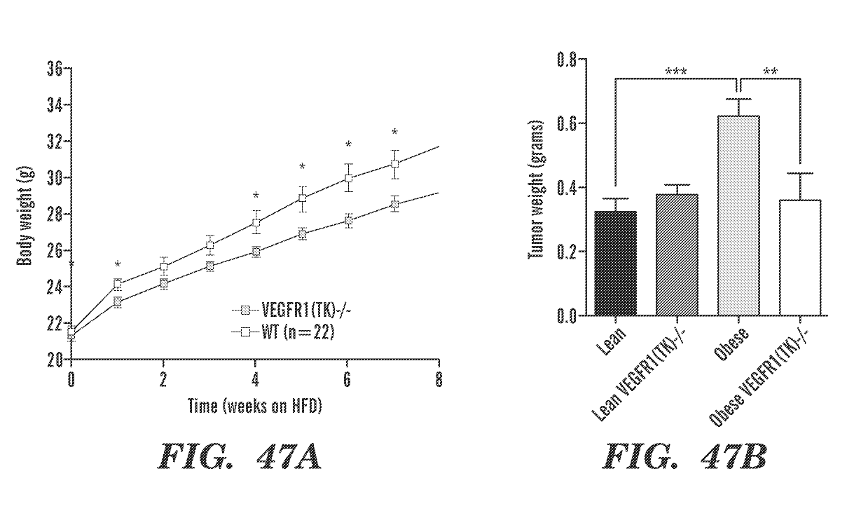

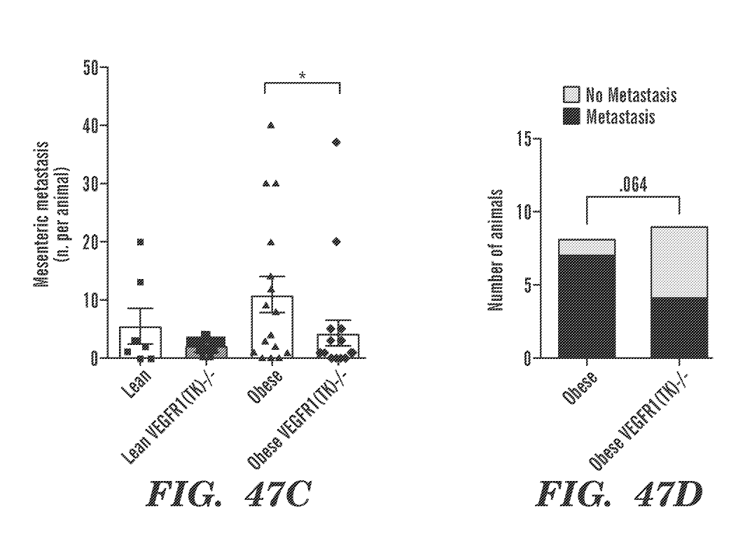

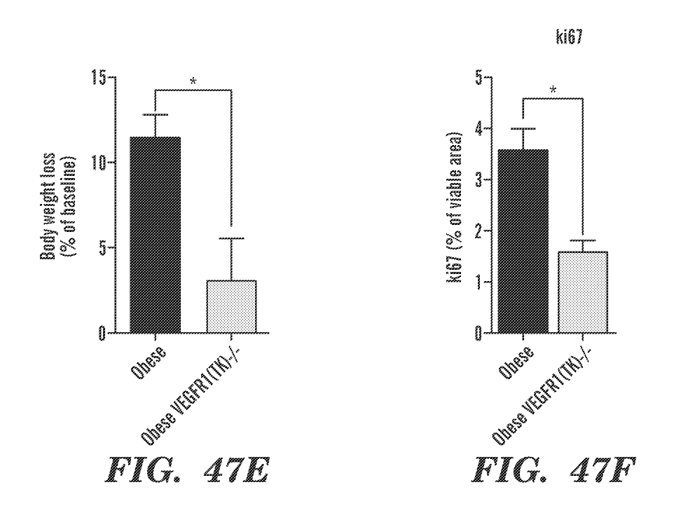

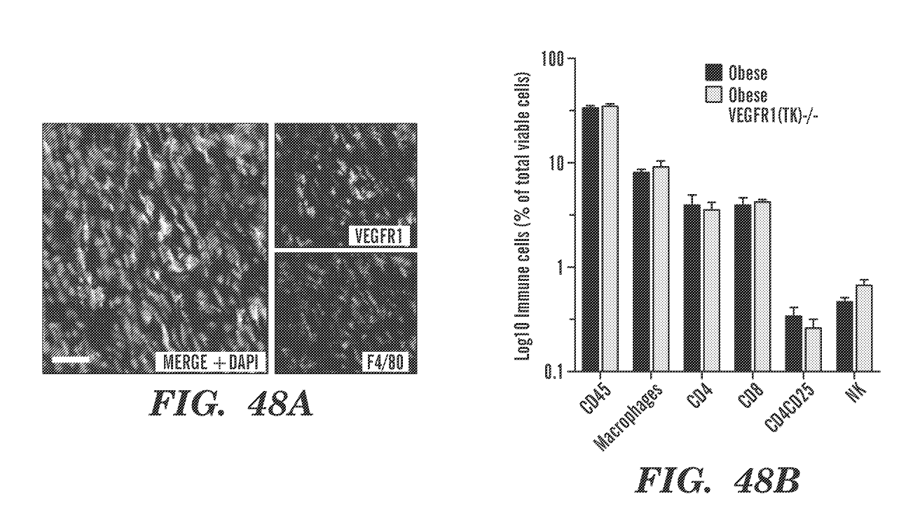

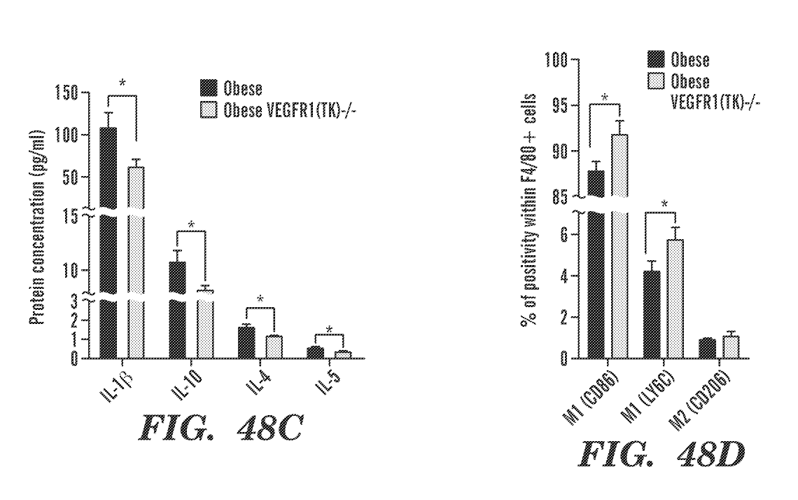

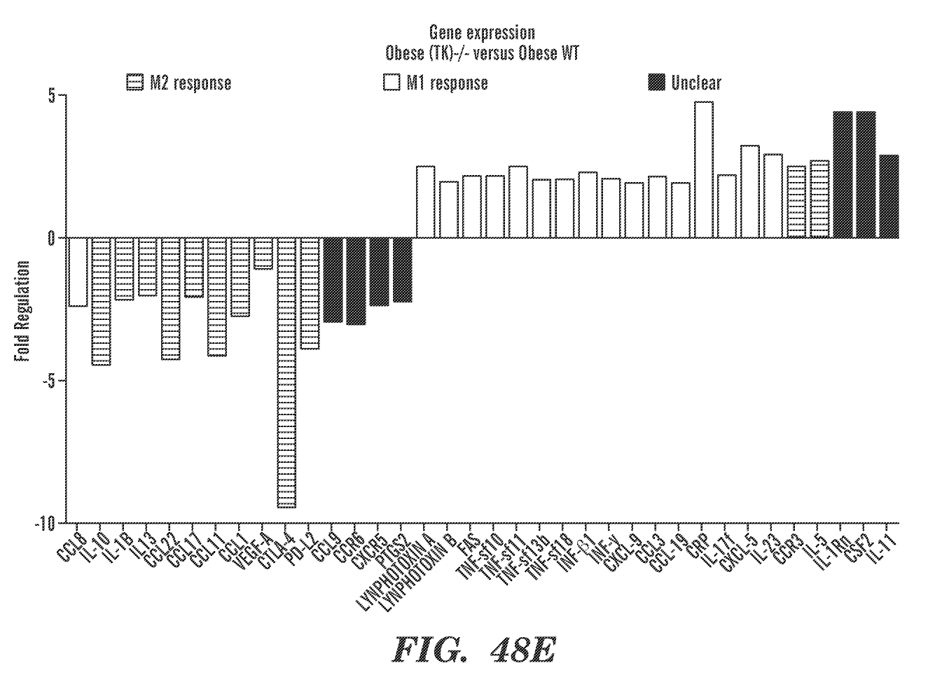

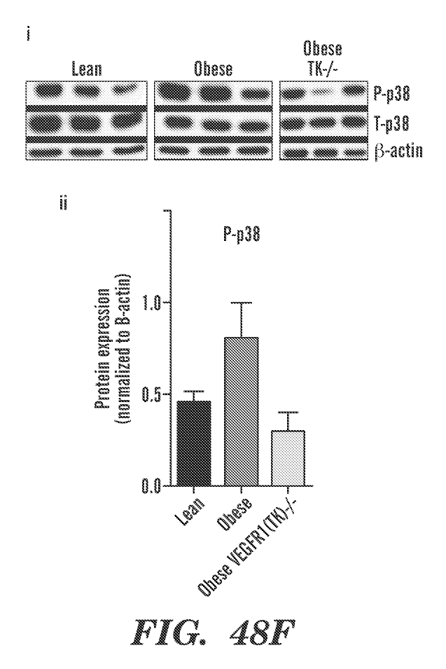

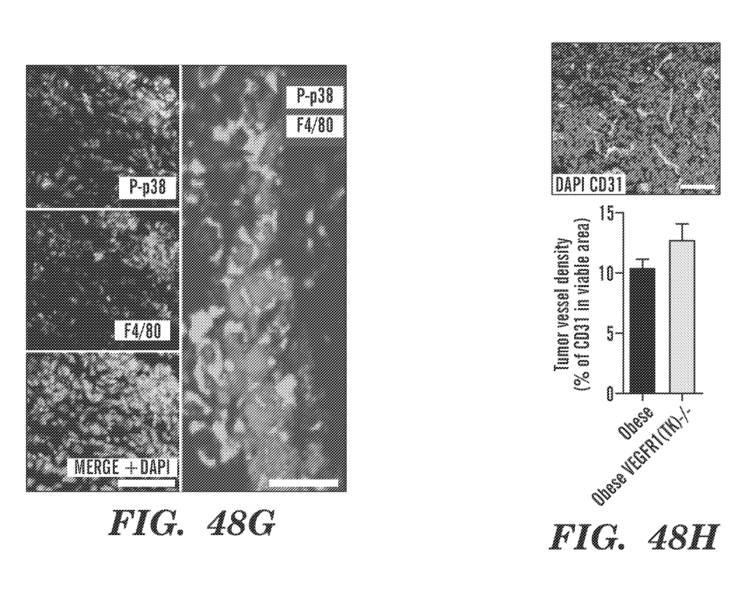

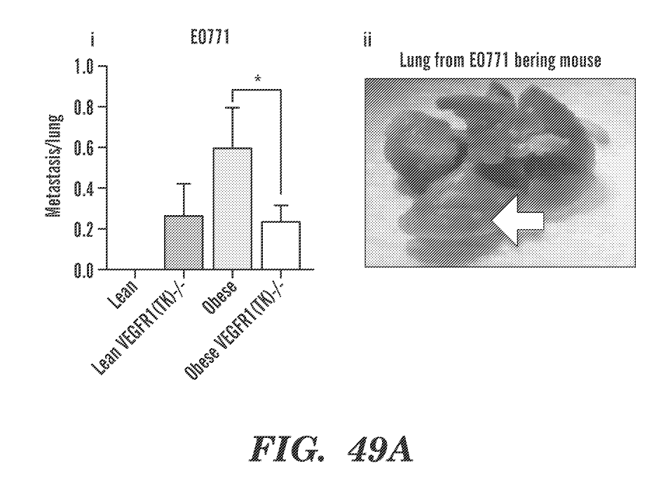

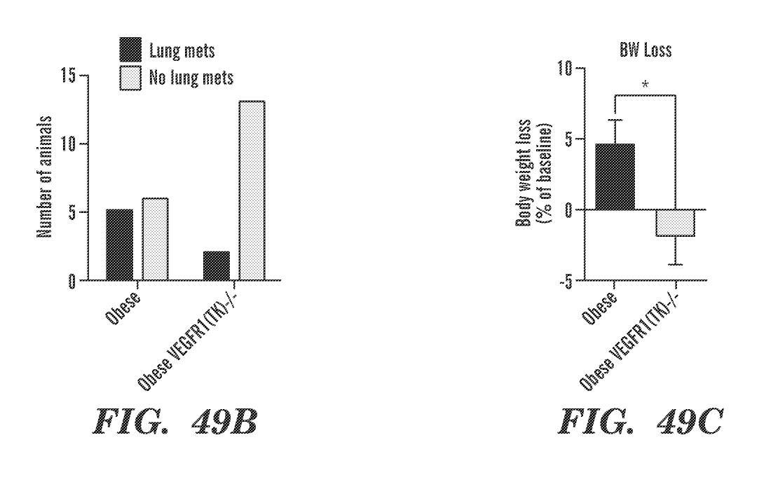

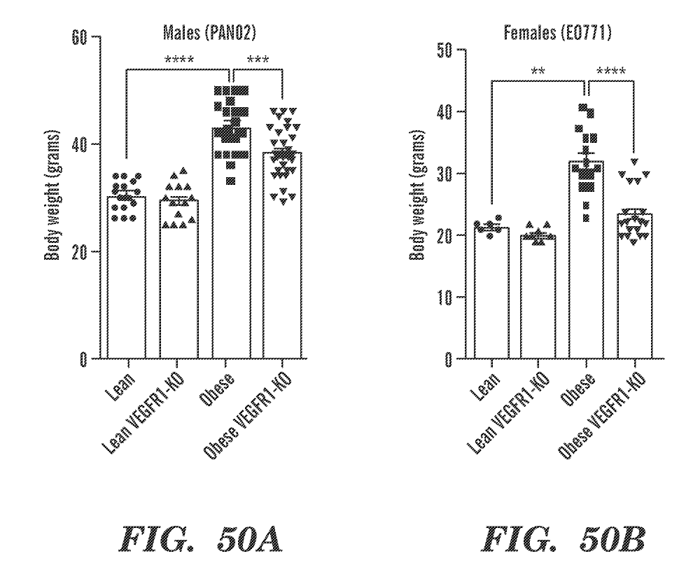

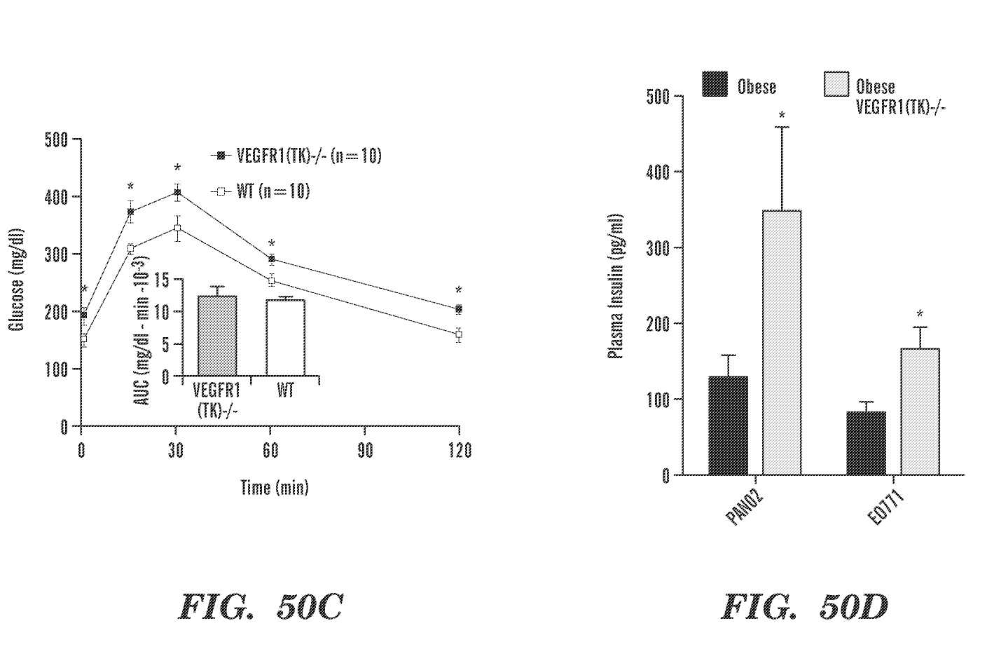

In some embodiments, each of R^{1a} and R^{1b} is hydrogen. In some embodiments, each of R^{2a} and R^{2b} is independently hydrogen or C₁-C₆ alkyl. In some embodiments, each of R^{2a} and R^{2b} is independently C₁-C₄ alkyl. In some embodiments, each of R^{2a} and R^{2b} is independently C₁-C₂ alkyl. In some embodiments, each of R^{2a} and R^{2b} is independently methyl. In some embodiments, each of R^{1a} and R^{1b} is hydrogen, and each of R^{2a} and R^{2b} is methyl. In some embodiments, the compound of Formula (I) is metformin, e.g., 3-(diaminomethylidene)-1,1-dimethylguanidine. In some embodiments, the compound of Formula (I) is metformin, e.g., as depicted in FIG. 32.

In some embodiments, each of R^{1a} and R^{1b} is hydrogen. In some embodiments, R^{2a} is hydrogen. In some embodiments, R^{2b} is arylalkyl. In some embodiments, R^{2b} is C₁-C₄ arylalkyl. In some embodiments, R^{2b} is C₁-C₂ arylalkyl. In some embodiments, R^{2b} is ethylphenyl. In some embodiments, each of R^{1a} and R^{1b} is hydrogen, R^{2a} is hydrogen, and R^{2b} is C₁-C₂ arylalkyl (e.g., ethylphenyl). In some embodiments, the compound of Formula (I) is phenformin, e.g., 1-(diaminomethylidene)-2-(2-phenylethyl)guanidine. In some embodiments, the compound of Formula (I) is phenformin, e.g., as depicted in FIG. 32.

In some embodiments, each of R^{1a} and R^{1b} is hydrogen. In some embodiments, R^{2a} is hydrogen. In some embodiments, R^{2b} is C₁-C₆ alkyl. In some embodiments, R^{2b} is C₁-C₄ alkyl (e.g., butyl). In some embodiments, each of R^{1a} and R^{1b} is hydrogen, R^{2a} is hydrogen, and R^{2b} is C₁-C₄ alkyl (e.g., butyl). In some embodiments, the compound of Formula (I) is buformin, e.g., 2-butyl-1-(diaminomethylidene)guanidine. In some embodiments, the compound of Formula (I) is buformin, e.g., as depicted in FIG. 32.

In some embodiments, each of R^{1a}, R^{1b} R^{2a,} and R^{2b} is hydrogen. In some embodiments, a compound of Formula (I) is biguanide, as depicted in FIG. 32.

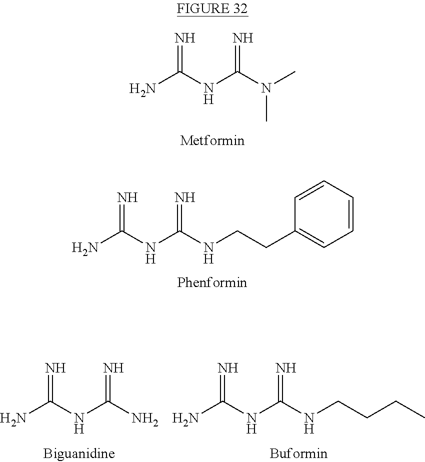

Metformin, also known as 3-(diaminomethylidene)-1,1-dimethylguanidine or N,N-Dimethylimidodicarbonimidic diamide, is a widely prescribed glucose-lowering drug for patients suffering from Type 2 diabetes.

In one embodiment, the amount of metformin administered may be a standard dose commonly used in therapeutic administration for treatment of type 2 diabetes, e.g., from about 1500 mg/day to about 2550 mg/day. For example, metformin is administered at 1500 mg/day, 1550 mg/day, 1600 mg/day, 1650 mg/day, 1700 mg/day, 1750 mg/day, 1800 mg/day, 1850 mg/day, 1900 mg/day, 1950 mg/day, 2000 mg/day, 2050 mg/day, 2100 mg/day, 2150 mg/day, 2200 mg/day, 2250 mg/day, 2300 mg/day, 2350 mg/day, 2400 mg/day, 2450 mg/day, 2500 mg/day or 2550 mg/day.

In certain embodiments, metformin is administered at a dose that is less than the standard of care dose or dosage formulation for lowering glucose levels or treatment of Type 2 diabetes. In certain embodiments, metformin is administered at a dose or dosage formulation that is less than 0.01, 0.05, 0.1, 0.2, 0.3, 0.4, 0.5, 0.6, 0.7, 0.8, 0.9, that of the standard of care (e.g., the lower standard of care dose). In one embodiment, the dose or dosage formulation is in the range of, for example, 0.01-0.9-fold, 0.02-0.8-fold, 0.05-0.7-fold, 0.1-0.5 fold, 0.1-0.2-fold, that of the standard of care dose or dosage formulation. Standard of care doses or dosage formulation of metformin is available in the art, some of which are exemplified herein.

In yet other embodiments, metformin is administered at a dose or dosage formulation that is greater than the standard of care dose or dosage formulation for lowering glucose levels or treatment of Type 2 diabetes (e.g., a dose or dosage form that is greater than 1.1, 1.5, 1.7, 2, 3, 4, 5, 10-fold or higher, that of the standard of care dose for treatment of Type 2 diabetes). In one embodiment, the dose or dosage formulation is in the range of, for example, 1.1 to 10-fold, 1.5-5-fold, 1.7 to 4-fold, or 2-3-fold, that of the standard of care dose or dosage formulation. Standard of care doses or dosage formulation of metformin is available in the art, some of which are exemplified herein.

Anti-Hypertensive and/or Collagen-Modifying Agents

The methods provided herein are directed to administration of a metformin agent in combination with an AHCM described herein, e.g., for treating or preventing a disease or disorder, e.g., a cancer or a fibrotic disorder described herein. The method can include one, two, three or more AHCM agents, alone or in combination with one or more therapeutic agents described herein (e.g., a microenvironment modulator, an anti-inflammatory agent, an immune-checkpoint inhibitor, or an additional therapy, e.g., a cancer or anti-fibrotic therapy).

In certain embodiments, the AHCM agent used in the methods and compositions of the invention can be chosen from one or more of: an antagonist of renin angiotensin aldosterone system (“RAAS antagonist”), an angiotensin converting enzyme (ACE) inhibitor, an angiotensin II receptor blocker (AT₁ blocker), a thrombospondin 1 (TSP-1) inhibitor, a transforming growth factor beta 1 (TGF-β1) inhibitor, and a connective tissue growth factor (CTGF) inhibitor. Exemplary antagonists of renin angiotensin aldosterone system (RAAS) include, but are not limited to, aliskiren (TEKTURNA®, RASILEZ®), remikiren (Ro 42-5892), enalkiren (A-64662), SPP635, and derivatives thereof.

Exemplary angiotensin converting enzyme (ACE) inhibitors include, but are not limited to, benazepril (LOTENSIN®), captopril (CAPOTEN®), enalapril (VASOTEC®), fosinopril (MONOPRIL®), lisinopril (PRINIVIL®, ZESTRIL®), moexipril (UNIVASC®), perindopril (ACEON®), quinapril (ACCUPRIL®), ramipril (ALTACE®), trandolapril (MAVIK®), and derivatives thereof.

Exemplary angiotensin II receptor blockers (AT₁ blockers) include, but are not limited to, losartan (COZAAR®), candesartan (ATACAND®), eprosartan mesylate (TEVETEN®), EXP 3174, irbesartan (AVAPRO®), L158,809, olmesartan (BENICAR®), saralasin, telmisartin (MICARDIS®), valsartan (DIOVAN®), and derivatives thereof.

In one embodiment, the AT₁ blocker is losartan, or a derivative thereof. Losartan is an anti-hypertensive agent with miminal safety risks (Johnston C I (1995) Lancet 346: 1403-1407). Furthermore, in addition to its antihypertensive properties, losartan is also an antifibrotic agent that has been shown to reduce the incidence of cardiac and renal fibrosis (Habashi J P, et al. (2006) Science 312: 117-121; and. Cohn R D, et al. (2007) Nat Med 13: 204-210). The antifibrotic effects of losartan are caused, in part, by the suppression of active transforming growth factor-β1 (TGF-β1) levels via an angiotensin II type I receptor (AGTR1) mediated down-regulation of TGF-β1 activators like thrombospondin-1 (TSP-1) (Habashi J P, et al. (2006) Science 312: 117-121; Cohn R D, et al. (2007) Nat Med 13: 204-210; Lavoie P, et al. (2005) J Hypertens 23: 1895-1903; Chamberlain J S (2007) Nat Med 13: 125-126; and Dietz H C (2010) J Clin Invest 120: 403-407).

Exemplary thrombospondin 1 (TSP-1) inhibitors include, but are not limited to, ABT-510, CVX-045, LSKL, and derivatives thereof.

Exemplary transforming growth factor beta 1 (TGF-β1) inhibitors include, but are not limited to, CAT-192, fresolimumab (GC1008), LY 2157299, Peptide 144 (P144), SB-431542, SD-208, compounds described in U.S. Pat. No. 7,846,908 and U.S. Patent Application Publication No. 2011/0008364, and derivatives thereof.

Exemplary connective tissue growth factor (CTGF) inhibitors include, but are not limited to, DN-9693, FG-3019, and compounds described in European Patent Application Publication No. 1839655, U.S. Pat. No. 7,622,454, and derivatives thereof.

Exemplary beta-blockers include, but are not limited to, atenolol (TENORMIN®), betaxolol (KERLONE®), bisoprolol (ZEBETA®), metoprolol (LOPRESSOR®), metoprolol extended release (TOPROL XI®), nadolol (CORGARD®), propranolol (INDERAL®), propranolo long-acting (INDERAL LA®), timolol (BLOCADREN®), acebutolol (SECTRAL®), penbutolol (LEVATOL®), pindolol, carvedilol (COREG®), labetalol (NORMODYNE®, TRANDATE®), and derivatives thereof.

In one embodiment, the AHCM agent is a TGF-β1 inhibitor, e.g., an anti- TGF-β1 antibody, a TGF-1 peptide inhibitor. In certain embodiment, the TGF-β1 inhibitor is chosen from one or more of: CAT-192, fresolimumab (GC1008), LY 2157299, Peptide 144 (P144), SB-431542, SD-208, compounds described in U.S. Pat. No. 7,846,908 and U.S. Patent Application Publication No. 2011/0008364, or a derivative thereof.

Suitable doses for administration of the AHCM agent can be evaluated based on the standard of care anti-hypertensive doses of the AHCM agents are available in the art.

Exemplary standard of care anti-hypertensive and anti-heart failure doses and dosage formulations for AT₁ inhibitors in humans are as follows: 25-100 mg day⁻¹ of losartan (available in a dosage form for oral administration containing 12.5 mg, 25 mg, 50 mg or 100 mg of losartan); 4 to 32 mg day⁻¹ of candesartan (ATACAND®) (e.g., available in a dosage form for oral administration containing 4 mg, 8 mg, 16 mg, or 32 mg of candesartan); 400 to 800 mg day⁻¹ of eprosartan mesylate (TEVETEN®) (e.g., available in a dosage form for oral administration containing 400 or 600 mg of eprosartan); 150 to 300 mg day⁻¹ of irbesartan (AVAPRO®) (e.g., available in a dosage form for oral administration containing 150 or 300 mg of irbesartan); 20 to 40 mg day⁻¹ of olmesartan (BENICAR®) (available in a dosage form for oral administration containing 5 mg, 20 mg, or 40 mg of olmesartan); 20 to 80 mg day⁻¹ of telmisartin (MICARDIS®) (e.g., available in a dosage form for oral administration containing of 20 mg, 40 mg or 80 mg of telmisartin); and 80 to 320 mg day⁻¹ of valsartan (DIOVAN®) (e.g., available in a dosage form for oral administration containing 40 mg, 80 mg, 160 mg or 320 mg of valsartan).

Exemplary standard of care anti-hypertensive and anti-heart failure doses and dosage formulations for ACE inhibitors in humans are as follows: 10 to 40 mg day⁻¹ of benazepril (LOTENSIN®) (Lotensin (benazepril) is supplied as tablets containing 5 mg, 10 mg, 20 mg, or 40 mg of benazepril hydrochloride for oral administration); 25 to 100 mg day⁻¹ of captopril (CAPOTEN®) (available in a dosage form for oral administration containing 12.5 mg, 25 mg, 50 mg or 100 mg of captopril); 5 to 40 mg day⁻¹ of enalapril (VASOTEC®) (available in a dosage form for oral administration containing 2.5 mg, 5 mg, 10 mg or 20 mg of enalapril; 10 to 40 mg day⁻¹ of fosinopril (MONOPRIL®) (available in a dosage form for oral administration containing 10 mg, 20 mg, or 40 mg of fosinopril); 10 to 40 mg day⁻¹ of lisinopril (PRINIVIL®, ZESTRIL®) (available in a dosage form for oral administration containing 2.5 mg, 5mg, 10 mg, 20 mg, 30 mg or 40 mg of lisinopril); 7.5 to 30 mg day⁻¹ of moexipril (UNIVASC®) (available in a dosage form for oral administration containing 7.5 mg or 15 mg of Moexipril); 4 to 8 mg day⁻¹ of perindopril (ACEON®) (available in a dosage form for oral administration containing 2 mg, 4 mg or 8 mg of perindopril), 10 to 80 mg day⁻¹ of quinapril (ACCUPRIL®) (available in a dosage form for oral administration containing 5 mg, 10 mg, 20 mg, or 40 mg of quinapril); 2.5 to 20 mg day⁻¹ of ramipril (ALTACE®) (available in a dosage form for oral administration containing 1.25 mg, 2.5 mg, 5mg, or 10 mg of ramipril); 1 to 4 mg day⁻¹ of trandolapril (MAVIK®) (available in a dosage form for oral administration containing 1 mg, 2 mg, or 4 mg of trandolapril).

In one embodiment, the AHCM agent is administered at a standard of care anti-hypertensive and anti-heart failure doses and dosage formulations, e.g., a dose or dosage formulation as described herein.

In certain embodiments, a sub-anti-hypertensive dose or dosage formulation of the AHCM agent is desirable, e.g., a dose of the AHCM agent that is less than the standard of care dose or dosage formulation. In one embodiment, the sub-anti-hypertensive dose or dosage formulation has a minimal effect in blood pressure in a hypertensive subject (e.g., decreases the mean arterial blood pressure in a hypertensive subject by less than 20%, 10%, or 5% or less). In certain embodiments, the AHCM agent is administered at a dose or dosage formulation that is less than 0.01, 0.05, 0.1, 0.2, 0.3, 0.4, 0.5, 0.6, 0.7, 0.8, 0.9, that of the standard of care anti-hypertensive dose (e.g., the lower standard of care dose). In one embodiment, the dose or dosage formulation is in the range of, for example, 0.01-0.9-fold, 0.02-0.8-fold, 0.05-0.7-fold, 0.1-0.5 fold, 0.1-0.2-fold, that of the standard of care dose or dosage formulation for anti-hypertensive or anti-heart failure use. Standard of care doses or dosage formulation of the AHCM are available in the art, some of which are exemplified herein.

In yet other embodiments, the AHCM agent is administered at a dose or dosage formulation that is greater than the standard of care dose or dosage formulation for anti-hypertensive or anti-heart failure use (e.g., a dose or dosage form that is greater than 1.1, 1.5, 1.7, 2, 3, 4, 5, 10-fold or higher, that of the standard of care dose for anti-hypertensive or anti-heart failure use). In one embodiment, the dose or dosage formulation is in the range of, for example, 1.1 to 10-fold, 1.5-5-fold, 1.7 to 4-fold, or 2-3-fold, that of the standard of care dose or dosage formulation for anti-hypertensive or anti-heart failure use. Standard of care doses or dosage formulation of the AHCM are available in the art, some of which are exemplified herein.

The standard of care dose and dosage forms are provided herein for a number of AHCMs, e.g., losartan. In an embodiment, the dose and/or dosage form is less than (or higher than) the standard of care dose and/or dosage form. In an exemplary embodiment, it is less than 0.01, 0.02, 0.05, 0.1, 0.2, 0.5, 0.7, 0.8, 0.9-fold, that of the standard of care dose or dosage form. In embodiments, the dose or dosage form contains an amount of AHCM that is within a range of the reduced amounts of the standard of care dose and/or dosage form. E.g., an AHCM dosage form that is 0.01-0.9-fold, 0.02-0.8-fold, 0.05-0.7-fold, 0.1-0.5 fold, 0.1-0.2-fold, that of the standard of care dose or dosage form. In certain embodiments, the range of the dose or the dosage form is 0.5-2.0 times a reduced dose or dosage form recited herein, so long as the dose or dosage form value is less than the standard of care dose or dosage form. By way of example, a standard of care dosage form for losartan is 12.5 mg. Thus, in embodiments, the dosage form is 0.125 mg (0.01×12.5 mg); 0.625 mg (0.05×12.5 mg); 1.25 mg (0.1×12.5 mg); 2.5 mg (0.2×12.5 mg); or 6.25 mg (0.5×12.5 mg). In an embodiment, the AHCM dosage form is in the range 0.5-2.0 (0.125 mg)=0.0625-0.25 mg; 0.5-2.0 (0.625 mg)=0.312-1.25 mg; and so on, so long as the dose or dosage form value is less than the standard of care dose or dosage form. This calculation can be applied to any standard of care dose and/or dosage form for any AHCM described herein. In certain embodiment, the value is less than the standard of care values. In other embodiments, the value is greater than the standard of care values.

In one embodiment, the dose of the AHCM agent is calculated based on the severity of the fibrosis in the tumor sample.

In some embodiments, the dose of the AHCM agent can be a sub-anti-hypertensive dose, which does not have any anti-tumor effect, e.g., no significant effect on inhibiting or preventing tumor growth or progression when administered alone. In some embodiments, the dose of the AHCM agent can be comparable to or greater than the standard of care dose or dosage formulation for anti-hypertensive or anti-heart failure use, and does not have any anti-tumor effect, e.g., no significant effect on inhibiting or prevening tumor growth or progression when administered alone.

### Microenvironment Modulators

The methods provided herein are directed to administration of a metformin agent in combination with microenvironment modulator or other stomral modulator described herein, e.g., for treating or preventing a disease or disorder, e.g., a cancer, or a fibrotic or inflammatory disorder described herein. The method can include one, two, three or more microenvironment modulators or other stromal modulator, alone or in combination with one or more therapeutic agents described herein (e.g., an AHCM, an anti-inflammatory agent, an immune-checkpoint inhibitor, or an additional therapy, e.g., a cancer or anti-fibrotic therapy).

In certain embodiments, the combinations described herein can be further administered in combination with a microenvironment modulator. The combined administration of the the microenvironment modulator can be used to further enhance the efficacy (e.g., penetration and/or diffusion), of the combination therapies described herein in a tumor or tumor vasculature in a subject. Such combination may cause one or more of: reduce solid stress (e.g., growth-induced solid stress in tumors); decrease tumor fibrosis; reduce interstitial hypertension or interstitial fluid pressure (IFP); increase interstitial tumor transport; increase tumor or vessel perfusion; increase vascular diameters and/or enlarge compressed or collapsed blood vessels; reduce or deplete one or more of: cancer cells, or stromal cells (e.g., tumor associated fibroblasts or immune cells); decrease the level or production of extracellular matrix components, such as fibers (e.g., collagen, procollagen), and/or polysaccharides (e.g., glycosaminoglycans such as hyaluronan or hyaluronic acid); decrease the level or production of collagen or procollagen; decreases the level or production of hyaluronic acid; increases tumor oxygenation; decreases tumor hypoxia; decreases tumor acidosis; enables immune cell infiltration; decreases immunosuppression; increases antitumor immunity; decreases cancer stem cells (also referred to herein as tumor initiating cells), thereby enhacing the penetration and/or distribution of the therapy, e.g., the cancer therapy.

Exemplary microenvironment modulators are disclosed herein, and include, but are not limited to, an anti-angiogenic therapy, for example, an inhibitor of vascular endothelial growth factor (VEGF) pathway; an agent that decreases the level or production of hyaluronic acid; an inhibitor of the hedgehog pathway; an agent that improves drug penetration in tumors. In one embodiment, the agent is a disulfide-based cyclic RGD peptide peptide (iRGD) or an analogue thereof; a taxane therapy (e.g., taxane-induced apoptosis); an agent that decreases the level or production of collagen or procollagen; an anti-fibrotic agent and/or a profibrotic pathway inhibitor.

In one embodiment, the anti-angiogenic agent is chosen from a VEGF-inhibitor, an inhibitor of the angiopoietin-Tie-2 pathway (e.g., an Ang-1 or an Ang-2 inhibitor), or sorafenib. Examples of anti-angiopoietin/Tie-2 pathway agents (or inhibitors of the angiopoietin-Tie-2 pathway) include, but are not limited to, AMG 386, CVX-060, CVX-241, MEDI-3617, REGN910, AMG-780, CEP-1198, ARRY-614, MGCD265, Regorafenib, and combinations thereof. In one embodiment, the anti-angiogenic agent can be an inhibitor of tyrosine or Serine/Threonin kinases such as VEGFR, PDGFR, c-kit receptors, b-Raf, or combinations thereof. Additional examples of anti-angiogenic agents include, but are not limited to, agents that inhibit oncogene activation (e.g., anti-EGFR such as gefitinib; anti-HER2 such as Trastuzumab; anti-P13K-AKT-mTOR such as NVPBEZ235, P1-103, Palomid-529, Nelfinavir; anti-Ras such as FTIs); agents that target androgens (e.g., Castration or endocrine therapy); agents that inhibits inflammotry cytokine-induced VEGF activation; anti-P1GF agents; anti-integrin agents (e.g., Cilengitide); agents that targets PHD2/HIF pathway; anti-Rgs5 agents; Ang-1 agonistic agents; SEMA3A/NRP-1 agonistic agents; PDGF-B agonistic agents; eNOS agonistic agents; PDGF-C agonistic agents; PDGF-D agonistic agents, IFN-β agonistic agents; TSP-1 agonistic agents; anti-TNFα/TNFR agents; anti-TGFβ/TGFR agents; anti-VE-PTP agents; anti-MMP agents (e.g., anti-MMP-2; anti-MMP-9; anti-MMP-14); WNT agonistic agents; extracellular matrix-inducing agents (e.g., fibronectin; laminin; netrin-1; thrombospondin 1, etc.); Notchl agonistic agents; Frizzled agonistic agents; and a combination of two or more thereof. Agents for anti-angiogenic/vascular normalization strategies as described in Goel et al. (2011) Physiol Rev. 91: 1071-1121, and Jain (2014) Cancer Cell 26(5): 605-622, the contents of which are incorporated herein by reference, can also be used as an anti-angiogentic agent for the compositions and methods described herein.

In one embodiment, the anti-angiogenic agent, e.g., sorafenib is administered to a subject at a vascular/stromal normalizing dose (e.g., a sub-anti-angiogenic dose, also referred to herein as a “low dose”) as a particle or a free agent, e.g., as described herein in the context of treatment of fibrotic conditions or disorders (e.g., liver diseases or disorders, kidney fibrosis, cardiovascular diseases, or idiopathic pulmonary fibrosis).

In one embodiment, the anti-angiogenic agent is administered at a vascular/stromal normalizing dose. A vascular/stromal normalizing dose can have an angiogenic effect. In one embodiment, the vascular/stromal normalizing dose of the second agent results in one or more of: (i) increase in hepatic vascular function; (ii) repair of hepatic blood vessels; (iii) increase in vascular normalization; (iv) reduction in pore size; (v) reduction in hypoxic tissue; (vi) increase in perfusion of the diseased liver tissue; (vii) restoration of agent delivery; (viii) improved stromal signaling; or (ix) improved or normalized angiocrine signaling. In one embodiment, the effect of the “vascular/stromal normalizing” is detected by one or more of: angiography imaging, immunostaining of level of hypoxia (e.g., using pimonidazol-FITC), increased sinusoidal perfusion, or increased stromal/angiocrine signaling.

In one embodiment, the anti-angiogenic agent is administered at a sub-anti-angiogenic dose, also referred to herein as a “low dose”). A “sub-anti-angiogenic dose,” as used herein, refers to a dose of an anti- angiogenic agent that is typically less than the lowest dose that would be used to have a detectable anti-angiogenic effect in a subject. A sub-anti-angiogenic dose can have an angiogenic effect. In one embodiment, the sub-anti-angiogenic dose of the second agent results in one or more of: (i) increased hepatic vascular function; (ii) repaired hepatic blood vessels; (iii) increased vascular normalization; (iv) reduced pore size; (v) reduced hypoxic tissue; (vi) increased perfusion of the diseased liver tissue; or (vii) restored agent delivery. In one embodiment, the effect of the “sub-anti-angiogenic dose” is detected by one or more of: angiography imaging, immunostaining of level of hypoxia (e.g., using pimonidazol-FITC), or increased sinusoidal perfusion e.g., as shown in the appended Examples.

In some embodiments, the anti-angiogenic agent is sorafenib. In some embodiments, sorafenib is administered at a low dose, e.g., a dose of sorafenib that is less than the standard of care dose, e.g., less than an anti-angiogenic or anti-vascularization dose. In one embodiment, sorafenib is administered at a dose or dosage formulation that is less than 0.01, 0.05, 0.1, 0.2, 0.3, 0.4, 0.5, 0.6, 0.7, 0.8, 0.9, that of the standard of care dose. In one embodiment, the dose or dosage formulation is in the range of, for example, 0.01-0.9-fold, 0.02-0.8-fold, 0.05-0.7-fold, 0.1-0.5 fold, 0.1-0.2-fold, that of the standard care dose or dosage formulation. Standard of care doses or dosage formulations of sorafenib are available in the art, some of which are exemplified herein.

In an exemplary embodiment, the low dose or dosage formulation of sorafenib is provided in a dose ranging from 0.1 mg/kg to 4 mg/kg, 0.1 mg/kg to 1 mg/kg, 0.2 mg/kg to 4 mg/kg, 0.2 mg/kg to 1 mg/kg, 0.2 mg/kg to 0.5 mg/kg, 0.3 mg/kg to 4 mg/kg, or 0.3 mg/kg to 1 mg/kg. In an embodiment, the low dose or dosage formulation of sorafenib is provided in a dosage of about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg, 2 mg/kg, 3 mg/kg, or 4 mg/kg, but less than 4.2 mg/kg. Alternatively, the low dose or dosage formulation of sorafenib is administered at a dose ranging from 1 to 250 mg, 1 to 150 mg, 1 to 100 mg, 1 to 50 mg, 1 to 20 mg, 1 to 10 mg, or 1 to 5 mg. For example, the low dose or dosage formulation of sorafenib is administered at a dose of less than 250 mg, e.g., about 200 mg, 150 mg, 100 mg, 90 mg, 80 mg, 70 mg, 60 mg, 50 mg, 45 mg, 40 mg, 35 mg, 30 mg, 25 mg, 20 mg, 15 mg, 10 mg, or 5 mg. In an embodiment, the formulation of sorafenib is an oral composition. In another embodiment, the dosage formulation of sorafenib is administered once a day or more than once a day, e.g., twice a day.

In other embodiments, the anti-angiogenic agent is an inhibitor of vascular endothelial growth factor (VEGF) pathway. Exemplary VEGF pathway inhibitors include, but are not limited to, an antibody against VEGF (e.g., bevacizumab); a VEGF receptor inhibitor (e.g., an inhibitor of VEGFR-1 inhibitor, a VEGFR-2 inhibitor, or a VEGFR-3 inhibitor (e.g., VEGFR inhibitors such as Cediranib (AZD2171)); a VEGF trap (e.g., a fusion protein that includes a VEGFR domain (e.g., a VEGFR1 domain 2 and a VEGFR2 domain 3) fused to an Fc fragment of an IgG); and an anti-VEGF aptamer (or a pegylated derivative thereof (e.g., MACUGEN®). Any of these inhibitors can be administered at a sub-anti-angiogenic dose as described above for sorafenib. e.g., a dose of that is less than the standard of care dose, e.g., an anti-angiogenic or anti-vascularization dose. In one embodiment, the inhibitor is administered at a dose or dosage formulation that is less than 0.01, 0.05, 0.1, 0.2, 0.3, 0.4, 0.5, 0.6, 0.7, 0.8, 0.9, that of the standard of care dose. In one embodiment, the dose or dosage formulation is in the range of, for example, 0.01-0.9-fold, 0.02-0.8-fold, 0.05-0.7-fold, 0.1-0.5 fold, 0.1-0.2-fold, that of the standard care dose or dosage formulation. Standard of care doses or dosage formulations of the inhibitor are available in the art, some of which are exemplified herein.

In other embodiments, the anti-angiogenic agent is inhibitor of the angiopoietin-Tie-2 pathway (e.g., an Ang-1 or an Ang-2 inhibitor. In one embodiment, the inhibitor is a dual inhibitor of VEGF and an angiopoietin (also known as a double anti-angiogenic protein or DAAP). In one embodiment, the inhibitor is an antibody against Ang-1 or Ang-2 or both. In other embodiments, the inhibitor is a peptibody that neutralizes Ang-1 or Ang-2. In one embodiment, the inhibitor is administered at a dose or dosage formulation that is less than 0.01, 0.05, 0.1, 0.2, 0.3, 0.4, 0.5, 0.6, 0.7, 0.8, 0.9, that of the standard of care dose. In one embodiment, the dose or dosage formulation is in the range of, for example, 0.01-0.9-fold, 0.02-0.8-fold, 0.05-0.7-fold, 0.1-0.5 fold, 0.1-0.2-fold, that of the standard care dose or dosage formulation. Standard of care doses or dosage formulations of the inhibitor are available in the art, some of which are exemplified herein.

In another embodiment, the microenvironment modulator includes an agent that decreases the level or production of hyaluronic acid (HA). Enzymatic targeting of the stroma using systemic administration of a pegylated derivative of hyaluronidase (PEGPH20) has been shown to ablate stromal HA in a model for pancreatic ductal adenocarcinoma (PDA) and increase vessel diameter in pancreatic tumors; hyaluronidase derivatives, in combination with standard chemotherapeutic agents (e.g., gemcitabine), can remodel the tumor microenvironment and increase overall survival (see e.g., Provenzano, P. et al. (2012) Cancer Cell 21: 418-429). Thus, combined administration of the AHCM and the microenvironment modulator can be used to enhance penetration and/or diffusion of a cancer therapy in a tumor or tumor vasculature, by for example, decreasing certain matrix components, e.g., HA, in the stroma. Exemplary HA-depleting agents include, but are not limited to, an anti-hyaluronan enzymatic therapy such as hyaluronidase or a derivative thereof (e.g., pegylated recombinant human hyaluronidase) (e.g., PH20, PEGPH20); and an antibody against hyaluronic acid.

In another emdodiment, the microenvironment modulator includes an inhibitor of the hedgehog pathway. Hedgehog inhibitors have been shown to increase vessel density in pancreatic tumors (Olive, K. P. et al. (2009) Science 324: 1457-61), presumably by reducing stromal cell density and solid stress. Exemplary hedgehog inhibitors include, but are not limited to, IPI-926, GDC-0449, cylopamine or an analogue thereof, and GANT58.

In another embodiment, the microenvironment modulator includes an agent that improves drug penetration in tumors. In one embodiment, the agent is a disulfide-based cyclic RGD peptide peptide (iRGD) or an analogue thereof (e.g., described in Sugahara, K N et al. (2010) Science 328: 1031-5; Ye, Y. et al. (2011) Bioorg Med Chem Lett. 21(4): 1146-50).

In yet another embodiment, the microenvironment modulator includes a taxane therapy (e.g.,taxane-induced apoptosis as described in Griffon-Etienne, G. et al. (1999) Cancer Res. 59(15): 3776-82).

In another embodiment, the microenvironment modulator includes an agent that modulates (e.g, inhibits) a hypoxia inducible factor (HIF), for example, an agent that inhibits hypoxia-inducible factors 1α and 2α (HIF-1α and HIF-2α). HIF activity has been shown to be involved in inflammation (e.g., rheumatoid arthritis) and angiogenesis associated with cancer tumor growth. HIF inhibitors, such as phenethyl isothiocyanate (PEITC) are under investigation for anti-cancer effects (Syed Alwi S S, et al. (2010) Br. J. Nutr. 104 (9): 1288-96; Semenza G L (2007). Drug Discov. Today 12 (19-20): 853-9; Melillo G (2006). Mol. Cancer Res. 4 (9): 601-5. In one embodiment, the agent is an antibody against an HIF. In another embodiment, the agent is an HIF chemical inhibitor, such as phenethyl isothiocyanate (PEITC).

In another embodiment, the microenvironment modulator includes an agent that decreases the level or production of collagen or procollagen. For example, an agent that degrades collagen, e.g., collagenase.

In one embodiment, the combinations described herein can be further administered in combination with a microenvironment modulator chosen from an anti-fibrotic agent or an inhibitor of a profibrotic pathway (a “profibrotic pathway inhibitor”) (e.g., a pathway dependent-or independent of TGF-beta and/or CTGF activation). In one embodiment, the combinations described herein are administered in combination with one or more of: an inhibitor of endothelin-1, PDGF, Wnt/beta-catenin, IGF-1, TNF-alpha, and/or IL-4. In another embodiment, the combinations described herein are administered in combination with an inhibitor of endothelin-1 and/or PDGF. In other embodiments, the combinations described herein are administered in combination with an inhibitor of one or more of chemokine receptor type 4 (CXCR4) (e.g., AMD3100, MSX-122); stromal-derived-factor-1(SDF-1) (e.g., tannic acid); hedgehog (e.g., IPI-926, GDC-0449, cylopamine or an analogue thereof, or GANT58).

In certain embodiments, an inhibitor of a CXCR4 receptor and/or its ligand, SDF-1, is administered in combination with a therapy (e.g., a cancer or hyperproliferative therapy as described herein). Certain embodiments may further include administration of a further AHCM and/or a microenvironment modulator as described herein. Without wishing to be bound by theory, inhibition of CXCR4 receptor and/or its ligand, SDF-1, alone or in combination with the combination therapies described herein, e.g., an angiotensin II receptor blocker, can be used to reduce the desmoplasia in certain fibrotic or desmoplastic cancers, e.g., a fibrotic or a desmoplastic solid tumor, such as pancreatic cancers (e.g., pancreatic ductal adenocarcinoma (PDAC)). For example, activation of SDF-1a/CXCR4 and angiotensin II (ATII) signaling pathways is known to promote carcinoma activated fibroblasts (CAF) recruitment, activation, and matrix production in PDAC. Hypoxia, which is associated with PDAC, can induce SDF-1a and CXCR4 expression in cancer cells and CAFs through HIF-1a activation (Schioppa, T., et al. (2003) J Exp Med, 198: 1391-1402) while promoting growth and metastasis (Chang, Q., et al. (2011) Cancer Research, 71: 3110-3120). These effects arise, at least in part, through SDF-1a/CXCR4-dependent activation of CAFs (Gao, Z. et al. (2010) Pancreatology 10: 186-193; Moriyama, T. et al. (2010) Cancer 116: 3357-3368) and a CD133+/CXCR4+ cancer stem cell population (Hermann, P. C. et al. (2007) Cell Stem Cell 1: 313-323), which also confers chemoresistance (Singh, S. et al. (2010) Br J Cancer 103: 1671-1679). High SDF-1a levels (Liang, J. J., et al. (2010) Cancer Epidemiology Biomarkers & Prevention 19: 2598-2604) and CXCR4 levels (Marechal, R. et al. (2009) Br J Cancer, 100: 1444-1451) can be predictive of poor prognosis in PDAC patients. On the other hand, ATII signaling can stimulate CAF proliferation (Hama, K. et a;. (2006) Biochemical and Biophysical Research Communications, 340: 742-750; Hama, K. et al. (2004) Biochem Biophys Res Commun. 315: 905-911; Shimizu, K. et al. (2008) J Gastroenterol Hepatol, 23 Suppl 1: S119-121), and ATII signaling through ATII-receptor type 1 (AT1) can stimulate CAF matrix production via TGF-β1 and ERK-dependent mechanisms (Rodriguez-Vita, J. et al. (2005) Circulation 111: 2509-2517; Yang, F. et al. (2009) Hypertension, 54: 877-884). ATII also induces TGF-β1 (Elenbaas, B. and Weinberg, R. A. (2001) Experimental Cell Research, 264: 169-184) and SDF-1a (Chu, P. Y. et al. (2010) Am J Pathol, 176: 1735-1742) expression by both cancer cells and CAFs, which can promote CAF proliferation and matrix production. Thus, inhibition of a CXCR4 receptor and/or its ligand, SDF-1, can be used (alone or with an inhibitor of ATII signaling) to enhance the distribution of a therapy in fibrotic or desmoplastic cancers.

Exemplary SDF-1/CXCR4 inhibitors that can be used include, but are not limited to, 2,2′-bicyclam; 6,6′- bicyclam; AMD3100 (IUPAC name: 1,1′-[1,4-phenylene-bis(methylene)]-bis-1,4,8,11-tetraazacyclotetradecane), as described in e.g., U.S. Pat. Nos. 5,021,409, 6,001,826 and 5,583,131; Plerixafor (trade name: Mozobil; IUPAC name: 1,1′-[1,4-Phenylenebis(methylene)]bis [1,4,8,11-tetraazacyclotetradecane); CXCR4 peptide inhibitors or analogs, e.g., T-140 analogs (e.g., 4F-benzoyl-TN14003, TC14012, TE14011, TC14003), CTCE-0214; CTCE-9908; and CP-1221, as well as other inhibitors such as antibodies against SDF-1 or CXCR4, RNA inhibitors (e.g., antisense, siRNAs), among others. Exemplary inhibitors are described in, for example, Tamamura, H. et al. Org. Biomol. Chem. 1: 3656-3662, 2003; FEBS Letter 550: 1-3 (2003): 79-83; Wong, D. et al. (2008) Clin. Cancer Res. 14(24): 7975-7980; US Patent Publications 2010/0055088; 2009/0221683; 2004/0209921, 2005/0059702, 2005/0043367, 2005/0277670, 2010/0178271, and 2003/0220341; U.S. Pat. Nos. 5,021,409, 6,001,826, 5,583,131, and Patent Publications WO 03/011277, WO 01/85196; WO 99/50461; WO 01/94420; WO 03/090512, each of which is incorporated herein by reference in their entirety.

### Anti-Inflammatory Agents

The methods provided herein are directed to administration of a metformin agent in combination with an anti-inflammatory agent described herein, e.g., for treating or preventing a disease or disorder, e.g., a cancer or a fibrotic disorder described herein. The method can include one, two, three or more anti-inflammatory agents, alone or in combination with one or more therapeutic agents described herein (e.g., an AHCM agent, a microenvironment modulator, an immune-checkpoint inhibitor, or an additional therapy, e.g., a cancer or anti-fibrotic therapy).

In one embodiment, the anti-inflammatory agent is an agent that blocks, inhibits, or reduces inflammation or signaling from an inflammatory signaling pathway. In one embodiment, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23, interferons (IFNs), e.g., IFNα, IFNβ, IFNγ, IFN-γ inducing factor (IGIF), transforming growth factor-β (TGF-β), transforming growth factor-α (TGF-α), tumor necrosis factors TNF-α, TNF-β, TNF-RI, TNF-RII, CD23, CD30, CD40L, EGF, G-CSF, GDNF, PDGF-BB, RANTES/CCL5, IKK, NF-κB, TLR2, TLR3, TLR4, TL5, TLR6, TLR7, TLR8, TLR8, TLR9, and/or any cognate receptors thereof.

In one embodiment, the anti-inflammatory agent is an IL-1 or IL-1 receptor antagonist, such as anakinra (KINIRET®), rilonacept, or canakinumab.

In one embodiment, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061.

In one embodiment, the anti-inflammatory agent is a TNF-α antagonist, e.g., an anti-TNFα antibody, such as infliximab (REMICADE®), golimumab (SIMPONI®), adalimumab (HUMIRA®), certolizumab pegol (CIMZIA®) or etanercept.

In one embodiment, the anti-inflammatory agent is a corticosteroid. Exemplary corticosteroids include, but are not limited to, cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PREZONE®), and prednisone (DELTASONE®, LIQUID PRED®, METICORTEN®, ORASONE®)), and bisphosphonates (e.g., pamidronate (AREDIA®), and zoledronic acid (ZOMETA®).

In another embodiment, the anti-inflammatory agent is a non-steroidal anti-inflammatory drug (NSAID). Exemplary anti-inflammatory agents (e.g., NSAIDs) include, but are not limited to, aspirin, ibuprofen, naproxen, celecoxib, diclofenac, diflunisal, etodolac, fenoprofen, flurbiprofen, ketoprofen, ketorolac, mefenamic acid, meloxicam, nabumetone, oxaprozin, piroxicam, sulindac, and tolmetin. In an embodiment, the anti-inflammatory agent is an immune selective anti-inflammatory derivative (ImSAID).

### Immune-Checkpoint Inhibitors

The methods provided herein are directed to administration of a metformin agent in combination with an immune-checkpoint inhibitor described herein, e.g., for treating or preventing a disease or disorder, e.g., a canceror a fibrotic disorder described herein. The method can include one, two, three or more immune-checkpoint inhibitors, alone or in combination with one or more therapeutic agents described herein (e.g., an AHCM agent, a microenvironment modulator, an anti-inflammatory agent, or an additional therapy, e.g., a cancer or anti-fibrotic therapy).

Immune checkpoints refer to inhibitory pathways in the immune system that are crucial for maintaining self-tolerance (i.e., prevention of autoimmunity) and for protection of tissues from damage during response to pathogenic infections. In cancer, tumors can often hijack immune-checkpoint pathways in order to promote immune resistance and evade attack by the immune system. Thus, blockade of these immune checkpoints, e.g., by immune-checkpoint inhibitors, is desirable to enhance anti-tumor immunity and improve cancer therapies.

Immune checkpoint inhibitors, as described herein, refer to molecules that block, inhibit, or reduce activity of one or more immune checkpoint proteins. The inhibitors may be an antibody, an antigen binding fragment thereof, an immunoadhesin, a fusion protein, or an oligopeptide. Examples of immune-checkpoint molecules include, but are not limited to, PD-1, PD-L1, PD-L2, CTLA4, B7-H3, B7-H4, HVEM, BTLA, a killer-cell immunoglobulin-like receptor (KIR), LAG3, TIM3, CEACAM-1, CEACAM-3, CEACAM-5, GALS, VISTA, TIGIT, LAIR1, CD160, 2B4, TGFRbeta, and A2aR.

In some embodiments, the immune checkpoint inhibitor is a PD-1 inhibitor. Antibodies, antibody fragments, and other inhibitors of PD-1 and its ligands (e.g., PD-L1 or PD-L2) are available in the art and may be used combination with metformin as described herein. Exemplary anti-PD-1 antibodies include, but are not limited to, nivolumab (also known as MDX-1106 or BMS-936558), pembrolizumab (formerly known as lambrolizumab, also known as Merck 3475 or MK03475), and pidilizumab (also known as CT- 011). Nivolumab (clone 5C4) and other human monoclonal antibodies that specifically bind to PD-1 are disclosed in US 8,008,449 and WO2006/121168. Pidilizumab and other humanized anti-PD-1 monoclonal antibodies are disclosed in WO2009/101611. Pembrolizumab and other humanized anti-PD-1 antibodies are disclosed in U.S. Pat. No. 8,354,509 and WO2009/114335. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence), such as AMP-224.

In some embodiments, the immune checkpoint inhibitor is a PD-L1 inhibitor. Antibodies, antibody fragments, and other inhibitors of PD-L1 are available in the art and may be used combination with metformin as described herein. Exemplary anti-PD-L1 antibodies include, but are not limited to, YW243.55.S70 (as described in PCT Publication No. WO2010/077634), MPDL3280A (as described in U.S. Pat. No. 7,943,743 and U.S. Publication No. 20120039906), MEDI-4736, MSB-0010718C, or MDX-1105 (also referred to as BMS-936559, as described in WO2007/005874).

In some embodiments, the immune checkpoint inhibitor is a TIM3 inhibitor. Antibodies, antibody fragments, and other inhibitors of TIM3 and its ligands are available in the art and may be used combination with metformin as described herein. For example, antibodies, antibody fragments, small molecules, or peptide inhibitors that target TIM3 binds to the IgV domain of TIM3 to inhibit interaction with its ligands can be administered in combination with a metformin agent as described herein. Exemplary TIM3 inhibitors include, but are not limited to the antibodies and peptides disclosed in WO2013/006490 and US20100247521); anti-TIM3 inhibitors such as humanized versions of RMT3-23 (as disclosed in Ngiow et al., 2011, Cancer Res, 71:3540-3551) and clone 8B.2C12 (disclosed in Monney et al., 2002, Nature, 415:536-541). Bi-specific antibodies that inhibit TIM3 and PD-1 are disclosed in US20130156774.

In some embodiments, the immune checkpoint inhibitor is a LAG3 inhibitor. Antibodies, antibody fragments, and other inhibitors of LAG3 and its ligands are available in the art and may be used combination with metformin as described herein. Exemplaryanti-LAG3 antibodies include, but are not limited to monoclonal antibody BMS-986016 (Bristol-Myers Squib), IMP701 (Immutep), IMP731 (Immutep and GlaxoSmithKline), and antibodies disclosed in WO2010/019570. Other LAG3 inhibitors include IMP321 (Immutep), which is a recombinant fusion protein of a soluble portion of LAG3 and 1e: that binds to MHC class 11 molecules and activates antigen presenting cells (APC).

In some embodiments, the immune checkpoint inhibitor is a CEACAM inhibitor, e.g., a CEACAM-1 inhibitor, a CEACAM-3 inhibitor, and/or a CEACAM-5 inhibitor. Antibodies, antibody fragments, and other inhibitors of CEACAM are available in the art and may be used combination with metformin as described herein. Exemplary anti-CEACAM-1 antibodies include, but are not limited to, antibodies described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146), or crossreacts with CEACAM-1 and CEACAM-5 as described in, e.g., WO 2013/054331 and US 2014/0271618.

### Additional Combination Therapies

In embodiments described herein, a metformin agent is administered in combination with one or more i) AHCM; ii) microenvironment modulator or stromal modulator; iii) anti-inflammatory agent; and/or iv) immune-checkpoint inhibitor as described herein, and optionally, one or more additional therapies, e.g., a cancer therapy, e.g., a cancer therapeutic, radiation therapy, PDT, surgery, or immune therapy; or an anti-fibrotic therapy.

In other embodiments described herein, the combinations described herein are administered in combination with a cancer therapeutic, e.g., a chemotherapeutic agent, a biologic described herein.

In other embodiments, the combinations described herein are administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the combinations described herein are administered in conjunction with a biologic. Biologics useful in the treatment of cancers are known in the art and a binding molecule of the invention may be administered, for example, in conjunction with such known biologics.

For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the binding molecules of the invention may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® Velcade (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In other embodiments, the combinations described herein are administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP ASO4 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In other embodiments, the combinations described herein are administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, combinations described herein are administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17: 617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte - Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRON™), and pegfilgrastim (NEULASTA™), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADUR™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PREZONE®), and prednisone (DELTASONE®, LIQUID PRED®, METICORTEN®, ORASONE®)), and bisphosphonates (e.g., pamidronate (AREDIA®), and zoledronic acid (ZOMETA®))

In some embodiments, the combinations described herein are used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-13 inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK^{TM}), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074,nSorafenib Tosylate(Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In one embodiment, the combinations described herein are administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10: 415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

It will be appreciated that anti-tumor antibodies labeled with isotopes have been used successfully to destroy cells in solid tumors, as well as lymphomas/leukemias in animal models, and in some cases in humans. Exemplary radioisotopes include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. The radionuclides act by producing ionizing radiation which causes multiple strand breaks in nuclear DNA, leading to cell death. The isotopes used to produce therapeutic conjugates typically produce high energy α-or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

It will also be appreciated that, in accordance with the teachings herein, binding molecules can be conjugated to different radiolabels for diagnostic and therapeutic purposes. To this end the aforementioned U.S. Pat. Nos. 6,682,134, 6,399,061, and 5,843,439 disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

Without wishing to be bound by theory, hypoxia and/or limited perfusion are believed to cause immunosuppression and/or limit the efficacy of certain immune therapies. The combinations described herein can be used to improve the efficacy of said immune therapies. Examples of immune therapies include, but are not limited to, CTLA-4 blockade (e.g., an anti-CTLA-4 antibody (e.g., ipilimumab)); immune-based therapies (including, e g , immune or dendritic cell- based vaccines and antagonists of immune inhibitory signals or checkpoints); cancer vaccines, e.g., Sipuleucel-T (APC8015, trade name Provenge, manufactured by Dendreon Corporation) is a therapeutic cancer vaccine for prostate cancer (CaP)); and adoptive T-cell-based therapies. Exemplary immune-based therapies include, but are not limited to, e g , immune or dendritic cell- based vaccines (Seton-Rogers, S. (2012) Nature Reviews Cancer 12:230-231; Palucka, K. et al. (2012) Nature Reviews Cancer 12: 265-277); effector memory CD8+ T cells (Bird, L. (2012) Nature Reviews Immunology 12: 227); engineered tumor cells to activate Toll like Receptors (TLRs) and NOD-like Receptors (NLRs) (Leavy, O. (2012) Nature Reviews Immunology 12: 227); antagonists of immune inhibitory signals or checkpoints (Pardoll, D. M. (2012) Nature Reviews Cancer 12: 252-264).

In yet other embodiments, the combinations described herein are administered in combination with the use of PDT. In certain embodiments, PDT includes administration of a photosensitizing agent (e.g., a porhyrin, a porpyrin precursor, a chorlin, or a phthalocyanine) followed by irradiation at a wavelength corresponding to an absorbance band of the sensitizer. In the presence of oxygen, a series of events lead to one or more of: cell death (e.g., tumor cell death), damage to the microvasculature, or induction of a local inflammatory reaction). PDT is reviewed in, e.g., Agostinis, P. et al. (2011) CA Cancer J. Clin. 61: 250-281.

In other embodiments, the combinations described herein are administered in combination with an inhibitor of a cancer stem cell (also referred to herein as a “cancer initiating cell”). Without wishing to be bound by theory, hypoxia and cancer drugs (including anti-angiogenic drugs) and radiation therapy are believed to increase the number of cancer stem cells. Combinations described herein are administered in combination withan inhibitor of a cancer stem cell, which can be used to reduce the production of these stem cells. Exemplary inhibitors of cancer stem cells that can be used in combination include, but are not limited to, hedgehog (e.g., SMO) antagonists; and Wnt pathway antagonists (e.g., antibody, OMP-18R5).

In other embodiments, the combinations described herein are administered in combination with a liver disorder therapy described herein.

In an embodiment, the liver disorder therapy includes anti-inflammatory agents such as angiotensin converting enzyme antagonists; angiotensin II type I receptor antagonists, glucocorticoids, e.g., aldosterone, bentamethasone, cortisol, cortisone, deoxycorticosterone, dexamethasone, methylprednisolone, prednisone, prednisolone, or triamcinolone; pentoxiphylline (PTX); TNF alpha inhibitors, e.g., pentoxyphylline, adalimumab (HUMIRA®), entanercept (ENBREL®), infliximab (REMICADE®); salicylates, e.g., aspirin, diflunisal (DOLOBID™) salsalate (DISALCID™), choline magnesium trisalicylate (TRILISATE™); propionic acid derivatives, e.g., ibuprofen, naproxen, fenoprofen, ketoprofen, oxaprozin, loxoprofen; acetic acid derivatives, e.g., indomethacin, tolmetin, slindac, etodolac; selective Cox-2 inhibitors, e.g., celecoxib, rofecoxib, valdecoxib, parecoxib, lumiracoxib, etoricoxib; oxicam derivatives, e.g., piroxicam, meloxicam, tenoxicam, droxicam, lornoxicam, isoxicam; anthranilic acid derivates (fenamates), e.g., mefenamic acid, meclofenamic acid, flufenamic acid, tolfenamic acid; sulfonanilides, e.g., nimesulide; H-harpagide; and ursodeoxycholic acid, and derivatives thereof.

In an embodiment, the liver disorder therapy includes immunosuppressive agents such as glucocorticoids, e.g., aldosterone, bentamethasone, cortisol, cortisone, deoxycorticosterone, dexamethasone, methylprednisolone, prednisone, prednisolone, or triamcinolone; Mycophenolate mofetil (MMF); rapamycin (sirolimus); fingolimod; calcineurin inhibitors, e.g., ciclosporin (SANDIMMUNE®), tacrolimus (PROGRAF®); and cytostatics, e.g., cyclophosphamide, methotrexate, azathioprine, mitomycin C, bleomycin, and anthracyclines.

In an embodiment, the liver disorder therapy includes agents for treating metabolic disorders, e.g., orlistat (XENICAL®), rosiglitazone (AVANDIA®), vildagliptin, pioglitzaone (ACTOS®), gemfibrozil (Lopid®), thiazolidinediones.

In an embodiment, the liver disorder therapy includes anti-viral agents such as interferons, e.g., interferon 2b, pegylated interferon 2a; nucleoside reverse transcriptase inhibitors (NRTIs), e.g., adefovir, entecavir, lamivudine, ribavirin, telbivudine, and tenofovir; and nucleoside analogs, e.g., 5-iodo 2′-deoxyuridine 5′ monophosphate.

In an embodiment, the liver disorder therapy includes antioxidants β-carotene, biotin, vitamin A, vitamin C, vitamin E, selenium, methionine, allopurinol, desferrioxamine, N-acetylcysteine, manganese, copper, magnesium, folic acid and coenzyme Q.

In an embodiment, the liver disorder therapy includes hepatoprotectants, such as hepatocyte growth factor (HGF) and HGF variants, e.g., HGF deletion variants, HGF synthetic mimetics (Kim et al., 2005, Am J Pathol; Ueki et al., 1999, Nat Med; and Masunaga et al., 1998, Eur J Pharmacol); insulin-like growth factor I; caspase inhibitors, e.g., IDN-6556; farnesoid X receptor (FXR) ligands, e.g., chenodoxycholic acid (CDCA).

In an embodiment, the liver disorder therapy includes agents for palliative care, e.g., medications and procedures that are noncurative but alleviate symptoms, pain, or stress of the liver disorder. Other lifestyle changes may be used concurrently with liver disorder therapies to improve treatment of the disease, such as weight loss, adjustment of diet, nutritional therapy or supplementation, abstinence from alcohol or reduction in alcohol consumption, and abstinence from smoking.

In other embodiments, the combinations described herein are administered in combination with a liver fibrotic therapy, e.g., composition(s) for treatment of liver fibrosis includes, but is not limited to, adefovir dipivoxil, candesartan, colchicine, combined ATG, mycophenolate mofetil, and tacrolimus, combined cyclosporine microemulsion and tacrolimus, elastometry, everolimus, FG-3019, Fuzheng Huayu, GI262570, glycyrrhizin (monoammonium glycyrrhizinate, glycine, L-cysteine monohydrochloride), interferon gamma-lb, irbesartan, losartan, oltipraz, ORAL IMPACT®, peginterferon alfa-2a, combined peginterferon alfa-2a and ribavirin, peginterferon alfa-2b (SCH 54031), combined peginterferon alpha-2b and ribavirin, praziquantel, prazosin, raltegravir, ribavirin (REBETOL®, SCH 18908), ritonavir-boosted protease inhibitor, pentoxyphilline, tacrolimus, tauroursodeoxycholic acid, tocopherol, ursodiol, warfarin, and combinations thereof.

An example of suitable therapeutics for use in combination with the composition(s) for treatment described herein, but is not limited to, 18-FDG, AB0024, ACT-064992 (macitentan), aerosol interferon-gamma, aerosolized human plasma-derived alpha-1 antitrypsin, alphal-proteinase inhibitor, ambrisentan, amikacin, amiloride, amitriptyline, anti-pseudomonas IgY gargle, ARIKACE™, AUREXIS® (tefibazumab), AZAPRED, azathioprine, azithromycin, azithromycin, AZLI, aztreonam lysine, BIBF1120, Bio-25 probiotic, bosentan, Bramitob®, calfactant aerosol, captopril, CC-930, ceftazidime, ceftazidime, cholecalciferol (Vitamin D3), ciprofloxacin (CIPRO®, BAYQ3939), CNTO 888, colistin CF, combined Plasma Exchange (PEX), rituximab, and corticosteroids, cyclophosphamide, dapsone, dasatinib, denufosol tetrasodium (INS37217), dornase alfa (PULMOZYME®), EPI-hNE4, erythromycin, etanercept, FG-3019, fluticasone, FTI, GC1008, GS-9411, hypertonic saline, ibuprofen, iloprost inhalation, imatinib mesylate (GLEEVEC®), inhaled sodium bicarbonate, inhaled sodium pyruvate, interferon gamma-lb, interferon-alpha lozenges, isotonic saline, IWO01, KB001, losartan, lucinactant, mannitol, meropenem, meropenem infusion, miglustat, minocycline, Moli1901, MP-376 (levofloxacin solution for inhalation), mucoid exopolysaccharide P. aeruginosa immune globulin IV, mycophenolate mofetil, nacetylcysteine, N-acetylcysteine (NAC), NaCl 6%, nitric oxide for inhalation, obramycin, octreotide, oligoG CF-5/20, Omalizumab, pioglitazone, piperacillin-tazobactam, pirfenidone, pomalidomide (CC-4047), prednisone, prevastatin, PRM-151, QAX576, rhDNAse, SB656933, SB-656933-AAA, sildenafil, tamoxifen, technetium [Tc-99m] sulfur colloid and Indium [In-111] DTPA, tetrathiomolybdate, thalidomide, ticarcillin-clavulanate, tiotropium bromide, tiotropium RESPIMAT® inhaler, tobramycin (GERNEBCIN®), treprostinil, uridine, valganciclovir (VALCYTE®), vardenafil, vitamin D3, xylitol, zileuton, and combinations thereof.

An example of suitable therapeutics for use in combination with the combinations described herein includes, but is not limited to, cyclosporine, cyclosporine A, daclizumab, everolimus, gadofoveset trisodium (ABLAVAR®), imatinib mesylate (GLEEVEC®), matinib mesylate, methotrexate, mycophenolate mofetil, prednisone, sirolimus, spironolactone, STX-100, tamoxifen, TheraCLEC™, and combinations thereof.

### Therapeutic Methods

In one aspect, the invention relates to a method of treating a disorder, e.g., a hyperproliferative disorder (e.g., a cancer), a fibrotic disorder, or an inflammatory disorder by administering to a patient a metformin agent alone or in combination with one or more of: i) an anti-hypertensive and/or collagen-modifying (AHCM) agent; ii) a microenvironment modulator (e.g., an anti-angiogenic agent) and/or other stromal modulator; iii) an anti-inflammatory agent; or iv) an immune checkpoint inhibitor. as described herein.

By “in combination with,” it is not intended to imply that the therapy or the therapeutic agents must be administered at the same time and/or formulated for delivery together, although these methods of delivery are within the scope of the invention. The pharmaceutical compositions can be administered concurrently with, prior to, or subsequent to, one or more other additional therapies or therapeutic agents. In general, each agent will be administered at a dose and/or on a time schedule determined for that agent. In will further be appreciated that the additional therapeutic agent utilized in this combination can be administered together in a single composition or administered separately in different compositions. The particular combination to employ in a regimen will take into account compatibility of the inventive pharmaceutical composition with the additional therapeutically active agent and/or the desired therapeutic effect to be achieved.

In general, it is expected that additional therapeutic agents utilized in combination be utilized at levels that do not exceed the levels at which they are utilized individually. In some embodiments, the levels utilized in combination will be lower than those utilized individually.

As used herein, and unless otherwise specified, the terms “treat” “treating” and “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

For example, in the case of treating cancer, in some embodiments, therapeutic treatment can refer to inhibiting or reducing tumor growth or progression after administration in accordance with the methods or administration with the pharmaceutical compositions described herein. For example, tumor growth or progression is inhibited or reduced by at least about 10%, at least about 15%, at least about 20%, at least about 30%, at least about 40%, or at least about 50%, after treatment. In another embodiment, tumor growth or progression is inhibited or reduced by more than 50%, e.g., at least about 60%, or at least about 70%, after treatment. In one embodiment, tumor growth or progression is inhibited or reduced by at least about 80%, at least about 90% or greater, as compared to a control (e.g. in the absence of the pharmaceutical composition described herein).

In another embodiment, the therapeutic treatment refers to alleviation of at least one symptom associated with cancer. Measurable lessening includes any statistically significant decline in a measurable marker or symptom, such as measuring a cancer biomarker, such as serum/plasma cancer biomarker in a blood sample, after treatment. In one embodiment, at least one cancer biomarker or sympton is alleviated by at least about 10%, at least about 15%, at least about 20%, at least about 30%, at least about 40%, or at least about 50%. In another embodiment, at least one cancer biomarker or sympton is alleviated by more than 50%, e.g., at least about 60%, or at least about 70%. In one embodiment, at least one cancer biomarker or sympton is alleviated by at least about 80%, at least about 90% or greater, as compared to a control (e.g. in the absence of the pharmaceutical composition described herein).

As used herein, unless otherwise specified, the terms “prevent,” “preventing” and “prevention” contemplate an action that occurs before a patient begins to suffer from the regrowth of the cancer and/or which inhibits or reduces the severity of the cancer.

As used herein, and unless otherwise specified, a “therapeutically effective amount” of a compound is an amount sufficient to provide a therapeutic benefit in the treatment of the disorder (e.g., cancer), or to delay or minimize one or more symptoms associated with the disorder (e.g., cancer). A therapeutically effective amount of a compound means an amount of therapeutic agent, alone or in combination with other therapeutic agents, which provides a therapeutic benefit in the treatment or management of the disorder. The term “therapeutically effective amount” can encompass an amount that improves overall therapy, reduces or avoids symptoms or causes of the disorder (e.g., cancer), or enhances the therapeutic efficacy of another therapeutic agent.

As used herein, and unless otherwise specified, a “prophylactically effective amount” of a compound is an amount sufficient to prevent a disorder (e.g., regrowth of the cancer, or one or more symptoms associated with the cancer, or prevent its recurrence). A prophylactically effective amount of a compound means an amount of the compound, alone or in combination with other therapeutic agents, which provides a prophylactic benefit in the prevention of the disorder. The term “prophylactically effective amount” can encompass an amount that improves overall prophylaxis or enhances the prophylactic efficacy of another prophylactic agent.

As used herein, the term “patient” or “subject” refers to an animal, typically a human (i.e., a male or female of any age group, e.g., a pediatric patient (e.g, infant, child, adolescent) or adult patient (e.g., young adult, middle-aged adult or senior adult) or other mammal, such as a primate (e.g., cynomolgus monkey, rhesus monkey); commercially relevant mammals such as cattle, pigs, horses, sheep, goats, cats, and/or dogs; and/or birds, including commercially relevant birds such as chickens, ducks, geese, and/or turkeys, that will be or has been the object of treatment, observation, and/or experiment. When the term is used in conjunction with administration of a compound or drug, then the patient has been the object of treatment, observation, and/or administration of the compound or drug. The methods and/or pharmaceutical compositions described herein can also be used to treat domesticated animals or pets such as cats and dogs.

As used herein, “cancer” and “tumor” are synonymous terms.

As used herein, “cancer therapy” and “cancer treatment” are synonymous terms.

As used herein, “chemotherapy,” “chemotherapeutic,” “chemotherapeutic agent” and “anti-cancer agent” are synonymous terms.

### Administration of Combination Therapies

In certain embodiments, a metformin agent and i) an AHCM agent, ii) a microenvironment modulator or stromal modulator, iii) an anti-inflammatory agent, and/or iv) an immune-checkpoint agent, as described herein, are administered concurrently (e.g., administration of the two or more agents at the same time or day, or within the same treatment regimen) and/or sequentially (e.g., administration of one agent over a period of time followed by administration of another agent for a second period of time, or within different treatment regimens). In other embodiments, administration of two or more agents occur in overlapping treatment regimens (e.g., administration of one agent is initiated before the completion of the treatment regimen of another agent, or the administration of one agent is completed before the termination of the treatment regimen of another agent).

In one embodiment, the metformin agent is administered prior to the i) AHCM, ii) microenvironment modulator, iii) anti-inflammatory agent, and/or iv) immune checkpoint inhibitor. In other embodiments, the metformin agent is administered prior to the i) AHCM, ii) microenvironment modulator, iii) anti-inflammatory agent, and/or iv) immune checkpoint inhibitor, and followed by concurrent administration of the metformin agent and the i) AHCM, ii) microenvironment modulator, iii) anti-inflammatory agent, and/or iv) immune checkpoint inhibitor.

In certain embodiments, the metformin agent and the i) AHCM, ii) microenvironment modulator, iii) anti-inflammatory agent, and/or iv) immune checkpoint inhibitor are administered concurrently. For example, in certain embodiments, the metformin agent and the i) AHCM, ii) microenvironment modulator, iii) anti-inflammatory agent, and/or iv) immune checkpoint inhibitor are administered at the same time, on the same day, or within the same treatment regimen. In certain embodiments, the metformin agent is administered before the i) AHCM, ii) microenvironment modulator, iii) anti-inflammatory agent, and/or iv) immune checkpoint inhibitor on the same day or within the same treatment regimen.

In certain embodiments, the metformin agent is concurrently administered with the i) AHCM, ii) microenvironment modulator, iii) anti-inflammatory agent, and/or iv) immune checkpoint inhibitor for a period of time, after which point treatment with the i) AHCM, ii) microenvironment modulator, iii) anti-inflammatory agent, and/or iv) immune checkpoint inhibitor is stopped and treatment with the metformin agent continues.

In other embodiments, the metformin agent is concurrently administered with the AHCM, anti-angiogenic agent, anti-inflammatory agent, and/or immune checkpoint inhibitor i) AHCM, ii) microenvironment modulator, iii) anti-inflammatory agent, and/or iv) immune checkpoint inhibitor for a period of time, after which point treatment with metformin agent is stopped and treatment with the i) AHCM, ii) microenvironment modulator, iii) anti-inflammatory agent, and/or iv) immune checkpoint inhibitor continues.

In certain embodiments, the metformin agent and the i) AHCM, ii) microenvironment modulator, iii) anti-inflammatory agent, and/or iv) immune checkpoint inhibitor are administered sequentially. For example, in certain embodiments, the metformin agent is administered after the treatment regimen of the i) AHCM, ii) microenvironment modulator, iii) anti-inflammatory agent, and/or iv) immune checkpoint inhibitor has ceased. In certain embodiments, the i) AHCM, ii) microenvironment modulator, iii) anti-inflammatory agent, and/or iv) immune checkpoint inhibitor is administered after the treatment regimen of the metformin agent has ceased.

In some embodiments, the metformin agent and the i) AHCM, ii) microenvironment modulator, iii) anti-inflammatory agent, and/or iv) immune checkpoint inhibitor can be administered in a pulse administration. In other embodiments, they can be administered as a pulse-chase administration, e.g., where an metformin agent is administered for a brief period of time (pulse), followed by administration of an i) AHCM, ii) microenvironment modulator, iii) anti-inflammatory agent, and/or iv) immune checkpoint inhibitor for a longer period of time (e.g., chase), or vice versa.

In yet other embodiments, the method includes administering the metformin agent and the AHCM, anti-angiogenic agent, anti-inflammatory agent, and/or immune checkpoint inhibitor i) AHCM, ii) microenvironment modulator, iii) anti-inflammatory agent, and/or iv) immune checkpoint inhibitor prior to, concurrently with, or sequentially to a second, third or more additional therapy (e.g., cancer therapy or anti-fibrotic therapy as described herein).

The combination therapies described herein can be administered to the subject systemically (e.g., orally, parenterally, subcutaneously, intravenously, rectally, intramuscularly, intraperitoneally, intranasally, transdermally, or by inhalation or intracavitary installation). Typically, the metformin agent is administered orally. In certain embodiments, the metformin agent and/or the i) AHCM, ii) microenvironment modulator, iii) anti-inflammatory agent, and/or iv) immune checkpoint inhibitor are administered locally or intratumorally (e.g., via an oncolytic virus).

In some embodiments, the metformin agent is administered as a pharmaceutical composition comprising one or more i) AHCM, ii) microenvironment modulator, iii) anti-inflammatory agent, and/or iv) immune checkpoint inhibitor, and a pharmaceutically acceptable excipient.

In other embodiments, the metformin agent and the i) AHCM, ii) microenvironment modulator, iii) anti-inflammatory agent, and/or iv) immune checkpoint inhibitor are administered as separate compositions, e.g., pharmaceutical compositions. In other embodiments, the metformin agent and the i) AHCM, ii) microenvironment modulator, iii) anti-inflammatory agent, and/or iv) immune checkpoint inhibitor are administered separately, but via the same route (e.g., orally or intravenously). In some embodiments, the metformin agent and the i) AHCM, ii) microenvironment modulator, iii) anti-inflammatory agent, and/or iv) immune checkpoint inhibitor are administered by different routes (e.g., metformin agent is administered orally; the i) AHCM, ii) microenvironment modulator, iii) anti-inflammatory agent, and/or iv) immune checkpoint inhibitor is administered subcutaneously or intravenously). In still other instances, the metformin agent and the i) AHCM, ii) microenvironment modulator, iii) anti-inflammatory agent, and/or iv) immune checkpoint inhibitor are administered in the same composition, e.g., pharmaceutical composition.

### Evaluation of Metformin and Combination Therapies

In an embodiment the method comprises a metformin agent and is administered in a dosage sufficient to result in improvement of a disorder-related parameter in said subject, as compared to a subject treated with said therapy but without administration of the metformin agent. “Disorder-related parameter,” as used herein, refers to a parameter that varies with the alleviation of the disorder or a symptom of the disorder.

In an embodiment, a metformin agent is administered and the improvement is as compared to a subject that was not treated with the metformin agent.

In an embodiment, a metformin agent and one or more of i) an AHCM described herein; ii) a microenvironment modulator or stromal modulator described herein; iii) an anti-inflammatory agent described herein; and iv) an immune-checkpoint inhibitor described herein are administered and the improvement is as compared to a subject treated with said therapy but without administration of the metformin agent.

In an embodiment, a metformin agent and an additional therapy, e.g., cancer therapeutic or anti-fibrotic therapeutic, are administered and the improvement is as compared to a subject treated with said therapy but without administration of the metformin agent.

In an embodiment, the parameter comprises relief of a symptom of said disorder.

In an embodiment, the parameter comprises outcome of a patient scored evaluation of symptoms or quality of life, e.g., a quality of life questionaire, e.g., outcome on an evaluation of number of meals consumed on the day prior to the evaluation. pain, weight loss or gain.

In an embodiment, the parameter comprises one or more or all of:

objective response rate (ORR);

progression free survival (PFS);

overall survival (OS), or

reduction in toxicity (whether or not accompanied by an increase on OS. ORR evaluations will differ between disorder but such evaluations are within the skill of the art. For an example, see Willett et al. (2009) Journal of Clinical Oncology 27: 3020-6, which discusses the use of pathological response estimated from evaluation of tissue after surgical resection. Evaluation of PFS is within the skill of the art. For an example, for brain tumors, APF6 (alive and progression-free at 6 months) has been used, see Batchelor et al. (2010) J. Clinical Oncology 28: 2817-23. For radiation therapy, criteria such as “Disease-free survival” and “Freedom from metastasis” have been used, see, e.g., Willett et al, 2010, The Oncologist,15: 845-851. Some evaluations of ORR and PFS rely on imaging methods, e.g., PET, PET-MRI, or PET-CT. Evaluation of toxicity will vary by disorder and treatment modality. One example can be seen in Willett et al. (2010) The Oncologist 15: 845-851.

In an embodiment, the parameter comprises one or more or all of: a) drug concentration, e.g., at a disorder or disease site, e.g., in a solid tumor; b) tumor response; c) blood perfusion, e.g., at a disorder or disease site, e.g., in a solid tumor; d) oxygenation, e.g., at a disorder or disease site, e.g., in a solid tumor; e) interstitial fluid pressure at a disorder or disease site, e.g., in a solid tumor; or f) extracellular matrix content or composition, e.g., level of collagen, hyaluronic acid.

In an embodiment, the parameter is evaluated by a non-invasive method, e.g., a magnetic resonance method, e.g., MRI or MRS, PET, or SPECT.

In an embodiment, the disorder is, e.g., cancer, said parameter is drug concentration, e.g., at a disorder or disease site, e.g., in a solid tumor. In some embodiments the parameter cam be evaluated by a method described herein, e.g., with any of PET-CT, e.g., generally as described in Saleem et al. (2000) The Lancet 355: 2125-2131, MRS, e.g., generally as described in Meisamy et al. (2004) Radiology 233: 424-431, or SPECT, e.g., generally as described in Perik et al. (2006) Journal of Clinical Oncology 24: 2276-2282.

In an embodiment, the disorder is, e.g., cancer, said parameter is blood perfusion, e.g., at a disorder or disease site, e.g., in a solid tumor. In some embodiments, the parameter can be evaluated by a method described herein, e.g. MRI, e.g., generally as described in Sorensen et al. (2012) Cancer Research 72: 402-407, or perfusion CT e.g., generally as described in Park et al. (2009) Radiology 250: 110-117, or Doppler ultrasound generally as described in Singh et al. (2010) European J. of Radiology 75: e158-162.

In an embodiment, the disorder is, e.g., cancer, said parameter is oxygenation, e.g., at a disorder or disease site, e.g., in a solid tumor. In some embodiments, the parameter can be evaluated by a method described herein, e.g., PET, PET-CT, e.g. generally as described in Rajendran et al. (2006) Clinical Cancer Research 12: 5435-5441, or Eppendorf electrode, e.g. generally described in Le et al. (2007) International J. of Radiation Oncology Biology Physics 69: 167-175, or immunohistochemistry, e.g. generally described in Rademakers et al. (2011) BMC Cancer 11: 167.

In an embodiment, the disorder is, e.g., cancer, said parameter is metabolic activity, e.g., at a disorder or disease site, e.g., in a solid tumor In some embodiments the parameter can be evaluated by a method described herein, e.g., functional MRI, or PET, PET-MRI, PET-CT, e.g. generally as described in Shankar et al. (2006) The Journal of Nuclear Medicine 47: 1059-1066. In an embodiment the disorder is, e.g., cancer, said parameter is interstitial fluid pressure, e.g., at a disorder or disease site, e.g., in a solid tumor. In some embodiments, the parameter can be evaluated by a method described herein, e.g., the wick-in- needle technique, e.g., generally as described in Boucher et al. (1991) Cancer Research 51: 6691-6694.

In an embodiment, the disorder is a hyperproliferative fibrotic disease and said parameter is amount of connective tissue matrix or blood perfusion.

In an embodiment, the disorder is an inflammatory disorder, said parameter is amount of connective tissue matrix. In some embodiments, the parameter can be evaluated immunohistochemically.

In an embodiment, the disorder is an autoimmune disorder, said parameter is amount of connective tissue matrix. In some embodiments, the parameter can be evaluated immunohistochemically.

In some embodiments, the parameter is evaluated in a sample from said subject, e.g., a tumor sample, e.g., a biopsy, or a blood or serum sample.

In an embodiment, the parameter comprises one or more or all of:

a) drug concentration, e.g., as evaluated by HPLC, or or NMR, e.g., evaluated generally as described in Olive et al. (2009) Science 324: 1475, HPLC with tandem MS, generally as described in Hu et al. (2011) JNCI 103: 893-905, or by histological measures, e.g., fluorescence imaging of fluorescent drugs, generally as described in Primeau et al. (2005) Clinical Cancer Research 11: 8782-8788;

b) collagen content, e.g., as evaluated by total collagen content measured by hydroxyproline content, e.g., generally as described in Netti et al. (2000) Cancer Research 60: 2497-2503, or immunohistochemistry by antibody staining, e.g., generally as described in Pluen et al. (2001) PNAS 98: 4628-4633;

c) hyaluronan content, e.g., as evaluated by hyaluronan-binding protein labeling of tissue sections, as generally described in Pluen et al. (2001) PNAS 98:4628-4633, or glycosaminoglycan analysis in tissue extracts, e.g., generally as described in Netti et al. (2000) Cancer Research 60: 2497-2503;

d) pathological response, e.g., the prevalence of tumor cells in a sample, e.g., evaluated generally as described in Minckwitz et al. (2012) Journal of Clinical Oncology published as 10.1200/JCO.2011.38.8595;

e) vessel morphology, e.g., size, can be evaluated generally as described in Provenzano et al. (2012) Cancer Cell 21: 418-429, patency (fraction of perfused vessels), e.g., evaluated generally as described in Jacobetz et al. (2012) Gut published on line March 30, 2012, network structure, e.g., evaluated as generally described in Baish et al. (2011) PNAS 108: 1799-1803, luminal opening (measure of perfusion), e.g., evaluated generally as described in Padera et al. (2004) Nature 427: 695, or vessel structure (normalization), e.g., evaluated generally as described in Mazzone et al. (2009) Cell 136: 839-851; or

f) hypoxia, e.g., generally as described in Rademakers et al. (2011) BMC Cancer 11: 167 or Le et al. (2007) International J. of Radiation Oncology Biology Physics 69: 167-175. Hypoxia can be evaluated in a number of ways,e.g.: by a pimonizadole method, see, e.g., Kaanders, J. H. et al. (2002) Cancer Res. 62, 7066-7074; an EFS method, see, e.g., Evans, S. M. et al. (2007) Int. J. Radiat. Oncol. Biol. Phys. 69, 1024-1031; a CA9 method, see, e.g., Koukourakis, M. I. et al., (2006) J. Clin. Oncol. 24, 727-735; a LOX method, see, e.g., Erler, J. T. et al.,. (2006) Nature 440, 1222-1226; a HIF method, see, e.g., Bos, R. et al. (2003) Cancer 97,1573-1581, Yan,et al. (2009) Br. J. Cancer 101, 1168-1174, or Koukourakis, M. I. et al., (2006) J. Clin. Oncol. 24, 727-735; or an electrode method, see, e.g., Nordsmark, M. et al. (2005) Radiother. Oncol. 77, 18-24, Brizel, D. M. et al. (1996) Cancer Res. 56, 941-943, Movsas, B. et al. (2002) Urology 60, 634-639, or Fyles, A. et al. (2002) J. Clin. Oncol. 20, 680-687. See generally Table 2 of Wilson and Hays (2011) Nature Rev Cancer, 11: 393-410

In an embodiment, the parameter is evaluated by immunostaining.

In an embodiment, the parameter comprises one or more or all of:

serum degraded collagen (ICTP), or collagen synthesis (PIP), e.g., evaluated generally as described in Lopez et al. (2001) Circulation 104: 286-291;

serum hyaluronan, e.g., evaluated generally as described in Miele et al. (2009) Translational Research 154: 194-201; or

serum or plasma pro-fibrotic factors (connective tissue growth factor (CTGF), transforming growth factor-beta (TGF-beta), interleukin-1-4,-6,-8, -10 and -13, platelet—derived growth factor (PDGF), stromal cell-derived factor 1 (SDF1), e.g., evaluated generally as described in Harti et al. (2006) American J. of Respiratory Medicine 173: 1371-1376.

In an embodiment, the parameter is drug concentration and said parameter is evaluated by a chromatographic method, e.g., HPLC.

In an embodiment, the disorder is a hyperproliferative fibrotic disease and the parameter is fibrosis.

In an embodiment, the disorder is an inflammatory disorder and the parameter is fibrosis.

In an embodiment, the disorder is an autoimmune disorder and the parameter is fibrosis.

In an embodiment the parameter is a morphological parameter, e.g., evaluated at a disorder or disease site, e.g., in a solid tumor and comprises one or more or all of:

collagen morphology, e.g., evaluated generally as described in Diop-Frimpong et al. (2011) PNAS 108: 2909-2914;

collagen or hyaluronan content, e.g., evaluated generally as described in Pluen et al. (2001) PNAS 98: 4628-4633;

vessel patency (fraction of perfused vessels), e.g., evaluated generally as described in Jacobetz et al. (2012) Gut published on line March 30, 2012; or vessel diameter or size evaluated ,e.g., evaluated generally as described in Provenzano et al. (2012) Cancer Cell 21: 418-429.

### Disorders

Methods described herein comprise administration of a metformin in combination with one or more of i) an AHCM agent, ii) a microenvironment modulator or stromal modulator, iii) an anti-inflammatory agent, or iv) an immune-checkpoint inhibitor, as described herein, for treating or preventing a disorder, e.g., a hyperproliferative disorder (e.g., a cancer) or a fibrotic or an inflammatory condition or disorder described herein.

In certain embodiments, the disorder is chosen from one or more of a hyperproliferative disorder, a cancer (e.g., a solid or fibrotic cancer), a fibrotic disorder or condition, an inflammatory disorder or condition, or an autoimmune disorder.

### Hyperproliferative Disorders and Cancer

In certain embodiments, the cancer is an epithelial, mesenchymal or hematologic malignancy. In certain embodiments, the cancer treated is a solid tumor (e.g., carcinoid, carcinoma or sarcoma), a soft tissue tumor (e.g., a heme malignancy), and a metastatic lesion, e.g., a metastatic lesion of any of the cancers disclosed herein. In one embodiment, the cancer treated is a fibrotic or desmoplastic solid tumor, e.g., a tumor having one or more of: limited tumor perfusion, compressed blood vessels, fibrotic tumor interstitium, or increased interstitial fluid pressure. In one embodiment, the solid tumor is chosen from one or more of pancreatic (e.g., pancreatic adenocarcinoma or pancreatic ductal adenocarcinoma), breast, colon, colorectal, lung (e.g., small cell lung cancer (SCLC) or non-small cell lung cancer (NSCLC)), skin, ovarian, liver cancer, esophageal cancer, endometrial cancer, gastric cancer, head and neck cancer, kidney, or prostate cancer.

By “hyperproliferative cancerous disease or disorder” is meant all neoplastic cell growth and proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and “cancer.”

As used herein, the terms “cancer,” “tumor” or “tumor tissue” refer to an abnormal mass of tissue that results from excessive cell division, in certain cases tissue comprising cells which express, over-express, or abnormally express a hyperproliferative cell protein. A cancer, tumor or tumor tissue comprises “tumor cells” which are neoplastic cells with abnormal growth properties and no useful bodily function. Cancers, tumors, tumor tissue and tumor cells may be benign or malignant. A cancer, tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, p Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

In other embodiements, the metformin agent, as described above and herein, is used to treat a hyperproliferative disorder, e.g., a hyperpoliferative connective tissue disorder (e.g., a hyperproliferative fibrotic disease). In one embodiment, the hyperproliferative fibrotic disease is multisystemic or organ-specific. Exemplary hyperproliferative fibrotic diseases include, but are not limited to, multisystemic (e.g., systemic sclerosis, multifocal fibrosclerosis, sclerodermatous graft-versus-host disease in bone marrow transplant recipients, nephrogenic systemic fibrosis, scleroderma), and organ-specific disorders (e.g., fibrosis of the eye, lung, liver, heart, kidney, pancreas, skin and other organs). In other embodiments, the disorder is chosen from liver cirrhosis or tuberculosis.

In other embodiment, the subject treated has a hyperproliferative genetic disorder, e.g., a hyperproliferative genetic disorder chosen from Marfan's syndrome or Loeys-Dietz syndrome. Losartan has been shown to treat human Marfan syndrome, a connective tissue disorder caused by mutations in the gene that encodes the extracellular matrix protein, fibrillin-1 (Dietz, H.C. et al. (2010) New Engl J Med 363(9):852-863). Fibrillin-1 comprises the microfibrils of elastic tissue and a component of many other connective tissues. Affected patients with Marfan syndrome have blood vessel abnormalities such as aortic aneurysms. The vascular disease can result in blood vessel rupture and death in childhood and later in life. Dietz et al. first found in mouse models of Marfan syndrome that excessive activation of latent TGF-β has an important role in the pathophysiology. They used losartan in the affected mice and showed striking effects in improving blood vessel architecture and prevented the development of aortic aneurysms. They have also used losartan to treat children with Marfan syndrome and demonstrated that the drug can strikingly prevent progression of aortic and muscular lesions. Aortic diseases other than Marfan syndrome can also benefit from the use of losartan. Inhibition of activation of latent TGF-β locally and decreasing circulating levels of active TGF-β thus can have effects on components of connective tissues other than collagen in the extracellular matrix of cancer tissues that alter delivery and efficacy of nanotherapeutics.

In other embodiments, the hyperproliferative disorder (e.g., the hyperproliferative fibrotic disorder) is chosen from one or more of chronic obstructive pulmonary disease, asthma, aortic aneurysm, radiation-induced fibrosis, skeletal-muscle myopathy, diabetic nephropathy, and/or arthritis.

Additional exemplary hyperproliferative disorders that can be treated by the methods and compositions of the invention are disclosed in Sounni, N. E. et al. (2010) Diseases Models & Mechanisms 3: 317-332.

In yet other embodiments, the disorder is chosen from an inflammatory or an autoimmune disorder chosen from multiple sclerosis, inflammatory bowel disease, scleroderma, lupus, rheumatoid arthritis or osteoarthritis.

In certain embodiments, the inflammatory disorder is an inflammatory disorder of: the gastrointestinal tract or a gastrointestinal organ, e.g., colitis, Crohn's disease, inflammatory bowel disease (IBD), Barrett's esophagus and chronic gastritis; the lung (e.g., asthma, chronic obstructive pulmonary disease (COPD); the skin (e.g., psoriasis), the cardiovascular system (e.g., atherosclerosis, cholesterol metabolic disorders, oxygen free radical injury, ischemia), the nervous system (e.g., Alzheimer's disease, multiple sclerosis), liver (e.g., hepatitis), kidney (e.g., nephritis), and the pancreas (e.g., pancreatitis).

In other embodiments, the inflammatory disorder is associated with an autoimmune disorder, e.g., arthritis (including rheumatoid arthritis, juvenile rheumatoid arthritis, osteoarthritis, psoriatic arthritis, lupus-associated arthritis, autoimmune thyroiditis or ankylosing spondylitis); scleroderma; lupus; systemic lupus erythematosis; HIV; Sjogren's syndrome; vasculitis; multiple sclerosis; dermatitis (including atopic dermatitis and eczematous dermatitis), myasthenia gravis, inflammatory bowel disease (IBD), Crohn's disease, colitis, diabetes mellitus (type I); acute inflammatory conditions (e.g., endotoxemia, sepsis and septicaemia, toxic shock syndrome and infectious disease); transplant rejection and allergy.

### Liver Conditions or Disorders

Examples of liver cancers include: hepatocellular carcinoma (HCC), primary liver cell carcinoma, hepatoma, fibrolamellar carcinoma, focal nodular hyperplasia, cholangiosarcoma, intrahepatic bile duct cancer, angiosarcoma or hemangiosarcoma, hepatic adenoma, hepatic hemangiomas, hepatic hamartoma, hepatoblastoma, infantile hemangioendothelialoma, mixed tumors of the liver, tumors of mesenchymal tissue, and sarcoma of the liver. Examples of cancers that may metastasize to the liver include: breast cancer, colorectal cancer, esophageal cancer, kidney or renal cancer, lung cancer, ovarian cancer, pancreatic cancer, rectal cancer, skin cancer (e.g., melanoma), gastric or stomach cancer (including gastrointestinal cancer), and uterine cancer.

In an embodiment, the liver disorder is a fibrotic disorder or connective tissue disorder affecting the function or physiology of the liver. In one embodiment, the fibrotic disorder or connective tissue disorder can be systemic (affecting the whole body), multi-organ, or organ-specific (e.g., liver-specific). Examples of fibrotic liver disorders include liver fibrosis (hepatic fibrosis), liver cirrhosis, and any disorder associated with accumulation of extracellular matrix proteins, e.g., collagen, in the liver, liver scarring, and/or abnormal hepatic vasculature. Liver fibrosis is caused by liver inflammation or damage which triggers the accumulation of extracellular matrix proteins, including collagens, and scar tissue in the liver. Liver cirrhosis is the end stage of liver fibrosis, involves regenerative nodules (as a result of repair processes), and is accompanied with the distortion of the hepatic vasculature. Liver fibrotic disorders are most commonly caused by chronic viral infection (e.g., hepatitis B, hepatitis C), alcoholism, and fatty liver disease.

Examples of fatty liver diseases include fatty liver (or FLD), alcoholic liver disease, non-alcoholic fatty liver disease (NAFLD), non-alcoholic steatohepatitis (NASH), alcoholic steatohepatitis, simple steatosis, Reye's syndrome, and any disorder associated with abnormal retention of lipids in liver cells.

In one embodiment, the liver disease is NASH.

Metabolic disorders can also affect the liver and cause liver damage. Examples of metabolic disorders of the liver or affecting the liver include hemachromatosis, diabetes, obesity, hypertension, dyslipidemia, galactosemia, and glycogen storage disease.

Autoimmune disorders of the liver or affecting the liver can include systemic disorders or disorders that primarily affect an organ other than the liver, but with secondary effects to liver cells or liver function. Examples of such autoimmune disorders include autoimmune hepatitis (AIH), autoimmune liver disease, lupoid hepatitis, systemic lupus erythematosus, primary biliary cirrhosis (PBC), scleroderma, and systemic scerlosis.

Disorders associated with inflammation of the liver include steatohepatitis, primary sclerosing cholangitis (PSC), ulcerative colitis, Crohn's disease, inflammatory bowel disease, or any disorder associated with inflammation in the liver.

In an embodiment, the liver disorder is associated with an inherited or congenital disease, e.g., Wilson's disease, Gilbert's disease, Byler syndrome, Greenland-Eskimo familial cholestasis, Zellweger's syndrome, Alagilles syndrome (ALGS), progressive familial intrahepatic cholestasis (PFIC), or alpha 1-antitrypsin deficiency, cystic fibrosis, Indian childhood cirrhosis, and hereditary hemochromatosis.

In an embodiment, the liver disorder is associated with pancreatic or biliary tract damage or disorders, e.g., cerebrotendinous, xanthomatosis, gall stones, choledocholithiasis, obstruction of the biliary tree, biliary trauma, biliary atresia, pancreatitis, primary biliary cirrhosis, primary sclerosing cholangitis, cholestasis, cholestasis of pregnancy, or any disorder associated with the obstruction or damage to the biliary system or the pancreas.

In an embodiment, liver disorders can be induced by infection, for example, by viral infections such as hepatitis A virus, hepatitis B virus, hepatitis C virus, hepatitis D virus (hepatitis delta virus), hepatitis E virus, Epstein-Barr adenovirus, or cytomegalovirus; or parasitic infection, such as schistosomiasis.

In an embodiment, liver disorders can be induced by drugs, such as acetaminophen (e.g., paracetamol, TYLENOL®, or PANADOL®), nonsteroidal anti-inflammatory drugs (NSAIDS, e.g., aspirin and phenylbutazone, ibruprofen, piroxicam, diclofenac, sulindac, and indomethacin), glucocorticoids, anti-tuberculosis drugs (e.g., isoniazid), antibiotics, anesthetics, antihypertensives (e.g., statins), oral contraceptives, dietary aids, or herbal supplements (e.g., ackee fruit, bajiaolian, boragecamphor, copaltra, comfrey, cycasin, kava leaves, pyrrolizidine alkaloids, horse chestnut leaves, valerian); or toxins, such as arsenic, carbon tetrachloride, vinyl chloride, aflatoxins.

In an embodiment, liver disorders also include disorders or conditions induced by injury to the liver or affecting the liver, including drug toxicity, alcoholism, ischemia, malnutrition, or physical trauma.

Other liver disorders include hepatic vein thrombosis, Budd-Chiari syndrome, portal hypertension, hepatic encephalopathy, and hepatomegaly (or enlarged liver).

### Fibrotic Conditions or Disorders

In another aspect, the invention features a method of treating or preventing a fibrotic condition or disorder in a subject. The method includes administering a composition described herein (e.g., a metformin agent and one or more of: an AHCM, a microenvironment modulator or other stromal modulator, an inflammatory agent, and/or an immune-checkpoint inhibitor) alone or in combination with another agent or therapeutic modality, to a subject in need thereof, in an amount sufficient to decrease or inhibit the fibrotic condition in the subject.

In certain embodiments, reducing fibrosis, or treatment of a fibrotic condition, includes reducing or inhibiting one or more of: formation or deposition of tissue fibrosis; reducing the size, cellularity (e.g., fibroblast or immune cell numbers), composition; or cellular content, of a fibrotic lesion; reducing the collagen or hydroxyproline content, of a fibrotic lesion; reducing expression or activity of a fibrogenic protein; reducing fibrosis associated with an inflammatory response; decreasing weight loss associated with fibrosis; or increasing survival.

In certain embodiments, the fibrotic condition is primary fibrosis. In one embodiment, the fibrotic condition is idiopathic. In other embodiments, the fibrotic condition is associated with (e.g., is secondary to) a disease (e.g., an infectious disease, an inflammatory disease, an autoimmune disease, a malignant or cancerous disease, and/or a connective disease); a toxin; an insult (e.g., an environmental hazard (e.g., asbestos, coal dust, polycyclic aromatic hydrocarbons), cigarette smoking, a wound); a medical treatment (e.g., surgical incision, chemotherapy or radiation), or a combination thereof. In certain embodiments, the fibrotic condition is a fibrotic condition of the lung, a fibrotic condition of the liver (e.g., as described herein), a fibrotic condition of the heart or vasculature, a fibrotic condition of the kidney, a fibrotic condition of the skin, a fibrotic condition of the gastrointestinal tract, a fibrotic condition of the bone marrow or a hematopoietic tissue, a fibrotic condition of the nervous system, a fibrotic condition of the eye, or a combination thereof.

In certain embodiments, the fibrotic condition is a fibrotic condition of the lung. In certain embodiments, the fibrotic condition of the lung is chosen from one or more of: pulmonary fibrosis, idiopathic pulmonary fibrosis (IPF), usual interstitial pneumonitis (UIP), interstitial lung disease, cryptogenic fibrosing alveolitis (CFA), bronchiectasis, and scleroderma lung disease. In one embodiment, the fibrosis of the lung is secondary to a disease, a toxin, an insult, a medical treatment, or a combination thereof. For example, the fibrosis of the lung can be associated with (e.g., secondary to) one or more of: a disease process such as asbestosis and silicosis; an occupational hazard; an environmental pollutant; cigarette smoking; an autoimmune connective tissue disorders (e.g., rheumatoid arthritis, scleroderma and systemic lupus erythematosus (SLE)); a connective tissue disorder such as sarcoidosis; an infectious disease, e.g., infection, particularly chronic infection; a medical treatment, including but not limited to, radiation therapy, and drug therapy, e.g., chemotherapy (e.g., treatment with as bleomycin, methotrexate, amiodarone, busulfan, and/or nitrofurantoin). In one embodiment, the fibrotic condition of the lung treated with the methods of the invention is associated with (e.g., secondary to) a cancer treatment, e.g., treatment of a cancer (e.g., squamous cell carcinoma, testicular cancer, Hodgkin's disease with bleomycin). In one embodiment, the fibrotic condition of the lung is associated with an autoimmune connective tissue disorder (e.g., scleroderma or lupus, e.g., SLE).

Pulmonary fibrosis can occur as a secondary effect in disease processes such as asbestosis and silicosis, and is known to be more prevalent in certain occupations such as coal miner, ship workers and sand blasters where exposure to environmental pollutants is an occupational hazard (Green, F H et al. (2007) Toxicol Pathol. 35: 136-47). Other factors that contribute to pulmonary fibrosis include cigarette smoking, and autoimmune connective tissue disorders, like rheumatoid arthritis, scleroderma and systemic lupus erythematosus (SLE) (Leslie, K O et al. (2007) Semin Respir Crit Care Med. 28: 369-78; Swigris, J J et al. (2008) Chest. 133: 271-80; and Antoniou, K M et al. (2008) Curr Opin Rheumatol. 20: 686-91). Other connective tissue disorders such as sarcoidosis can include pulmonary fibrosis as part of the disease (Paramothayan, S et al. (2008) Respir Med. 102: 1-9), and infectious diseases of the lung can cause fibrosis as a long term consequence of infection, particularly chronic infections.

Pulmonary fibrosis can also be a side effect of certain medical treatments, particularly radiation therapy to the chest and certain medicines like bleomycin, methotrexate, amiodarone, busulfan, and nitrofurantoin (Catane, R et al. (1979) Int J Radiat Oncol Biol Phys. 5: 1513-8; Zisman, D A et al. (2001) Sarcoidosis Vasc Diffuse Lung Dis. 18: 243-52; Rakita, Let al. (1983) Am Heart J. 106: 906-16; Twohig, K J et al. (1990) Clin Chest Med. 11:31-54; and Witten C M. (1989) Arch Phys Med Rehabil. 70: 55-7). In other embodiments, idiopathic pulmonary fibrosis can occur where no clear causal agent or disease can be identified. Genetic factors can play a significant role in these cases of pulmonary fibrosis (Steele, MP et al. (2007) Respiration 74: 601-8; Brass, D M et al. (2007) Proc Am Thorac Soc. 4: 92-100 and du Bois R M. (2006) Semin Respir Crit Care Med. 27: 581-8).

In other embodiments, pulmonary fibrosis includes, but is not limited to, pulmonary fibrosis associated with chronic obstructive pulmonary disease (COPD), acute respiratory distress syndrome, scleroderma, pleural fibrosis, chronic asthma, acute lung syndrome, amyloidosis, bronchopulmonary dysplasia, Caplan's disease, Dressler's syndrome, histiocytosis X, idiopathic pulmonary haemosiderosis, lymphangiomyomatosis, mitral valve stenosis, polymyositis, pulmonary edema, pulmonary hypertension (e.g., idiopathic pulmonary hypertension (IPH)), pneumoconiosis, radiotherapy (e.g., radiation induced fibrosis), rheumatoid disease, Shaver's disease, systemic lupus erythematosus, systemic sclerosis, tropical pulmonary eosinophilia, tuberous sclerosis, Weber-Christian disease, Wegener's granulomatosis, Whipple's disease, or exposure to toxins or irritants (e.g., pharmaceutical drugs such as amiodarone, bleomycin, busulphan, carmustine, chloramphenicol, hexamethonium, methotrexate, methysergide, mitomycin C , nitrofurantoin, penicillamine, peplomycin, and practolol; inhalation of talc or dust, e.g., coal dust, silica). In certain embodiments, the pulmonary fibrosis is associated with an inflammatory disorder of the lung, e.g., asthma, and/or COPD.

In certain embodiments, the fibrotic condition is a fibrotic condition of the liver. In certain embodiments, the fibrotic condition of the liver is chosen from one or more of: fatty liver disease, steatosis (e.g., nonalcoholic steatohepatitis (NASH), cholestatic liver disease (e.g., primary biliary cirrhosis (PBC)), cirrhosis, alcohol induced liver fibrosis, biliary duct injury, biliary fibrosis, or cholangiopathies. In other embodiments, hepatic or liver fibrosis includes, but is not limited to, hepatic fibrosis associated with alcoholism, viral infection, e.g., hepatitis (e.g., hepatitis C, B or D), autoimmune hepatitis, non-alcoholic fatty liver disease (NAFLD), progressive massive fibrosis, exposure to toxins or irritants (e.g., alcohol, pharmaceutical drugs and environmental toxins). Additional examples of liver conditions and disorders are provided in the Sections entitled “Liver Conditions or Disorders,” provided herein.

In certain embodiments, the fibrotic condition is a fibrotic condition of the kidney. In certain embodiments, the fibrotic condition of the kidney is chosen from one or more of: renal fibrosis (e.g., chronic kidney fibrosis), nephropathies associated with injury/fibrosis (e.g., chronic nephropathies associated with diabetes (e.g., diabetic nephropathy)), lupus, scleroderma of the kidney, glomerular nephritis, focal segmental glomerular sclerosis, IgA nephropathyrenal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, HIV-associated nephropathy, or fibrosis associated with exposure to a toxin, an irritant, or a chemotherapeutic agent. In one embodiment, the fibrotic condition of the kidney is scleroderma of the kidney. In some embodiments, the fibrotic condition of the kidney is transplant nephropathy, diabetic nephropathy, lupus nephritis, focal segmental glomerulosclerosis (FSGS), endothelial/thrombotic microangiopathy injury, scleroderma of the kidney, HIV-associated nephropathy (HIVVAN), or exposure to toxins, irritants, chemotherapeutic agents.

In certain embodiments, the fibrotic condition is a fibrotic condition of the bone marrow or a hematopoietic tissue. In certain embodiments, the fibrotic condition of the bone marrow is an intrinsic feature of a chronic myeloproliferative neoplasm of the bone marrow, such as primary myelofibrosis (also referred to herein as agnogenic myeloid metaplasia or chronic idiopathic myelofibrosis). In other embodiments, the bone marrow fibrosis is associated with (e.g., is secondary to) a malignant condition or a condition caused by a clonal proliferative disease. In other embodiments, the bone marrow fibrosis is associated with a hematologic disorder (e.g., a hematologic disorder chosen from one or more of polycythemia vera, essential thrombocythemia, myelodysplasia, hairy cell leukemia, lymphoma (e.g., Hodgkin or non-Hodgkin lymphoma), multiple myeloma or chronic myelogeneous leukemia (CML)). In yet other embodiments, the bone marrow fibrosis is associated with (e.g., secondary to) a non-hematologic disorder (e.g., a non-hematologic disorder chosen from solid tumor metastasis to bone marrow, an autoimmune disorder (e.g., systemic lupus erythematosus, scleroderma, mixed connective tissue disorder, or polymyositis), an infection (e.g., tuberculosis or leprosy), or secondary hyperparathyroidism associated with vitamin D deficiency. In some embodiments, the fibrotic condition is idiopathic or drug-induced myelofibrosis. In some embodiments, the fibrotic condition of the bone marrow or hematopoietic tissue is associated with systemic lupus erythematosus or scleroderma.

In other embodiments, the fibrotic condition is associated with leprosy or tuberculosis.

In certain embodiments, the fibrotic condition is a fibrotic condition of the bone marrow. In certain embodiments, the fibrotic condition of the bone marrow is myelofibrosis (e.g., primary myelofibrosis (PMF)), myeloid metaplasia, chronic idiopathic myelofibrosis, or primary myelofibrosis. In other embodiments, bone marrow fibrosis is associated with a hematologic disorder chosen from one or more of hairy cell leukemia, lymphoma, or multiple myeloma.

In other embodiments, the bone marrow fibrosis is associated with one or more myeloproliferative neoplasms (MPN) chosen from: essential thrombocythemia (ET), polycythemia vera (PV), mastocytosis, chronic eosinophilic leukemia, chronic neutrophilic leukemia, or other MPN.

In one embodiment, the fibrotic condition is primary myelofibrosis. Primary myelofibrosis (PMF) (also referred to in the literature as idiopathic myeloid metaplasia, and Agnogenic myeloid metaplasia) is a clonal disorder of multipotent hematopoietic progenitor cells (reviewed in Abdel-Wahab, 0. et al. (2009) Annu. Rev. Med. 60: 233-45; Varicchio, L. et al. (2009) Expert Rev. Hematol. 2(3):315-334; Agrawal, M. et al. (2010) Cancer 1-15).

In certain embodiments, the fibrotic condition is a fibrotic condition of the heart. In certain embodiments, the fibrotic condition of the heart is myocardial fibrosis (e.g., myocardial fibrosis associated with radiation myocarditis, a surgical procedure complication (e.g., myocardial post-operative fibrosis), infectious diseases (e.g., Chagas disease, bacterial, trichinosis or fungal myocarditis)); granulomatous, metabolic storage disorders (e.g., cardiomyopathy, hemochromatosis); developmental disorders (e.g, endocardial fibroelastosis); arteriosclerotic, or exposure to toxins or irritants (e.g., drug induced cardiomyopathy, drug induced cardiotoxicity, alcoholic cardiomyopathy, cobalt poisoning or exposure). In certain embodiments, the myocardial fibrosis is associated with an inflammatory disorder of cardiac tissue (e.g., myocardial sarcoidosis). In some embodiments, the fibrotic condition is a fibrotic condition associated with a myocardial infarction. In some embodiments, the fibrotic condition is a fibrotic condition associated with congestive heart failure.

In some embodiments, the fibrotic condition is associated with an autoimmune disease selected from scleroderma or lupus, e.g., systemic lupus erythematosus.

In some embodiments, the fibrotic condition is systemic. In some embodiments, the fibrotic condition is systemic sclerosis (e.g., limited systemic sclerosis, diffuse systemic sclerosis, or systemic sclerosis sine scleroderma), nephrogenic systemic fibrosis, cystic fibrosis, chronic graft vs. host disease, or atherosclerosis.

In some embodiments, the fibrotic condition is scleroderma. In some embodiments, the scleroderma is localized, e.g., morphea or linear scleroderma. In some embodiments, the condition is a systemic sclerosis, e.g., limited systemic sclerosis, diffuse systemic sclerosis, or systemic sclerosis sine scleroderma.

In other embodiment, the fibrotic condition affects a tissue chosen from one or more of muscle, tendon, cartilage, skin (e.g., skin epidermis or endodermis), cardiac tissue, vascular tissue (e.g., artery, vein), pancreatic tissue, lung tissue, liver tissue, kidney tissue, uterine tissue, ovarian tissue, neural tissue, testicular tissue, peritoneal tissue, colon, small intestine, biliary tract, gut, bone marrow, hematopoietic tissue, or eye (e.g., retinal) tissue.

In some embodiments, the fibrotic condition is a fibrotic condition of the eye. In some embodiments, the fibrotic condition is glaucoma, macular degeneration (e.g., age-related macular degeneration), macular edema (e.g., diabetic macular edema), retinopathy (e.g., diabetic retinopathy), or dry eye disease.

In certain embodiments, the fibrotic condition is a fibrotic condition of the skin. In certain embodiments, the fibrotic condition of the skin is chosen from one or more of: skin fibrosis (e.g., hypertrophic scarring, keloid), scleroderma, nephrogenic systemic fibrosis (e.g., resulting after exposure to gadolinium (which is frequently used as a contrast substance for MRIs) in patients with severe kidney failure), and keloid.

In certain embodiments, the fibrotic condition is a fibrotic condition of the gastrointestinal tract. In certain embodiments, the fibrotic condition is chosen from one or more of: fibrosis associated with scleroderma; radiation induced gut fibrosis; fibrosis associated with a foregut inflammatory disorder such as Barrett's esophagus and chronic gastritis, and/or fibrosis associated with a hindgut inflammatory disorder, such as inflammatory bowel disease (IBD), ulcerative colitis and Crohn's disease. In some embodiments, the fibrotic condition of the gastrointestinal tract is fibrosis associated with scleroderma.

In one embodiment, the fibrotic condition is a chronic fibrotic condition or disorder. In certain embodiments, the fibrotic condition is associated with an inflammatory condition or disorder. In some embodiments, the fibrotic and/or inflammatory condition is osteomyelitis, e.g., chronic osteomyelitis.

In some embodiments, the fibrotic condition is an amyloidosis. In certain embodiments, the amyloidosis is associated with chronic osteomyelitis.

### Pharmaceutical Compositions

The compositions described herein can be incorporated into a variety of formulations for administration. More particularly, the compositions can be formulated into pharmaceutical compositions by combination with appropriate, pharmaceutically acceptable carriers or diluents, and can be formulated into preparations in semi-solid, liquid or gaseous forms; such as capsules, powders, granules, gels, slurries, ointments, solutions, suppositories, injections, inhalants and aerosols. As such, administration of the compositions can be achieved in various ways, including oral, buccal, rectal, parenteral, intraperitoneal, intradermal, transdermal, intratracheal administration. Moreover, the compositions can be administered in a local rather than systemic manner, in a depot or sustained release formulation.

In addition, the compositions can be formulated with common excipients, diluents or carriers, and compressed into tablets, or formulated as elixirs or solutions for convenient oral administration, or administered by the intramuscular or intravenous routes. The compositions can be administered transdermally, and can be formulated as sustained release dosage forms and the like. Compositions can be administered alone, in combination with each other, or they can be used in combination with other known compounds (discussed herein).

Suitable formulations for use in the present invention are found in Remington's Pharmaceutical Sciences (1985). Moreover, for a review of methods for drug delivery, see, Langer (1990) Science 249: 1527-1533. The pharmaceutical compositions described herein can be manufactured in a manner that is known to those of skill in the art, e.g., by mixing, dissolving, granulating, dragee-making, levigating, emulsifying, encapsulating, entrapping or lyophilizing processes. The following methods and excipients are merely exemplary and are in no way limiting.

For oral administration, the compositions can be formulated by combining with pharmaceutically acceptable carriers that are known in the art. Such carriers enable the compounds to be formulated as pills, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compositions with an excipient and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compositions can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions or emulsions in oily or aqueous vehicles, and can contain formulator agents such as suspending, stabilizing and/or dispersing agents.

The compositions can also be formulated in rectal compositions such as suppositories or retention enemas, e.g., containing conventional suppository bases such as cocoa butter, carbowaxes, polyethylene glycols or other glycerides, all of which melt at body temperature, yet are solidified at room temperature.

In addition, the compositions can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Lipid particles (e.g., liposomes) and emulsions are known examples of delivery vehicles or carriers for hydrophobic drugs. Long-circulating, e.g., stealth, liposomes can be employed. Such liposomes are generally described in U.S. Pat. No. 5,013,556. The compounds of the present invention can also be administered by controlled release means and/or delivery devices such as those described in U.S. Pat. Nos. 3,845,770; 3,916,899; 3,536,809; 3,598,123; and 4,008,719.

Pharmaceutical compositions suitable for use in the present invention include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein. In general, a suitable daily dose of an AHCM agent and/or a cancer therapeutic can be that amount of the compound which is the lowest dose effective to produce a therapeutic effect. Such an effective dose can generally depend upon the factors described above.

The subject receiving this treatment is any animal in need, including primates, in particular humans, equines, cattle, swine, sheep, poultry, dogs, cats, mice and rats.

The compounds can be administered daily, every other day, three times a week, twice a week, weekly, or bi-weekly. The dosing schedule can include a “drug holiday,” i.e., the drug can be administered for two weeks on, one week off, or three weeks on, one week off, or four weeks on, one week off, etc., or continuously, without a drug holiday. The compounds can be administered orally, intravenously, intraperitoneally, topically, transdermally, intramuscularly, subcutaneously, intranasally, sublingually, or by any other route.

Since metformin agent is administered in combination with other treatments (such as additional chemotherapeutics, radiation or surgery) the doses of each agent or therapy can be lower than the corresponding dose for single-agent therapy. The determination of the mode of administration and the correct dosage is well within the knowledge of the skilled clinician.

In certain embodiments, metformin agent (alone or in combination with the therapeutic agents described herein is formulated for oral, subcutaneous, intravenous or intraperitoneal administration. In one embodiment, metformin (alone or in combination with the therapeutic agents described herein is formulated for oral administration (e.g., an oral tablet or pill).

Substantially continuous administration of metformin agent (alone or in combination with the therapeutic agents described herein) can cause a greater reduction in collagen content and/or tumor size than single or pulsatile administration of the metformin agent. Thus, it may be desirable to formulate and/or administered the metformin agent (alone or in combination with the therapies described herein) substantially continuously.

In one embodiment, the metformin agent (alone or in combination) is administered substantially continuously over a pre-determined period of, or at least 15, 30, 45 minutes; a period of, or at least, 1, 5, 10, 24 hours; a period of, or at least, 2, 5, 10, 14 days; a period of, or at least, 3, 4, 5, 6, 7, 8 weeks; a period of, or at least 2, 3, 4, 5, 6, 7, 8, 9, 10, 11 months; a period of, or at least, 1, 2, 3, 4, 5 years, or longer. The delivery method can be optimized such that a metformin agent dose as described herein (alone or in combination) is administered and/or maintained in the subject for a pre-determined period (e.g., a period as described herein).

The metformin agent or any of the AHCM and/or microenvironment modulators is in a controlled- or extended release formulation, dosage form, or device. Exemplary formulations and devices for controlled or extended release are known in the art. For example, formulations containing polymer matrices, such as hydroxypropylmethyl cellulose, gels, osmotic systems, liposomes and combination thereof can be used to provide the desired release kinetics.

In one embodiment, the metformin agent or any of the AHCM and/or microenvironment modulators is administered via an implantable infusion device, e.g., a pump (e.g., a subcutaneous pump), an implant or a depot. Implantable infusion devices typically include a housing containing a liquid reservoir which can be filled transcutaneously by a hypodermic needle penetrating a fill port septum. The medication reservoir is generally coupled via an internal flow path to a device outlet port for delivering the liquid through a catheter to a patient body site. Typical infusion devices also include a controller and a fluid transfer mechanism, such as a pump or a valve, for moving the liquid from the reservoir through the internal flow path to the device's outlet port.

### Particles

One or more of AHCM, microenvironment modulatiors, metformin, and other agents described herein, can be packaged in particles, e.g., nanoparticles.

Typically nanoparticles are from 10, 15, 20, 25, 30, 35, 45, 50, 75, 100, 150 or 200 nm or 200-1,000, e.g., 10, 15, 20, 25, 30, 35, 45, 50, 75, 100, 150, or 200, or 20 or 30 or 50-400 nm in diameter. Smaller particles tend to be cleared more rapidly form the system. Drugs can be intrapped within or coupled, e.g., covalent coupled, or otherwise adhered, to nanoparticles.

Lipid- or oil-based nanoparticles, such as liposomes and solid lipid nanoparticles and can be used to can be used to deliver agents described herein. DOXIL® is an example of a liposomic nanoparticle. Solid lipid nanoparticles for the delivery on anti-cancer agents are described in Serpe et al. (2004) Eur. J. Pharm. Bioparm. 58: 673-680 and Lu et al. (20060 Eur. J. Pharm. Sci. 28: 86-95. Polymer-based nanoparticles, e.g., PLGA-based nanoparticles can be used to deliver agents described herein. These tend to rely on biodegradable backbone with the theraeutic agent intercalated (with or without covalent linkage to the polymer) in a matrix of polymer. PLGA is a widely used in polymeric nanoparticles, see Hu et al. (2009) J. Control. Release 134:55-61; Cheng et al. (2007) Biomaterials 28: 869-876, and Chan et al. (2009) Biomaterials 30: 1627-1634. PEGylated PLGA-based nanoparticles can also be used to deliver anti-cancer agents, see, e.g., Danhhier et al., (2009) J. Control. Release 133: 11-17, Gryparis et al (2007) Eur. J. Pharm. Biopharm. 67: 1-8. Metal-based, e.g., gold-based nanoparticles can also be used to deliver anti-cancer agents. Protein-based, e.g., albumin-based nanoparticles can be used to deliver agents described herein. E.g., an agent can be bound to nanoparticles of human albumin. An exemplary anti-cancer agent/protein nanoparticle is Abraxane®, in which paclitaxel is pund to nanoparticles of albumin.

Nanoparticles can employ active targeting, passive targeting or both. Active targeting can rely on inclusion of a ligand that binds with a target at or near a preselected site, e.g., a solid tumor. Passive targeting nanoparticles can diffuse and accumulate at sites of interest, e.g., sites characterized by excessively leaky micorvasculatiure, e.g., as seen in tumors and sites of inflammation.

A broad range of nanoparticles are known in the art. Exemplary approaches include those described in WO2010/005726, WO2010/005723 WO2010/005721, WO2010/121949, WO2010/0075072, WO2010/068866, WO2010/005740, WO2006/014626, U.S. Pat. No. 7,820,788, and U.S. Pat. No. 7,780,984, the contents of which are incorporated herein in reference by their entirety.

Examples of non-targeted polymeric nanoparticles include, but are not limited to, a polymeric-micelle composed of PEG and polylactic acid (PLA) (e.g., Cynviloq® which encapsulates paclitaxel (developed by Sorrento Therapeutics)); a polymeric PEG-polyamino acid (e.g., NC-6004, which encapsulates cisplatin (developed by NanoCarrier Co.)); a polymeric PEG-polyaspartate (e.g., NK105, which encapsulates paclitaxel (developed by Nippon Kayaku Co.); a polymeric PEG-polyaspartate (e.g., NK911, which encapsulates doxorubicin (developed by Nippon Kayaku Co.); a cyclodextrin polymeric nanoparticle (CDP) as described in, e.g., U.S. Pat. No. 8,389,499, U.S. Pat. No. 8,314,230, U.S. Pat. No. 8,603,454, U.S. Pat. No. 8,404,799, all of which are incorporated herein by reference. Exemplary cyclodextin-PEG polymers comprising various chemotherapeutic drugs include CDPs bound to camptothecin (e.g., CRLX101 to treat relapsed renal cell carcinoma, ovarian cancer, rectal cancer), and CDPs bound to docetaxel (e.g., CRLX301).

Polymeric nanoparticles described herein can include a targeting agent, e.g., an antibody or antibody fragment, or a ligand, e.g., transferrin or other receptor ligand. Examples of targeted polymeric nanoparticles include, but are not limited to, polylactic co-glycolic acid (PLGA) nanoparticles encapsulating a reverse micelle, in which the interior is hydrophilic and the exterior is hydrophobic. Either part of the micelle may be loaded with therapeutics. The PLGA matrix is coated with PEG for immune shielding, and a targeting ligand (e.g., one or more of an antibody, an antibody fragment, or a ligand (e.g., a receptor ligand)); the ligand is linked to a PEG molecule to direct the particles within the body. These targeted nanoparticles are described in, e.g., U.S. Pat. No. 8,193,334, U.S. Pat. No. 7,534,449, and Hrkach, J. et al. Preclinical development and clinical translation of a PSMA-targeted docetaxel nanoparticle with a differentiated pharmalogical profile. (2012) Science Translational Medicine 4: 1-12, all of which are incorporated herein by reference. Exemplary PLGA nanoparticle containing a docetaxel payload interior and a prostate-specific membrane antigen (PSMA)-targeting ligand ACUPA (a PSMA substrate analog) on the surface is BIND-014, which is used to treat solid tumors (developed by BIND Bioscience).

Additional examples of targeted polymeric nanoparticles include, but are not limited to, transferring-targeted polymeric (e.g., cyclodextrin) nanoparticles (e.g., CALAA-01, which includes a siRNA and was developed by Calando Pharmaceuticals to treat solid tumors).

### Metal/Carbon-Based Nanoparticles

In other embodiments, the AHCM, the microenvironment modulator, the therapy, or all can be packaged in a metal/carbon-based nanoparticle. Alternatively, or in combination, the AHCM and/or the microenvironment modulator (or a nanoparticle containing the same) can be used in combination with one or more art-known metal/carbon-based nanoparticles. These nanoparticles can contain a metal (gold, titanium) or carbon-based inner shell, surrounded by, e.g., an AHCM, a cytotoxic drug and/or a polymer, such as PEG, for immune shielding and/or targeted delivery.

Exemplary metal/carbon-based nanoparticle include a colloidal gold nanoparticle (e.g., 30-50 nm) coated with PEG-thiol as described in, e.g., U.S. Pat. No. 7,229,841, U.S. Pat. No. 7,387,900 and U.S. Pat. No. 6,274,552, incorporated herein by reference. Specific products of the Aurimune platform being developed by CytImmune include CYT-6091, which is a first generation Aurimune nanoparticle bearing tumor necrosis factor (TNF) on the surface; CYT-21000, which is a second generation Aurimune nanoparticle bearing TNF and Taxol on the surface, and CYT-61000 (particle bearing interferon) and CYT-71000 (particle bearing gemcitabine).

### Synthetic Vaccine Particles (SVPs)

In other embodiments, the AHCM, the microenvironment modulator, the therapy, or any combination thereof, can be packaged in an SVP. Alternatively, or in combination, the AHCM and/or the microenvironment modulator (or a nanoparticle containing the same) can be used in combination with one or more SVPs. This platform involves a nanoparticle polymer embedded with B cell antigens; the nanoparticle encapsulates either T-cell antigens (e.g., disease-specific epitopes or protein antigens) or immunomodulators (e.g., TLR agonists or checkpoint inhibitors), as described in, e.g., US 20130028941, US 20140199340 and US 20120301498. Exemplary products developed by Select a Biosciences include SEL-212 and SEL-068.

### Light-Activated Drug Delivery

In other embodiments, the AHCM, the microenvironment modulator, the therapy, or any combination thereof, can be packaged in a light-activated drug delivery. Alternatively, or in combination, the AHCM and/or the microenvironment modulator (or a nanoparticle containing the same) can be used in combination with one or more light-activated nanoparticles. Fluorescent, porous silica nanoparticles filled with various chemotherapeutics (e.g. camptothecin) or nucleic acids. The pores in the particles are capped with “nanovalves” to prevent leakage. Upon exposure to exogenous two-photon radiation (laser light), the nanovalves open and release drug cargo. Typically, the nanoparticles treat tumors within 4 cm of skin surface due to ability of laser to penetrate skin, and fluorescent labels allow for tracking of nanoparticles through the body. The technology is described, e.g., in US 20120207795, US 20100310465, and Croissant, J., et al. Two-photon-triggered drug delivery via fluorescent nanovalves. (2014) Small. 10:1752-1755, all of which are incorporated herein by reference.

### Nanodiamonds

In other embodiments, the AHCM, the microenvironment modulator, the therapy, or any combination thereof, can be packaged in one or more nanodiamonds. Alternatively, or in combination, the AHCM and/or the microenvironment modulator (or a nanoparticle containing the same) can be used in combination with one or more nanodiamonds. Nanodiamonds, carbon-based particles about, e.g., 4-5 nm in diameter, can be bound to a broad range of drug compounds. Binding seems to be through hydrophobic interactions between particle surface and drug molecules. Current uses include doxorubin linked nanodiamonds and duanorubicin-linked nanodiamonds for leukemia.

Other particles, e.g., encapsulated and/or carrier—targeted particles are within the scope of the invention.

### Methods for Patient Selection

Also provided herein are methods for identifying or selecting a subject that is in need of improved delivery and/or efficacy of a therapy (e.g., a cancer therapy, or an anti-fibrotic or anti-inflammatory therapy). In embodiments, the methods described herein can be used to identify or select a subject that would respond to treatment with a metformin agent, alone or in combination with i) an AHCM agent; ii) a microenvironment modulator or other stromal modulator; iii) an anti-inflammatory agent; and/or iv) an immune-checkpoint inhibitor, as described herein, e.g., such that the delivery of an additional therapy, e.g., a cancer or an anti-fibrotic therapy, is improved.

The method includes identifying the subject as having a desmoplastic disorder (e.g., a cancer or a fibrotic or inflammatory disorder). Methods for identifying the subject as having a desmoplastic disorder, e.g., a cancer or a fibrotic or inflammatory disorder, are known in the art. Such methods include detection of desmoplasia, e.g., fibrosis, such as an increase in the level or production of extracellular matrix components, e.g., collagen, or hyaluronic acid; increased angiotensin II (AngII) type-1 receptor (AT1) signaling; and/or increased expression, production, and/or secretion of pro-inflammatory cytokines, e.g., interleukin-1β (IL-1β).

In one embodiment, the subject is, or is identified as being, overweight or obese, and has a fibrotic or desmoplastic tumor, e.g., a tumor having one or more of: limited tumor perfusion, compressed blood vessels, high interstitial fluid pressure (IFPs), increased hypoxia, or fibrotic tumor interstitium. In certain embodiments, the subject is overweight or obese, and has a tumor having (e.g., elevated levels of) extracellular matrix components, such as fibers (e.g., collagen, procollagen), fibroblasts (e.g., elevated levels of cancer associated fibroblasts (CAFs) or increased activity of CAFs) and/or polysaccharides (e.g., glycosaminoglycans such as hyaluronan or hyaluronic acid).

The method further includes evaluating, e.g., acquiring a value for, a weight/metabolic-related parameter, for the subject. Examples of a weight/metabolic-related parameter includes body mass index (BMI).

In one embodiment, the subject is, or is identified as being, overweight or obese. Assessment of overweight and obesity can be determined by the classification of body mass index (BMI) as defined by “Clinical Guidelines on the Identification, Evaluation, and Treatment of Overweight and Obesity in Adults” from the National Institutes of Health. Body mass index is obtained by dividing a subject's weight, e.g., in kilograms (kg) by the square of the subject's height, e.g., in meters (m). Subjects with a BMI 18.5 to 24.9 are typically classified as normal weight, while subjects with a BMI 25.0 to 29.9 are classified as overweight. Subjects with a BMI 30.0 or greater are classified as obese, and can be subdivided into three classes: Class I (BMI=30.0 to 34.9; Class II (BMI=35.0 to 39.9); and Class III (BMI is greater or equal to 40).

In one embodiment, the subject is overweight, e.g., the subject has a BMI of greater than or equal to 25.0 but less than or equal to 29.9.

In another embodiment, the subject is, or is identified as being, obese, e.g., the subject has a BMI of greater than or equal to 30, e.g., greater than 30, greater than 35, greater than 40, greater than 45, or greater than 50.

Obesity can also be associated with one or more of: desmoplasia, e.g., in adipose tissues and the pancreas; dysfunctional adipocytes, e.g., hypertrophied adipocytes; increased hypoxia;

fibrosis; accumulation of fat, e.g., steatosis; increased angiotensin II (AngII) type-1 receptor (AT1) signaling; and/or increased expression, production, and/or secretion of pro-inflammatory cytokines, e.g., interleukin-1β (IL-1β).

In an embodiment, the subject is, or is identified as being, overweight or obese, and has a fibrotic or a hyperproliferative cancerous condition described herein. In an embodiment, the subject is, or is identified as being, overweight or obese and has a fibrotic disorder described herein. In an embodiment, the subject is, or is identified as being, overweight or obese and has an inflammatory disorder described herein.

### Embodiments of Various Aspects Described Herein can be Defined in any of the Following Numbered Paragraphs:

1. A method of improving the delivery and/or efficacy of a cancer therapy or an anti-fibrotic therapy, or treating or preventing a cancer or a fibrotic disorder, in a subject, comprising:

identifying the subject as being in need of improved delivery and/or efficacy of a cancer therapy or an anti-fibrotic therapy;

responsive to said identification, administering a metformin agent to the subject, wherein the metformin agent is administered in combination with one, two, three or more of:

(i) an anti-hypertensive and/or a collagen modifying agent (AHCM) (e.g., an angiotensin receptor blocker (ARB));

(ii) a microenvironment modulator (e.g., an anti-angiogenic inhibitor, e.g., a low-dose anti-angiogenic inhibitor) and/or other stromal modulators;

(iii) an anti-inflammatory agent (e.g., a cytokine inhibitor); or

(iv) an inhibitor of an immune checkpoint molecule; and

optionally, administering the cancer therapy or the anti-fibrotic therapy, under conditions sufficient to treat or prevent the cancer or the fibrotic disorder, in the subject, or to improve the delivery and/or efficacy of the cancer therapy or anti-fibrotic therapy provided to the subject.

2. A method of improving the delivery and/or efficacy of a cancer therapy or an anti-fibrotic therapy, or treating or preventing a cancer or a fibrotic disorder, in a subject, comprising administering a metformin agent to the subject in combination with an AHCM (e.g., an ARB), and

optionally, administering the cancer or the anti-fibrotic therapy, under conditions sufficient to treat or prevent the cancer therapy or the fibrotic disorder, in the subject, or to improve the delivery and/or efficacy of the cancer therapy or the anti-fibrotic therapy provided to the subject.

3. A method of improving the delivery and/or efficacy of a cancer therapy or an anti-fibrotic therapy, or treating or preventing a cancer or a fibrotic disorder, in a subject, comprising:

identifying the subject as being in need of improved delivery and/or efficacy of a cancer therapy or an anti-fibrotic therapy;

responsive to said identification, administering a metformin agent to the subject, wherein the metformin agent is administered in combination with a microenvironment modulator (e.g., an anti-angiogenic inhibitor, e.g., a low-dose anti-angiogenic inhibitor) and/or other stromal modulators, and

optionally, administering the cancer therapy or the anti-fibrotic therapy, under conditions sufficient to treat or prevent the cancer or the fibrotic disorder, in the subject, or to improve the delivery and/or efficacy of the cancer therapy or the anti-fibrotic therapy provided to the subject.

4. A method of improving the delivery and/or efficacy of a cancer therapy or an anti-fibrotic therapy, or treating or preventing a cancer or a fibrotic disorder, in a subject, comprising administering a metformin agent to the subject, in combination with, an anti-inflammatory agent (e.g., a cytokine inhibitor), and

optionally, administering the cancer therapy or the anti-fibrotic therapy, under conditions sufficient to treat or prevent the cancer or the fibrotic disorder, in the subject, or to improve the delivery and/or efficacy of the cancer therapy or the anti-fibrotic therapy provided to the subject.

5. A method of improving the delivery and/or efficacy of a cancer therapy or an anti-fibrotic therapy, or treating or preventing a cancer or a fibrotic disorder, in a subject, comprising administering a metformin agent to the subject, in combination with, an inhibitor of an immune checkpoint molecule, and

optionally, administering the cancer or the anti-fibrotic therapy, under conditions sufficient to treat or prevent the cancer or fibrotic disorder, in the subject, or to improve the delivery and/or efficacy of the cancer therapy or the anti-fibrotic therapy provided to the subject.

6. A method of evaluating, e.g., identifying and/or stratifying, responsiveness of a subject, e.g., a cancer patient, for a metformin therapy and/or an AHCM therapy, said method comprising:

acquiring a value for, a weight/metabolic-related parameter (e.g., BMI) for the subject; and, responsive to a determination of a weight/metabolic-related parameter indicative of overweight or obesity (e.g., BMI value greater than 25), performing one, two, three or more of:

(i) identifying the subject as being likely to respond to the metformin therapy and/or the AHCM therapy;

(ii) stratifying the subject, or a patient populations (e.g., stratifying the subject) as being likely to respond (e.g., responders vs. non-responders) to the metformin therapy and/or the AHCM therapy;

(iii) more effectively monitor the metformin therapy and/or the AHCM therapy; or

(iv) administering the metformin agent, alone or in combination with, one, two, three or more of: (i) an AHCM (e.g., an ARB); (ii) a microenvironment modulator (e.g., an anti-angiogenic inhibitor), and/or other stromal modulators; (iii) an anti-inflammatory agent (e.g., a cytokine inhibitor); or (iv) an inhibitor of an immune checkpoint molecule.

7. The method of any of paragraphs 1-6, further comprising dentifying the subject as having a desmoplastic disorder (e.g., a cancer or a fibrotic disorder).

8. The method of any of the paragraphs 1-7, further comprising identifying the subject as being overweight or obese, e.g., as having a BMI greater than 25.

9. The method of paragraph 8, wherein responsive to said identification, the metformin agent, alone or in combination with, one, two, three or more of: (i) an AHCM (e.g., an ARB); (ii) a microenvironment modulator (e.g., an anti-angiogenic inhibitor) and/or other stromal modulators; (iii) an anti-inflammatory agent (e.g., a cytokine inhibitor); or (iv) an inhibitor of an immune checkpoint molecule, is administered.

10. The method of any of paragraphs 1-5 or 7-9, further comprising evaluating, e.g., acquiring a value for, a weight/metabolic-related parameter (e.g., BMI) for the subject, wherein:

(i) responsive to a determination of a weight/metabolic-related parameter indicative of overweight or obesity (e.g., BMI value greater than 25), the metformin agent, alone or in combination with, one, two, three or more of: (i) an AHCM (e.g., an ARB); (ii) a microenvironment modulator (e.g., an anti-angiogenic inhibitor, e.g., a low-dose anti-angiogenic inhibitor) and/or other stromal modulators; (iii) an anti-inflammatory agent (e.g., a cytokine inhibitor); or (iv) an inhibitor of an immune checkpoint molecule, is administered; or

(ii) responsive to a determination of a weight/metabolic-related parameter indicative of normal or underweight in the subject, the metformin agent, alone or in combination with, one, two, three or more of: (i) an AHCM (e.g., an ARB); (ii) a microenvironment modulator (e.g., an anti-angiogenic inhibitor, e.g., a low-dose anti-angiogenic inhibitor) and/or other stromal modulators; (iii) an anti-inflammatory agent (e.g., a cytokine inhibitor); or (iv) an inhibitor of an immune checkpoint molecule, is discontinued or not administered.

11. A combination or composition (e.g., one or more compositions or dosage forms), that includes a metformin agent in combination with one, two, three or more of:

(i) an AHCM (e.g., an ARB);

(ii) a microenvironment modulator (e.g., an anti-angiogenic inhibitor, e.g., a low-dose anti-angiogenic inhibitor) and/or other stromal modulators;

(iii) an anti-inflammatory agent (e.g., a cytokine inhibitor); or

(iv) an inhibitor of an immune checkpoint molecule; and optionally, a cancer or an anti-fibrotic therapy.

12. The method or composition of any of paragraphs 1-11, wherein the metformin agent is a biguanide or any functional analog, derivative, or salt thereof.

13. The method or composition of any of paragraphs 1-12, wherein the metformin agent comprises two linked guanidine moieties.

14. The method or composition of any of paragraphs 1-13, wherein the metformin agent is chosen from metformin, phenformin, buformin, and biguanide, or any functional analog, derivative, or salt of any of the aforesaid compounds.

15. The method or composition of any of paragraphs 1-14, wherein the metformin agent is described by a compound of Formula (I):

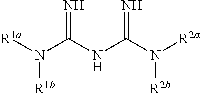

or a pharmaceutically acceptable salt thereof, wherein each of R^{1a}, R^{1b}, R^{2a}, and R^{2b} is independently hydrogen, C₁-C₆ alkyl, cycloalkylalkyl, or arylalkyl.

16. The method or composition of paragraph 15, wherein each of R^{1a} and R^{1b} is hydrogen; each of R^{2a} and R^{2b} is independently hydrogen or C₁-C₆ alkyl; each of R^{2a} and R^{2b} is independently C₁-C₄ alkyl; each of R^{2a} and R^{2b} is independently C₁-C₂ alkyl. In some embodiments, each of R^{2a} and R^{2b} is independently methyl; each of R^{1a} and R^{1b} is hydrogen, and each of R^{2a} and R^{2b} is methyl.

17. The method or composition of any of paragraphs 1-16, wherein the metformin agent is metformin, e.g., 3-(diaminomethylidene)-1,1-dimethylguanidine.

18. The method or composition of any of paragraphs 1-17, wherein the AHCM is chosen from one or more of: an angiotensin II receptor blocker (AT₁ blocker or ARB), an renin antagonist, an antagonist of renin angiotensin aldosterone system (“RAAS antagonist”), an angiotensin converting enzyme (ACE) inhibitor, a thrombospondin 1 (TSP-1) inhibitor, a transforming growth factor beta 1 (TGF-β1) inhibitor, a connective tissue growth factor (CTGF) inhibitor, a stromal cell-derived growth factor 1 alpha (SDF-1a) inhibitor, e.g., a CXCR-4 antagonist, or an agonist of AT₂ receptor; or a combination of two or more of the above.

19. The method or composition of paragraphs 18, wherein the AT₁ blocker or ARB is chosen from one or more of: losartan (COZAAR®), candesartan (ATACAND®), eprosartan mesylate (TEVETEN®), EXP 3174, irbesartan (AVAPRO®), L158,809, olmesartan (BENICAR®), saralasin, telmisartin (MICARDIS®), valsartan (DIOVAN®), or a derivative thereof.

20. The method or composition of paragraph 18, wherein the RAAS antagonist is chosen from one or more of: aliskiren (TEKTURNA®, RASILEZ®), remikiren (Ro 42-5892), enalkiren (A-64662), SPP635, or a derivative thereof.

21. The method or composition of paragraph 18, wherein the ACE inhibitor is chosen from one or more of: benazepril (LOTENSIN®), captopril (CAPOTEN®), enalapril (VASOTEC®), fosinopril (MONOPRIL®), lisinopril (PRINIVIL®, ZESTRIL®), moexipril (UNIVASC®), perindopril (ACEON®), quinapril (ACCUPRIL®), ramipril (ALTACE®), trandolapril (MAVIK®), or a derivative thereof.

22. The method or composition of paragraph 18, wherein the TSP-1 inhibitor is chosen from one or more of: ABT-510, CVX-045, LSKL, or a derivative thereof.

23. The method or composition of paragraph 18, wherein the TGF-β1 inhibitor, e.g., an anti-TGF-β1 antibody, a TGF-β1 peptide inhibitor is chosen from one or more of: CAT-192, fresolimumab (GC1008), LY 2157299, Peptide 144 (P144), SB-431542, SD-208.

24. The method or composition of paragraph 18, wherein the CTGF inhibitor is chosen from one or more of: DN-9693, or FG-3019.

25. The method or composition of paragraph 18, wherein the SDF-1a inhibitor is an anti-SDF1a antibody or fragment thereof or Plerixafor (AMD3100).

26. The method or composition of any of paragraphs 1-19, wherein the microenvironment modulator chosen from one or more of an anti-angiogenic therapy, an agent that decreases the level or production of hyaluronic acid, an inhibitor of the hedgehog pathway, an agent that improves drug penetration in tumors (e.g., a disulfide-based cyclic RGD peptide peptide (iRGD) or an analogue thereof), a taxane therapy, an agent that modulates (e.g, inhibits) a hypoxia inducible factor (HIF) (e.g., HIF-1α and HIF-2α), an agent that decreases the level or production of collagen or procollagen, an anti-fibrotic agent (e.g., a pirfenidone (PFD, 5-methyl-1-phenyl-2-(1H)-pyridone); or a combination of two or more of the above;

the agent is an other stromal modulator chosen from an inhibitor of a receptor for a VEGF ligand (e.g., a Flt-1, -2, and/or -3 receptor), an inhibitor of an FGF receptor, a c-Met/HGF receptor inhibitor, a TNFR inhibitor, a cytokine/cytokine receptor inhibitor, a JAK/STAT3 inhibitor, an Osteopontin (SPP1) modulator, a Bone morphogenic protein (BMPs) inhibitor, an inhibitor of FAK, a CSF-1R inhibitor, a c-Kit inhibitor, a DDR1 inhibitor, a metabolic inhibitor, a mitochondrial inhibitor, Metformin/Phenformin, an oxidative phosphorylation inhibitor (OX-Phos inhibitor); or a combination of two or more of the above; or

a combination of the microenvironment modulator or the other stromal modulator.

27. The method or composition of any of paragraphs 1-26, wherein the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23, interferons (IFNs), e.g., IFNα, IFNβ, IFNγ, IFN-γ inducing factor (IGIF), transforming growth factor- β (TGF-β), transforming growth factor-α (TGF-α), tumor necrosis factors TNF-α, TNF-β, TNF-RI, TNF-RII, CD23, CD30, CD40L, EGF, G-CSF, GDNF, PDGF-BB, RANTES/CCL5, IKK, NF-κB, TLR2, TLR3, TLR4, TL5, TLR6, TLR7, TLR8, TLR8, TLR9, and/or any cognate receptors thereof.

28. The method or composition of any of paragraphs 1-27, wherein the cancer therapy is an anti-cancer agent chosen from an alkylating agent, a vascular disrupting agent, a microtubule targeting agent, a mitotic inhibitor, a topoisomerase inhibitor, an anti-angiogenic agent or an anti-metabolite.

29. The method or composition of paragraph 28, wherein the anti-cancer agent is a taxane (e.g., paclitaxel, docetaxel, larotaxel or cabazitaxel).

30. The method or composition of paragraph 28, wherein the anti-cancer agent is an anthracycline (e.g., doxorubicin), a platinum-based agent (e.g., cisplatin or oxaliplatin), or a pyrimidine analog (e.g., gemcitabine).

31. The method or composition of paragraph 28, wherein the anti-cancer agent is chosen from camptothecin, irinotecan, rapamycin, FK506, 5-FU, leucovorin, or a combination thereof.

32. The method or composition of any of paragraphs 1-31, wherein the inhibitor of the immune checkpoint molecule is chosen from an inhibitor of PD-1, PD-L1, LAG-3, TIM-3 or CTLA4, or any combination thereof.

33. The method or composition of any of paragraphs 1-32, wherein the subject has a tumor containing an extracellular matrix component chosen from collagen, procollagen and/or hyaluronan (HA).

34. The method or composition of any of paragraphs 1-33, wherein the cancer is chosen from one or more of pancreatic, breast, colorectal, colon, lung, skin, ovarian, prostate, cervix, gastric, gastrointestinal, stomach, head and neck, kidney, brain cancer, liver cancer, or a metastatic lesion thereof.

35. The method or composition of any of paragraphs 1-34, wherein the fibrotic disorder is a fibrotic condition or disorder of the lung, a fibrotic condition of the liver, a fibrotic condition of the heart or vasculature, a fibrotic condition of the kidney, a fibrotic condition of the skin, a fibrotic condition of the gastrointestinal tract, a fibrotic condition of the bone marrow or a hematopoietic tissue, a fibrotic condition of the nervous system, a fibrotic condition of the eye, or a combination thereof.

36. The method of any of paragraphs 1-35, wherein the metformin agent, the AHCM, the microenvironment modulator and/or the other stromal modulator is administered prior to and/or in combination with the cancer or the fibrotic therapy.

37. The method or composition any of paragraphs 1-36, wherein the cancer therapy is chosen from one or more of anti-cancer agents, photodynamic therapy, an immunotherapy (e.g., an immune-cell therapy or adoptive immunotherapy), surgery and/or radiation.

38. The method of any of paragraphs 1-37, wherein the metformin agent, the AHCM, the microenvironment modulator and/or the other stromal modulator is administered at least one, two, three, or five days; or one, two, three, four, five or more weeks, prior to the cancer or fibrotic therapy, 39. The method of any of paragraphs 1-38, wherein the metformin, the AHCM, the microenvironment modulator, or the other stromal modulator is administered as a particle or in free form in a dosage sufficient to improve the delivery or effectiveness of the cancer or fibrotic therapy.

40. The method of any of paragraphs 1-39, wherein when the AHCM is administered as a particle, and optionally said AHCM is present at a dose that is higher than the standard or care dose of the AHCM in free form (e.g., at least 20%, 30%, 40%, 50% or higher dose).

41. The method of any of paragraphs 1-40, wherein the subject has a pre-neoplastic condition or a pre-disposition to cancer.

42. The method of any of paragraphs 1-41, wherein the subject is at risk of having, or has a solid, fibrotic tumor.

43. The method of any of paragraphs 1-42, wherein the subject is overweight.

44. The method of any of paragraphs 1-43, wherein the subject is obese.

45. The method of any of paragraphs 1-44, wherein when the subject is treated with an AHCM, the method further comprising administering to the subject an anti-angiogenic agent, e.g., a VEGF/VEGFR inhibitor.

46. The method of any of paragraphs 1-45, wherein the AHCM, the microenvironment modulator and/or the other stromal modulator is maintained for a preselected portion of the time the subject receives cancer therapy.

47. The method of any of paragraphs 1-46, wherein the AHCM, the microenvironment modulator and/or the other stromal modulator is maintained for the entire period in which the cancer therapy is administered.

48. The method of any of paragraphs 1-47, wherein the AHCM, the microenvironment modulator and/or the other stromal modulator is administered continuously over a period of at least 1, 5, 10, or 24 hours; at least 2, 5, 10, or 14 days; at least 2, 3, 4, 5 or 6 weeks; at least 2, 3, 4, 5 or 6 months; or at least 1, 2, 3, 4 or 5 years.

49. The method of any of paragraphs 1-48, wherein the AHCM, the microenvironment modulator and/or the other stromal modulator is administered after cessation of the therapy, e.g., the cancer therapy.

50. The method of any of paragraphs 1-49, wherein the AHCM is formulated for oral, subcutaneus, intravenous continuous delivery; or is administered as a sustained release formulation.

51. The method or composition of any of paragraphs 1-50, wherein the cancer therapy is chosen from one or more of:

(i) a cancer therapeutic chosen from a viral cancer therapeutic agent, a lipid nanoparticle of an anti-cancer therapeutic agent, a polymeric nanoparticle of an anti-cancer therapeutic agent, an antibody against a cancer target, a dsRNA agent, an antisense RNA agent, or a chemotherapeutic agent;

(ii) an immunotherapy (e.g., an immune-cell therapy or adoptive immunotherapy);

(iii) radiation,

(iv) surgery,

(v) a photodynamic therapy; or

(vi) any combination of (i)-(v).

52. The method or composition of paragraph 51, wherein the lipid nanoparticle is chosen from pegylated liposomal doxorubicin (DOXIL®) or liposomal paclitaxel (e.g., Abraxane®).

53. The method or composition of paragraph 51, wherein the chemotherapeutic agent is chosen from gemcitabine, cisplatin, epirubicin, 5-fluorouracil, paclitaxel, oxaliplatin, or leucovorin.

54. The method or composition of paragraph 51, wherein the antibody against the cancer target is chosen from an antibody against HER-2/neu, HER3, VEGF, or EGFR.

55. The method or composition of any of paragraphs 1-54, wherein the cancer therapy is a tyrosine kinase inhibitor chosen from sunitinib, erlotinib, gefitinib, sorafenib, icotinib, lapatinib, neratinib, vandetanib, BIBW 2992 or XL-647, or an anti-EGFR antibody chosen from cetuximab, panitumumab, zalutumumab, nimotuzumab necitumumab or matuzumab.

56. The method or composition of paragraph 51, wherein the chemotherapeutic agent is a cytotoxic or a cytostatic agent.

57. The method or composition of paragraph 51, wherein the chemotherapeutic agent is chosen from an antimicrotubule agent, a topoisomerase inhibitor, a taxane, an antimetabolite, a mitotic inhibitor, an alkylating agent, or an intercalating agent.

58. The method or composition of any of paragraphs 1-57, wherein the cancer therapy is chosen from one of more of: an anti-angiogenic agent, or a vascular targeting agent or a vascular disrupting agent.

59. The method of any of paragraphs 1-58, wherein the AHCM, the microenvironment modulator, the other stromal modulator, or the cancer therapy is administered to the subject by a systemic administration chosen from oral, parenteral, subcutaneous, intravenous, rectal, intramuscular, intraperitoneal, intranasal, transdermal, or by inhalation or intracavitary installation.

60. The method or composition of any of paragraphs 1-59, wherein the particle a polymeric particle or a lipid particle.

61. The method or composition of paragraph 60, wherein the polymeric particle comprises a polymer selected from the group consisting of poly(lactic acid)-b-poly(ethylene glycol) (PLA-PEG), poly(lactic acid)-b-poly(ethylene glycol) (PLGA-PEG), dextran, and (cyclodextrin)-co-poly(ethylene glycol) (CDP).

62. The method or composition of any of paragraphs 1-61, wherein the liver disorder is a fibrotic liver disorder include chosen from liver fibrosis (hepatic fibrosis), liver cirrhosis, or any disorder associated with accumulation of extracellular matrix proteins, e.g., collagen, in the liver, liver scarring, and/or abnormal hepatic vasculature.

63. The method or composition of any of paragraphs 1-62, wherein the cancer is a liver cancer chosen from a hepatocellular carcinoma (HCC), primary liver cell carcinoma, hepatoma, fibrolamellar carcinoma, focal nodular hyperplasia, cholangiosarcoma, intrahepatic bile duct cancer, angiosarcoma or hemangiosarcoma, hepatic adenoma, hepatic hemangiomas, hepatic hamartoma, hepatoblastoma, infantile hemangioendothelialoma, mixed tumors of the liver, tumors of mesenchymal tissue, or sarcoma of the liver.

64. A method for treating or preventing a liver disorder or condition in a subject., comprising administering to the subject an AHCM and a vascular/stromal normalizing dose (e.g., a sub-anti-angiogenic dose) of a second agent chosen from one or more of: anti-angiogenic agent, sorafenib or an inhibitor of the angiopoietin-Tie-2 pathway (e.g., an Ang-1 or an Ang-2 inhibitor), thereby treating or preventing the liver disorder or condition.

65. The method of paragraph 64, wherein the second agent is administered at a sub-anti-angiogenic dose.

66. The method of paragraph 64, wherein the second agent is sorafenib and is administered at a low dose, e.g., a dose of sorafenib that is less than the standard of care dose, e.g., an anti-angiogenic or anti-vascularization dose.

67. The method of paragraph 64, wherein the second agent is an inhibitor of the angiopoetin-Tie-2 pathway (e.g., an Ang-1 or an Ang-2 inhibtor), and is administered at a low dose, e.g., a dose of an inhibitor of the angiopoietin-Tie-2 pathway (e.g., an Ang-1 or an Ang-2 inhibtor) that is less than the standard of care dose, e.g., an anti-angiogenic or anti-vascularization dose.

68. The method of paragraph 66, wherein sorafenib or the inhibitor of the angiopoietin-Tie-2 pathway (e.g., an Ang-1 or an Ang-2 inhibtor) is administered at a dose or dosage formulation that is less than 0.01, 0.05, 0.1, 0.2, 0.3, 0.4, 0.5, 0.6, 0.7, 0.8, 0.9, that of the standard of care dose.

69. The method of paragraph 64, wherein the second agent is an inhibitor of vascular endothelial growth factor (VEGF) pathway chosen from an antibody against VEGF (e.g., bevacizumab); a VEGF receptor inhibitor (e.g., an inhibitor of VEGFR-1 inhibitor, a VEGFR-2 inhibitor, or a VEGFR-3 inhibitor (e.g., VEGFR inhibitors such as Cediranib (AZD2171)); a VEGF trap (e.g., a fusion protein that includes a VEGFR domain (e.g., a VEGFR1 domain 2 and a VEGFR2 domain 3) fused to an Fc fragment of an IgG); and an anti-VEGF aptamer (or a pegylated derivative thereof (e.g., MACUGEN®), and is administered at a dose that is less than the standard of care dose, e.g., an anti-angiogenic or anti-vascularization dose.

70. The method of paragraph 64, wherein the second agent is a (sorafenib) similarly-targeted pathway modulator chosen from an inhibitor of tyrosine or Ser/Thr kinase chosen from VEGFR, PDGFR, c-kit receptors, or b-Raf, and is administered at a dose or dosage formulation that is less than 0.01, 0.05, 0.1, 0.2, 0.3, 0.4, 0.5, 0.6, 0.7, 0.8, 0.9, that of the standard of care dose.

### EXAMPLES

The invention now being generally described, it will be more readily understood by reference to the following examples, which are included merely for purposes of illustration of certain aspects and embodiments of the present invention, and are not intended to limit the invention.

### Example 1

### Metformin Reduces Desmoplasia in Pancreatic Cancer by Reprogramming Stellate Cells and Tumor-Associated Macrophages

Background: Pancreatic ductal adenocarcinoma (PDAC) is a highly desmoplastic tumor with a dismal prognosis for most patients. Fibrosis and inflammation are hallmarks of tumor desmoplasia. It has previously been demonstrated that preventing the activation of pancreatic stellate cells (PSCs) and alleviating desmoplasia are beneficial strategies in treating PDAC. Metformin is a widely used glucose-lowering drug. It is also frequently prescribed to diabetic pancreatic cancer patients and has been shown to associate with better outcome. However the underlying mechanisms of this benefit remain unclear. Metformin has been found to modulate the activity of stellate cells in other disease settings. In this study we examine the effect of metformin on PSC activity, fibrosis and inflammation in PDACs.

Methods/Results: In overweight diabetic PDAC patients and pre-clinical mouse models, treatment with metformin reduced levels of tumor extracellular matrix (ECM) components, in particular hyaluronan (HA). In vitro, metformin reduced TGF-β signaling and the production of HA and collagen-I in cultured PSCs. Furthermore, metformin alleviates tumor inflammation by reducing the expression of inflammatory cytokines as well as infiltration and M2 polarization of tumor-associated macrophages (TAMs) in vitro and in vivo. These effects on macrophages in vitro appear to be associated with a modulation of the AMPK/STAT3 pathway by metformin. Finally, it was shown that in preclinical models that the alleviation of desmoplasia by metformin was associated with a reduction in extracellular matrix (ECM) remodeling, epithelial-to-mesenchymal transition (EMT) and ultimately systemic metastasis.

Conclusion: Metformin alleviates the fibro-inflammatory microenvironment in obese/diabetic individuals with pancreatic cancer by reprogramming PSCs and TAMs, which correlates with reduced disease progression. Metformin should be tested/explored as part of the treatment strategy in overweight diabetic PDAC patients.

### Introduction

The prognosis for patients with pancreatic cancer is dismal, with an overall five-year rate survival of 7% (1). Obesity and type-2 diabetes mellitus (DM2) have become a pandemic worldwide (2, 3). Recent studies have demonstrated that these metabolic abnormalities are associated with the increased incidence, progression and poor prognosis of PDAC (4-9). At diagnosis, approximately half of PDAC patients are overweight or obese, and up to 80% of patients present with diabetes or glucose intolerance (10-17). DM2 and obesity may promote PDAC through pro-tumorigenic insulin and insulin-like growth factor-1 (IGF-1) (18-20) as well as chronic inflammation (21, 22). Hence, pharmacological interventions that target diabetes/obesity may also produce anti-tumor effects. One such agent, currently under intense investigation, is metformin, the most widely prescribed anti-diabetic generic drug which is also frequently administered to diabetic PDAC patients (23). Metformin has been shown to improve treatment outcomes in preclinical models of PDAC (24-30), and to reduce the incidence of pancreatic cancer in diabetic patients as well as improve survival (reduced risk of death by 32%) in newly diagnosed cases (31-33). However, the mechanisms of action of metformin in pancreatic cancer are not well understood. In vitro studies have addressed the impact of metformin on transcription factors, microRNAs, DNA damage, cancer stem cells and metabolism (34-38). In addition, metformin has been shown to modulate the function of hepatic stellate cells, reduce oxidative stress in cancer-associated fibroblasts, and decrease tumor inflammation (34, 35, 39, 40). Studies have shown that reprogramming PSCs reduces the production of extracellular matrix (ECM) components such as collagen-I and hyaluronan (HA), and slows the progression of pancreatic cancer (41-45). However, the impact of metformin on PSC activation, production of ECM components and tumor desmoplasia has never been described.

The aim of this study is to elucidate the functional mechanisms of metformin within the PDAC fibro-inflammatory tumor microenvironment (26, 46-48). In vivo studies of pancreatic cancers to date have been mainly performed in xenograft models in normal weight/non-diabetic mice where metformin has been shown to be less effective (26, 46-48). Hence, two immunocompetent syngeneic mouse models that closely mimic obesity/DM2 were used, to better represent pancreatic cancer patient populations at diagnosis. Furthermore, these mouse models were complemented with in vitro studies as well as with analysis of human samples of pancreatic cancer from normal weight and overweight/obese patients. It was found that metformin directly reprograms PSCs and TAMs and alleviates desmoplasia and inflammation in obese/diabetic models of PDAC. These effects of metformin correlated with reduced ECM remodeling and epithelial-to-mesenchymal transition (EMT), ultimately affecting metastasis. It was confirmed that ECM levels in human PDAC samples in overweight and obese patients were indeed lower in the patient population treated with metformin

### Materials and Methods

### Animal Experiments

Wild-type (WT) C57BL/6 and FVB male mice were originally obtained from The Jackson Laboratory (The Jackson Laboratory, Bar Harbor, Me.) and bred and maintained in our defined-flora animal facility. Mice were maintained on a 12-h light-dark cycle in a temperature-controlled barrier facility, with ad libitum access to food and acidified water. To generate obese/diabetic mouse models, mice (6-weeks old) were given a 60% fat diet (D12492, Research Diets, New Brunswick, N.J.) for 10 weeks as previously described (49-51). For tumor experiments, the PAN02 and AK4.4 syngeneic PDAC models were used in C57BL/6 and FVB immunocompetent mice, respectively. PAN02 cells (SMAD4-m174) (52) were obtained from ATCC. AK4.4 cells (KrasG12D and p53^{+/-}) were isolated from mice generating spontaneous pancreatic tumors (Pt∫1-Cre/LSL-Kras^{G12D}/p53^{Loxi-}) (53). Orthotopic pancreatic tumors were generated by implanting a small piece (1 mm³) of viable tumor tissue (from a source tumor in a separate donor animal) into the pancreas of a 6-8- week-old male lean or obese FVB (AK4.4 model) or C57BL/6 (PAN02 model) mouse. Both tumor models used were authenticated by IDEXX Laboratories. (PAN02: IDEXX RADIL Case # 22366-2013. AK4.4: IDEXX RADIL Case # 27818-2014).

### Pancreatic Tumor Growth Studies

At 7 days post implantation, mice bearing orthotopic PAN02 or AK4.4 pancreatic tumors were randomized into control or metformin treatment groups. At day 21, plasma and tumor samples were collected, and tumors were weighed and processed for further analysis.

### Metformin Treatment

The standard dose of metformin for treating humans is 1000 to 2500 mg, usually given twice daily. In the present study, metformin was administered at 300 mg/kg in drinking water. This can be translated to the human equivalent dose by using the Reagan-Shaw method (54). According to the formula for the human equivalent dose [(mg/kg)=animal dose (mg/kg)×animal (km)/human (km). Km for a 60 kg human adults equals 37 and for a 20 g mouse equals 3], the human equivalent of murine dose of 300 mg/kg is 1459 mg for an average sized 60 kg adult human. Therefore, the selected dose in the present study is within the safe therapeutic range recorded in humans. Furthermore, the present study determined the therapeutic period to be 2 weeks to evaluate the antitumor effect of metformin. This was compatible with the therapeutic periods reported in previous studies (55, 56). Fresh metformin was administered in drinking water every 3 days. The amount added to each animal cage was calculated based on the average daily water intake for that cage during a period of 2 weeks prior to treatment initiation, and adjusted every three days based on water consumption and body weight of the animals. The approximate plasma concentration of metformin in patients with type 2 diabetes taking this drug is 0.05 mM, although it may accumulate in tissues and reach higher concentrations locally (35). For in vitro experiments, a range of concentration from 0.05 to 25 mM depending on the cell line used, as discussed below.

### Effect of Metformin on PSCs and Macrophages in Vitro

Standard MTT assays were performed on PSCs and macrophages treated with metformin in a range of 0.05-25 mM, to examine the potential effects on cell viability. RAW 264.7 (mouse leukemic monocyte-macrophages) were obtained from ATCC and used to assess the effect of metformin on macrophages in vitro. Cells were seeded in 10 cm² petri dishes in serum/serum-free media and treated with metformin for 48 h at concentrations ranging from 0.05 to 0.4 mM (concentrations that do not substantially affect cell viability). Following treatment, cells were collected for RNA and protein extraction in order to perform subsequent analysis of gene expression of cytokines and polarization markers, and for analysis of signaling and metabolic pathways. Human PSCs were seeded in 10 cm² petri dishes in media with 2.5% serum and treated with metformin for 48 h at concentrations ranging from 0.1 to 10 mM. Cells were collected for protein extraction for analysis of the activation of fibrosis-related pathways. Additionally, PSCs were seeded in an 8-well chamber slide (20,000 cells/well), treated with metformin (1 mM, a concentration that does not affect cell viability) for 48 h, and immunofluorescent staining was performed following standard protocols. The cells were fixed with 4% paraformaldehyde and blocked with 5% normal donkey serum for 1 h. They were incubated with the designated primary antibodies overnight at 4° C. then for 2 h with the appropriate secondary antibodies at RT. PBS was used for all washes and the stained samples were mounted with Prolong Gold with DAPI. Images were acquired using a confocal microscope. The antibodies used and image acquisition settings are described below. Both cell types were grown in Dulbecco's modified Eagle's medium (Sigma, St. Louis, Mo.) supplemented with 5% (v/v) heat inactivated fetal bovine serum (FBS; Sigma), 100 units/ml penicillin and streptomycin. Cells were cultured at 37° C. in a humidified atmosphere including 5% CO₂.

### Human Samples

Human samples of pancreatic cancer were obtained from the Massachusetts General Hospital tissue repository. Written informed consent from the donor or the next of kin was obtained for the use of these samples for research purposes. Tumors selected received no prior chemotherapy or radiation therapy before the surgical specimen was collected at the time of tumor resection. A total of 28 samples were randomly selected from this subset of samples. Body mass index was obtained for the respective sample. In 7 samples (25%), patents were medicated with metformin at the time of resection. Paraffin sections were stained for collagen-I and HA as described below. Images were acquired using confocal microscopy and quantified using Matlab. Data were analyzed anonymously.

### Gene Expression

Immediately following excision, tumor tissue was snap frozen and stored in liquid nitrogen. Total RNA was extracted and relative gene expression for macrophage M1/M2 markers was determined using a Sybr-green based standard protocol. In addition, total RNA was extracted and relative gene expression was determined using RT2 Profiler PCR Arrays system (Qiagen) on a Stratagene Mx3000P QPCR System using pre-made pathway-focused arrays (“Fibrosis”-Cat. Number: PAMM011Z; “Epithelial to Mesenchymal Transition (EMT)”-Cat. Number: PAMM090Z; “TGF-β signaling targets”-PAMM235Z; “Extracellular Matrix & Adhesion Molecules”-Cat. Number: PAMM013Z; and “Common cytokines ”-Cat. Number: PAMM021Z).

### Protein Expression

Western Blot Analysis

Each tumor sample was homogenized directly in lysis buffer for protein extraction. 20 ug of denatured protein per sample was loaded on 7%, 10% and 12% SDS-polyacrylamide gels. Antibodies used: phospho-p38 MAPKT^{180/Y182} and p38; phospho-JNK (SAPK/JNK)^{Thr183/Tyr185} and JNK; phospho-AKT^{Ser473} and AKT, phospho-ERK(p44/42 MAPK)^{T202/Y204} and ERK; phospho-NF-κB^{Ser536} and NK-κB; p65^{Ser536}; phospho-Smad2^{Ser465/467} and Smad2; phospho-IGF-I Receptor β^{Tyr1135} and IGF; Phospho-IRS-1^{Ser612} and IRS; IR; phpspho-stat3^{Tyr705} and stat3; Phospho-AMPKα^{Thr172} and Ampkα; phospho-Ampkβ^{Ser108} and Ampk; phospho-ACC^{Ser79} and ACC; Phospho-PDGF Receptor β^{Tyr751} and PDGF Receptor β and snail, c-cadherin and vimentin, TGF-β, β-catenin, twist, LC3B. All antibodies listed above were obtained from Cell Signaling Technology (Beverly, Mass.), and diluted 1:1000 with the exception of phospho-JNK (SAPK/JNK)^{Thr183/Tyr185}, Phospho-NF-κB p65^{Ser536}, phospho-Smad2^{Ser465/467}, phospho-IGF-I Receptor β^{Tyr1135}, Cleaved caspase-3, Phospho-IRS-1^{Ser612}. Other antibodies used were: αSMA and phospho-Insulin Receptor^{Y972} (1:1000 and 1:500, Abcam, Mass.); col-1 (1:1000); MMP-9 and MMP-2 (1:500 and 1:200, EMD Millipore-Billerica, Mass.), AT1 (1:1000, LifeSpan BioSciences Inc, WA), ZEB1 (1:1000, Novus Biologicals, CO), and β-actin (1:5000, Sigma, Mo.). Quantification of protein expression relative to total receptor or β-actin was obtained using Image J software.

Multiplex Array

Each tumor sample was homogenized directly in lysis buffer for protein extraction. 2 ug/ul of sample was used. A multi-plex inflammatory multiple cytokines protein array was used (V-PLEX Proinflammatory Panell mouse kit, Cat. Number: K15048D) for ELISA analysis.

### Immunofluorescence/Immunohistochemistry

For analysis of frozen sections of mouse tumor tissues, the tumors were excised and frozen in optimal cutting temperature compound (Tissue-Tek). Transverse tumor sections, 10 μm thick, were immunostained with specific antibodies. To obtain mosaic images of tumor sections, an Olympus FV1000 confocal laser-scanning microscope was used. A 10× air objective acquired 1260-μm square tiles, and an automated stage scanned throughout the entire cross-section of tumor tissue. The imaged tiles were stitched into a final mosaic image using Olympus software. Antigen expression was quantified by measuring the area occupied by the stain of interest normalized by the area of DAPI-stained nuclei (i.e., unit less), and analyzed using a custom algorithm in MATLAB (The MathWorks). Identical settings and thresholds for analysis were used for all tumors. Antibodies used for immunofluorescence were the following: Collagen-I [LF-68 antibody, 1:50 dilution, provided by Dr Larry Fisher (NIDCR)]; Hyluronan (biotinylated hyaluronan proteoglycan fragment, 385911, Calbiochem, 1:200 dilution); αSMA (C6198 antibody, Sigma, 1:500 dilution); and F4/80 (MCA497A488 antibody, ABDserotec, 1:200 dilution). Cy3-, Cy5- or FITC-conjugated secondary antibodies were used for the detection of signals by confocal microscopy. Slides were counterstained with DAPI for nuclear staining.

### MMP Activity Assay

Each tumor sample was homogenized directly in lysis buffer for protein extraction. A standard commercial assay (Abcam ab112146) was used to detect the general activity of MMPs in the tumor samples. Tumor lysates were incubated with a fluorescent substrate [fluorescence resonance energy transfer (FRET) peptide] for 1, 2, and 16 hours, and fluorescence was measured using a fluorescent microplate reader.

### Flow Cytometry

Tumor-bearing mice were perfused through intracardiac injection of PBS and sacrificed. Pancreatic tumor tissues were harvested, minced, and digested at 37° C. for 1 h with DMEM containing collagenase type 1A (1.5 mg/mL), hyaluronidase (1.5 mg/mL), and DNase (2 ug/mL). The digestion mixtures were filtered through 70-μm cell strainers. Single-cell suspensions were incubated with rat anti-mouse CD16/CD32 mAb for blocking and stained with fluorochrome-conjugated antibodies in cold buffer (1% BSA, 0.1% NaN3 in PBS). 7-amino-actinomycin D (7AAD) reagent (eBioscience) was added to the stained tubes per manufacturer's instruction immediately before running the flow analysis. Flow cytometry data were acquired on an LSRII flow cytometer (Becton Dickinson) and were analyzed with FACSDiva software. FSC-A vs. FSC-W and SSC-A vs. SSC-W were applied to discriminate the doublet/aggregated events. The following monoclonal anti-mouse antibodies were used: CD45-PE, CD45-PE-Cy7, CD45-FITC, CD11b-APC-Cy7, CD11b-APC, F4/80-APC (BD Biosciences) and F4/80-FITC and F4/80-PE (eBioscience).

### Macrophage Isolation:

After cutting the tumor tissue into pieces 0.5 mm in diameter, tumors were dissociated with collagenase and hyaluronidase for one hour. Dissociated tumors were washed with sorting buffer (0.5% BSA in PBS) and passed through cell strainers before they were blocked using Fcr and subsequently incubated with a F4/80 biotinylated primary antibody (eBiosciences) for 15 min. Anti-biotin conjugated magnetic beads (Miltenyl Biotech) were added to the cell suspension and incubated for 15 min before exposing the cells to magnetic columns (MACS LS-columns from Miltenyl Biotech) for cell separation.

### Statistical Analysis

Statistical differences between groups were assessed by a two-tailed student t test. Two-way analysis of variance was used when comparing body weigh over time between the control and metformin treated groups. Incidence of metastasis and wall invasion was analyzed using chi-square. All statistical analyses were performed using Prism Graphpad software. All results were considered statistically significant when the P value was less than 0.05 when calculated with the appropriate statistical test. Results are presented as the mean±standard error.

### Results

### Metformin Reduces Desmoplasia in Mouse and Human PDACs

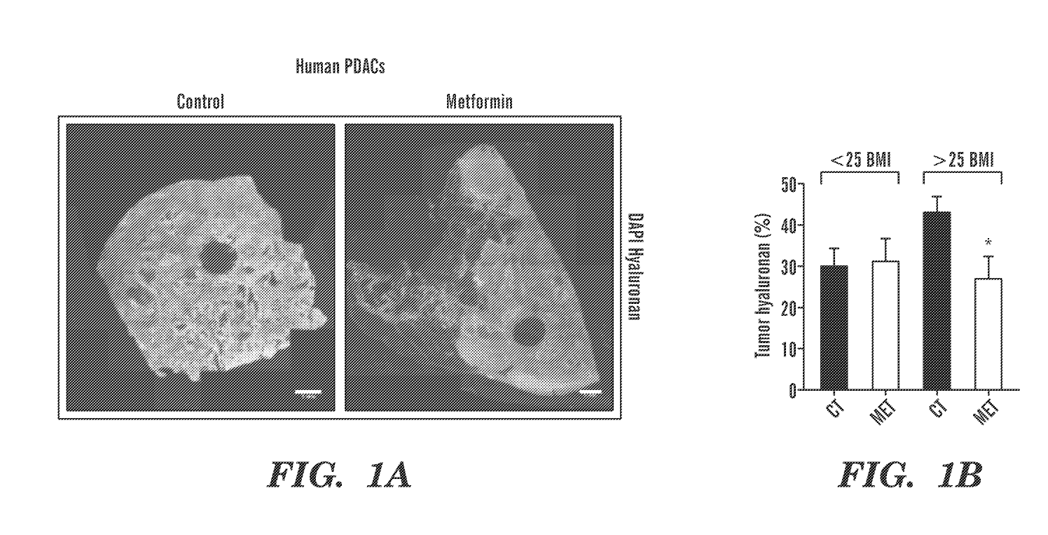

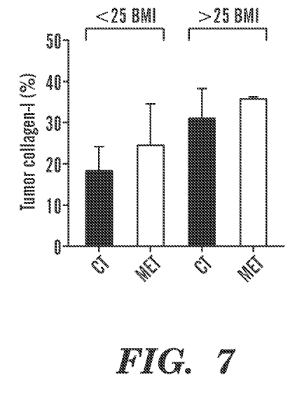

The reduction of desmoplasia—lowering ECM components such as HA and collagen-I—in PDACs potentiate anti-tumor treatments (41-43). Hence, the relationship between metformin treatment and desmoplasia in PDACs was examined in chemo/radiotherapy naïve PDAC surgical samples. It was found that in overweight or obese patients under treatment with metformin, levels of HA were 30% lower than in patients not taking metformin (FIG. 1A-1B). Interestingly, no difference in HA levels was observed in metformin treated patients of normal weight. On the other hand, treatment with metformin appeared to have no impact on collagen-I expression in either body weight group (FIG. 7).

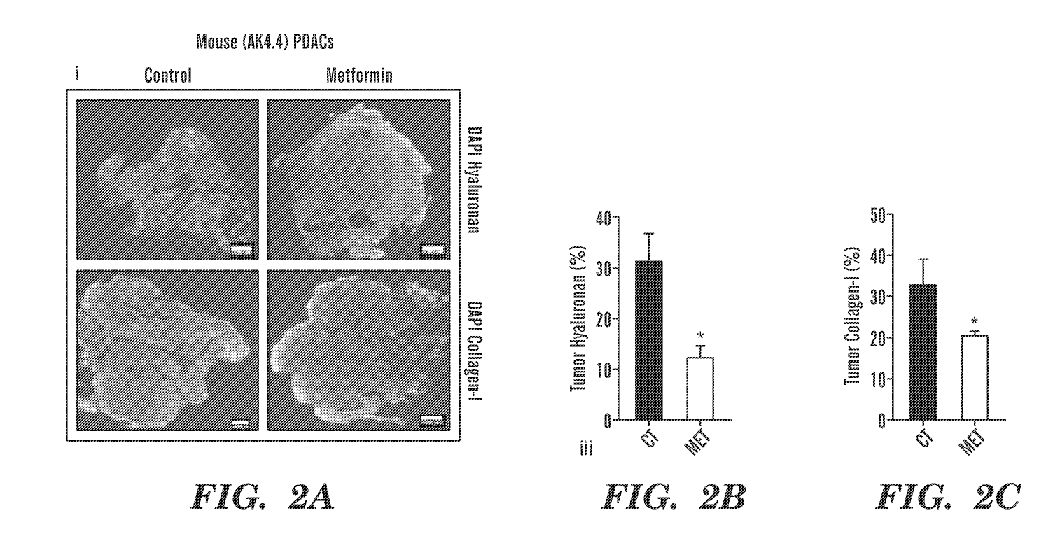

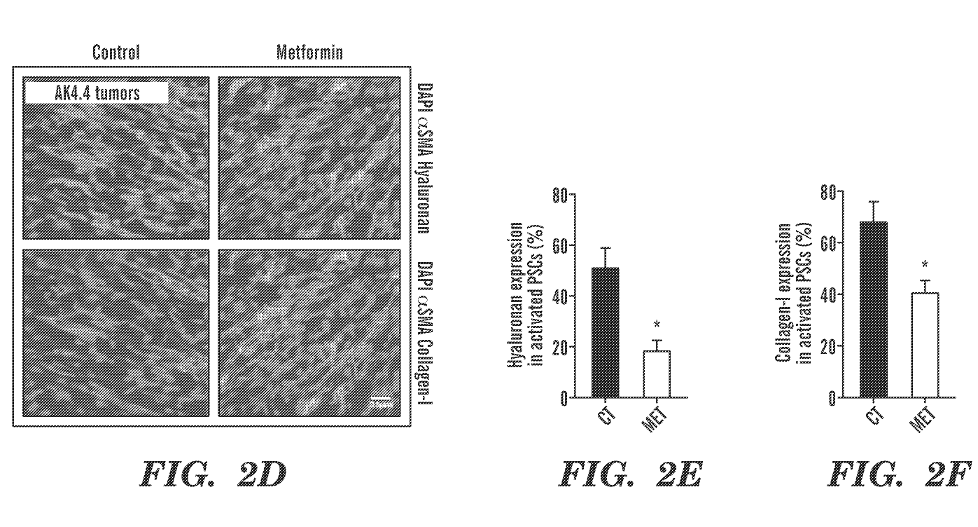

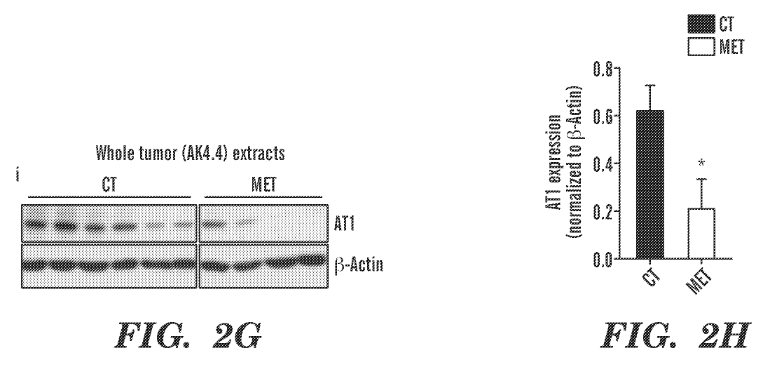

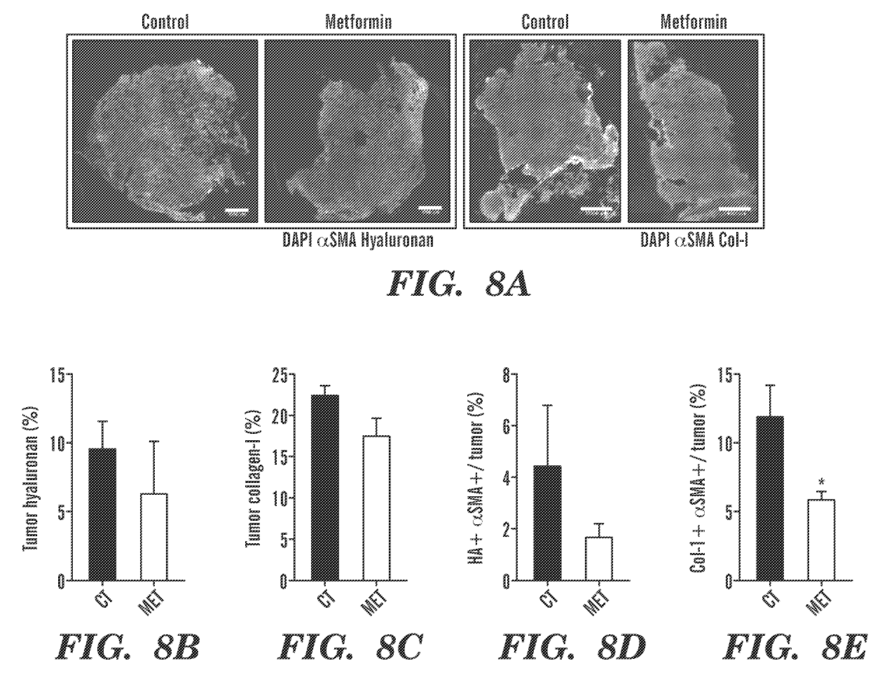

To evaluate whether these findings could be recapitulated in pre-clinical models, FVB and C57BL/6 mice fed with a high-fat diet were analyzed. A high-fat diet induces obesity and metabolic abnormalities typical of DM2, elevated glucose, insulin and IGF-1 (50, 57-60) in these strains. After 10 weeks on the high-fat diet, AK4.4 and PAN02 tumors were orthotopically implanted in obese FVB and C57BL/6 mice, respectively. The animals were randomly assigned to metformin in drinking water (300 mg/Kg) or no treatment at day 7 until day 21, when plasma and tumors were collected. Indeed, treatment with metformin correlated with reduced expression of HA by 64% (FIG. 2A-2B) and of collagen-I by 35% (FIGS. 2A and 2C) in AK4.4. tumors. Furthermore, the percentage of activated PSCs (as determined by the expression of alpha-smooth muscle antigen, αSMA) that co-express HA and collagen-I decreased by 58 and 38%, respectively (FIGS. 2D, 2E, and 2F). In a second, less desmoplastic model (PAN02), metformin decreased the expression of HA by 40% and of collagen-I by 22%, although it did not reach statistical significance (FIG. 8A-8C). Nonetheless, metformin significantly reduced the density of collagen-I positive activated PSCs in tumors by 54% in this model (FIG. 8E). The density of HA positive activated-PSCs in PAN02 tumors was also reduced by 57% (FIG. 8C), but did not reach significance. Angiotensin-1 (AT1) is critical for HA and collagen-I production in PDACs(43). Indeed, metformin was able to reduce the expression of AT1 (FIGS. 2G and 2H). Taken together, these data indicate that metformin reduces desmoplasia in PDACs in overweight/obese hosts.

### Metformin Affects Desmoplasia by Directly Reducing TGF-β Signaling and Production of Collagen-I/HA by PSCs

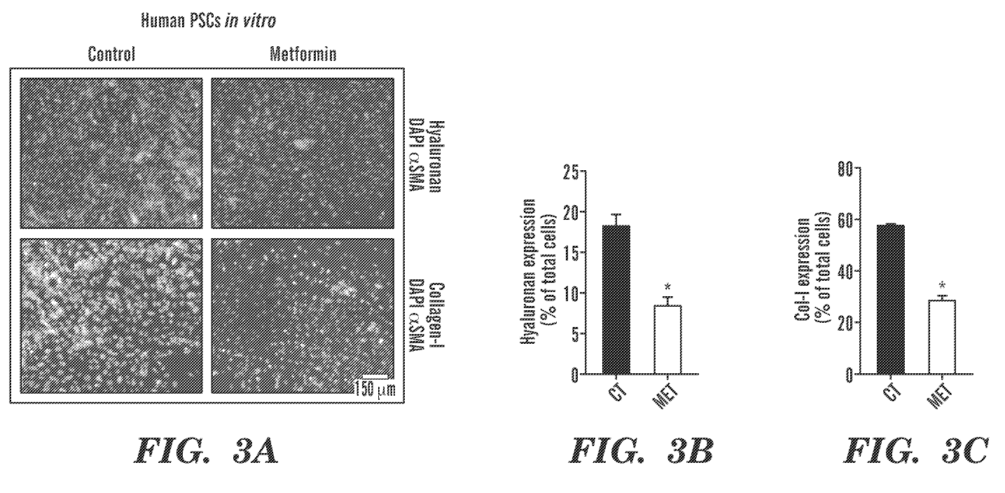

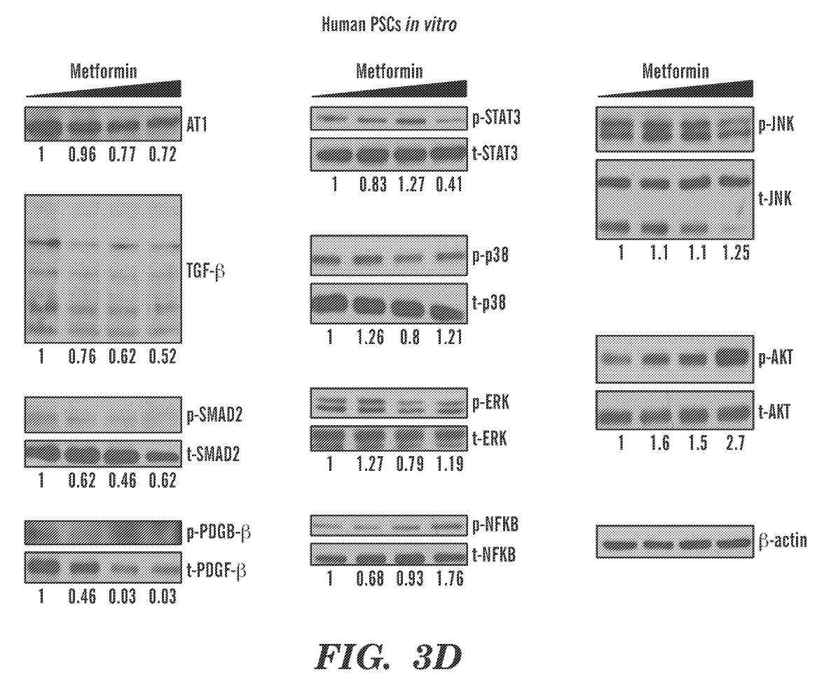

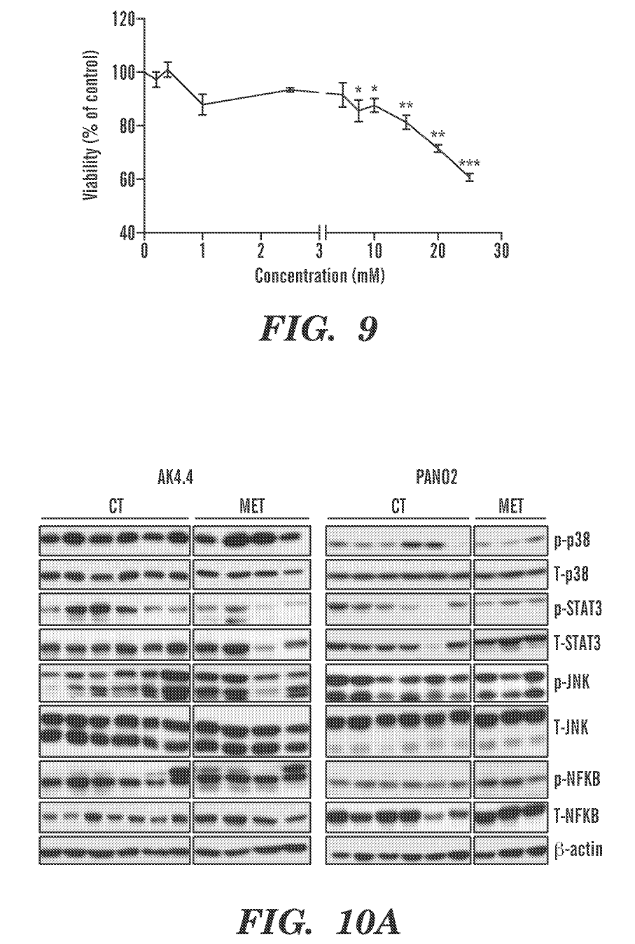

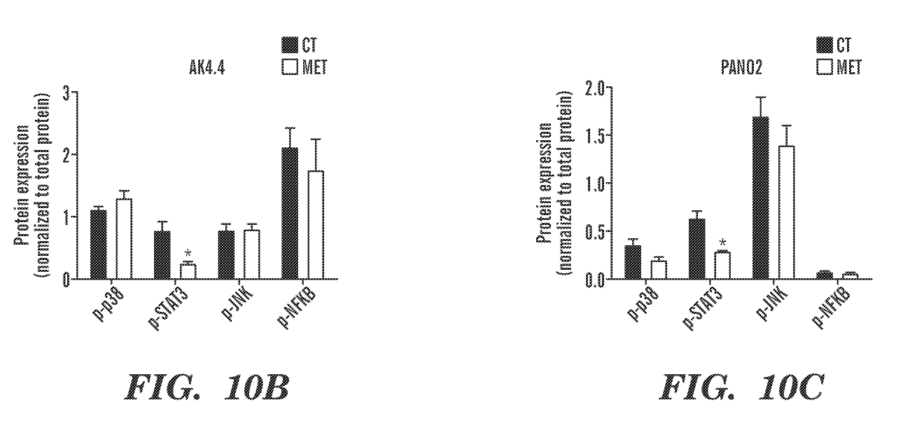

Collagen-I and HA are essentially produced by activated PSCs in PDACs (43, 61). In order to determine if metformin treatment can directly reduce collagen-I/HA expression in PSCs, PSCs were incubated in vitro with metformin. At a dose (1 mM) that does not substantially affect the viability of PSCs (FIG. 9), metformin decreased the expression of HA and collagen-I, indicating reduced production of these critical ECM components (FIG. 3A-3C). Furthermore, metformin (0.1-1mM) reduced expression of Angiotensin-II receptor 1 (AT1), TGF-β and downstream signaling via SMAD-2, as well as PDGF-I3, all key players in ECM production by PSCs (FIG. 3D). In addition, metformin also affected canonical signaling pathways that promote PSC activation and fibrosis (62), in particular ERK, p38, and STAT3, although at relatively higher doses (1-10 mM) (FIG. 3D). Importantly, in whole tumors, metformin decreased activation of STAT3 in both models, with a trend for reduced p38 in PAN02 (FIG. 10A-10C), suggesting that metformin is able to accumulate at concentrations high enough to affect these signaling pathways in vivo. Taken together, these data indicate that metformin affects desmoplasia by directly reducing AT1/TGF-β/STAT3 signaling and production of collagen-I/HA by PSCs.

Metformin also Improves Desmoplasia by Preventing Recruitment and M2 Polarization of Macrophages in PDACs.

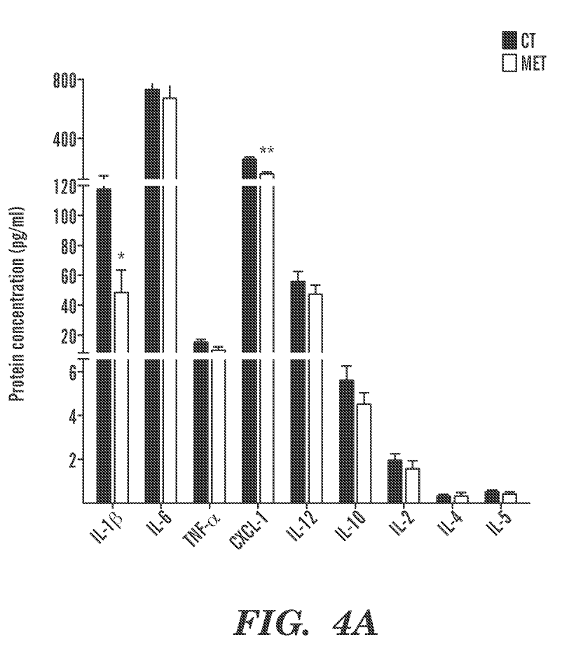

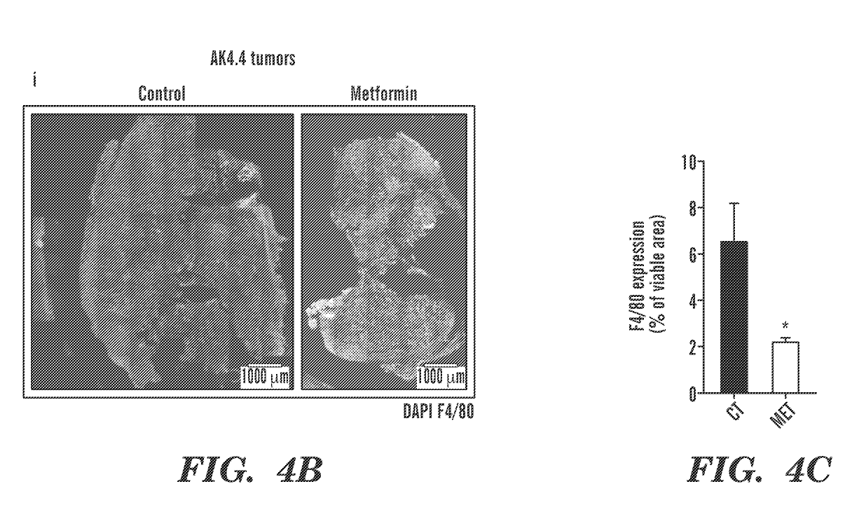

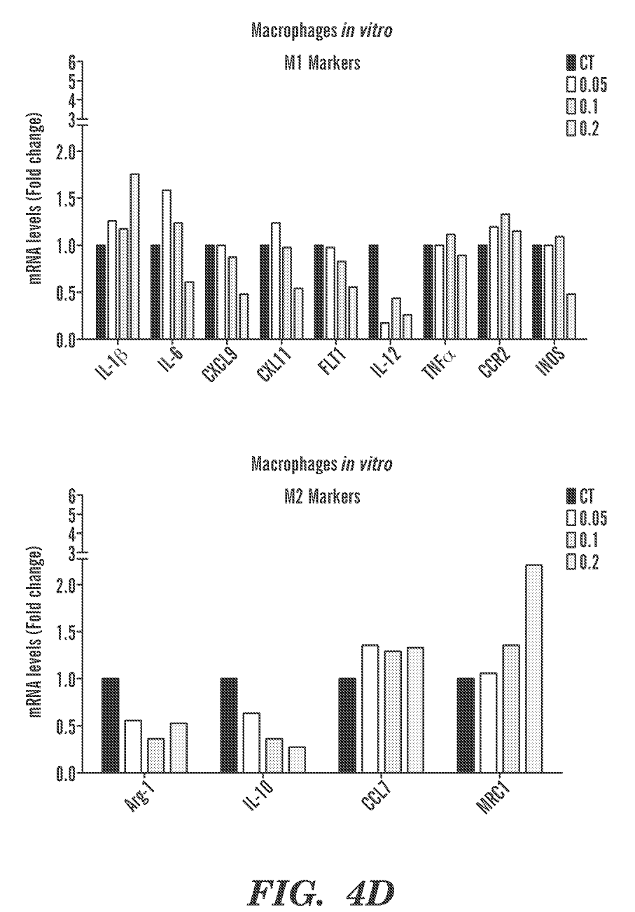

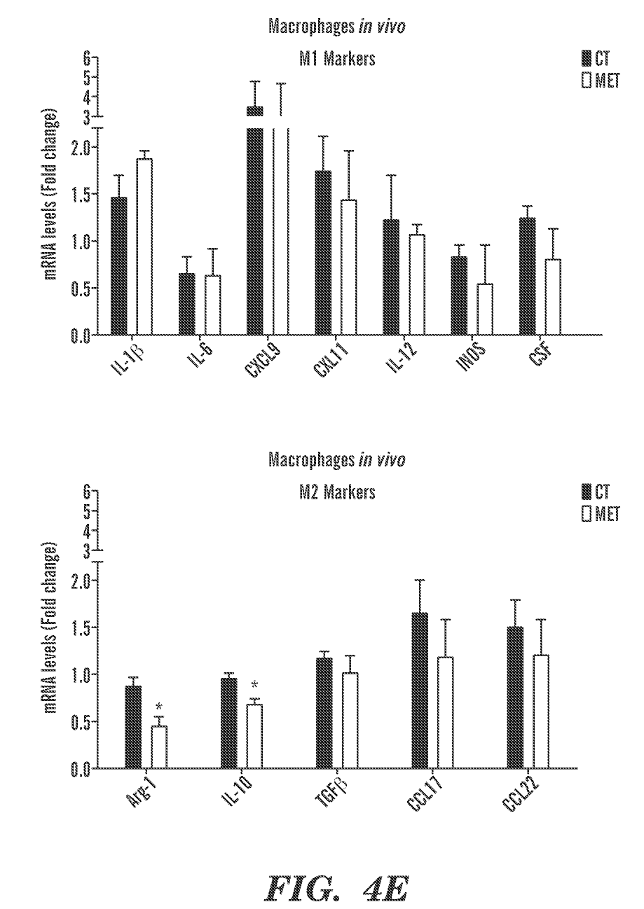

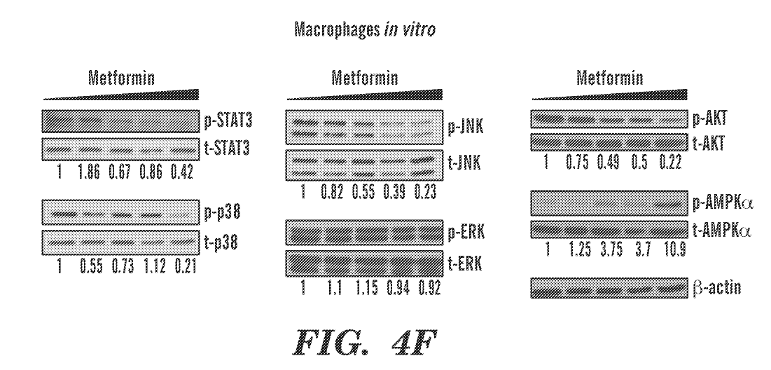

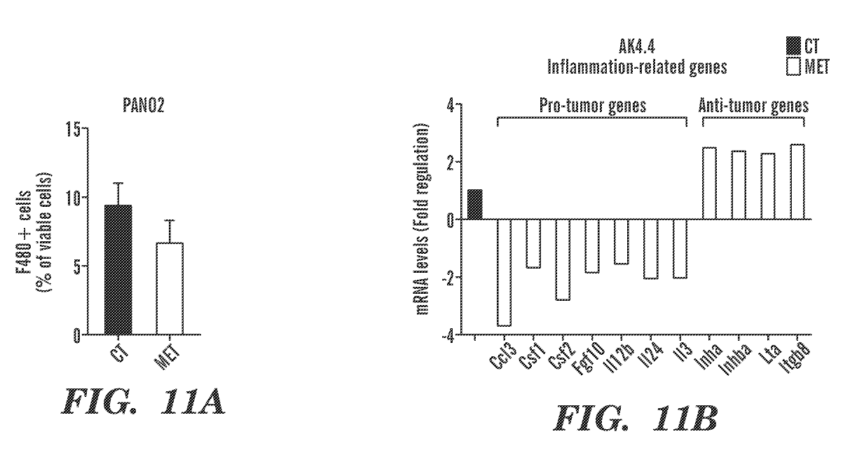

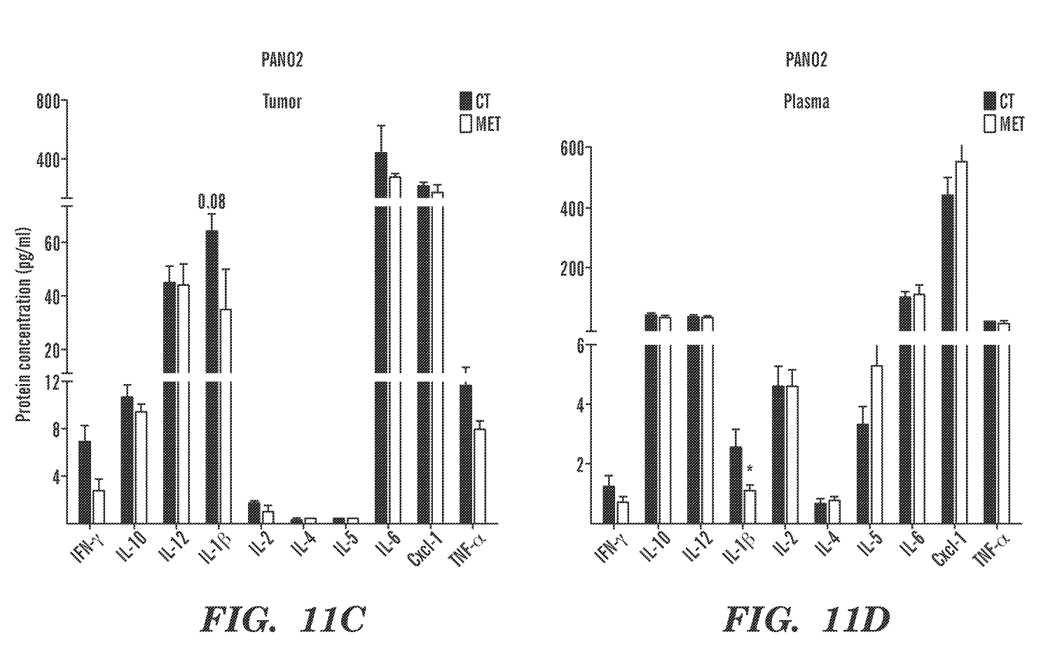

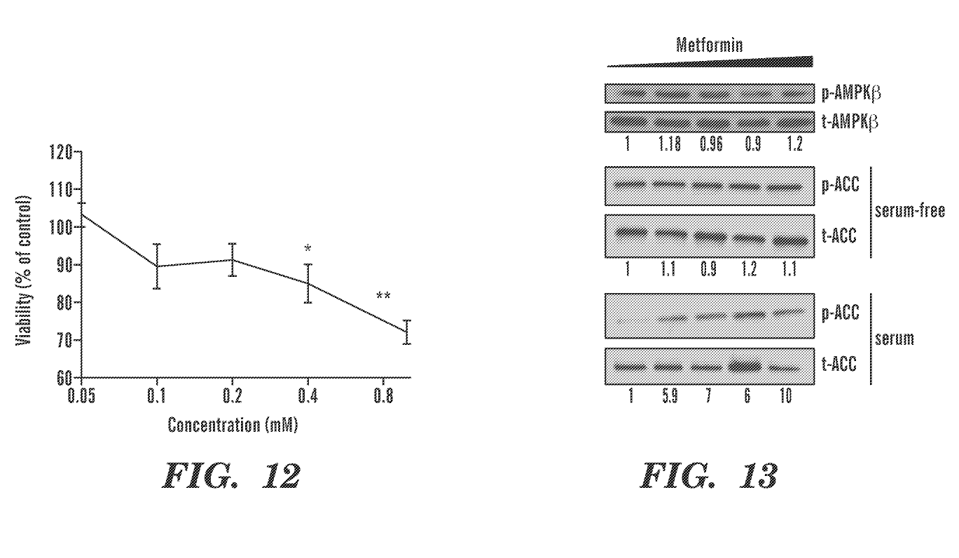

The inflammation that occurs in PDACs is a major component of desmoplasia (63). In particular, tumor-associated macrophages (TAMs) are a major source of cytokines that aggravate desmoplasia in PDACs (64) and negatively affect disease outcome (65). Therefore, the effect of metformin on TAM infiltration in tumors was determined. TAM levels were 60% lower in the AK4.4 model with metformin treatment (FIGS. 4B and 4C), and tended to lower (˜30%, not significant) in the PAN02 model (FIG. 11A). To determine a direct effect of metformin on macrophages, macrophages were incubated with metformin in vitro for 48 h at increasing concentrations. Metformin affected the viability of macrophages at doses of 0.4 mM or higher (FIG. 12). Metformin at a concentration of 0.05 mM (similar to the concentration measured in plasma of patients taking metformin) reduced M2 markers such as Arg-1 and IL-10 while metformin at doses higher than 0.2 mM reduced both M1 and M2 markers (FIG. 4D). In flow-sorted TAMs from PAN02 tumors in vivo, an effect of metformin on the expression of M2 markers Arg-1 (˜1/2) and IL-10 (˜2/3) was determined without significantly affecting the expression of M1 markers (FIG. 4E).

Several canonical and non-canonical signaling pathways can be activated in PDACs during inflammation and promote expression of M2 markers on TAMs (66-69). Consistent with the effects on TAMs, metformin reduced activation of STAT3, JNK, AKT and p38 in macrophages in vitro at concentrations lower than 0.2 mM (FIG. 4F). As mentioned above, in whole tumors metformin decreased activation of STAT3 in both models, with a trend for reduced p38 in PAN02, in line with in vitro results (FIG. 10A-10C). It has been shown that STAT3 activity is decreased by metformin via activation of metabolic energy sensor AMP-activated protein kinase (AMPK) in multiple cell types (70). Indeed, the inhibitory effects of metformin on STAT3 signaling in macrophages in vitro associated with activation of AMPKα and downstream enzyme Acetyl-CoA Carboxylase (ACC) (the latter evident only in serum added media) in these cells (FIGS. 4F and 13).

Taken together, these data indicate that metformin reduces TAM infiltration as well as expression of M2 markers, which may be mediated at least in part via AMPK/STAT3 signaling inhibition in macrophages. In addition, inflammation in tumors is characterized by excess of inflammatory cytokines that promote desmoplasia, and metformin has been shown to affect multiple inflammatory mediators (34, 35). Here, it was found that metformin reduced the expression of IL-1β and CXCL-1 in AK4.4 tumors (FIG. 4A). A reduction of IL-1β after metformin treatment also occurred in the PAN02 tumors (FIGS. 11C and 11D). In addition, a broader panel of inflammation-related genes revealed that metformin reduced multiple genes involved in TAM recruitment and function (FIG. 11B). In conclusion, metformin reduces the production of desmoplastic cytokines (e.g. IL-1β) as well as infiltration and M2 polarization of TAMs, which may be mediated at least in part via AMPK/STAT3 signaling inhibition in macrophages.

### Metformin Reduces ECM Remodeling, EMT and Metastasis

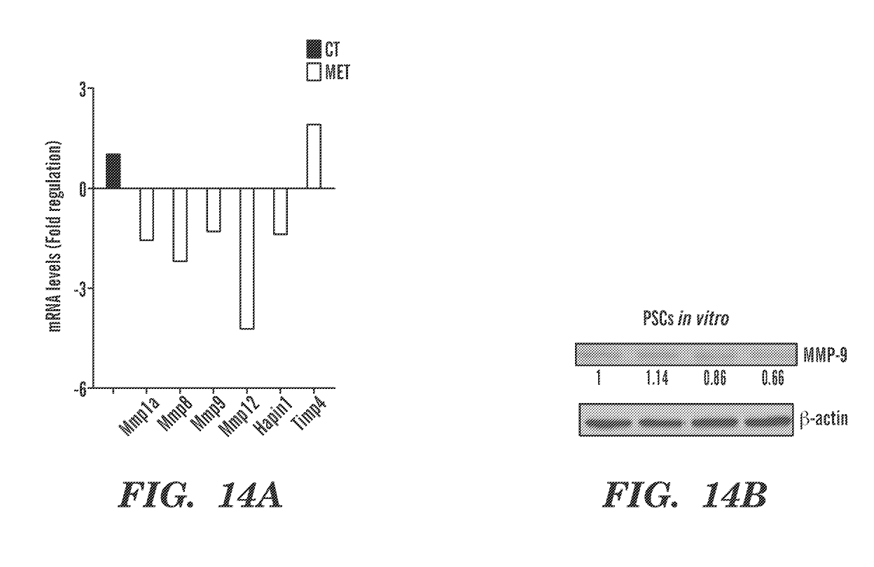

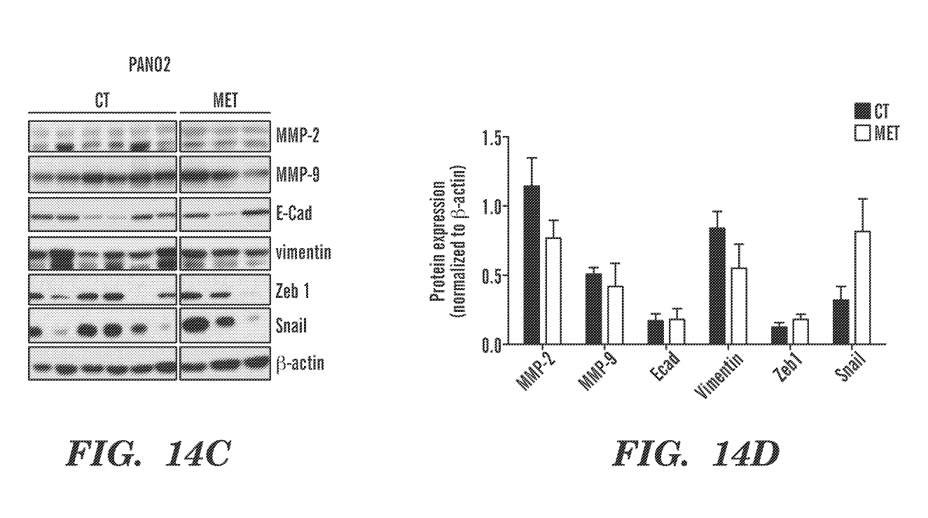

In addition to producing ECM components, PSCs also promote ECM remodeling and EMT to facilitate invasion and metastasis (43, 71, 72). Hence, it was determined whether the effects on PSCs by metformin also extend to these processes. Indeed metformin reduced the expression in AK4.4 tumors of multiple genes involved in ECM remodeling (including MMPs) and EMT (FIG. 5A). In addition, metformin treatment upregulated genes that prevent ECM remodeling (FIG. 5A). Although to a lesser extent, similar findings were observed in PAN02 tumors (FIG. 14A). At the protein level, we also observed a reduction of metalloproteinase 9 (MMP9, FIGS. 5B and 5C) by 70% in the AK4.4 model with metformin treatment. Average MMP-2 levels were also approximately half (not significant) in metformin treated PAN02 tumors (FIG. 14C-14D). Consistently, it was confirmed in vitro that metformin decreased protein levels of MMP9 in PSCs (FIG. 14B). In addition, MMP activity in tumors was also decreased in metformin treated animals compared to control mice (FIG. 5D). In addition to ECM remodeling, EMT was also affected. At the protein expression level, the EMT marker vimentin was decreased and E-cadherin was increased in AK4.4 with similar trends in the PAN02 model, confirming reduced EMT (FIG. 5B-5C, FIG. 14C-14D). Consistent with these effects on the tumor microenvironment, metformin reduced the incidence of metastasis (number of mice affected) (FIG. 5E) as well as the average number of mesenteric peritoneal and retroperitoneal wall metastasis per mouse (FIG. 5F). These effects were particularly evident in the more metastatic model PAN02, although similar trends were obtained for the less metastatic AK4.4 model.

### Effects of Metformin on Desmoplasia are Independent of Global Metabolic Effects

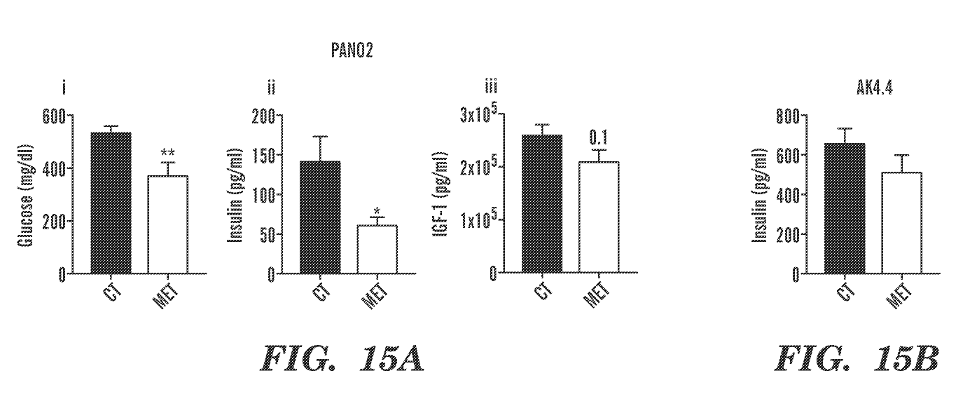

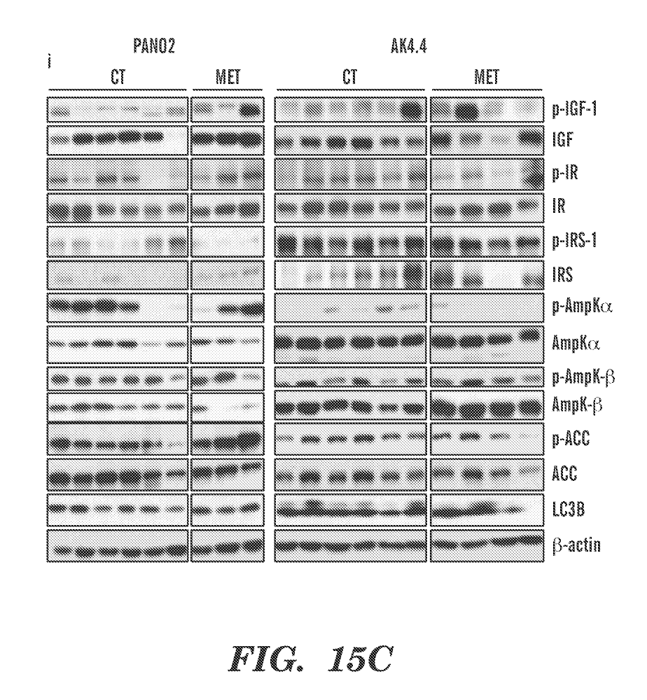

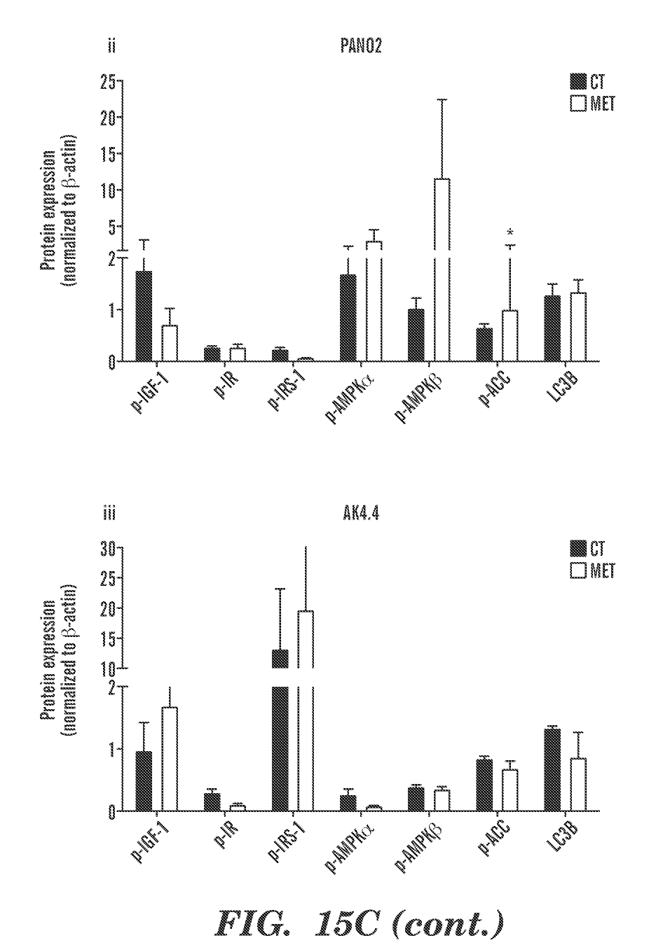

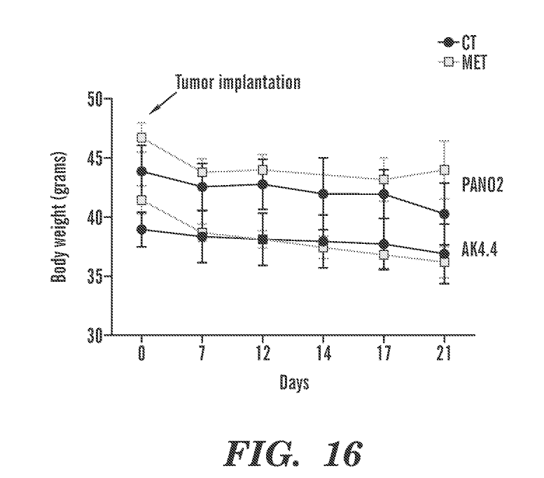

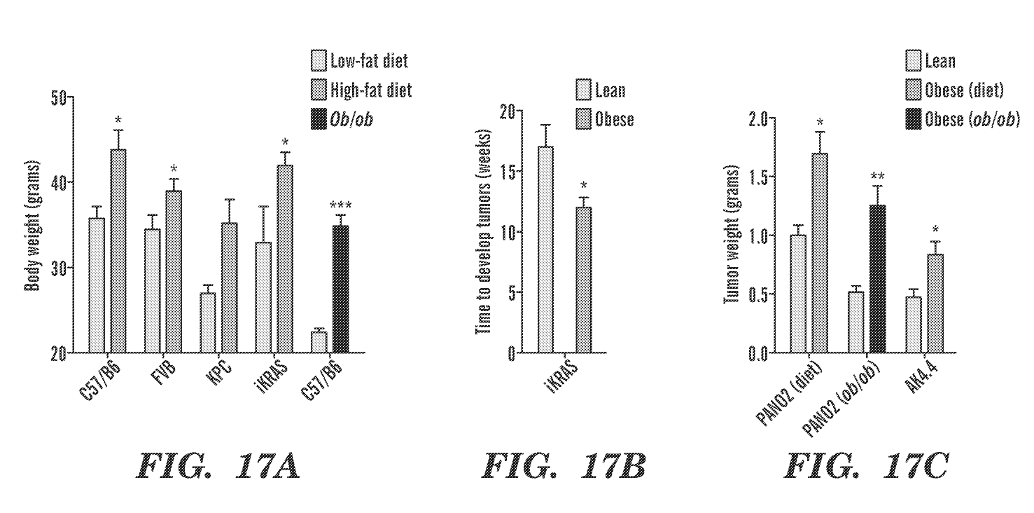

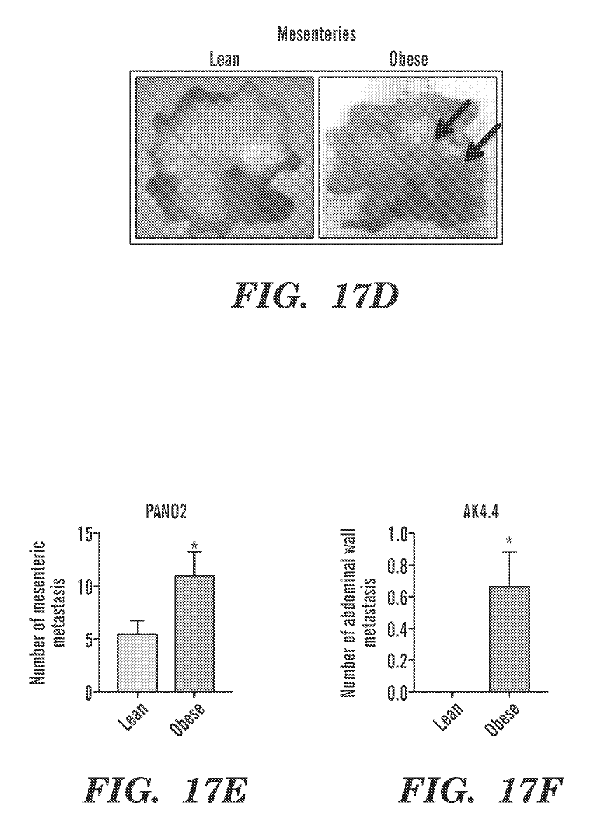

As expected, metformin reduced systemic levels of glucose and Insulin/IGF-I in the PAN02 model (FIG. 15A), with similar trends occurring for Insulin levels in the AK4.4 model (FIG. 15B). At the tumor level, metformin induced a trend for reduced activation and phosphorylation of the insulin-like growth factor receptor-1 (IGFR-1) and downstream mediator insulin receptor substrate 1 (IRS-1) (FIG. 15Ci-ii). In addition, activation of ACC was increased by metformin, with similar trends for AMPKα and AMPKβ (FIG. 15Ci-ii). However, metformin did not affect the activation of metabolic pathways in AK4.4 tumors (FIG. 15Ci and 15Ciii), where it more dramatically improved the tumor microenvironment. In addition, the expression of the autophagy marker L3CB was unchanged by metformin in both models (FIG. 15Ci-iii). Furthermore, no change in body weight was observed in either model (FIG. 16). These data suggests that the effects of metformin on desmoplasia and metastasis do not correlate with global activation of metabolic pathways or body weight. On the other hand, as shown above metformin induced specific changes in metabolic pathways in TAMs. Taken together, these findings indicate that a global change in the activity of metabolic pathways in tumors does not correlate with the effects of metformin on tumor desmoplasia.

### Discussion

Metformin improves cancer outcomes in preclinical models of PDAC (25, 26) and in diabetic patients with pancreatic cancer (32, 35) though the underlying mechanisms are not well understood. Hence, there is a need to continue to study and elucidate the mechanisms of action of metformin in PDACs. Fibrosis and inflammation are critical components of the desmoplasia which characterize PDACs (73), and studies have previously shown that reprogramming the critical instigator of the desmoplastic microenvironment—PSCs—can be an effective intervention in the treatment of PDACs (43, 61, 74). Furthermore, metastases in PDACs are facilitated by the active desmoplastic fibro-inflammatory microenvironment that promotes ECM remodeling, EMT and tumor invasion (43-45, 75). Uncovered in this study is a previously unknown role of metformin on the activity of PSCs, TAMs, tumor fibrosis and inflammation, and how it impacts systemic dissemination of the disease. In overweight and obese patients,which appear to have increased levels of ECM components in tumors, metformin treatment reduced tumor levels of HA. Metformin robustly affected HA as well as collagen-I, though to a lesser extent, in preclinical obese/diabetic mouse models of syngeneic PDACs. Furthermore, these effects were mediated by a direct effect on HA and collagen-I production by PSCs, which was associated with reduction of AT1/PDGF-β expression and TGF-β/SMAD-2 signaling. Although the precise mechanisms for the preferential effect of metformin on HA over collagen-I in all preclinical models and human cancer patients studied so far are not clear, it is an intriguing and potentially important finding. Nonetheless, we have recently observed that HA plays an equally important role as collagen-I in reducing therapy delivery and efficacy in PDACs (43, 76).

In addition to reducing fibrosis, it was found that metformin reduces the production of pro-metastatic cytokines. In both tumor models, metformin reduced the secretion of IL-1β, which has been shown in a PDAC model to promote metastasis (30). IL-1β in tumors is typically produced by PSCs, inflammatory and tumor cells, is involved in macrophage recruitment and PSC activation, and both IL-1β and CXCL-1 worsen desmoplasia (77-80). Furthermore, metformin reduced the levels of chemokines involved in TAM recruitment and function (e.g. CSFs, CCL-3) (81-83), and consistently, metformin reduced recruitment of TAMs and their expression of M2 markers in vivo and in vitro at clinically relevant doses. STAT3 promotes polarization of TAMs to an M2 phenotype (84), and It has been shown that STAT3 activity is decreased by metformin via activation of AMPKα in multiple cell types (70). Consistently, the effects of metformin on macrophage polarization associated with activation of AMPKα/ACC and reduction of STAT3 signaling.

Activated PSCs and M2 TAMs have been shown to promote ECM remodeling and EMT (85, 86). Consistent with the effects on these cells, metformin reduces ECM remodeling and EMT. This is also consistent with a recent report describing the effects of metformin on EMT in the PAN02 model (87). In addition, this data is consistent with the finding that metformin impeded TGF-β-promoted EMT in breast cancer cells (88). Importantly, ECM remodeling and EMT have been shown to promote tumor invasion and metastasis (43-45, 75), and as expected a decrease in metastasis in mice treated with metformin was observed.

The modulation of systemic and local metabolism has been the major focus of studies evaluating the effect of metformin in PDAC. Metformin could improve systemic levels of Insulin/IGF-1 and glucose, and affect Insulin/IGF-1 signaling and AMPK/ACC activation. However, this only occurred (and mildly) in one of the models (PAN02). This suggests that the effects of metformin on desmoplasia do not correlate with global activity of metabolic pathways. In fact, despite earlier reports suggesting that IGF-I may be involved in cancer risk and outcome (89), subsequent clinical studies failed to establish anti-IGF-I agents as cancer therapeutics (90). In addition, there is no convincing evidence for a carcinogenic role of any insulin derivative currently used in therapy for diabetes (91). Similarly, the beneficial effects of metformin did not correlate with levels of blood sugar in patients (33), and in a pre-clinical model Franco and colleagues have shown that the effects of metformin may be more dependent on the direct effect on tumors rather than on systemic metabolism (92). In addition, despite the report that autophagy can be affected by metformin to reduce tumor progression (93, 94), there was no evidence of this in this study. Of note, metformin did not affect body weight through the experiment (FIG. 13), suggesting a body weight-independent effect on tumor growth.

Importantly, two very recent studies—one retrospective (95) and the first prospective study (96) indicated that metformin might not be uniformly beneficial. The benefit in some but not all studies suggests that a subset of tumors may not respond to metformin and that a careful selection of patients may be required for metformin to be effective. It was found that metformin's effect on desmoplasia in patients only occurred when their BMI was higher than 25 (overweight and obese patients). This indicates that metformin may not be beneficial in normal weight patients, and suggests that BMI should be explored as a potential biomarker of response to this drug.

### Conclusion

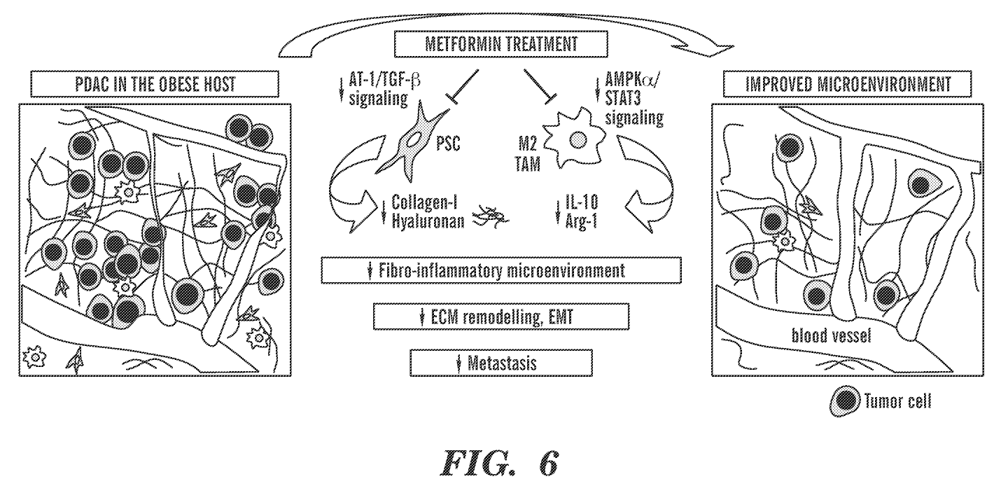

In conclusion, this study indicates that in an overweight/obese condition, metformin reprograms the fibro-inflammatory tumor microenvironment and ultimately reduces metastasis. Metformin directly reduces AT1/PDGF-β and TGF-β signaling and ECM production by PSCs—preferentially HA. Metformin also reduces inflammation—another key element of desmoplasia—through reduction of cytokine production, and recruitment and M2-polarization of TAMs. This was associated with AMPK activation and STAT3 signaling inhibition in macrophages. Finally, the alleviation of desmoplasia by metformin was associated with reduced ECM remodeling, EMT and systemic metastasis (FIG. 6). Importantly, the effects on desmoplasia observed in human samples seem restricted to an overweight/obese population, which appear to have tumors with increased content of ECM components. With nearly 200 trials ongoing to address the effect of metformin on diabetic and non-diabetic cancer patients, understanding the yet elusive mechanisms of action of metformin may provide an opportunity to uncover potential biomarkers of response and define strategies of patient stratification for the judicious use of this highly promising yet generic drug.

### REFERENCES FOR EXAMPLE 1

1. American Cancer Society: Cancer Facts and FIGS. 2015. http://wwwcancerorg/acs/groups/content/@editorial/documents/document/acspc-044552pdf.

2. McCarthy M. Nearly one in 10 US residents has diabetes, CDC reports. Bmj. 2014; 348:g3962. doi: 10.1136/bmj.g3962. PubMed PMID: 24923592.

3. Smyth S, Heron A. Diabetes and obesity: the twin epidemics. Nat Med. 2006; 12(1): 75-80. doi: 10.1038/nm0106-75. PubMed PMID: 16397575.

4. Genkinger J M, Spiegelman D, Anderson K E, Bernstein L, van den Brandt P A, Calle E E, et al. A pooled analysis of 14 cohort studies of anthropometric factors and pancreatic cancer risk. International journal of cancer Journal international du cancer. 2011; 129(7):1708-17. doi: 10.1002/ijc.25794. PubMed PMID: 21105029; PubMed Central PMCID: PMC3073156.

5. Kasenda B, Bass A, Koeberle D, Pestalozzi B, Borner M, Herrmann R, et al. Survival in overweight patients with advanced pancreatic carcinoma: a multicentre cohort study. BMC cancer. 2014; 14:728. doi: 10.1186/1471-2407-14-728. PubMed PMID: 25266049; PubMed Central PMCID: PMC4242603.

6. Hori M, Takahashi M, Hiraoka N, Yamaji T, Mutoh M, Ishigamori R, et al. Association of pancreatic Fatty infiltration with pancreatic ductal adenocarcinoma. Clinical and translational gastroenterology. 2014; 5:e53. doi: 10.1038/ctg.2014.5. PubMed PMID: 24622469; PubMed Central PMCID: PMC3972693.

7. Hursting S D, Dunlap S M. Obesity, metabolic dysregulation, and cancer: a growing concern and an inflammatory (and microenvironmental) issue. Annals of the New York Academy of Sciences. 2012; 1271:82-7. doi: 10.1111/j.1749-6632.2012.06737.x. PubMed PMID: 23050968; PubMed Central PMCID: PMC3485672.

8. Pischon T, Nothlings U, Boeing H. Obesity and cancer. Proc Nutr Soc. 2008; 67(2):128-45. PubMed PMID: 18412987.

9. Calle E E, Thun M J. Obesity and cancer. Oncogene. 2004; 23(38): 6365-78. PubMed PMID: 15322511.

10. Pannala R, Basu A, Petersen G M, Chari S T. New-onset diabetes: a potential clue to the early diagnosis of pancreatic cancer. The Lancet Oncology. 2009; 10(1): 88-95. doi: 10.1016/51470-2045(08)70337-1. PubMed PMID: 19111249; PubMed Central PMCID: PMC2795483.

11. Bartosch-Harlid A, Andersson R. Diabetes mellitus in pancreatic cancer and the need for diagnosis of asymptomatic disease. Pancreatology. 2010; 10(4): 423-8. doi: 10.1159/000264676. PubMed PMID: 20720443.

12. Karnevi E, Andersson R, Rosendahl A H. Tumour-educated macrophages display a mixed polarisation and enhance pancreatic cancer cell invasion. Immunology and cell biology. 2014; 92(6): 543-52. doi: 10.1038/icb.2014.22. PubMed PMID: 24662521.

13. Noto H, Goto A, Tsujimoto T, Noda M. Cancer risk in diabetic patients treated with metformin: a systematic review and meta-analysis. PloS one. 2012; 7(3):e33411. doi: 10.1371/journal.pone.0033411. PubMed PMID: 22448244; PubMed Central PMCID: PMC3308971.

14. Bao B, Wang Z, Li Y, Kong D, Ali S, Banerjee S, et al. The complexities of obesity and diabetes with the development and progression of pancreatic cancer. Biochimica et biophysica acta. 2011; 1815(2):135-46. doi: 10.1016/j.bbcan.2010.11.003. PubMed PMID: 21129444; PubMed Central PMCID: PMC3056906.

15. Li D. Diabetes and pancreatic cancer. Molecular carcinogenesis. 2012; 51(1): 64-74. doi: 10.1002/mc.20771. PubMed PMID: 22162232; PubMed Central PMCID: PMC3238796.

16. Li D, Morris J S, Liu J, Hassan M M, Day R S, Bondy M L, et al. Body mass index and risk, age of onset, and survival in patients with pancreatic cancer. JAMA:the journal of the American Medical Association. 2009; 301(24): 2553-62. doi: 10.1001/jama.2009.886. PubMed PMID: 19549972; PubMed Central PMCID: PMC2760963.

17. Chow W H, Gridley G, Nyren O, Linet M S, Ekbom A, Fraumeni J F, Jr., et al. Risk of pancreatic cancer following diabetes mellitus: a nationwide cohort study in Sweden. J Natl Cancer Inst. 1995; 87(12): 930-1. PubMed PMID: 7666483.

18. Godsland I F. Insulin resistance and hyperinsulinaemia in the development and progression of cancer. Clin Sci (Lond). 2010; 118(5): 315-32. doi: 10.1042/CS20090399. PubMed PMID: 19922415; PubMed Central PMCID: PMC2782313.

19. Leone A, Di Gennaro E, Bruzzese F, Avallone A, Budillon A. New perspective for an old antidiabetic drug: metformin as anticancer agent. Cancer treatment and research. 2014; 159:355-76. doi: 10.1007/978-3-642-38007-5_21. PubMed PMID: 24114491.

20. Foretz M, Guigas B, Bertrand L, Pollak M, Viollet B. Metformin: from mechanisms of action to therapies. Cell metabolism. 2014; 20(6):953-66. doi: 10.1016/j.cmet.2014.09.018. PubMed PMID: 25456737.

21. Costa C, Incio J, Soares R. Angiogenesis and chronic inflammation: cause or consequence? Angiogenesis. 2007; 10(3):149-66. Epub 2007/04/26. doi: 10.1007/s10456-007-9074-0. PubMed PMID: 17457680.

22. Incio J, Soares R. Obesity, Diabetes and Metabolic Syndrome Impact on TUMOR ANGIOGENESIS. In: Ruben R. Gonzalez-Perez B R R, editor. Tumor Angiogenesis Regulators: CRC Press; 2013.

23. Evans J M, Donnelly L A, Emslie-Smith A M, Alessi D R, Morris A D. Metformin and reduced risk of cancer in diabetic patients. Bmj. 2005; 330(7503):1304-5. doi: 10.1136/bmj.38415.708634.F7. PubMed PMID: 15849206; PubMed Central PMCID: PMC558205.

24. Zechner D, Radecke T, Amme J, Burtin F, Albert A C, Partecke L I, et al. Impact of diabetes type II and chronic inflammation on pancreatic cancer. BMC cancer. 2015; 15:51. doi: 10.1186/s12885-015-1047-x. PubMed PMID: 25885700; PubMed Central PMCID: PMC4336675.

25. Goodwin P J, Stambolic V. Obesity and insulin resistance in breast cancer-Chemoprevention strategies with a focus on metformin. Breast. 20 Suppl 3:S31-5. Epub Nov. 02, 2011 doi: 50960-9776(11)70291-0 [pii] 10.1016/S0960-9776(11)70291-0. PubMed PMID: 22015290.

26. Algire C, Zakikhani M, Blouin M J, Shuai J H, Pollak M. Metformin attenuates the stimulatory effect of a high-energy diet on in vivo LLC1 carcinoma growth. Endocrine-related cancer. 2008; 15(3):833-9. doi: 10.1677/ERC-08-0038. PubMed PMID: 18469156.

27. Kisfalvi K, Moro A, Sinnett-Smith J, Eibl G, Rozengurt E. Metformin inhibits the growth of human pancreatic cancer xenografts. Pancreas. 2013; 42(5):781-5. doi: 10.1097/MPA.0b013e31827aec40. PubMed PMID: 23462329; PubMed Central PMCID: PMC3681894.

28. Nair V, Pathi S, Jutooru I, Sreevalsan S, Basha R, Abdelrahim M, et al. Metformin inhibits pancreatic cancer cell and tumor growth and downregulates Sp transcription factors. Carcinogenesis. 2013; 34(12): 2870-9. doi: 10.1093/carcin/bgt231. PubMed PMID: 23803693; PubMed Central PMCID: PMC3845888.

29. Mohammed A, Janakiram N B, Brewer M, Ritchie R L, Marya A, Lightfoot S, et al. Antidiabetic Drug Metformin Prevents Progression of Pancreatic Cancer by Targeting in Part Cancer Stem Cells and mTOR Signaling. Translational oncology. 2013; 6(6):649-59. PubMed PMID: 24466367; PubMed Central PMCID: PMC3890699.

30. Tan X L, Bhattacharyya K K, Dutta S K, Bamlet W R, Rabe K G, Wang E, et al. Metformin Suppresses Pancreatic Tumor Growth With Inhibition of NFkappaB/STAT3 Inflammatory Signaling. Pancreas. 2015; 44(4):636-47. doi: 10.1097/MPA.0000000000000308. PubMed PMID: 25875801; PubMed Central PMCID: PMC4399019.

31. Choi Y, Kim T Y, Oh D Y, Lee K H, Han S W, Im S A, et al. The Impact of Diabetes Mellitus and Metformin Treatment on Survival of Patients with Advanced Pancreatic Cancer Undergoing Chemotherapy. Cancer research and treatment : official journal of Korean Cancer Association. 2015. doi: 10.4143/crt.2014.292. PubMed PMID: 25779362.

32. Sadeghi N, Abbruzzese J L, Yeung S C, Hassan M, Li D. Metformin use is associated with better survival of diabetic patients with pancreatic cancer. Clin Cancer Res. 2012; 18(10):2905-12. doi: 10.1158/1078-0432.CCR-11-2994. PubMed PMID: 22465831; PubMed Central PMCID: PMC3381457.

33. Li D, Yeung S C, Hassan M M, Konopleva M, Abbruzzese J L. Antidiabetic therapies affect risk of pancreatic cancer. Gastroenterology. 2009; 137(2):482-8. doi: 10.1053/j.gastro.2009.04.013. PubMed PMID: 19375425; PubMed Central PMCID: PMC2735093.

34. Nair V, Sreevalsan S, Basha R, Abdelrahim M, Abudayyeh A, Rodrigues Hoffman A, et al. Mechanism of metformin-dependent inhibition of mammalian target of rapamycin (mTOR) and Ras activity in pancreatic cancer: role of specificity protein (Sp) transcription factors. The Journal of biological chemistry. 2014; 289(40):27692-701. doi: 10.1074/jbc.M114.592576. PubMed PMID: 25143389; PubMed Central PMCID: PMC4183806.

35. Gong J, Robbins L A, Lugea A, Waldron R T, Jeon C Y, Pandol S J. Diabetes, pancreatic cancer, and metformin therapy. Frontiers in physiology. 2014; 5:426. doi: 10.3389/fphys.2014.00426. PubMed PMID: 25426078; PubMed Central PMCID: PMC4224068.

36. Choi Y K, Park K G. Metabolic roles of AMPK and metformin in cancer cells. Molecules and cells. 2013; 36(4):279-87. doi: 10.1007/s10059-013-0169-8. PubMed PMID: 23794020; PubMed Central PMCID: PMC3887985.

37. Dowling R J, Niraula S, Stambolic V, Goodwin P J. Metformin in cancer: translational challenges. Journal of molecular endocrinology. 2012; 48(3):R31-43. doi: 10.1530/JME-12-0007. PubMed PMID: 22355097.

38. Isoda K, Young J L, Zirlik A, MacFarlane L A, Tsuboi N, Gerdes N, et al. Metformin inhibits proinflammatory responses and nuclear factor-kappaB in human vascular wall cells. Arteriosclerosis, thrombosis, and vascular biology. 2006; 26(3):611-7. doi: 10.1161/01.ATV.0000201938.78044.75. PubMed PMID: 16385087.

39. Martinez-Outschoorn U E, Balliet R M, Rivadeneira D B, Chiavarina B, Pavlides S, Wang C, et al. Oxidative stress in cancer associated fibroblasts drives tumor-stroma co-evolution: A new paradigm for understanding tumor metabolism, the field effect and genomic instability in cancer cells. Cell cycle. 2010; 9(16):3256-76. doi: 10.4161/cc.9.16.12553. PubMed PMID: 20814239; PubMed Central PMCID: PMC3041164.

40. Qu H, Yang X. Metformin inhibits angiogenesis induced by interaction of hepatocellular carcinoma with hepatic stellate cells. Cell biochemistry and biophysics. 2015; 71(2):931-6. doi: 10.1007/s12013-014-0287-8. PubMed PMID: 25326336.

41. Chauhan V P, Boucher Y, Ferrone C R, Roberge S, Martin J D, Stylianopoulos T, et al. Compression of pancreatic tumor blood vessels by hyaluronan is caused by solid stress and not interstitial fluid pressure. Cancer Cell. 2014; 26(1):14-5. doi: 10.1016/j.ccr.2014.06.003. PubMed PMID: 25026209; PubMed Central PMCID: PMC4381566.

42. Diop-Frimpong B, Chauhan V P, Krane S, Boucher Y, Jain R K. Losartan inhibits collagen I synthesis and improves the distribution and efficacy of nanotherapeutics in tumors. Proceedings of the National Academy of Sciences of the United States of America. 2011; 108(7):2909-14. doi: 10.1073/pnas.1018892108. PubMed PMID: 21282607; PubMed Central PMCID: PMC3041115.

43. Chauhan V P, Martin J D, Liu H, Lacorre D A, Jain S R, Kozin S V, et al. Angiotensin inhibition enhances drug delivery and potentiates chemotherapy by decompressing tumor blood vessels. Nature Communications. 2013; 4:2516. Epub Oct. 1, 2013. doi: 10.1038/ncomms3516.

44. Jain R K. An Indirect Way to Tame Cancer. Scientific American. 2014; 310(2):46-53.

45. Jain R K, Martin J D, Stylianopoulos T. The role of physical forces in tumor growth and therapy. Annual Review of Biomedical Engineering. 2014; 16:321-46.

46. Algire C, Amrein L, Bazile M, David S, Zakikhani M, Pollak M. Diet and tumor LKB1 expression interact to determine sensitivity to anti-neoplastic effects of metformin in vivo. Oncogene. 2011; 30(10):1174-82. doi: 10.1038/onc.2010.483. PubMed PMID: 21102522.

47. Algire C, Amrein L, Zakikhani M, Panasci L, Pollak M. Metformin blocks the stimulative effect of a high-energy diet on colon carcinoma growth in vivo and is associated with reduced expression of fatty acid synthase. Endocrine-related cancer. 2010; 17(2):351-60. doi: 10.1677/ERC-09-0252. PubMed PMID: 20228137.

48. Mashhedi H, Blouin M J, Zakikhani M, David S, Zhao Y, Bazile M, et al. Metformin abolishes increased tumor (18)F-2-fluoro-2-deoxy-D-glucose uptake associated with a high energy diet. Cell cycle. 2011; 10(16):2770-8. PubMed PMID: 21811094.

49. Tam J, Duda D G, Perentes J Y, Quadri R S, Fukumura D, Jain R K. Blockade of VEGFR2 and not VEGFR1 can limit diet-induced fat tissue expansion: role of local versus bone marrow-derived endothelial cells. PloS one. 2009; 4(3):e4974. doi: 10.1371/journal.pone.0004974. PubMed PMID: 19333381; PubMed Central PMCID: PMC2659427.

50. Fukumura D, Ushiyama A, Duda D G, Xu L, Tam J, Krishna V, et al. Paracrine regulation of angiogenesis and adipocyte differentiation during in vivo adipogenesis. Circulation research. 2003; 93(9):e88-97. doi: 10.1161/01.RES.0000099243.20096.FA. PubMed PMID: 14525808; PubMed Central PMCID: PMC2755542.

51. Surwit R S, Feinglos M N, Rodin J, Sutherland A, Petro A E, Opara E C, et al. Differential effects of fat and sucrose on the development of obesity and diabetes in C57BL/6J and A/J mice. Metabolism: clinical and experimental. 1995; 44(5):645-51. PubMed PMID: 7752914.

52. Wang Y, Zhang Y, Yang J, Ni X, Liu S, Li Z, et al. Genomic sequencing of key genes in mouse pancreatic cancer cells. Current molecular medicine. 2012; 12(3):331-41. PubMed PMID: 22208613; PubMed Central PMCID: PMC3799885.

53. Bardeesy N, Aguirre A J, Chu G C, Cheng K H, Lopez L V, Hezel A F, et al. Both p16(Ink4a) and the p19(Arf)-p53 pathway constrain progression of pancreatic adenocarcinoma in the mouse. Proc Natl Acad Sci USA. 2006; 103(15):5947-52. doi: 10.1073/pnas.0601273103. PubMed PMID: 16585505; PubMed Central PMCID: PMC1458678.

54. Reagan-Shaw S, Nihal M, Ahmad N. Dose translation from animal to human studies revisited. FASEB J. 2008; 22(3):659-61. doi: 10.1096/fj.07-9574LSF. PubMed PMID: 17942826.

55. Liu J, Li M, Song B, Jia C, Zhang L, Bai X, et al. Metformin inhibits renal cell carcinoma in vitro and in vivo xenograft. Urologic oncology. 2013; 31(2):264-70. doi: 10.1016/j.urolonc.2011.01.003. PubMed PMID: 21676631.

56. Rattan R, Graham R P, Maguire J L, Giri S, Shridhar V. Metformin suppresses ovarian cancer growth and metastasis with enhancement of cisplatin cytotoxicity in vivo. Neoplasia. 2011; 13(5):483-91. PubMed PMID: 21532889; PubMed Central PMCID: PMC3084625.

57. Martin T L, Alquier T, Asakura K, Furukawa N, Preitner F, Kahn B B. Diet-induced obesity alters AMP kinase activity in hypothalamus and skeletal muscle. The Journal of biological chemistry. 2006; 281(28):18933-41. doi: 10.1074/jbc.M512831200. PubMed PMID: 16687413.

58. Montgomery M K, Hallahan N L, Brown S H, Liu M, Mitchell T W, Cooney G J, et al. Mouse strain-dependent variation in obesity and glucose homeostasis in response to high-fat feeding. Diabetologia. 2013; 56(5):1129-39. doi: 10.1007/s00125-013-2846-8. PubMed PMID: 23423668.

59. Moore T, Beltran L, Carbajal S, Strom S, Traag J, Hursting S D, et al. Dietary energy balance modulates signaling through the Akt/mammalian target of rapamycin pathways in multiple epithelial tissues. Cancer prevention research. 2008; 1(1):65-76. doi: 10.1158/1940-6207.CAPR-08-0022. PubMed PMID: 19138937.

60. Metlakunta A S, Sahu M, Sahu A. Hypothalamic phosphatidylinositol 3-kinase pathway of leptin signaling is impaired during the development of diet-induced obesity in FVB/N mice. Endocrinology. 2008; 149(3):1121-8. doi: 10.1210/en.2007-1307. PubMed PMID: 18048492; PubMed Central PMCID: PMC2275357.

61. Xu Z H, Vonlaufen A, Phillips P A, Fiala-Beer E, Zhang X G, Yang L, et al. Role of Pancreatic Stellate Cells in Pancreatic Cancer Metastasis. Am J Pathol. 2010; 177(5):2585-96. doi: 10.2353/ajpath.2010.090899. PubMed PMID: WOS:000284182900043.

62. Mace T A, Ameen Z, Collins A, Wojcik S, Mair M, Young G S, et al. Pancreatic cancer-associated stellate cells promote differentiation of myeloid-derived suppressor cells in a STAT3-dependent manner Cancer Res. 2013; 73(10):3007-18. doi: 10.1158/0008-5472.CAN-12-4601. PubMed PMID: 23514705; PubMed Central PMCID: PMC3785672.

63. Pandol S, Edderkaoui M, Gukovsky I, Lugea A, Gukovskaya A. Desmoplasia of pancreatic ductal adenocarcinoma. Clinical gastroenterology and hepatology : the official clinical practice journal of the American Gastroenterological Association. 2009; 7(11 Suppl):544-7. doi: 10.1016/j.cgh.2009.07.039. PubMed PMID: 19896098.

64. Inman K S, Francis A A, Murray N R. Complex role for the immune system in initiation and progression of pancreatic cancer. World journal of gastroenterology : WJG. 2014; 20(32):11160-81. doi: 10.3748/wjg.v20.i32.11160. PubMed PMID: 25170202; PubMed Central PMCID: PMC4145756.

65. Chen S J, Zhang Q B, Zeng L J, Lian G D, Li J J, Qian C C, et al. Distribution and clinical significance of tumour-associated macrophages in pancreatic ductal adenocarcinoma: a retrospective analysis in China. Current oncology. 2015; 22(1):e11-9. doi: 10.3747/co.22.2150. PubMed PMID: 25684992; PubMed Central PMCID: PMC4324348.

66. Liu J, Liu L, Cui Y, Zhang J, Jiang H. p38 MAPK regulates Th2 cytokines release in PBMCs in allergic rhinitis rats. Journal of Huazhong University of Science and Technology Medical sciences=Hua zhong ke ji da xue xue bao Yi xue Ying De wen ban=Huazhong keji daxue xuebao Yixue Yingdewen ban. 2010; 30(2):222-5. doi: 10.1007/s11596-010-0218-x. PubMed PMID: 20407878.

67. Parhar K, Ray A, Steinbrecher U, Nelson C, Salh B. The p38 mitogen-activated protein kinase regulates interleukin- lbeta-induced IL-8 expression via an effect on the IL-8 promoter in intestinal epithelial cells. Immunology. 2003; 108(4):502-12. PubMed PMID: 12667212; PubMed Central PMCID: PMC1782920.

68. Perdiguero E, Sousa-Victor P, Ruiz-Bonilla V, Jardi M, Caelles C, Serrano A L, et al. p38/MKP-1-regulated AKT coordinates macrophage transitions and resolution of inflammation during tissue repair. The Journal of cell biology. 2011; 195(2):307-22. doi: 10.1083/jcb.201104053. PubMed PMID: 21987635; PubMed Central PMCID: PMC3198158.

69. Yu J H, Kim H. Oxidative stress and cytokines in the pathogenesis of pancreatic cancer. Journal of cancer prevention. 2014; 19(2):97-102. doi: 10.15430/JCP.2014.19.2.97. PubMed PMID: 25337577; PubMed Central PMCID: PMC4204162.

70. Li H, Lee J, He C, Zou M H, Xie Z. Suppression of the mTORC1/STAT3/Notch1 pathway by activated AMPK prevents hepatic insulin resistance induced by excess amino acids. American journal of physiology Endocrinology and metabolism. 2014; 306(2):E197-209. doi: 10.1152/ajpendo.00202.2013. PubMed PMID: 24302004; PubMed Central PMCID: PMC3920006.

71. Ramos-Nino M E. The role of chronic inflammation in obesity-associated cancers. ISRN oncology. 2013; 2013: 697521. doi: 10.1155/2013/697521. PubMed PMID: 23819063; PubMed Central PMCID: PMC3683483.

72. Cirri P, Chiarugi P. Cancer associated fibroblasts: the dark side of the coin. American journal of cancer research. 2011; 1(4):482-97. PubMed PMID: 21984967; PubMed Central PMCID: PMC3186047.

73. Feig C, Gopinathan A, Neesse A, Chan D S, Cook N, Tuveson D A. The pancreas cancer microenvironment. Clin Cancer Res. 2012; 18(16):4266-76. doi: 10.1158/1078-0432.CCR-11-3114. PubMed PMID: 22896693; PubMed Central PMCID: PMC3442232.

74. Jain R K. Normalizing tumor microenvironment to treat cancer: bench to bedside to biomarkers. Journal of Clinical Oncology. 2013; 31(17):2205-18.

75. Gilkes D M, Semenza G L, Wirtz D. Hypoxia and the extracellular matrix: drivers of tumour metastasis. Nat Rev Cancer. 2014; 14(6):430-9. doi: 10.1038/nrc3726. PubMed PMID: 24827502.

76. Chauhan V P, Jain R K. Strategies for advancing cancer nanomedicine. Nature Materials. 2013; 12(11):958-62. doi: 10.1038/nmat3792.

77. Rider P, Carmi Y, Guttman O, Braiman A, Cohen I, Voronov E, et al. IL-1alpha and IL-1beta recruit different myeloid cells and promote different stages of sterile inflammation. Journal of immunology. 2011; 187(9):4835-43. doi: 10.4049/jimmunol 1102048. PubMed PMID: 21930960.

78. Phillips P. Pancreatic stellate cells and fibrosis. In: Grippo P J, Munshi H G, editors. Pancreatic Cancer and Tumor Microenvironment. Trivandrum (India)2012.

79. Ijichi H. Inhibition of CXCLs/CXCR2 axis in the tumor microenvironment might be a potent therapeutics for pancreatic cancer. Oncoimmunology. 2012; 1(4):569-71. PubMed PMID: 22754790; PubMed Central PMCID: PMC3382906.

80. Erez N, Truitt M, Olson P, Arron S T, Hanahan D. Cancer-Associated Fibroblasts Are Activated in Incipient Neoplasia to Orchestrate Tumor-Promoting Inflammation in an NF-kappaB-Dependent Manner. Cancer Cell. 2010; 17(2):135-47. doi: 10.1016/j.ccr.2009.12.041. PubMed PMID: 20138012.

81. Hirsch H A, Iliopoulos D, Struhl K. Metformin inhibits the inflammatory response associated with cellular transformation and cancer stem cell growth. Proceedings of the National Academy of Sciences of the United States of America. 2013; 110(3):972-7. doi: 10.1073/pnas.1221055110. PubMed PMID: 23277563; PubMed Central PMCID: PMC3549132.

82. Mathsyaraja H, Thies K, Taffany D A, Deighan C, Liu T, Yu L, et al. CSF1-ETS2-induced microRNA in myeloid cells promote metastatic tumor growth. Oncogene. 2015; 34(28):3651-61. doi: 10.1038/onc.2014.294. PubMed PMID: 25241894; PubMed Central PMCID: PMC4369473.

83. Wu Y, Li Y Y, Matsushima K, Baba T, Mukaida N. CCL3-CCRS axis regulates intratumoral accumulation of leukocytes and fibroblasts and promotes angiogenesis in murine lung metastasis process. Journal of immunology. 2008; 181(9):6384-93. PubMed PMID: 18941229.

84. Gutschalk C M, Yanamandra A K, Linde N, Meides A, Depner S, Mueller M M. GM-CSF enhances tumor invasion by elevated MMP-2, -9, and -26 expression. Cancer medicine. 2013; 2(2):117-29. doi: 10.1002/cam4.20. PubMed PMID: 23634280; PubMed Central PMCID: PMC3639651.

85. Sica A, Mantovani A. Macrophage plasticity and polarization: in vivo veritas. J Clin Invest. 2012; 122(3):787-95. doi: 10.1172/JCI59643. PubMed PMID: 22378047; PubMed Central PMCID: PMC3287223.

86. Liu C Y, Xu J Y, Shi X Y, Huang W, Ruan T Y, Xie P, et al. M2-polarized tumor-associated macrophages promoted epithelial-mesenchymal transition in pancreatic cancer cells, partially through TLR4/IL-10 signaling pathway. Lab Invest. 2013; 93(7):844-54. doi: 10.1038/labinvest.2013.69. PubMed PMID: 23752129.

87. McCarroll J A, Naim S, Sharbeen G, Russia N, Lee J, Kavallaris M, et al. Role of pancreatic stellate cells in chemoresistance in pancreatic cancer. Frontiers in physiology. 2014; 5:141. doi: 10.3389/fphys.2014.00141. PubMed PMID: 24782785; PubMed Central PMCID: PMC3988387.

88. Cifarelli V, Lashinger L M, Devlin K L, Dunlap S M, Huang J, Kaaks R, et al. Metformin and Rapamycin Reduce Pancreatic Cancer Growth in Obese Prediabetic Mice by Distinct MicroRNA-Regulated Mechanisms. Diabetes. 2015. doi: 10.2337/db14-1132. PubMed PMID: 25576058.

89. Cufi S, Vazquez-Martin A, Oliveras-Ferraros C, Martin-Castillo B, Joven J, Menendez J A. Metformin against TGFbeta-induced epithelial-to-mesenchymal transition (EMT): from cancer stem cells to aging-associated fibrosis. Cell cycle. 2010; 9(22):4461-8. PubMed PMID: 21088486.

90. Furstenberger G, Senn H J. Insulin-like growth factors and cancer. The Lancet Oncology. 2002; 3(5):298-302. PubMed PMID: 12067807.

91. Goodwin P J, Stambolic V. Impact of the obesity epidemic on cancer. Annual review of medicine. 2015; 66:281-96. doi: 10.1146/annurev-med-051613-012328. PubMed PMID: 25423596.

92. Joost H G. Diabetes and cancer: epidemiology and potential mechanisms. Diabetes & vascular disease research. 2014; 11(6):390-4. doi: 10.1177/1479164114550813. PubMed PMID: 25268021.

93. Franco C C, Miranda R A, de Oliveira J C, Barella L F, Agostinho A R, Prates K V, et al. Protective effect of metformin against walker 256 tumor growth is not dependent on metabolism improvement. Cellular physiology and biochemistry : international journal of experimental cellular physiology, biochemistry, and pharmacology. 2014; 34(6):1920-32. doi: 10.1159/000366390. PubMed PMID: 25500480.

94. Yang S, Wang X, Contino G, Liesa M, Sahin E, Ying H, et al. Pancreatic cancers require autophagy for tumor growth. Genes & development. 2011; 25(7):717-29. doi: 10.1101/gad.2016111. PubMed PMID: 21406549; PubMed Central PMCID: PMC3070934.

95. Shi W Y, Xiao D, Wang L, Dong L H, Yan Z X, Shen Z X, et al. Therapeutic metformin/AMPK activation blocked lymphoma cell growth via inhibition of mTOR pathway and induction of autophagy. Cell death & disease. 2012; 3:e275. doi: 10.1038/cddis.2012.13. PubMed PMID: 22378068; PubMed Central PMCID: PMC3317343.

96. Roongruedee Chaiteerakij D B Z, Patrick A. Burch, Kari G. Chaffee, William R., Bamlet A L O, Lewis R. Roberts, Gloria M. Petersen. Metformin Use Does Not Increase Survival of Pancreatic Cancer Patients: A Cautionary Lesson. Abstract 8687]. AACR 2015; Philadelphia2015.

97. Kordes S, Pollak M N, Zwinderman A H, Mathot R A, Weterman M J, Beeker A, et al. Metformin in patients with advanced pancreatic cancer: a double-blind, randomised, placebo-controlled phase 2 trial. The Lancet Oncology. 2015. doi: 10.1016/S1470-2045(15)00027-3. PubMed PMID: 26067687.

### Example 2

### Obesity-Induced Inflammation and Desmoplasia Promote Pancreatic Cancer Progression and Resistance to Chemotherapy

Pancreatic cancer is the fourth-leading cause of cancer-associated death worldwide (1), with an overall five-year rate survival of 7% (2). The risk for pancreatic cancer is about 50% greater for individuals with obesity (body mass index>30), those with increased abdominal adiposity (3). Excess body weight also worsens the already dismal outcome of pancreatic ductal adenocarcinoma (PDAC) patients by increasing the relative risk of cancer mortality by more than 2-fold (McWilliams, R. R., et al. Cancer 116, 5054-5062 (2010); Li, D., et al. JAMA: the journal of the American Medical Association 301, 2553-2562 (2009); Bracci, P. M. Mol Carcinog 51, 53-63 (2012); and Smits, M. M. & van Geenen, E. J. Nature reviews. Gastroenterology & hepatology 8, 169-177 (2011)). As a consequence of the obesity pandemic, with nearly 70% of the United States adult population being either overweight or obese (Ogden, C. L., et al. JAMA: the journal of the American Medical Association 311, 806-814 (2014)), the majority of PDAC patients have excess weight at diagnosis. Understanding why obesity confers worse prognosis might lead to novel treatments and enhance the outcome of current therapies.

PDAC is a highly desmoplastic cancer characterized by an excessive extracellular matrix and activated PSCs (12, 13). Extracellular matrix components and PSCs promote direct prosurvival and pro-migratory signals to cancer cells (14-16). In addition, desmoplasia increases solid stress and stiffness (17, 18), and these mechanical changes create a formidable barrier to drug delivery. Solid stress compresses blood vessels and causes heterogeneous tumor perfusion, which results in poorer treatment outcomes (14, 19-22). Importantly, obesity itself is a pro-desmoplastic condition. In fact, the hypoxia that results from abnormal blood vessels and decreased blood flow due to the rapidly expanding adipose tissue in obesity causes adipocyte dysfunction and immune cell recruitment (23-25). The latter leads to cytokine production, inflammation, and ultimately fibrosis (23-25). In particular, obesity significantly enhances angiotensin II type-1 receptor (AT1) signaling in adipose tissues , a major pro-fibrotic pathway that becomes activated in a pro-inflammatory environment (26, 27). Obesity also leads to accumulation of fat in the normal pancreas (steatosis), which generates a similar inflammatory process with increased expression of cytokines, extracellular matrix remodeling, and fibrosis (9, 28-30). Importantly, PDACs in obese mice and patients also have an increased adipocyte content (31, 32). Of clinical relevance, the interaction of cancer cells with adipocytes—both in the form of accumulation of fat in pancreas and when cancer cells at the expanding edge of the tumor invade the local adipose tissue—is associated with worse outcomes in PDAC patients (9, 33). However, the role of adipocytes during obesity-induced PDAC progression remains unclear.

The experiments in this example test whether obesity-associated fatty infiltration in PDACs generates a proinflammatory and pro-fibrotic microenvironment in tumors, which promotes growth and progression, and hinders the delivery and efficacy of chemotherapy in human samples as well as in clinically relevant orthotopic and genetically engineered mouse models (GEMMs) of PDAC. To reduce the obesity-instigated desmoplastic reaction in PDAC, AT1 knockout (Agtr1a−/−) mice and a clinically-approved AT1 inhibitor (AT1 blocker, ARB) were used, which was previously found to reduce PSC activation, matrix expression, and solid stress in PDAC (21). In addition, it was determined whether the obesity exacerbated desmoplasia in PDAC results from increased inflammation, and we uncovered the cellular and molecular mechanisms involved.

### Materials and Methods

### Animal Experiments

Wild-type (WT) C57BL/6 and FVB male mice were originally obtained from Jackson Laboratory (The Jackson Laboratory, Bar Harbor, Me.) and bred and maintained in our defined-flora colony. KPC (Ptfl-Cre/LSL-KRAS^{G12D}/p53-R172H) and iKRAS (p48-Cre;R26-rtTa-IRES-EGFP;TetO-Kras^{G12D} mice) mice were obtained. To generate an obese model, mice (6-week old) were given either 10% or a 60% fat diet (D12450J and D12492, Research Diets, New Brunswick, N.J.) for 10 weeks (or until tumor collection in spontaneous models), as previously described (Surwit, R. S., et al. Metabolism: clinical and experimental 44, 645-651 (1995)). For implanted tumor experiments, AK4.4 cells (KrasG12D and p53 [symbol] /−) were isolated from mice generating spontaneous pancreatic tumors (Ptf1-Cre/LSL-Kras^{G12D}/p53Lox/ [symbol] ) (Bardeesy, N., et al. Proc Natl Acad SciUSA103, 5947-5952 (2006)). Orthotopic pancreatic tumors were generated by implanting a small piece (1 mm³) of viable tumor tissue (from a source tumor in a separate animal) into the pancreas of a 6-8 week-old male lean or obese FVB (AK4.4 model) or C57BL/6 (PAN02 model) mouse. PAN02 tumor chunks and AK4.4 cells were authenticated by IDEXX laboratories. (PAN02: IDEXX RADIL Case # 22366-2013. AK4.4: IDEXX RADIL Case #27818-2014). For AT1- and AT2-knockout studies, tumors were implanted into the pancreas of a 6-8-week-old male lean or obese C57BL/ 6, agtr1 a−/− or agtr2−/− mice. With exception of PAN02, these orthotopically grown pancreatic cancers are characterized by a dense collagenous stroma, a hallmark of pancreatic cancer desmoplasia (Diop-Frimpong, B., et al. Proc Natl Acad Sci USA 108, 2909-2914 (2011)).

### Human Samples

Human samples of pancreatic cancer were obtained from the MGH tissue repository under an active IRB protocol (Partners Healthcare IRB approval number: 2013P001969). Written informed consent from the donor or the next of kin was obtained for the use of these samples in research. Tumors selected received no prior chemotherapy or radiation therapy before the surgical specimen was collected at the time of tumor resection. Body mass index (BMI) was obtained for the respective sample. A total of 16 samples were randomly selected from this subset of samples (8 with BMI<25 and 8 with BMI>30). Paraffin sections were stained for collagen-I and HA as described below. Images are acquired using confocal microscopy and quantified using Matlab. Data were analyzed anonymously.

### Pancreatic Tumor Growth Studies

For all experiments unless specified below, mice bearing orthotropic PAN02 or AK4.4 pancreatic tumors were randomized into treatment groups (or no treatment) and tumors were collected at day 21 after implantation. For tumor growth study with chemotherapy, mice were divided into treatment groups 7 days after implantation, and treated with either 5-FU (30 mg/Kg i.v. every 4 days) or an equal volume of saline by intravenous injection on days 7, 11 and 15 after implantation, with tumors collected at day 19. For tumor growth study with losartan, mice were treated with losartan (90 mg/Kg i.p. every day) or an equal volume of PBS intraperitoneally starting on day 5 after implantation and for the duration of the study. In the combined experiment of losartan and 5-FU, the same protocol was used for each drug as described above. TAN depletion by a Ly6G specific inhibitor (BioExcell, 4 mg/Kg i.p. every 2 days) was administered to PAN02 bearing animals starting at day 1 or day 7, and to AK4.4 bearing animals at day 7. IL-1β inhibition (MM425B, Endogen/Pierce Biotechnology, 2 mg/Kg i.p. every 2 days) was administered to PAN02 bearing animals starting at day 7. At the completion of the study, adipose tissue and tumor samples were collected, weighed, and processed for further analysis. When spontaneous models were used, tumors were collected when palpable.

### Drug Preparation

Angiotensin inhibitor losartan was obtained as pills, crushed and dissolved in PBS over 24 h. The solution was then sterile filtered for injection. Doxorubicin and 5-FU were obtained as solutions for injection. All drugs were purchased from the pharmacy at Massachusetts General

### Hospital.

### Analysis of Desmoplasia

To assess obesity-induced ECM remodeling and fibrosis fibrillar collagen accumulation was quantified using second harmonic generation imaging, and collagen-I content, hyaluronan, MMPs levels and fibrosis related signaling pathways (e.g. AT-I, CTGF, TSP1, TGFβ1, and P38/ERK signaling) were measured by rtPCR array, immunohistochemistry and immunoblotting. The topographical distribution and density of cancer-associated fibroblasts (CAFs) in tumor sections was detected by staining for αSMA activated fibroblast marker. Vessel morphology and perfusion was assessed using standard histological analyses.

### Drug Delivery

Mice bearing orthotopic PAN02 were injected with 30 mg/kg of 5-FU 3 weeks after tumor implantation, administered retro-orbitally 30 min prior to tumor removal. The tissue was dabbed of excess blood and then snap-frozen in liquid nitrogen for analysis. 5-FU was isolated from the tissues and measured using liquid—liquid extraction followed by reverse-phase HPLC with tandem mass-spectrometry.

### Gene Expression

Immediately following excision, tumor tissue was snap frozen and stored in liquid nitrogen. Total RNA was extracted and relative gene expression was determined using RT2 Profiler PCR Arrays system (Qiagen) on a Stratagene Mx3000P QPCR System. The pre-made pathway-focused array used (mouse genes) was “Fibrosis” (Cat. Number: PAMM011Z).

### Protein Expression

Western Blot Analysis

Each tumor sample was homogenized directly in lysis buffer for protein extraction. 30 ug of denatured protein per sample was loaded on 7%, 10% and 12% SDS-polyacrylamide gels. Antibodies used: phospho-AKT^{Ser473} and AKT; phospho-p38 MAPKT^{180/Y182} and p38; phospho-pS6^{Ser235/236} and S6; phospho-ERK(p44/42 MAPK)^{T202/Y204} and ERK; MMP-9; phospho-4EBP1^{Thr37/46} and 4EBP1; phospho-JNK (SAPK/JNK)^{Thr183/Tyr185} and JNK; Phospho-NF-κB p65^{Ser536}; TGF-β; ATR1; Smad2; E-Cadherin; vimentin; snail; αSMA; col-1; GAPDH and β-actin. All Antibodies listed above were obtained from Cell Signaling Technology (Beverly, Mass.), and diluted 1:1000 with exception of phospho-JNK (SAPK/JNK)^{Thr183/Tyr185} (1:500) and Phospho-NF-κB p65^{Ser536} (1:500). Other antibodies used were: for αSMA (1:1000, abeam, MA); col-1 (1:1000); MMP-9 (1:500, EMD Millipore-Billerica, Mass.), GAPDH (1:2000, Ambion, N.Y.), β-actin (1:5000, Sigma, Mo.), α-tubilin (1:5000, Sigma, Mo.).

### Multiplex Array

Each tumor sample was homogenized directly in lysis buffer for protein extraction. 2 ug/ul of sample was used. The pre-made inflammatory multiple cytokines protein array was used (V-PLEX Proinflammatory Panell mouse kit, Cat. Number K15048D).

### Immunohistochemistry/Immunofluorescence

Vessel Perfusion and Hypoxia Histology

On the day of the last treatment, mice were slowly (B2 min) injected with 100 μl of 1 mg/ml biotinylated lectin (Vector Labs), administered via the retro-orbital sinus 5 min before tumour removal. For hypoxia studies, the mice were also injected with 60 mg/kg i.p. of pimonidazole at 10 mg/ml 45′ before tumour removal. The tumours were then excised and frozen in optimal cutting temperature compound (Tissue-Tek). Transverse tumour sections, 40 mm thick, were immuno-stained with antibodies to endothelial marker CD31 (MEC13.3 antibody, Bio-sciences (BD), 1:100 dilution) and counterstained with 40,6-diamidino-2-pheny- lindole (Vector Labs). Collagen-I and hyaluronan were detected using the LF-68 antibody (1:50 dilution) provided by Dr Larry Fisher (NIDCR) and a biotinylated hyaluronan proteoglycan fragment (385911, Calbiochem), respectively. Staining for αSMA (C6198 antibody, Sigma, 1:100 dilution), active TGF-b1 (G122A antibody, Promega, 1:15 dilution), CCN2 (TP-243 antibody, Toney Pines, 1:100 dilution), AT1 (ab18801 antibody, Abcam, 1:100 dilution) and AT2 (AAR-012 antibody, Alomone, 1:200 dilution) were carried out in 10 mm sections. For the detection of collagen I in AK4.4, KPC and human pancreatic ductal adenocarcinoma, the paraffin-embedded sections were treated with a pH-9.0 antigen retrieval solution and counterstained with haematoxylin.

Histological Image Analysis

Whole tumor mosaics from each slide were analyzed using a confocal microscope (Olympus, 10X air objective). For vascular analysis, vessels were skeletonized and segmented using a custom, semi-automated tracing program developed in MATLAB (The MathWorks), allowing the removal of structures under 30 pixels and regions of autofluorescence. For perfusion fraction, the number of vessels counted by this program with colocalization of lectin and CD31 staining was divided by the number of vessels counted with CD31 staining. For vessel metrics, including diameter and density, the program determined the average size of all counted vessels and their length, as well as the count per area. Identical analysis settings and thresholds were used for all tumours.

### Flow Cytometry

Tumor-bearing mice were perfused through intracardiac injection of PBS and sacrificed. Pancreatic tumor tissues were harvested, minced, and digested at 37° C. for 1 h with DMEM containing collagenase type 1A (1.5 mg/mL), hyaluronidase (1.5 mg/mL), and DNase (2 mg/mL). The digestion mixtures were filtered through 70-μm cell strainers. Single-cell suspensions were incubated with rat anti-mouse CD16/CD32 mAb for blocking and then stained with fluorochrome-conjugated antibodies in cold buffer cold buffer (1% BSA, 0.1% NaN3 in PBS). 7-amino-actinomycin D (7AAD) reagent (eBioscience) was added to the stained tubes per manufacturer's instruction just before running the flow analysis. Flow cytometry data were acquired on an LSRII flow cytometer (Becton Dickinson) and were analyzed with FACSDiva software. FSC-A vs. FSC-W and SSC-A vs. SSC-W was applied to discriminate the doublet/aggregated events. The following monoclonal anti-mouse antibodies were used: CD4-FITC, CD4-PE-Cy7, CD8a-FITC, CD8a-PE, CD45-PE, CD45-PE-Cy7, CD25-APC-Cy7, CD86, CD206, LY6C, and CD11b-APC-Cy7 (BD Biosciences) and F4/80-FITC and F4/80-PE (eBioscience).

### Statistical Analysis

Statistical analyses were performed using GraphPad Prism Version 6.0f. Error bars indicate the standard error of the mean of data from replicate experiments. Significance of difference between samples within figures was confirmed using unpaired t-tests, one-way anova, or two- way anova with Bonferroni correction for multiple comparisons, depending on the experimental setting. A p<0.05 value indicates significance.

### Results

### Diet- or Genetically-Induced Obesity Promotes Pancreatic Tumor Progression

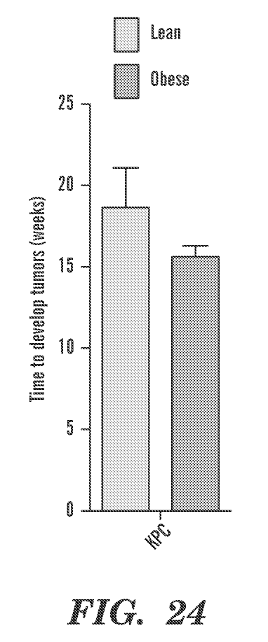

A high-fat diet was fedto four different strains of mice to generate diet-induced obesity (DIO) (FIG. 1A). In addition, a genetic model of leptin deficiency (ob/ob) was used (FIG. 1A). Consistent with previous studies (8, 31), obesity promoted tumor initiation and progression consistently across tumor models. Using spontaneous PDAC models—KPC (Ptf1-Cre/Kras^{LSL-G12D/+}/p53^{LSL-R172H/+}) and iKRAS (Ptf1-Cre/ROSA26-LSL-rtTa-IRES-eGFP/TetO-Kras^{TetO-LSL-G12D}/p53^{L/+}) mice (36-42), it was found that obese animals tended to develop tumors earlier than lean mice (FIG. 1B and FIG. 24). Furthermore, DIO and genetically-induced obesity accelerated the growth of implanted tumors in two orthotopic syngeneic PDAC models—PAN02 and AK4.4. Obese mice presented with increased tumor weight (FIG. 1C) as well as increased metastatic dissemination to the mesenteric peritoneum (PAN02) (FIG. 1D and FIG. 1E) or local infiltration of the retroperitoneum (AK4.4; mesenteric/peritoneal metastases are not detected in this model) (FIG. 1F) as compared to their lean counterparts. Taken together, these mouse models confirm the tumor-promoting effect of obesity on pancreatic cancer.

### Obesity Induces a Steatotic and Fibrotic Microenvironment in PDACs

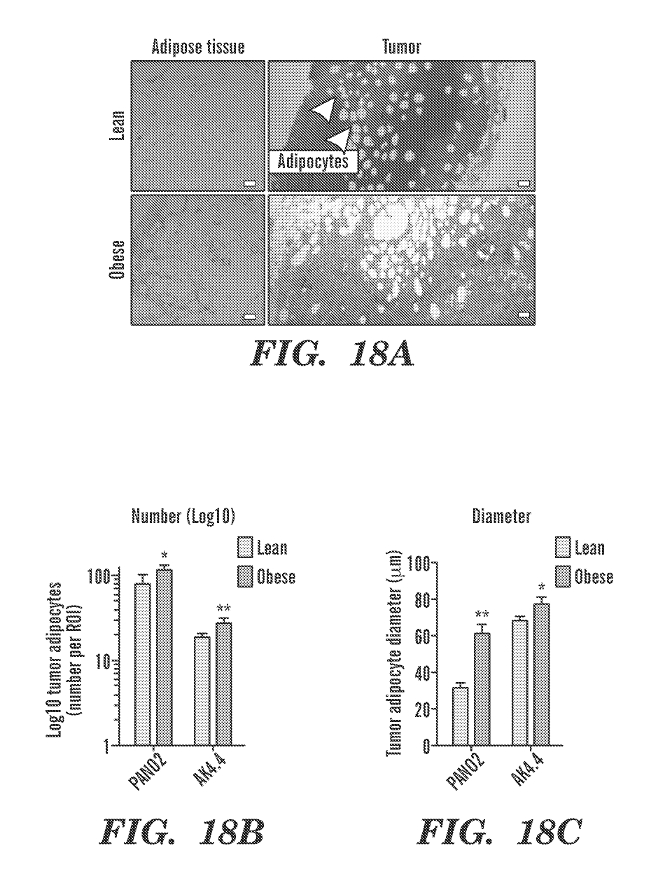

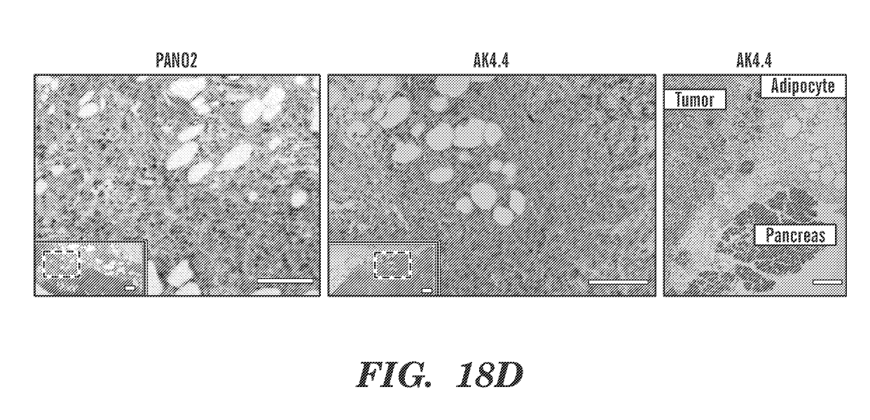

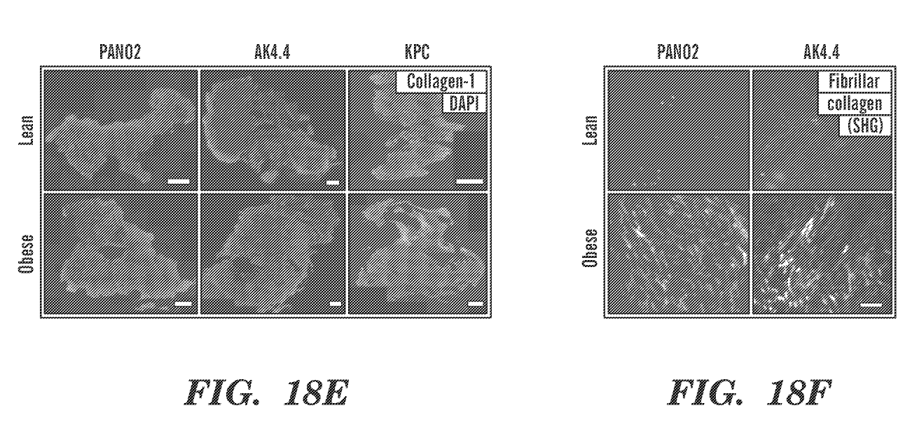

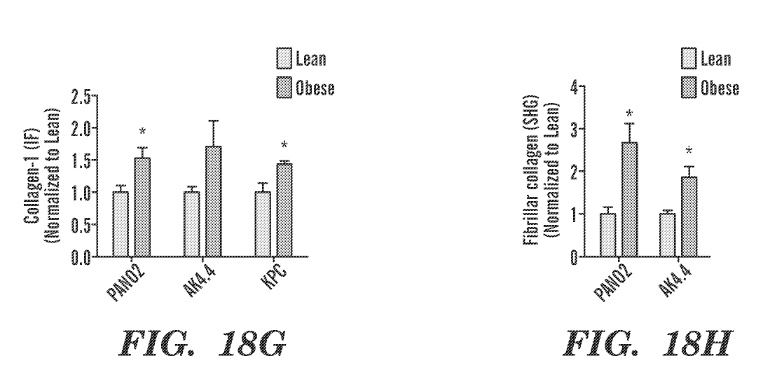

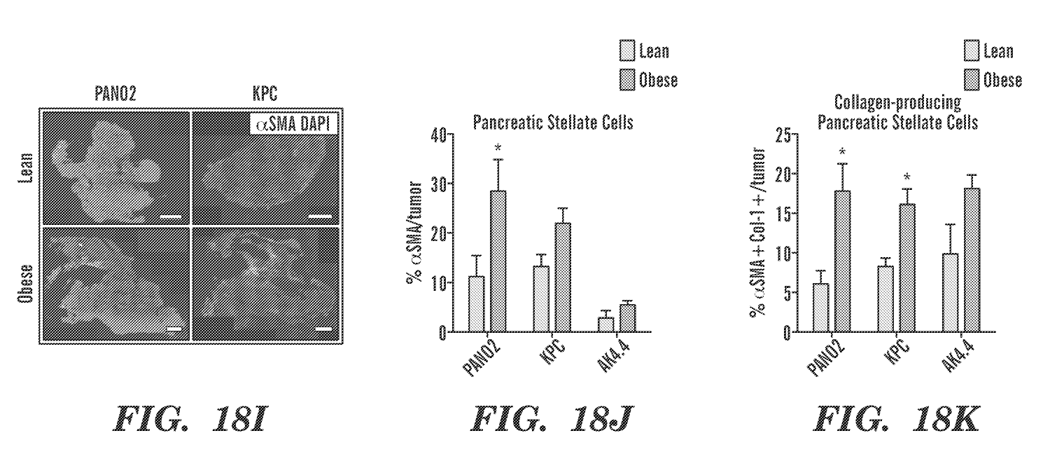

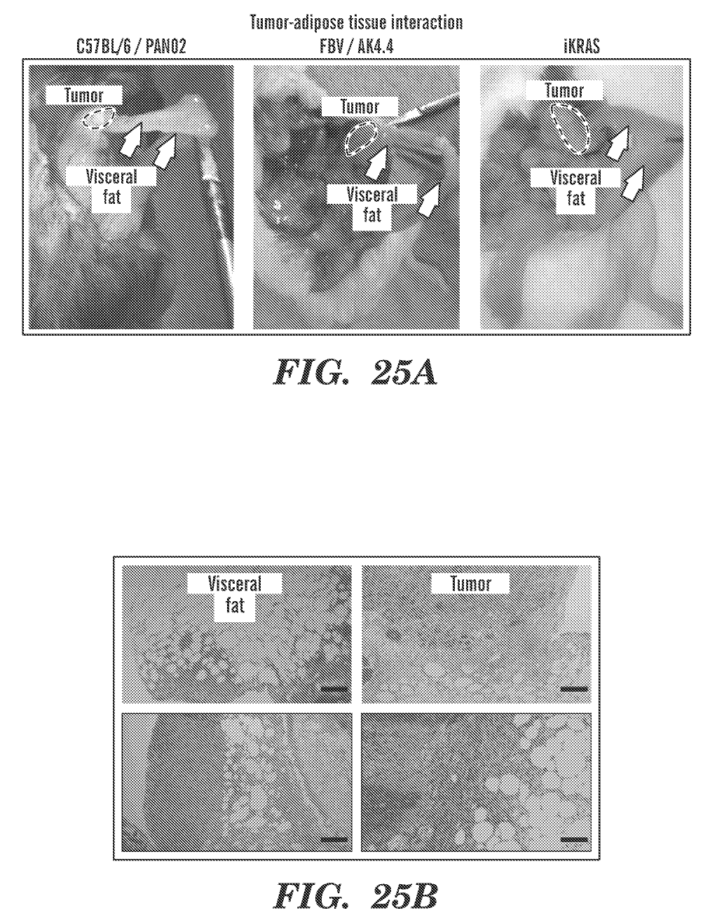

The dysfunctional hypertrophic adipocytes that accumulate in adipose and pancreatic tissues in obesity lead to the development of a local desmoplastic reaction characterized by fibrosis and inflammation (23-25). In tumors, desmoplasia stimulates growth and impairs response to chemotherapy via reduced vessel perfusion (21). The following experiments test whether obesity augments the desmoplasia in the pancreatic tumor microenvironment, ultimately fostering tumor progression. As expected (27), hypertrophic adipocytes and associated fibrosis were observed in the visceral adipose tissue of obese mice (FIG. 18A). Importantly, the tumor microenvironment also contained more and larger adipocytes (FIGS. 18A, 18B, and 18C). In part, this was due to tumors invading the neighboring visceral adipose tissue (FIG. 25A), as reported in pancreatic cancer patients (9, 33). Furthermore, Masson's trichrome staining revealed an abundance of fibrosis in the tumor areas enriched with adipocytes or alongside adjacent visceral adipose tissues (FIGS. 18A and 18D, FIG. 25B). These data suggest that, in obesity, PDACs adopt a fibrotic adipose microenvironment as they invade the adjacent adipose tissues.

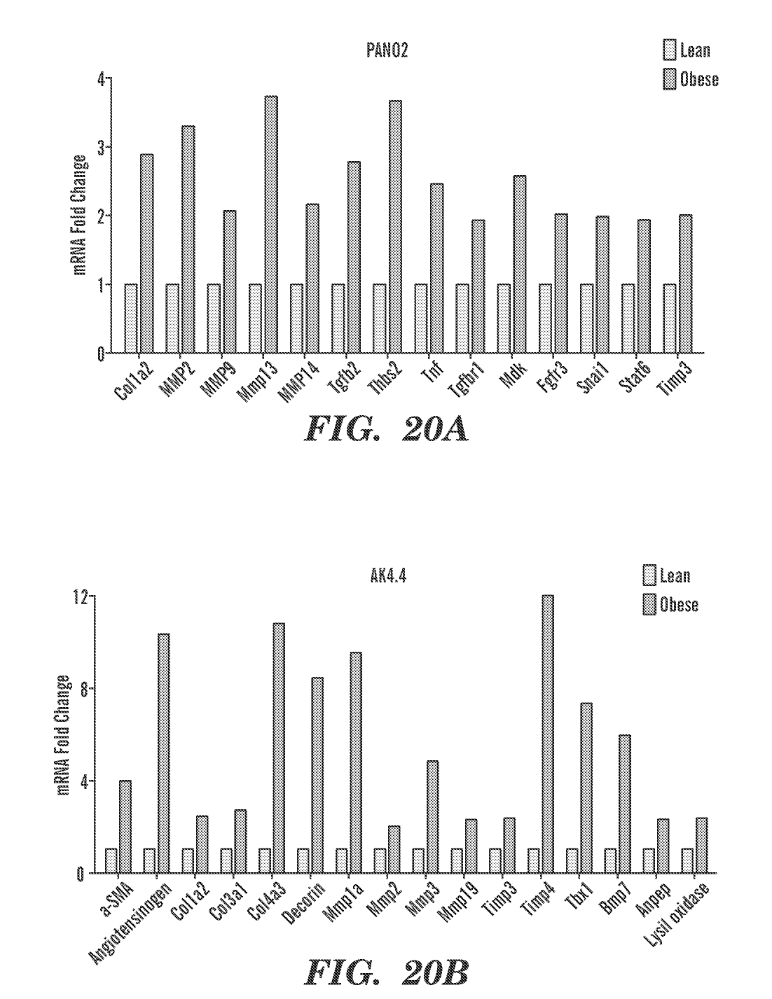

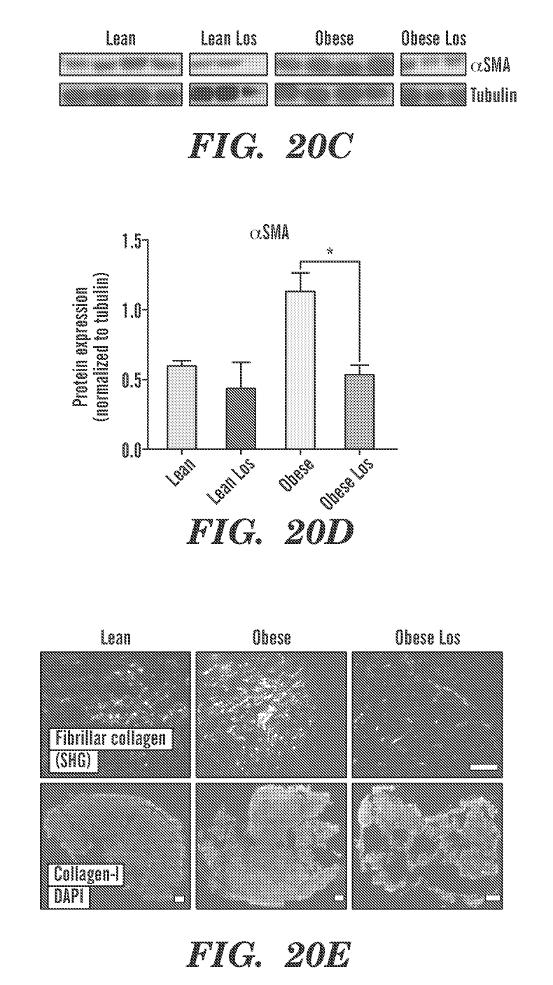

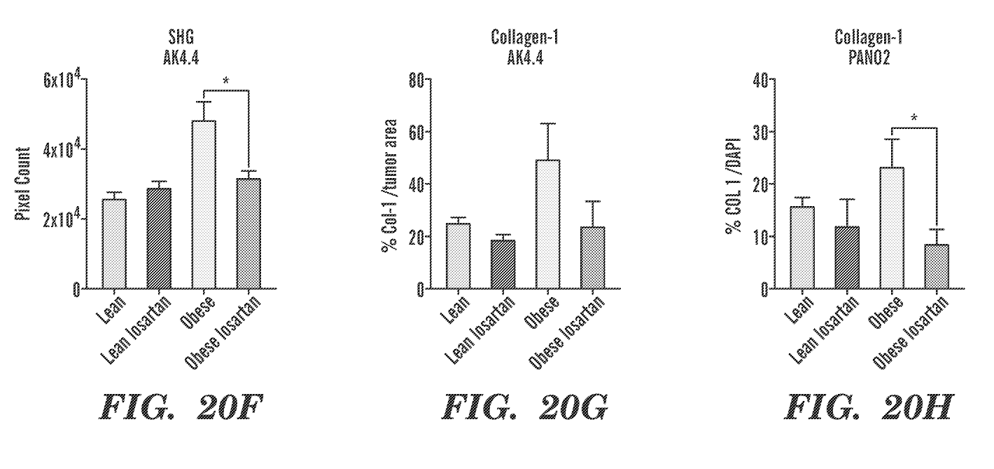

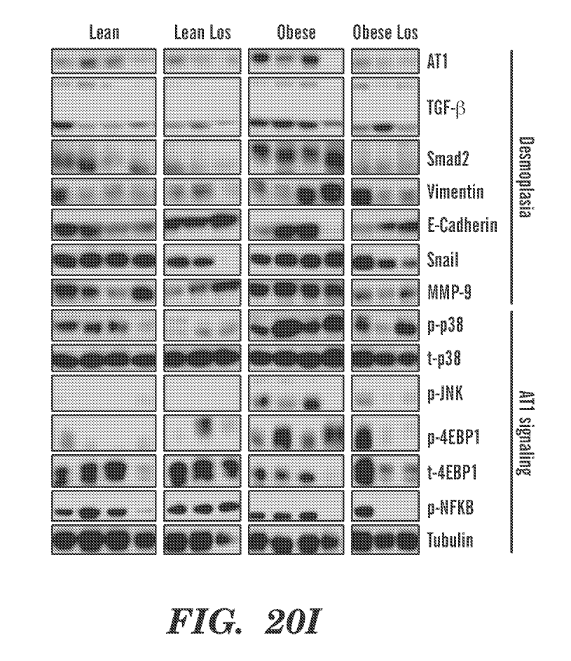

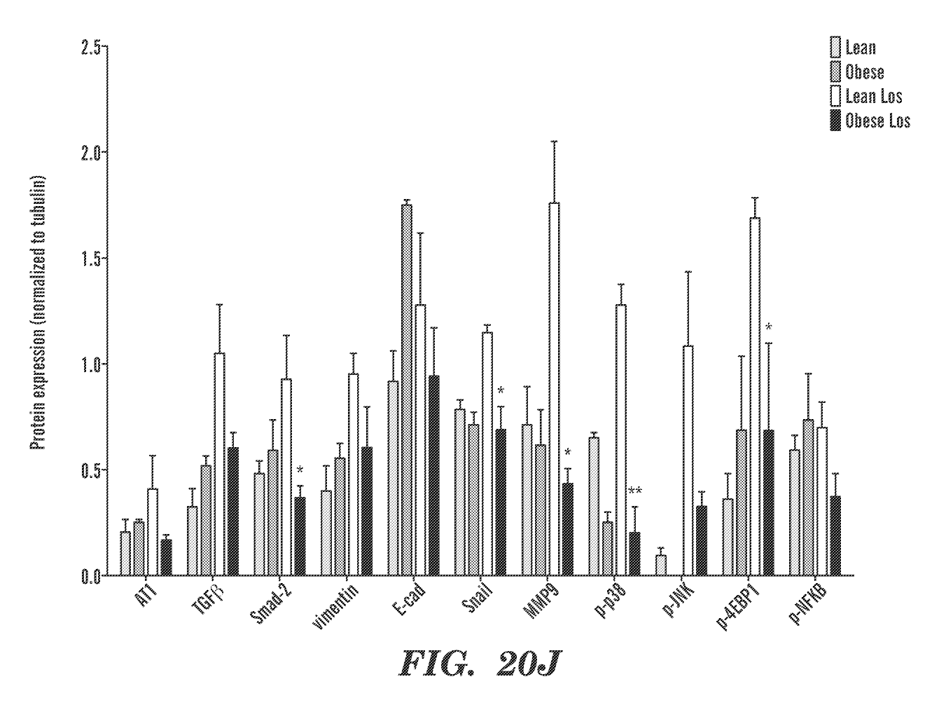

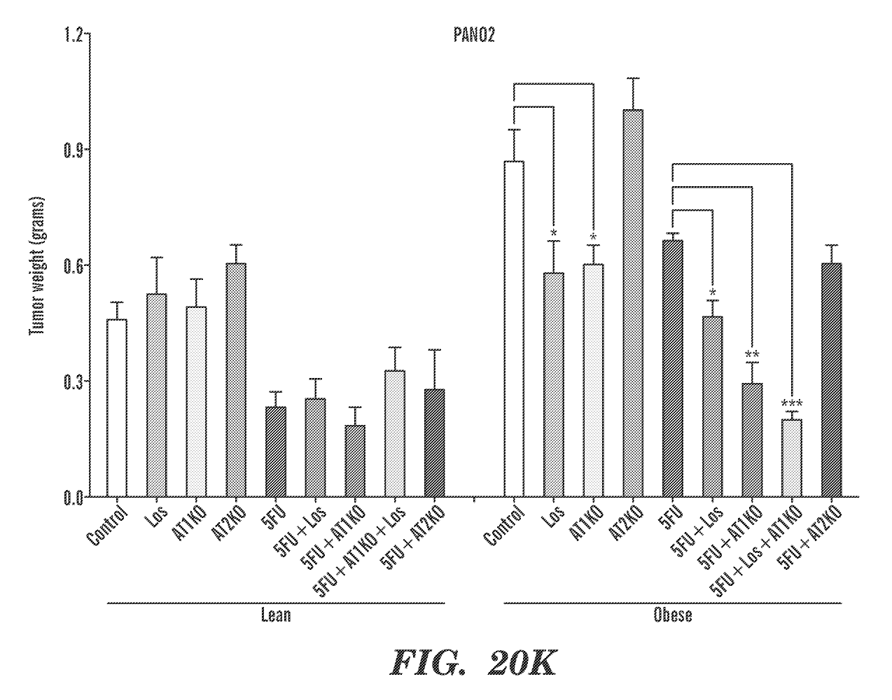

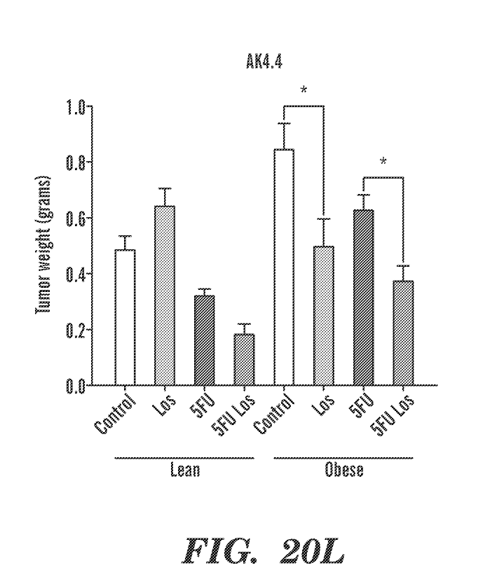

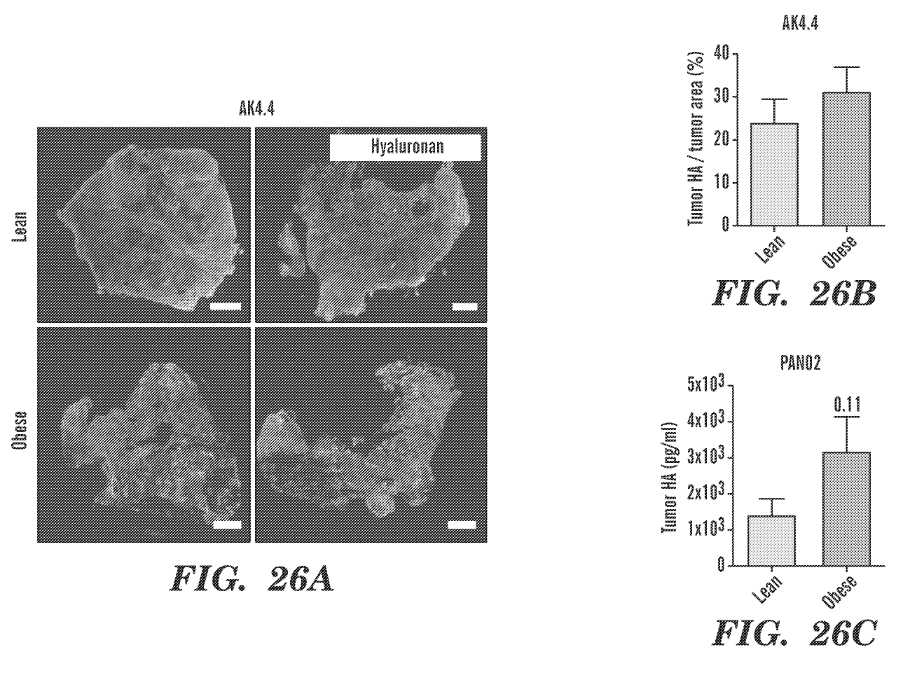

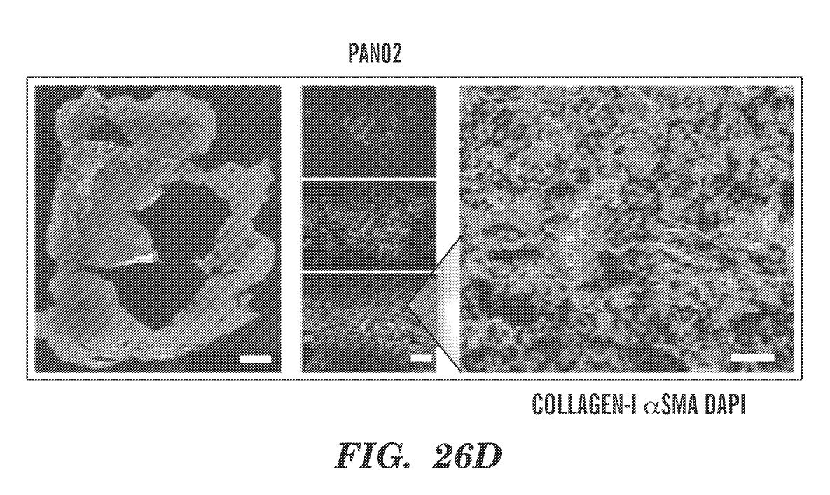

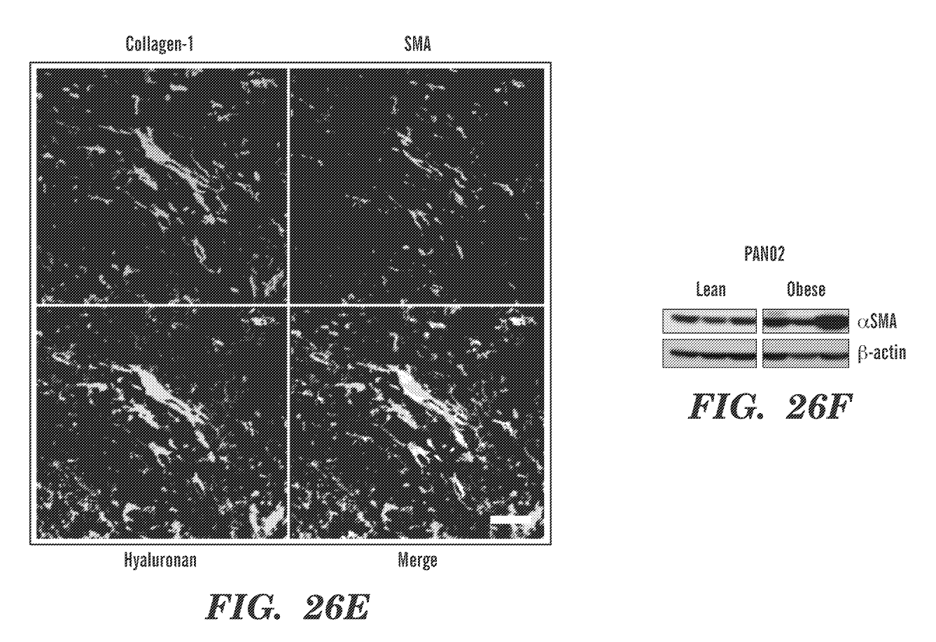

Next, it was determined whether this abundance of fibrotic adipocyte-rich areas in tumors from obese mice led to an overall increase in tumor fibrosis. Using second harmonic generation multiphoton microscopy (SHG) and immunofluorescence, obesity increased the expression of fibrillar collagen, including collagen-I, in both PAN02 and AK4.4 orthotopic PDACs as well as in the KPC PDAC model (FIGS. 18E, 18F, 18G, and 18H). A significant increase in hyaluronan (HA) levels in tumors from obese mice was not observed, although there was a similar trend (FIGS. 26A, 26B, and 26C). Similar to increased activation of stellate cells in adipose tissues and the steatotic pancreas in the obese setting, it was next determined whether the abundance of activated PSCs was also increased in tumors in obese mice. First, it was confirmed that αSMA-expressing activated PSCs indeed associate with collagen-I and HA expression in our PDAC models (FIGS. 26D and 26E). Western blotting and immunofluorescence staining revealed an increased density of activated PSCs in obese animals by almost two-fold in the AK4.4 and KPC models and three-fold in PAN02 (FIGS. 18I, 18J, 18J and FIG. 20C). As expected, the percentage of αSMA and collagen-I double positive PSCs (collagen-producing PSCs) also increased in obesity (FIGS. 18I, 18J, and 18K). Taken together, the tumors in the obese setting were enriched in enlarged adipocytes, activated PSCs, and collagen.

Obesity-Augmented Desmoplasia in PDACs Associates with Reduced Chemotherapy Efficacy

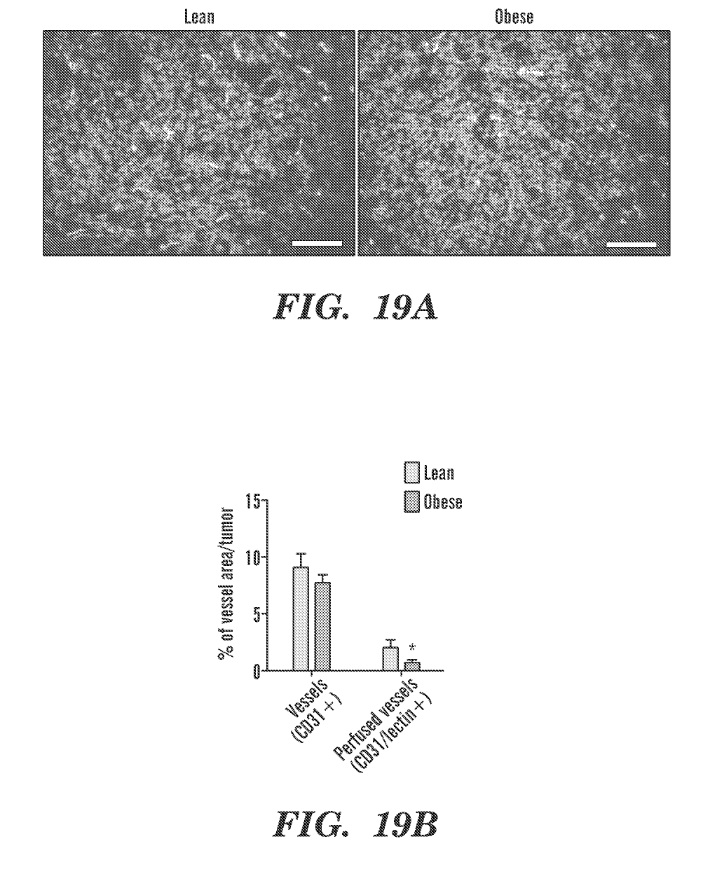

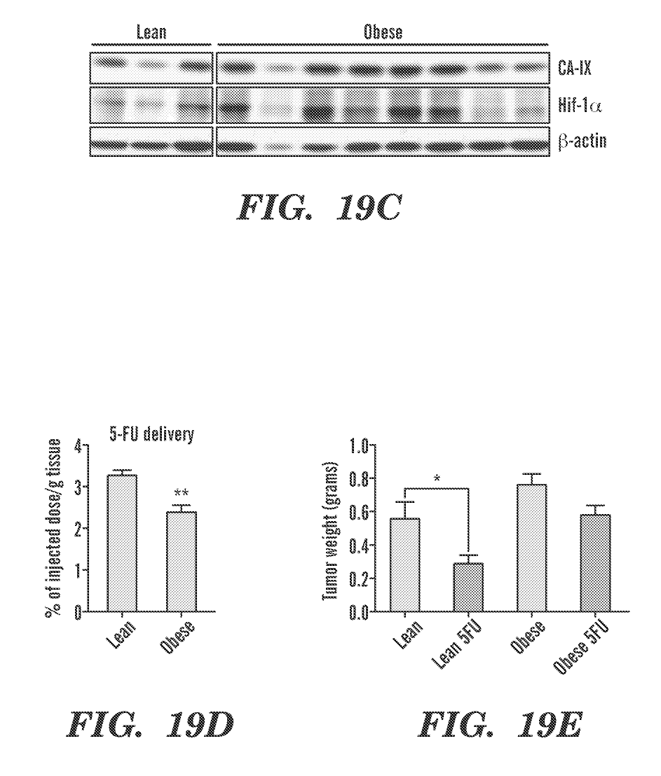

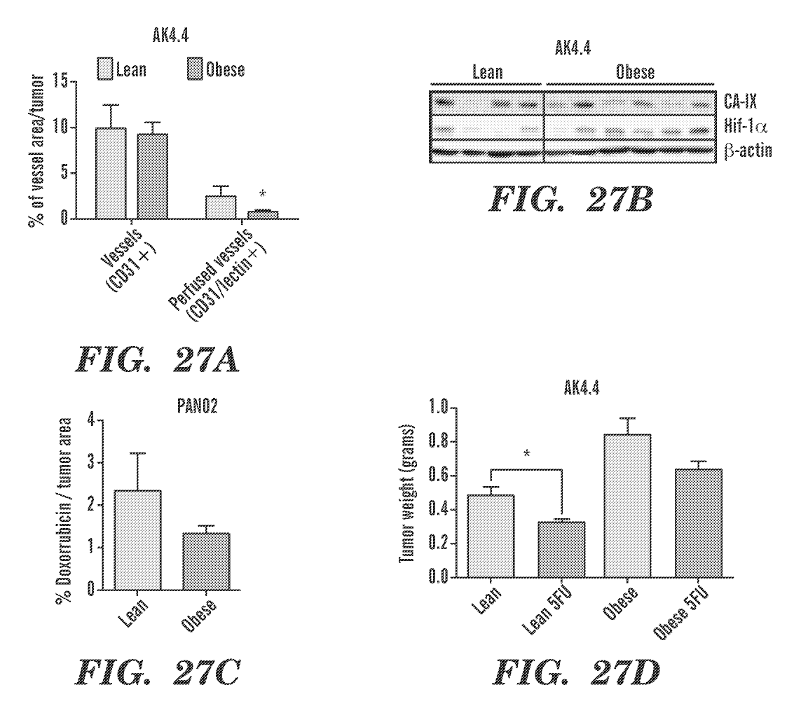

Desmoplasia reduces the delivery and efficacy of chemotherapy in PDAC by decreasing vessel perfusion (21). It was next tested whether obesity-augmented desmoplasia also affected the efficacy of chemotherapy. Indeed, tumors in obese animals had reduced perfusion and a concomitant increase in hypoxia (FIGS. 19A, 19B, 19C and FIGS. 27A and 27B) were observed. This was associated with increased stiffness and solid stress (not shown). To determine if reduced perfusion impairs delivery of chemotherapeutic agents, tumor-bearing mice were treated with 5- Fluorouracil (5-FU), an approved chemotherapeutic agent for PDAC (43). Delivery of 5-FU was decreased in tumors in obese mice (FIG. 19D). A similar trend was observed with doxorubicin (FIG. 27C). Furthermore, lean mice treated with 3 doses of 5-FU (30 mg/kg BW q4d) had a significant 50% reduction in tumor weight as compared to untreated controls in both PAN02 and AK4.4 models (FIG. 19E and FIG. 27D). In contrast, 5-FU did not significantly reduce tumor weight significantly under obese conditions (FIG. 19E and FIG. 27D). Collectively, these data shows that, in addition to directly promoting PDAC growth and metastasis, obesity reduces the response to chemotherapy.

### Blockade of AT1 Signaling Reduces Obesity-Induced Desmoplasia and Accelerated Tumor Progression, and Increases Response to Chemotherapy.

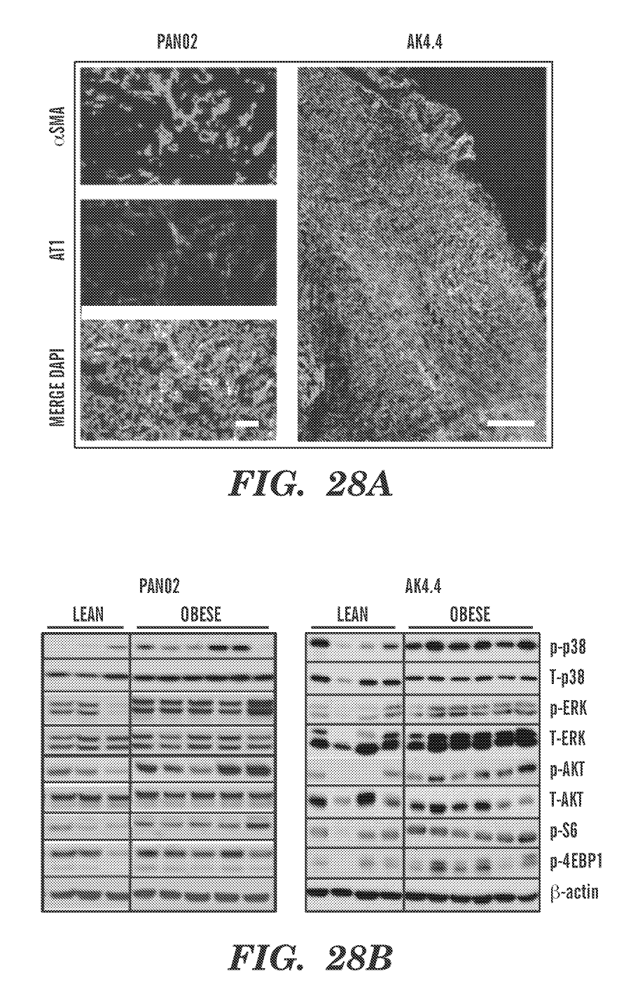

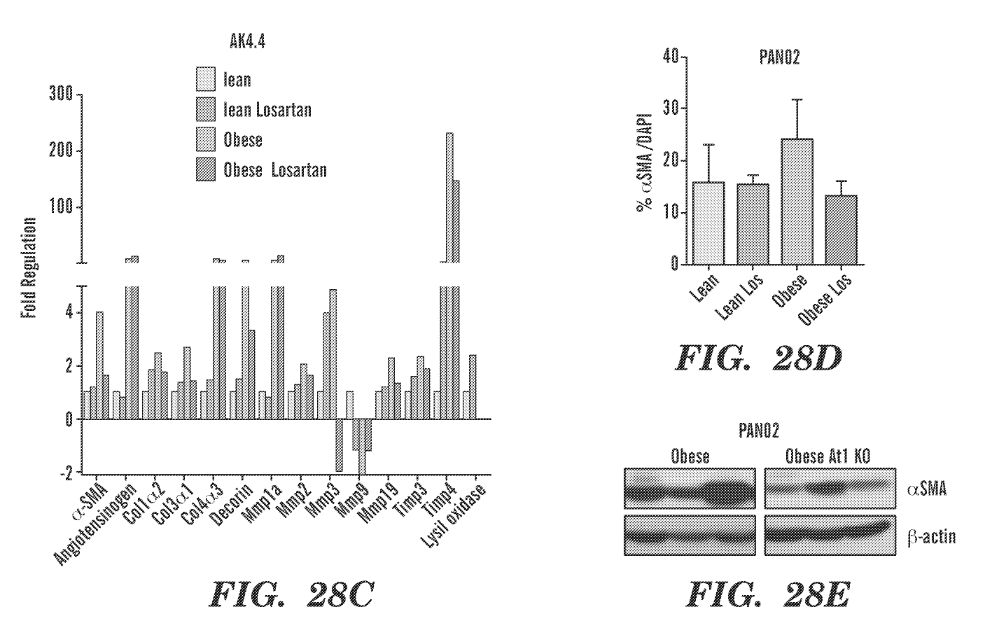

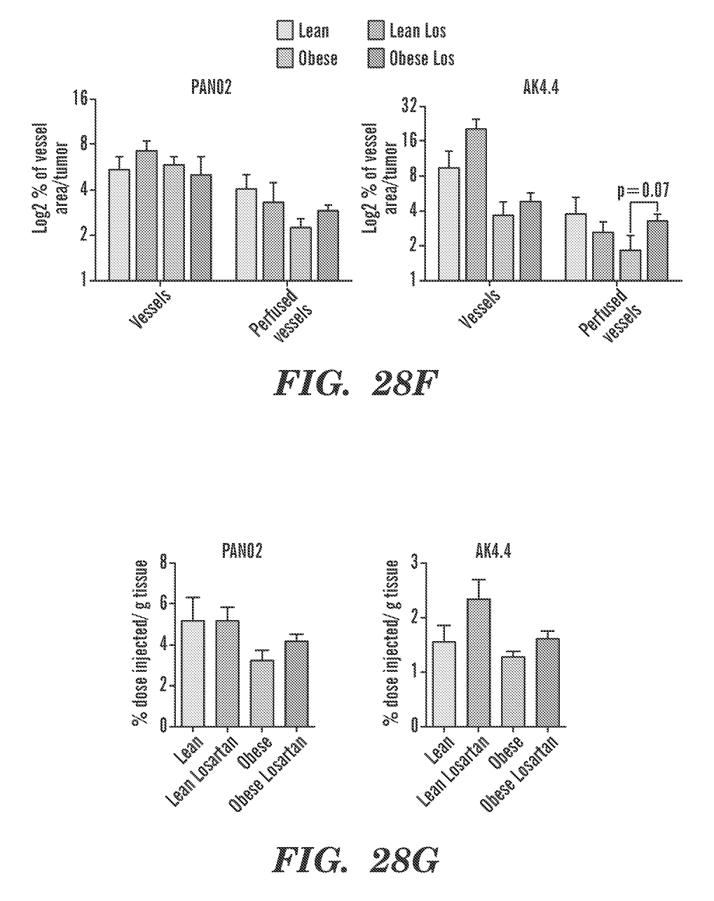

Tumor desmoplasia results in part from the activation of PSCs through AT1 signaling (21, 44). Expression of AT1 in PSCs was confirmed, which ranged from 70.3% (SEM=5.7) in PSCs in PAN02 tumors to 35.3% (SEM=12.8) in AK4.4 tumors (FIG. 28A). Consistent with the observed increase in activated PSC numbers in tumors from obese mice, the activation of AT1 downstream pathways such as p38, ERK, AKT and their targets pS6 and 4EBP1 (21, 45-47) were increased in tumors in obese mice in both models (FIG. 28B). In addition, many target genes of the AT1 pathway, including Coll a1, Mmp genes, and Tgfβ1 were upregulated in PAN02 and AK4.4 tumors from obese mice (FIGS. 20A and 20B). Next, it was tested whether blockade of AT1 signaling could reverse obesity-promoted desmoplasia using the ARB losartan and mice deficient in AT1 (Agtr1a^{-/-}). In both tumor models, losartan was able to reduce gene and protein expression of the activated PSC marker αSMA in tumors from obese animals but did so only mildly in lean animals (FIGS. 20C and 20D, FIGS. 28C and 28D). The reduction of activated PSCs was associated with a decrease in gene and protein expression of collagen-I as well as fibrillar collagen in tumors from obese, but not lean mice (FIGS. 20E, 20F, 20G, and 20H, and FIG. 28C). Moreover, losartan normalized the obesity-induced abnormal expression of several desmoplasia-related markers in tumors including AT1, TGF-β, SMAD2, vimentin, E-cadherin, snail, MMP9, and decorin as well as AT1 downstream signaling pathways (FIGS. 20I, 20J and FIG. 28C). These effects were only modest in lean animals, consistent with the small reduction in αSMA expression in this setting. Similarly, reduced αSMA expression was observed in the PAN02 tumors of obese Agtr1a^{-/-} mice as compared to these tumors in obese wild-type mice (FIG. 28E). Next, it was determined whether AT1 blockade could improve the response to chemotherapy particularly in highly desmoplastic tumors in the obese setting. Pharmacological and genetic AT1 blockade enhanced the response of the PAN02 model to chemotherapy in obese but not in lean animals (FIG. 20K). In the AK4.4 model, AT1 blockade was somewhat effective in the lean setting, but it improved therapeutic response in tumors to a greater extent in obese mice (FIG. 20L). In both AK4.4 and PAN02 models a trend for increased tumor perfusion as well as for increased delivery of chemotherapeutics by AT1 blockade was observed, particularly in obese mice (FIGS. 28F and 28G). In addition to AT1, PSCs also express the angiotensin II type-2 receptor (AT2), which has anti-fibrotic effects as opposed to the pro-fibrotic effects of AT1 signaling (21). However, here, PAN02 tumor response to chemotherapy in AT2−/− mice was similar to that in WT, regardless of diet group (FIG. 20K). Importantly, losartan monotherapy or genetic deletion of AT1 led to reduced tumor weight in obese mice, indicating that obesity-augmented desmoplasia also promotes tumor growth (FIGS. 20K and 20L). In summary, obesity associates with an increase in pro-fibrotic enlarged adipocytes, AT1- and αSMA- expressing (activated) PSCs, upregulation of signaling pathways involved in desmoplasia, collagen-I production, and ultimately an increase in tumor fibrosis and tumor growth. AT1 signaling inhibition is particularly effective in preventing these effects in the obese setting, leading to reduced tumor progression and re-sensitization of tumors to cytotoxic therapies in obese mice.

### Tumor-Associated Neutrophils Mediate Obesity-Induced Desmoplasia and Tumor Progression.

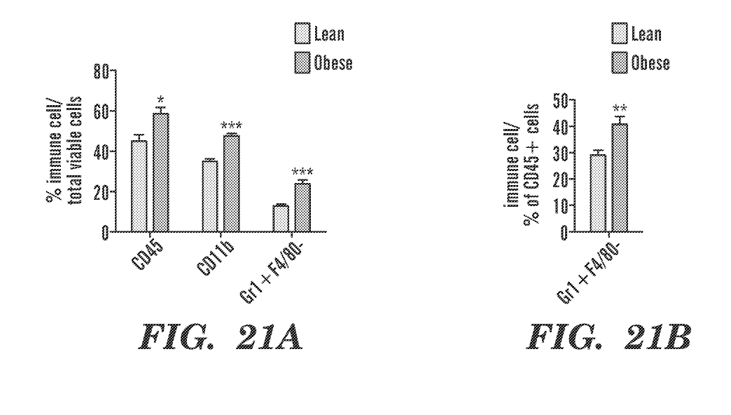

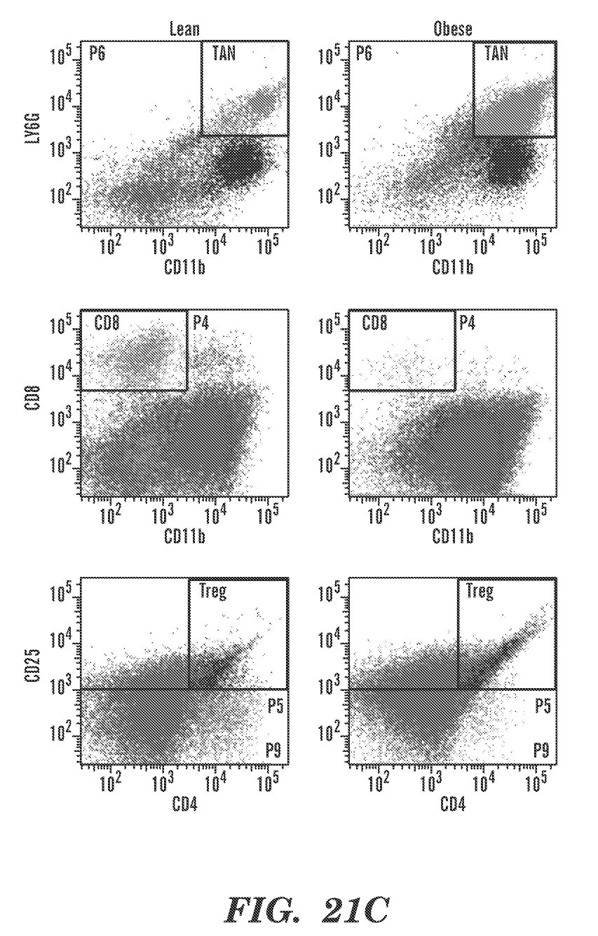

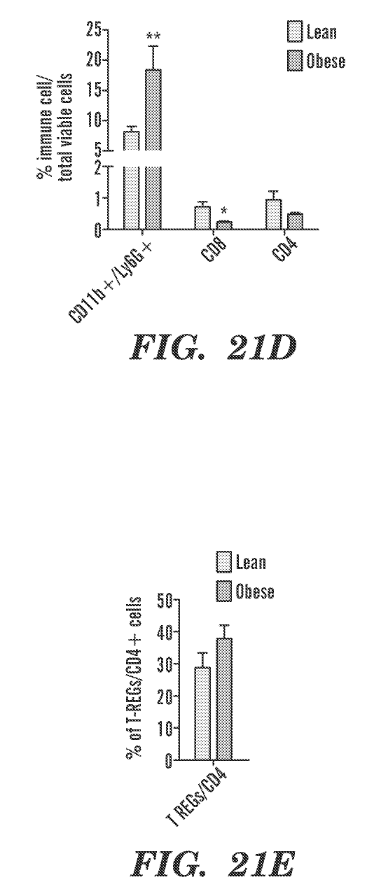

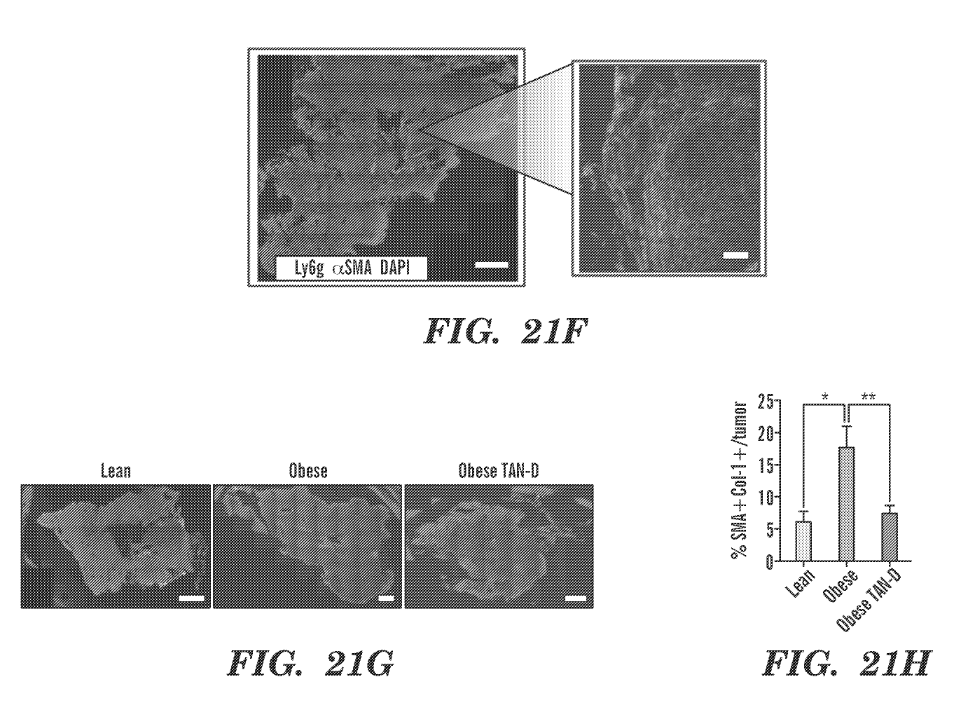

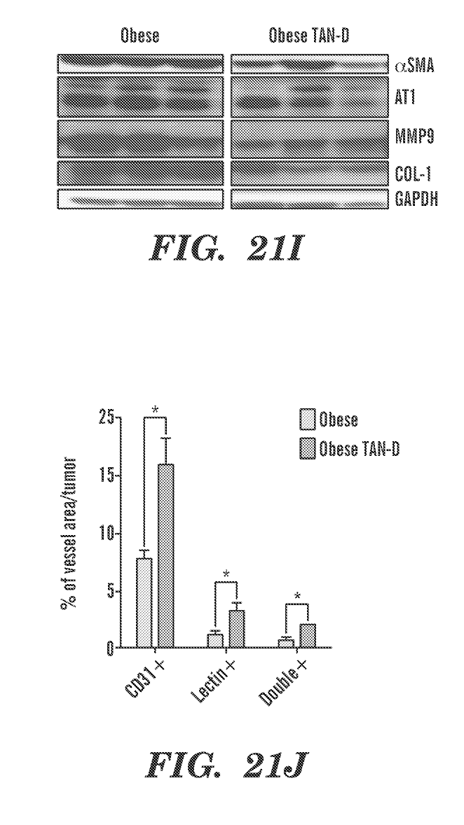

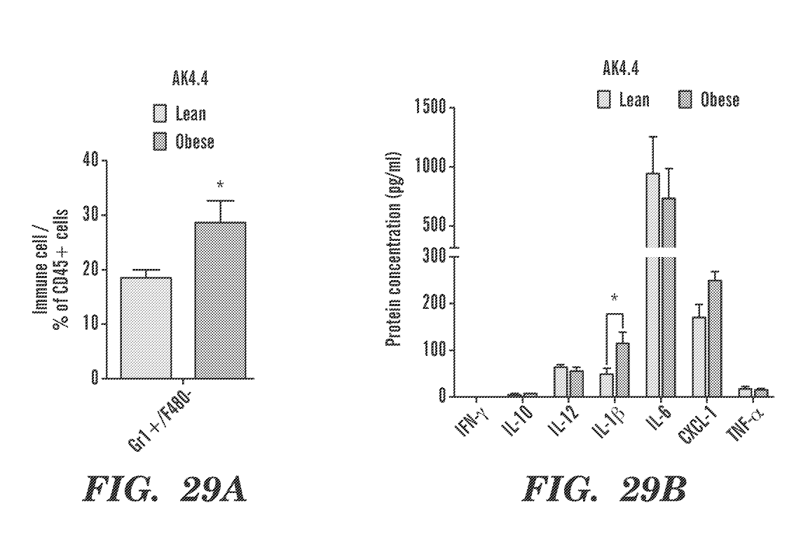

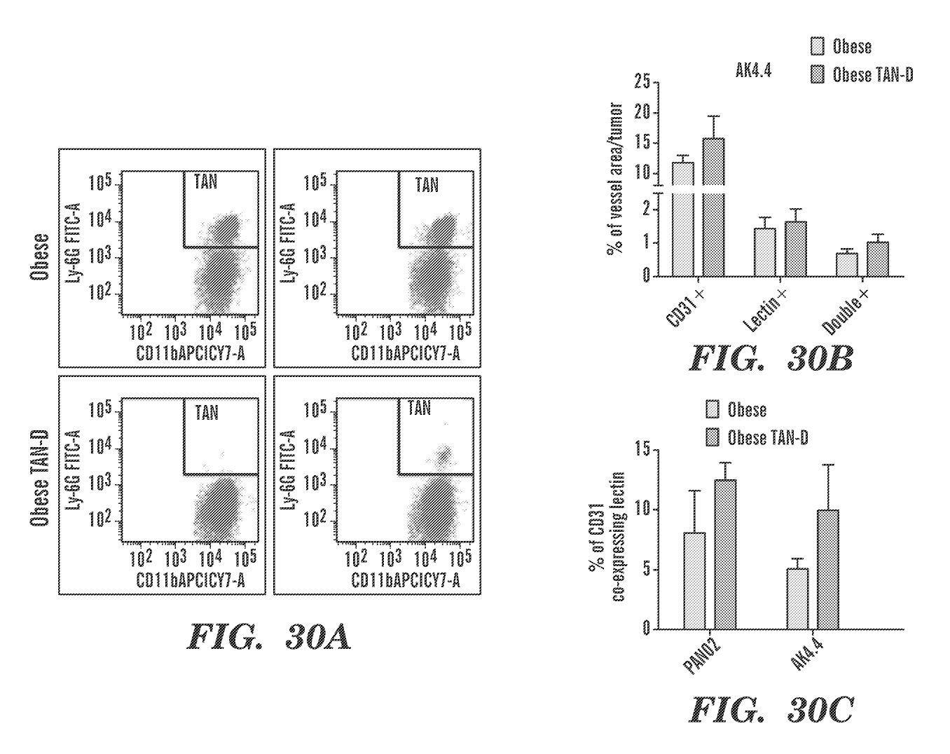

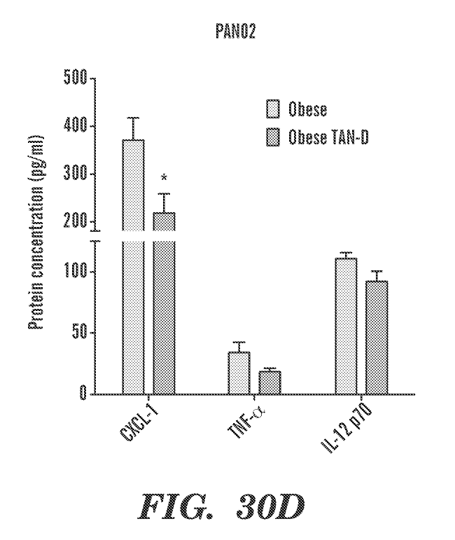

The fibrotic phenotype in adipose tissues and normal pancreas in obesity is largely the consequence of a persistent pro-inflammatory state, which is characterized by the production of cytokines by the hypoxic and dysfunctional hypertrophic adipocytes and recruitment of immune cells (24, 25, 40). Hence, it was next determined whether adipocyte-associated inflammation was responsible for the augmented tumor desmoplasia and accelerated tumor growth observed in obese animals. Flow cytometric analysis revealed that obesity promotes the infiltration of CD11b(+)Gr-1(+)F4/80(−) myeloid cells in two syngeneic PDAC models in obese mice (FIGS. 21A, 21B, and FIG. 29A). The majority of this increased cell population were Ly6G(+) tumor-associated neutrophils (TANs) was confirmed (FIGS. 21C and 21D). In addition, TAN recruitment was accompanied by a reduction in the CD8(+) cytotoxic T cell population in PAN02 tumors (FIGS. 21C and 21D), as well as by a trend toward an increased number of regulatory T cells (Tregs) (FIGS. 21C and 21E). This was associated with an increased expression of IL-4, IL-5, and IL-10 in tumors in both models, suggesting an immunosuppressive tumor microenvironment in obese animals. TAN depletion (TAN-D) in both PDAC models—using an anti-Ly6G antibody that resulted in a˜90% decrease in TANs (FIG. 30A)—reverted the increased tumor weight in obese mice to levels almost similar to lean mice in both models, confirming the relevance of these cells for tumor progression in the context of obesity (FIGS. 21C and 21E). This effect only occurred when TAN depletion was initiated at day 1 but not at day 7 of the experiment, indicating a relative importance of TANs in tumor progression at an early stage (FIGS. 21C and 21D).

In addition to a direct effect on tumor growth, it was determined whether TANs could affect desmoplasia. Preferential accumulation of TANs in areas with activated PSCs was observed (FIG. 21F), suggesting a paracrine crosstalk of TANs and PSCs. Consistently, TAN depletion decreased the number of activated PSCs in tumors from obese mice to the level observed in lean counterparts (FIGS. 21G, 21H, and 21I). Notably, the percentage of αSMA-positive PSCs that express collagen-1 (within the total αSMA positive PSC population) also dropped by 33% after TAN depletion (data not shown). Consistent with the reduction of activated PSCs, we observed reduction of AT1 expression, collagen production, and MMP9 expression in tumors from obese mice after TAN depletion (FIG. 21I). This corresponded to an increase in perfused vessels, although to a lesser extent in AK4.4 tumors (FIGS. 21J, FIGS. 30B and 30C). These data indicate that obesity increases PSC activation, collagen production, and tumor progression at least in part due to increased recruitment of TANs.

### IL-1β Mediates Obesity-Induced TAN Infiltration and Fibrosis in PDACs

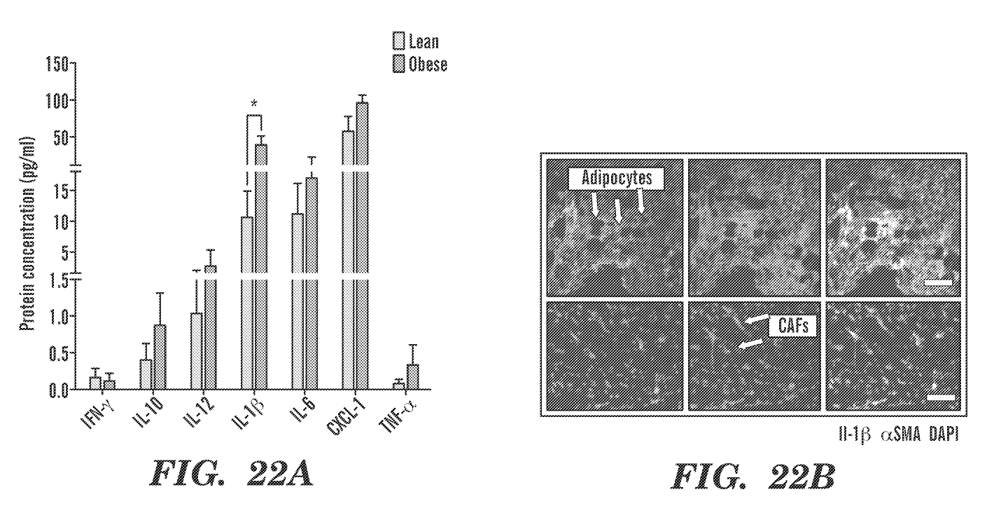

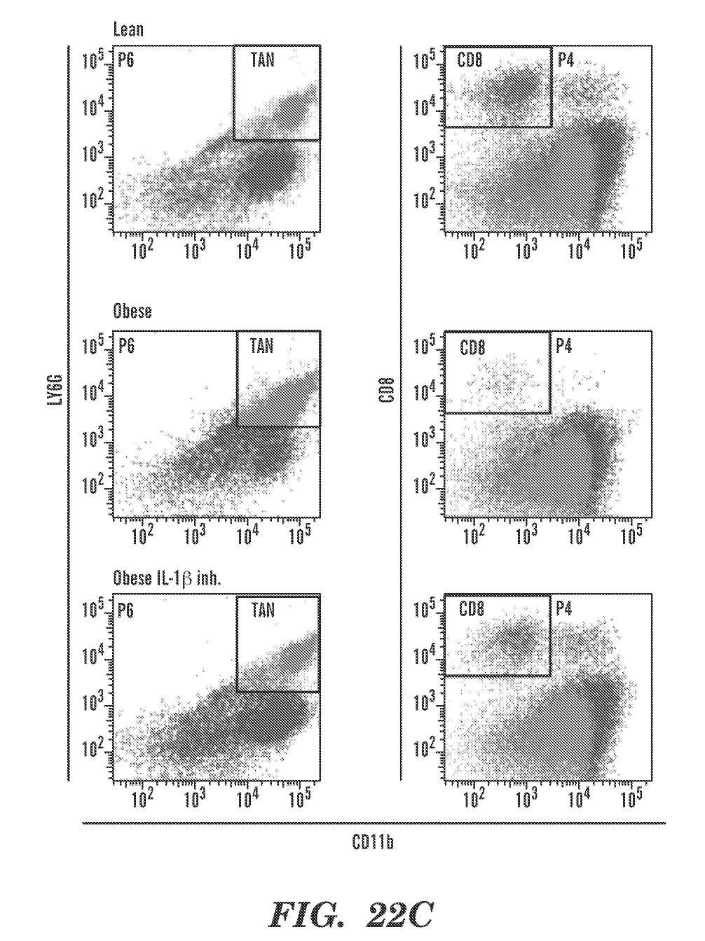

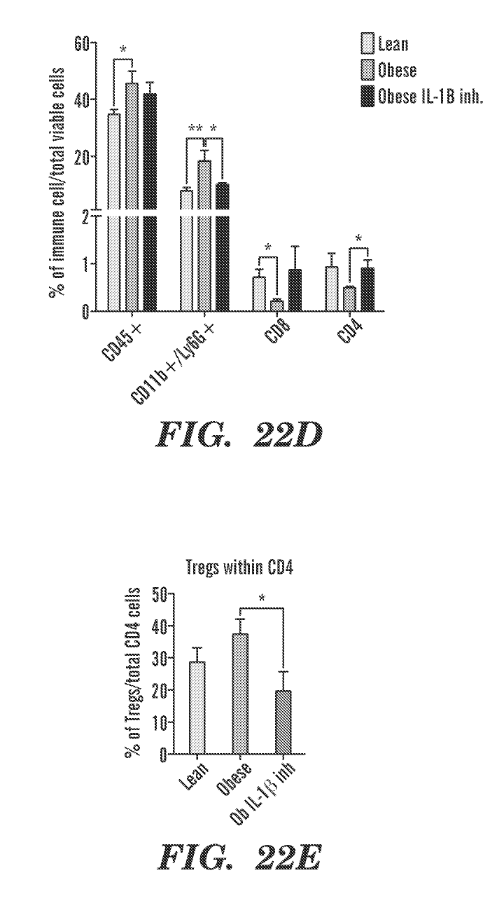

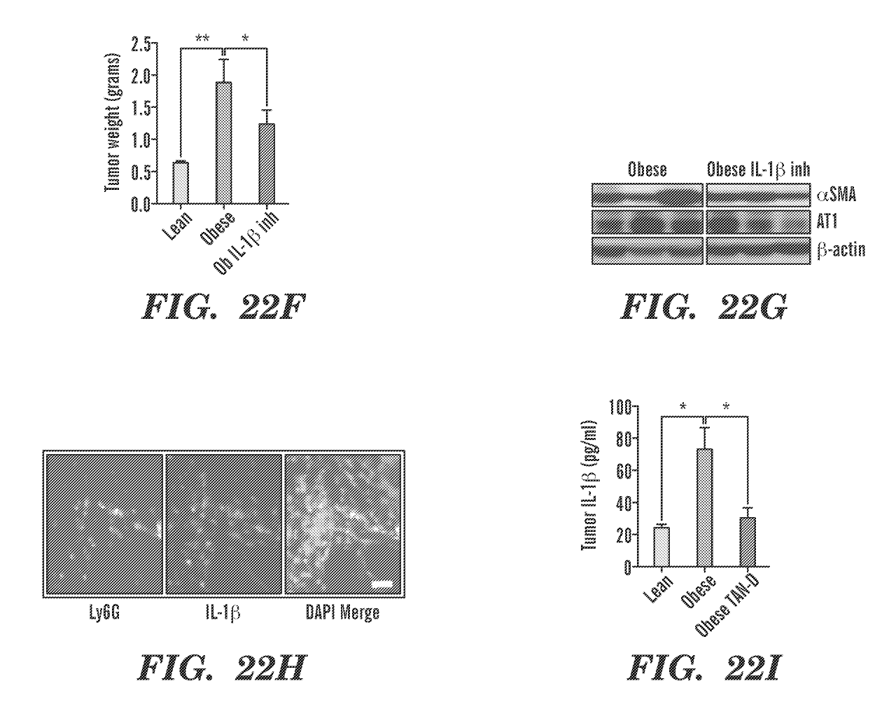

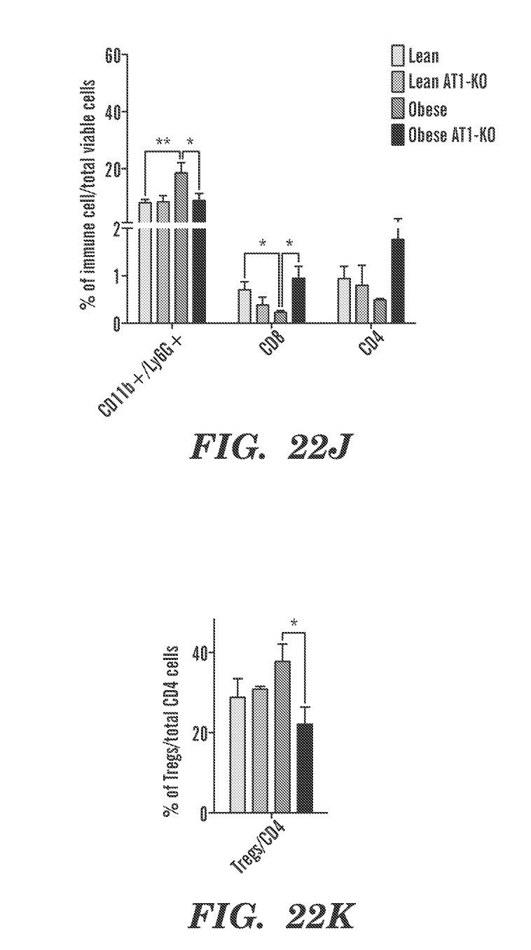

The pro-inflammatory/pro-fibrotic response and immune cell recruitment that occur in adipose tissue under obese condition is mediated by cytokine production from dysfunctional adipocytes, such as IL-1β and IL-6 (24, 25, 48). Hence, it was determined whether these inflammatory cytokines mediated obesity-induced fibrotic processes and TAN infiltration in the tumor microenvironment. Indeed PAN02 tumors from obese mice had a 5-fold increased expression of IL1-β, as well as a trend towards increased expression of IL-6, TNF-α, IL-12 and CXCL1 (FIG. 22A). Consistently, IL-1β was abundantly expressed by adipocytes in the adipocyte-rich areas where PSCs predominate (FIG. 22B, upper row). Using an IL-1β neutralizing antibody (MM425B, Endogen/Pierce Biotechnology, 2 mg/Kg i.p. q4d) in obese mice implanted with PAN02 tumors, a decrease in TAN infiltration (FIGS. 22C and 22D), as well as an increase in CD8(+) T cells and a decrease in Tregs was observed (FIG. 22E), suggesting reversal of the obesity-induced immunosuppressive microenvironment. Similar to the effects of TAN depletion, tumor growth in obese animals was reduced after IL-1β inhibition, and the expression levels of αSMA and AT1 also decreased (FIGS. 22F and 22g). In addition to adipocytes, IL-1β was also expressed in about 70% of TANs themselves (FIG. 22H), and TAN depletion reduced tumor IL-1β levels (FIG. 22I). This suggests the presence of an autocrine mechanism that enables further TAN recruitment and potentiates inflammation and fibrosis. Association of obesity with increased levels of IL-1β in tumors was confirmed in a second model, AK4.4 tumors (FIG. 29B). Other cytokines were also reduced with TAN depletion (CXCL1, and a trend for TNF-α and IL-12), indicating that these cytokines may also contribute to obesity-associated inflammation (FIG. 30D). Finally, αSMA-positive PSCs also abundantly expressed IL-1β (FIG. 22B, lower row).

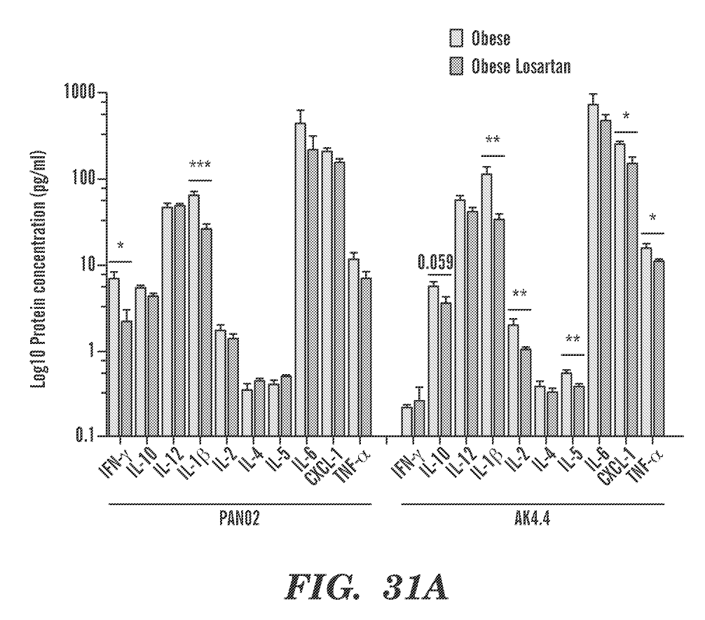

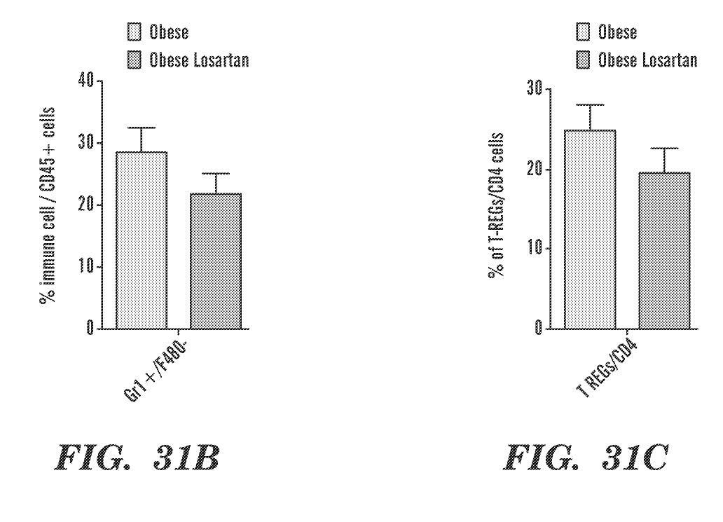

Since IL-1β recruits TANs, which localize in close proximity to PSCs, the ability of targeting PSCs to interfere with IL-1β production and TAN recruitment was examined in PAN02 tumors. Indeed, inhibition of PSC activation by genetic AT1 inhibition decreased IL-1β and TAN levels in obese but not lean mice (FIGS. 22J, 22K, and FIG. 31A). Consequently, recapitulating IL-1β inhibition or TAN depletion, AT1 blockade increased CD8(+) T cells and reduced Tregs in tumors from obese but not lean mice (FIGS. 22J and 22K. Similar trends were observed for losartan in the AK4.4 tumor model (FIGS. 31B and 31C). These effects on tumor-promoting IL-1β and immune cells are consistent with the prevention of obesity-induced tumor growth observed earlier (FIG. 20K). Taken together, these findings indicate that a crosstalk between fibrosis and inflammation induces the fibro-inflammatory microenvironment, promotes tumor progression, and causes chemotherapy resistance in obesity.

PDAC in Obese Patients Presents with Increased Adipocyte Area and Fibrosis

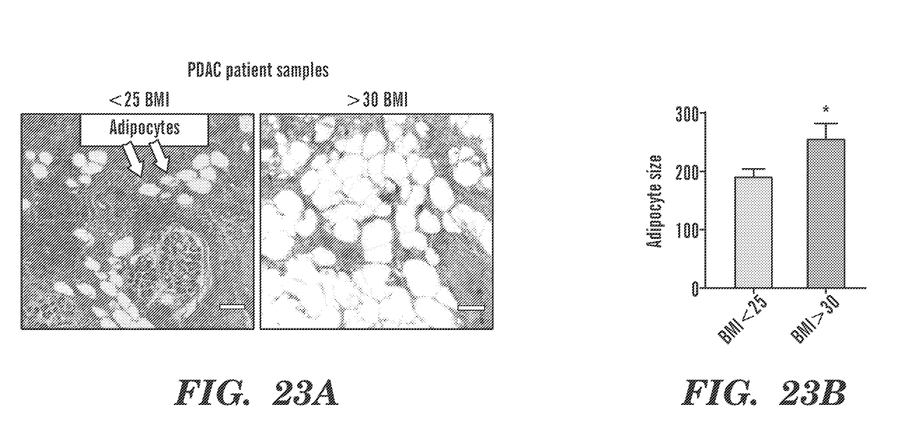

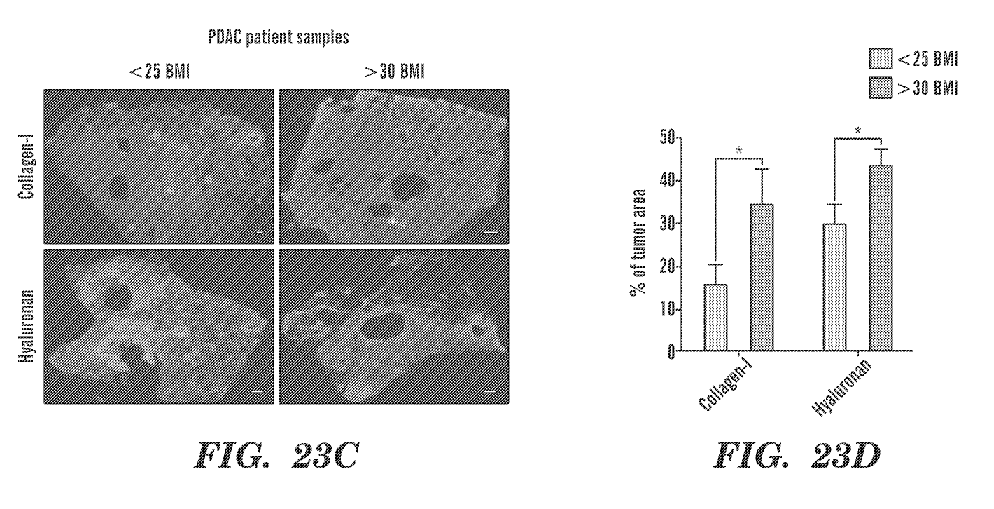

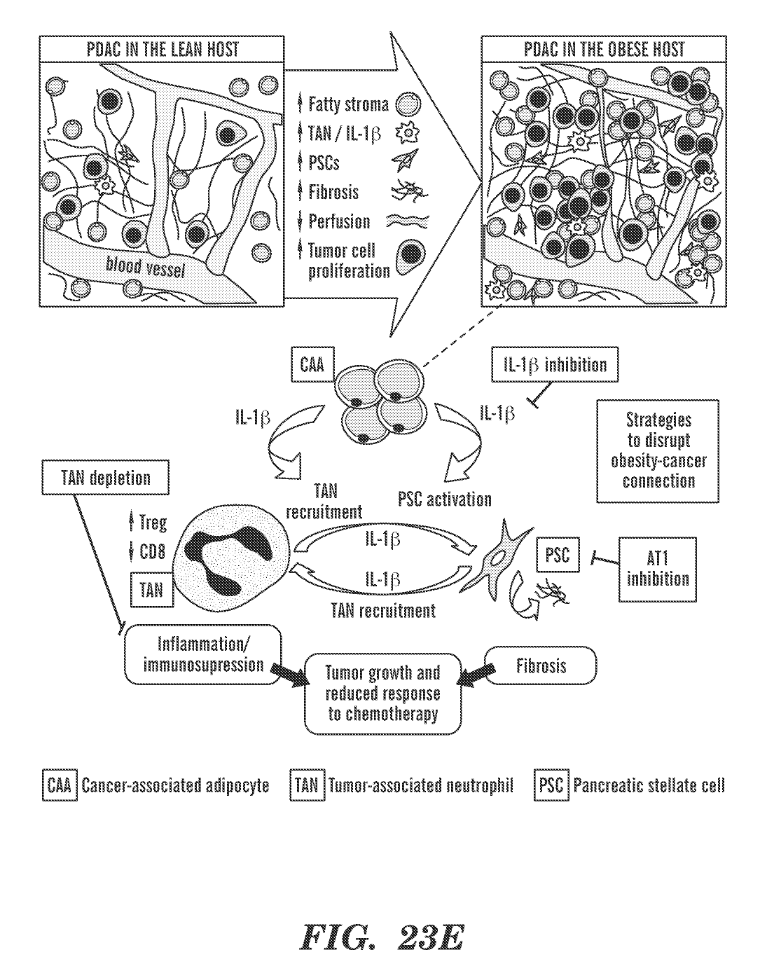

To validate the findings from mouse models of PDAC, human PDAC samples were analyzed from treatment-naïve patients that presented with a body mass index (BMI) either below 25 or above 30. As in the mouse models, tumors from obese (BMI>30) patients presented with hypertrophic adipocytes (FIGS. 23A and 23B) and more pronounced ECM deposition—as shown by increased collagen-I and hyaluronan expression (FIGS. 23C and 23D).

### Discussion

### Obesity Promotes AT-1-Dependent PSC Activation, Tumor Desmoplasia and Hinders Efficacy of Chemotherapy

Although preclinical models have shown that obesity promotes pancreatic tumor initiation, growth, and metastasis (31, 49-52), the underlying mechanisms remain largely elusive. Furthermore, no study has evaluated whether obesity interferes with the response to chemotherapy in pancreatic cancer. Here it is demonstrated for the first time that obesity worsens the fibro-inflammatory microenvironment in PDACs, which leads to increased tumor growth and metastasis and reduced delivery and efficacy of chemotherapy. PSCs and their activation via AT1 signaling are known to play a fundamental role in the production of ECM in pancreatic cancer (21). However, an alteration of PSC behavior in obesity had not been demonstrated. Here it is demonstrated that AT1 signaling mediates obesity-induced PSC activation, which results in the accumulation of ECM components such as collagen-I and HA. AT1 inhibition could normalize collagen-I levels by using a widely prescribed ARB (losartan) or genetic deficiency in mice. This occurred particularly in the more fibrotic tumors in obese mice, leading to a significantly improved response to chemotherapy in the obese but not lean setting. These data are consistent with fibrotic changes observed in early pancreatic lesions (PanIN) in mice fed a high-fat diet (51, 52), and with a recent publication showing the effect of obesity-altered ECM on breast tumorigenesis (53). However, the impact of obesity on desmoplasia and treatment outcome of PDACs was not homogeneous among different PDAC models. Indeed, differences between the lean and obese settings were more pronounced in PDACs with low baseline desmoplasia (e.g., PAN02) than in PDACs with high baseline desmoplasia (e.g., AK4.4).

### IL-1β-Mediated TAN Recruitment in Adipocyte-Rich Regions Augments PSC Activation and Desmoplasia in Obesity

Fibrosis is a natural consequence of chronic inflammation. Indeed, obesity promotes secretion of inflammatory cytokines from hypertrophic adipocytes in adipose tissues and the pancreas, and this ultimately leads to local tissue fibrosis (24, 25, 28, 30, 54). In addition, fibrosis is also accompanied by inflammation in early-stage neoplastic lesions in the pancreas in mice fed with a high-fat diet (51, 52). However, how hypertrophic adipocytes facilitate the interaction between inflammation and desmoplasia to ultimately promote progression of PDACs in the obese setting was not known. Similar to previous reports (31, 32, 34), we demonstrated that hypertrophic, and hence dysfunctional, adipocytes accumulate in both mouse tumors and patient samples. These intratumoral adipocytes, which have been referred to as cancer- associated adipocytes (CAAs) in breast cancer (34), localize to tumor extremities and can promote tumor invasion (35). Furthermore, it was shown that CAAs abundantly expressed IL-1β in PDACs, leading to increased total levels of this cytokine in tumors in obese mice. Not surprising given the ability of adipocytes to produce this cytokine, non-cancer obese patients have increased plasma levels of IL-1β (55). In this example, an accumulation of activated PSCs was observed around adipocyte-rich areas where adipocytes produce IL-1β abundantly. In line with IL-1β being a major activator of PSCs (56), blockade of IL-1β inhibits PSC activation in the obese PDACs. Given the increased adipocyte burden in tumors in obese animals, the overall PSC activation and fibrotic content in tumors was consequently increased. In addition to PSC activation, IL-1β could recruit/activate TANs. This is consistent with increased levels of myeloperoxidase—a marker for intrapancreatic neutrophil sequestration/activation—in association with IL-1β in the steatotic pancreas of obese mice (28, 57). Furthermore, it was observed that TANs can also secrete further IL-1β, which then activates PSCs and TANs themselves. Consequently, TAN specific depletion reduced PSC activation, AT1 signaling, MMP-9 expression and collagen-I production. It is important to note that the accumulation of PSCs in tumors in obese mice may have been caused in part by a dedifferentiation of adipocytes. During tumor invasion in breast cancer, the direct interaction between adipocytes and epithelial cancer cells promote phenotypic changes of CAAs, which lead to adipocyte dedifferentiation and ultimately to an accumulation of fibroblast-like cells (35, 58).

### TANs and IL-1β Play a Major Role on Immunosuppression and Tumor Progression in Obesity

Inflammation in obesity may also promote tumor progression via immunosuppression. The obesity-induced increase in TANs occurs concomitant with decreased CD8(+) cells and increased Tregs, typical of an immunosuppressive microenvironment that promotes tumor progression (59). Importantly, we found that depleting TAN in obese mice increased CD8(+) cells in tumors, as previously reported (60), and this associated with reduced tumor growth acceleration. This is consistent with the correlation between TAN infiltration and more aggressive types of pancreatic tumors (61). Importantly, and as expected due to TAN-recruiting effect of IL-1β, IL-1β inhibition recapitulates the effect of TAN depletion on the immune phenotype and tumor growth. Considering that the immune environment in obesity appears to be suppressed, and the unprecedented success of therapies that block immune checkpoint pathways (59), it would be interesting to evaluate whether these therapies will be particularly effective in obese hosts.

### A Reciprocal Crosstalk Between CAAs, TANs and PSCs is Enhanced in Obesity to Worsen Desmoplasia and Promote Tumor Growth

It was also found that PSC inactivation after AT1 blockade, in addition to decreasing obesity-associated desmoplasia, also reduces levels of IL-1α, decreases the infiltration of TANs and Tregs, increases the number of CD8(+) cells, and reduces obesity-associated tumor growth.

Thus, these results reveal that the interaction of inflammation and fibrosis is bidirectional-targeting inflammation reduces desmoplasia, and in turn targeting desmoplasia reduces inflammation, with both approaches reducing tumor growth. This is consistent with previous in vitro work demonstrating that neutrophils interact reciprocally with myofibroblasts and PSCs (62, 63). As discussed, these results reveal a crosstalk between CAAs, TANs, and PSCs that promotes tumor progression and desmoplasia in obese hosts, with IL-1β (secreted by all these cells) playing a major role in this cooperation (FIG. 23E). This explains that fat reserves can drive tumor growth (64). Importantly, in more than half of PDAC patients, adipocytes infiltrate>20% of pancreatic tissue (32), underlining the importance of the interactions of these cells in the tumor microenvironment. Indeed, increased intra-pancreatic fat correlates with increased incidence of PDAC and lymphatic spread of tumor cells and resulting decreased survival in pancreatic cancer patients (32, 65).

Interestingly, the effect of AT1 inhibition on improving response to chemotherapy in obese mice may occur not only via reduced desmoplasia, but also by directly affecting tumor growth. Since TANs and adipocytes also abundantly expressed AT1 (FIG. S9), AT1 blockade may be acting not just on PSCs, but also directly on those cells. Hence, AT-1 blockade would also directly reduce IL-1β production, TAN infiltration, tumor growth and the enhanced desmoplasia that occurs with obesity. In fact, AT1 is highly expressed in adipocytes (FIG. 32) (26) and increases its activity in adipose tissues in obesity (26, 27). Furthermore, as mentioned above adipocytes can dedifferentiate to become fibroblast-like cells capable of producing collagen in both tumor and non-tumor settings (66-68). Hence AT1 blockade could be affecting the central culprit of the fibro-inflammatory cycle generated in the obese setting—the CAA.

Of note, consistent with previous work in PAN02 tumors (69), by using both genetic and diet-induced obese mouse models, it was determined that obesity promotes tumor growth independently of diet. This was also confirmed by the finding that FVB mice which do not gain weight despite a high-fat diet (˜25% of mice), observed tumor growth similar to that in lean animals (not shown).

### Conclusions

Obesity is considered to be responsible for 14-20% of all cancer-related deaths in the United States (5). Consequently, the epidemic of obesity—which also affects the majority of PDAC patients—highlights the importance of understanding the pathophysiology underlying the obesity-cancer connection. Here, it is shown that obesity induces a pro-inflammatory and desmoplastic tumor microenvironment, which directly promotes tumor growth, as well as impairs the response to systemic treatment. Both of these two factors may explain the poor outcomes in obese patients. The finding that obesity-induced desmoplasia better responds to clinically available anti-fibrotic therapies (e.g., ARBs) as well as anti-inflammatory agents (e.g., IL-1β inhibitor), is extremely encouraging and calls for an investigation and clinical trials on the efficacy of these therapies in obese PDAC patients in combination with the current standard-of-care. Since epidemiological and molecular evidence suggests a link between obesity and other desmoplastic cancer types, the strategies established in this study may also apply to a broader patient population.

### REFERENCES FOR EXAMPLE 2

1. ACS CFF. Cancer Facts & Figures 2014. ACS, 2014; Available from: http://www.caricer.org/research/cancerfactsstatistics/cancerfactsfigures2014/
2. American Cancer Society: Cancer Facts and Figures 2015. http://wwwcancerorg/acs/groups/content/@editorial/documents/document/acspc-044552pdf.
3. Genkinger J M, Spiegelman D, Anderson K E, Bernstein L, van den Brandt P A, Calle E E, et al. A pooled analysis of 14 cohort studies of anthropometric factors and pancreatic cancer risk. International journal of cancer Journal international du cancer. 2011; 129:1708-17.
4. Yuan C, Bao Y, Wu C, Kraft P, Ogino S, Ng K, et al. Prediagnostic body mass index and pancreatic cancer survival. Journal of clinical oncology: official journal of the American Society of Clinical Oncology. 2013; 31:4229-34.
5. Calle E E, Rodriguez C, Walker-Thurmond K, Thun M J. Overweight, obesity, and mortality from cancer in a prospectively studied cohort of U.S. adults. N Engl J Med. 2003; 348:1625-38.
6. McWilliams R R, Matsumoto M E, Burch P A, Kim G P, Halfdanarson T R, de Andrade M, et al. Obesity adversely affects survival in pancreatic cancer patients. Cancer. 2010; 116:5054-62.
7. Li D, Morris J S, Liu J, Hassan M M, Day R S, Bondy M L, et al. Body mass index and risk, age of onset, and survival in patients with pancreatic cancer. JAMA: the journal of the American Medical Association. 2009; 301:2553-62.
8. Bracci P M. Obesity and pancreatic cancer: overview of epidemiologic evidence and biologic mechanisms. Molecular carcinogenesis. 2012; 51:53-63.
9. Smits M M, van Geenen E J. The clinical significance of pancreatic steatosis. Nature reviews Gastroenterology & hepatology. 2011; 8:169-77.
10. Goodwin P J, Stambolic V. Impact of the obesity epidemic on cancer. Annual review of medicine. 2015; 66:281-96.
11. Ogden C L, Carroll M D, Kit B K, Flegal K M. Prevalence of childhood and adult obesity in the United States, 2011-2012. JAMA: the journal of the American Medical Association. 2014; 311:806-14.
12. Dvorak H F. Tumors: wounds that do not heal. Similarities between tumor stroma generation and wound healing. N Engl J Med. 1986; 315:1650-9.
13. Whatcott C J, Diep C H, Jiang P, Watanabe A, LoBello J, Sima C, et al. Desmoplasia in primary tumors and metastatic lesions of pancreatic cancer. Clin Cancer Res. 2015.
14. Olive K P, Jacobetz M A, Davidson C J, Gopinathan A, McIntyre D, Honess D, et al. Inhibition of Hedgehog signaling enhances delivery of chemotherapy in a mouse model of pancreatic cancer. Science. 2009; 324:1457-61.
15. Hwang R F, Moore T, Arumugam T, Ramachandran V, Amos K D, Rivera A, et al. Cancer-associated stromal fibroblasts promote pancreatic tumor progression. Cancer Res. 2008; 68:918-26.
16. Neesse A, Frese K K, Bapiro T E, Nakagawa T, Sternlicht M D, Seeley T W, et al. CTGF antagonism with mAb FG-3019 enhances chemotherapy response without increasing drug delivery in murine ductal pancreas cancer. Proceedings of the National Academy of Sciences of the United States of America. 2013; 110:12325-30.
17. Stylianopoulos T, Martin J D, Chauhan V P, Jain S R, Diop Frimpong B, Bardeesy N, et al. Causes, consequences, and remedies for growth-induced solid stress in murine and human tumors. Proceedings of the National Academy of Sciences of the United States of America. 2012; 109:15101-8.
18. Chauhan V P, Boucher Y, Ferrone C R, Roberge S, Martin J D, Stylianopoulos T, et al. Compression of pancreatic tumor blood vessels by hyaluronan is caused by solid stress and not interstitial fluid pressure. Cancer Cell. 2014; 26:14-5.
19. Alvarez R, Musteanu M, Garcia-Garcia E, Lopez-Casas P P, Megias D, Guerra C, et al. Stromal disrupting effects of nab-paclitaxel in pancreatic cancer. British Journal of Cancer. 2013; 109:926-33.
20. Hidalgo M, Von Hoff D D. Translational Therapeutic Opportunities in Ductal Adenocarcinoma of the Pancreas. Clinical Cancer Research. 2012; 18:4249-56.
21. Chauhan V P, Martin J D, Liu H, Lacorre D A, Jain S R, Kozin S V, et al. Angiotensin inhibition enhances drug delivery and potentiates chemotherapy by decompressing tumor blood vessels. Nature Communications. 2013; 4:2516.
22. Endrich B, Reinhold H S, Gross J F, Intaglietta M. Tissue perfusion inhomogeneity during early tumor growth in rats. J Natl Cancer Inst. 1979; 62:387-95.
23. Costa C, Incio J, Soares R. Angiogenesis and chronic inflammation: cause or consequence? Angiogenesis. 2007; 10:149-66.
24. Hosogai N, Fukuhara A, Oshima K, Miyata Y, Tanaka S, Segawa K, et al. Adipose tissue hypoxia in obesity and its impact on adipocytokine dysregulation. Diabetes. 2007; 56:901-11.
25. Incio J, Soares R. Obesity, Diabetes and Metabolic Syndrome Impact on TUMOR ANGIOGENESIS. In: Ruben R. Gonzalez-Perez B R R, editor. Tumor Angiogenesis Regulators: CRC Press; 2013.
26. http://biogps.org. AGTR1 (angiotensin II receptor, type 1) gene expression, activity chart. 2015.
27. Sun K, Tordjman J, Clement K, Scherer P E. Fibrosis and adipose tissue dysfunction. Cell metabolism. 2013; 18:470-7.
28. Mathur A, Marine M, Lu D, Swartz-Basile D A, Saxena R, Zyromski N J, et al. Nonalcoholic fatty pancreas disease. HPB: the official journal of the International Hepato Pancreato Biliary Association. 2007; 9:312-8.
29. Matsuda A, Makino N, Tozawa T, Shirahata N, Honda T, Ikeda Y, et al. Pancreatic fat accumulation, fibrosis, and acinar cell injury in the Zucker diabetic fatty rat fed a chronic high-fat diet. Pancreas. 2014; 43:735-43.
30. Hursting S D. Minireview: the year in obesity and cancer. Molecular endocrinology. 2012; 26:1961-6.
31. Zyromski N J, Mathur A, Pitt H A, Wade T E, Wang S, Nakshatri P, et al. Obesity potentiates the growth and dissemination of pancreatic cancer. Surgery. 2009; 146:258-63.
32. Hori M, Takahashi M, Hiraoka N, Yamaji T, Mutoh M, Ishigamori R, et al. Association of pancreatic Fatty infiltration with pancreatic ductal adenocarcinoma. Clinical and translational gastroenterology. 2014; 5:e53.
33. Hefetz -Sela S, Scherer P E. Adipocytes: impact on tumor growth and potential sites for therapeutic intervention. Pharmacology & therapeutics. 2013; 138:197-210.
34. Dirat B, Bochet L, Dabek M, Daviaud D, Dauvillier S, Majed B, et al. Cancer associated adipocytes exhibit an activated phenotype and contribute to breast cancer invasion. Cancer Res. 2011; 71:2455-65.
35. Wang Y Y, Lehuede C, Laurent V, Dirat B, Dauvillier S, Bochet L, et al. Adipose tissue and breast epithelial cells: a dangerous dynamic duo in breast cancer. Cancer letters. 2012; 324:142-51.
36. Hingorani S R, Wang L, Multani A S, Combs C, Deramaudt T B, Hruban R H, et al. Trp53R172H and KrasG12D cooperate to promote chromosomal instability and widely metastatic pancreatic ductal adenocarcinoma in mice. Cancer cell. 2005; 7:469-83.
37. Kawaguchi Y, Cooper B, Gannon M, Ray M, MacDonald R J, Wright C V. The role of the transcriptional regulator Ptfl a in converting intestinal to pancreatic progenitors. Nature genetics. 2002; 32:128-34.
38. Tam J, Duda D G, Perentes J Y, Quadri R S, Fukumura D, Jain R K. Blockade of VEGFR2 and not VEGFR1 can limit diet-induced fat tissue expansion: role of local versus bone marrow-derived endothelial cells. PloS one. 2009; 4:e4974.
39. Fukumura D, Ushiyama A, Duda D G, Xu L, Tam J, Krishna V, et al. Paracrine regulation of angiogenesis and adipocyte differentiation during in vivo adipogenesis. Circulation research. 2003; 93:e88-97.
40. Kanasaki K, Koya D. Biology of obesity: lessons from animal models of obesity. Journal of biomedicine & biotechnology. 2011; 2011: 197636.
41. Koshiba T, Hosotani R, Wada M, Miyamoto Y, Fujimoto K, Lee J U, et al. Involvement of matrix metalloproteinase-2 activity in invasion and metastasis of pancreatic carcinoma. Cancer. 1998; 82:642-50.
42. Collins M A, Bednar F, Zhang Y, Brisset J C, Galban S, Galban C J, et al. Oncogenic Kras is required for both the initiation and maintenance of pancreatic cancer in mice. J Clin Invest. 2012; 122:639-53.
43. Neoptolemos J P, Moore M J, Cox T F, Valle J W, Palmer D H, McDonald A C, et al. Effect of adjuvant chemotherapy with fluorouracil plus folinic acid or gemcitabine vs observation on survival in patients with resected periampullary adenocarcinoma: the ESPAC 3 periampullary cancer randomized trial. JAMA: the journal of the American Medical Association. 2012; 308:147-56.
44. Apte M V, Park S, Phillips P A, Santucci N, Goldstein D, Kumar R K, et al. Desmoplastic reaction in pancreatic cancer—Role of pancreatic stellate cells. Pancreas. 2004; 29:179-87.
45. Yang F, Chung A C, Huang X R, Lan H Y. Angiotensin II induces connective tissue growth factor and collagen I expression via transforming growth factor-beta-dependent and -independent Smad pathways: the role of Smad3. Hypertension. 2009; 54:877-84.
46. Hama K, Ohnishi H, Yasuda H, Ueda N, Mashima H, Satoh Y, et al. Angiotensin II stimulates DNA synthesis of rat pancreatic stellate cells by activating ERK through EGF receptor transactivation. Biochemical and Biophysical Research Communications. 2004; 315:905-11.
47. Hama K, Ohnishi H, Aoki H, Kita H, Yamamoto H, Osawa H, et al. Angiotensin II promotes the proliferation of activated pancreatic stellate cells by Smad7 induction through a protein kinase C pathway. Biochemical and Biophysical Research Communications. 2006; 340:742-50.
48. Weisberg S P, McCann D, Desai M, Rosenbaum M, Leibel R L, Ferrante A W, Jr. Obesity is associated with macrophage accumulation in adipose tissue. J Clin Invest. 2003; 112:1796-808.
49. Murtaugh L C. Pathogenesis of pancreatic cancer: lessons from animal models. Toxicologic pathology. 2014; 42:217-28.
50. White P B, True E M, Ziegler K M, Wang S S, Swartz-Basile D A, Pitt H A, et al. Insulin, leptin, and tumoral adipocytes promote murine pancreatic cancer growth. Journal of gastrointestinal surgery: official journal of the Society for Surgery of the Alimentary Tract. 2010; 14:1888-93;discussion 93-4.
51. Philip B, Roland C L, Daniluk J, Liu Y, Chatterjee D, Gomez S B, et al. A high-fat diet activates oncogenic Kras and COX2 to induce development of pancreatic ductal adenocarcinoma in mice. Gastroenterology. 2013; 145:1449-58.
52. Khasawneh J, Schulz M D, Walch A, Rozman J, Hrabe de Angelis M, Klingenspor M, et al. Inflammation and mitochondrial fatty acid beta-oxidation link obesity to early tumor promotion. Proc Natl Acad Sci USA. 2009; 106:3354-9.
53. Seo B R, Bhardwaj P, Choi S, Gonzalez J, Andresen Eguiluz R C, Wang K, et al. Obesity-dependent changes in interstitial ECM mechanics promote breast tumorigenesis. Science translational medicine. 2015; 7:301 ra130.
54. Hursting S D, Dunlap S M. Obesity, metabolic dysregulation, and cancer: a growing concern and an inflammatory (and microenvironmental) issue. Annals of the New York Academy of Sciences. 2012; 1271:82-7.
55. Um J Y, Chung H S, Song M Y, Shin H D, Kim H M. Association of interleukin-1beta gene polymorphism with body mass index in women. Clinical chemistry. 2004; 50:647-50.
56. Masamune A, Satoh M, Kikuta K, Sakai Y, Satoh A, Shimosegawa T. Inhibition of p38 mitogen-activated protein kinase blocks activation of rat pancreatic stellate cells. The Journal of pharmacology and experimental therapeutics. 2003; 304:8-14.
57. Zyromski N J, Mathur A, Pitt H A, Lu D, Gripe J T, Walker J J, et al. A murine model of obesity implicates the adipokine milieu in the pathogenesis of severe acute pancreatitis. American journal of physiology Gastrointestinal and liver physiology. 2008; 295:G552-8.
58. Tan J, Buache E, Chenard M P, Dali-Youcef N, Rio M C. Adipocyte is a non-trivial, dynamic partner of breast cancer cells. The International journal of developmental biology. 2011; 55:851-9.
59. Coussens L M, Zitvogel L, Palucka A K. Neutralizing tumor-promoting chronic inflammation:a magic bullet? Science. 013;339:286-91.
60. Fridlender Z G, Sun J, Kim S, Kapoor V, Cheng G, Ling L, et al. Polarization of tumor-associated neutrophil phenotype by TGF-beta: “N1” versus “N2” TAN. Cancer Cell. 2009; 16:183-94.
61. Reid M D, Basturk O, Thirabanjasak D, Hruban R H, Klimstra D S, Bagci P, et al. Tumor-infiltrating neutrophils in pancreatic neoplasia. Modern pathology: an official journal of the United States and Canadian Academy of Pathology, Inc. 2011; 24:1612-9.
62. Chrysanthopoulou A, Mitroulis I, Apostolidou E, Arelaki S, Mikroulis D, Konstantinidis T, et al. Neutrophil extracellular traps promote differentiation and function of fibroblasts. The Journal of pathology. 2014; 233:294-307.
63. Zhu Q, Zhang X, Zhang L, Li W, Wu H, Yuan X, et al. The IL-6-STAT3 axis mediates a reciprocal crosstalk between cancer-derived mesenchymal stem cells and neutrophils to synergistically prompt gastric cancer progression. Cell death & disease. 2014; 5:e1295.
64. Williams S C. Link between obesity and cancer. Proc Natl Acad Sci USA. 2013; 110:8753-4.
65. Mathur A, Zyromski N J, Pitt H A, Al-Azzawi H, Walker J J, Saxena R, et al. Pancreatic steatosis promotes dissemination and lethality of pancreatic cancer. J Am Coll Surg. 2009; 208:989-94-6;
66. Jotzu C, Alt E, Welte G, Li J, Hennessy B T, Devarajan E, et al. Adipose tissue derived stem cells differentiate into carcinoma-associated fibroblast-like cells under the influence of tumor derived factors. Cellular oncology. 2011;34:55-67.
67. Park J, Euhus D M, Scherer P E. Paracrine and endocrine effects of adipose tissue on cancer development and progression. Endocr Rev. 2011; 32:550-70.
68. Matsumoto T, Kano K, Kondo D, Fukuda N, Iribe Y, Tanaka N, et al. Mature adipocyte-derived dedifferentiated fat cells exhibit multilineage potential. J Cell Physiol. 2008; 215:210-22.
69. White P B, Ziegler K M, Swartz Basile D A, Wang S S, Lillemoe K D, Pitt H A, et al. Obesity, but not high-fat diet, promotes murine pancreatic cancer growth. Journal of gastrointestinal surgery: official journal of the Society for Surgery of the Alimentary Tract. 2012; 16:1680-5.
70. Surwit R S, Feinglos M N, Rodin J, Sutherland A, Petro A E, Opara E C, et al. Differential effects of fat and sucrose on the development of obesity and diabetes in C57BL/6J and A/J mice. Metabolism: clinical and experimental. 1995; 44:645-51.
71. Bardeesy N, Aguirre A J, Chu G C, Cheng K H, Lopez L V, Hezel A F, et al. Both p16(Ink4a) and the p19(Arf) p53 pathway constrain progression of pancreatic adenocarcinoma in the mouse. Proc Natl Acad Sci USA. 2006; 103:5947-52.
72. Diop-Frimpong B, Chauhan V P, Krane S, Boucher Y, Jain R K. Losartan inhibits collagen I synthesis and improves the distribution and efficacy of nanotherapeutics in tumors. Proceedings of the National Academy of Sciences of the United States of America. 2011; 108:2909-14.

### Example 3

### Obesity Promotes Resistance to Anti-VEGF Therapy in Breast Cancer via Pro-Inflammatory and Angiogenic Pathways

Background: Most breast cancer (BC) patients are overweight or obese at the time of diagnosis. Obesity is associated with increased risk, recurrence, and worse prognosis of BC. It has been shown that obesity associates with worse outcome in metastatic kidney or colon cancer treated with bevacizumab. If and how excess body weight contributes to the failure of anti-VEGF therapy in BC is unknown.

It is unclear why anti-VEGF therapy (bevacizumab) has failed to improve survival in breast cancer (BC) patients. In metastatic kidney or colon cancer, obesity is associated with worse outcome in patients receiving bevacizumab [1-2]. The majority of BC patients are overweight or obese, and obesity is associated with poor prognosis [3]. However, the effect of obesity specifically on response to anti-VEGF therapy in BC is currently unknown.

In obesity, the expansion of adipose tissues occurs faster than the angiogenic process, leading to inadequate vascularization [3]. This, in addition to the limiting of oxygen diffusion to the center of enlarged adipocytes, results in the creation of a hypoxic microenvironment in breast adipose tissue and subsequent recruitment of inflammatory cells that, together with the dysfunctional hypertrophic adipocytes, secrete pro-inflammatory/angiogenic cytokines (adipokines) [4].

Importantly, in non-obese studies, inflammatory and angiogenic pathways, such as interleukin 6 (IL-6) and fibroblast growth factor-2 (FGF-2), have been shown to sustain angiogenesis and tumor progression despite VEGF blockade [5]. Given that adipose tissue is a large component of BC, we hypothesize that obesity-induced inflammation in the BC microenvironment promotes resistance to anti-VEGF therapies via increased inflammatory and angiogenic factors.

Results: Here we found that diet-induced obesity promoted resistance to anti-VEGF therapy in two syngeneic mouse breast cancer models. The effects of anti-VEGF therapy on tumor growth and metastasis, VEGF downstream signaling pathways and vessel density were significantly attenuated in obese mice. Under obesity condition, intra-tumor adipocytes increased. These adipocyte-rich regions in breast cancers were hypoxic and overexpress IL-6 or FGF-2 by adipocytes, fibroblasts, and myeloid cells. In IL-6 overexpressing obese breast cancer model (E0771), neutralization of IL-6, either genetically or pharmacologically, abrogated the obesity-induced resistance to anti-VEGF therapy seen in both primary and metastasis sites. This occurred due to a reversion of the obesity-augmented STAT3 signaling and cell proliferation, of hypoxia via vessel normalization, and of immunosuppression. In another breast cancer model (MCaIV), which overexpress FGF-2 under obesity, anti-FGF receptor antibody restored tumor sensitivity to anti-VEGF treatment in obesity.

Conclusion: Our findings indicate that obesity promotes resistance to anti-VEGF therapy in breast cancer via the production of pro-inflammatory and angiogenic factors that circumvent the loss of VEGF signaling.

### REFERENCE FOR EXAMPLE 3

1. Guiu, B., et al. “Visceral fat area is an independent predictive biomarker of outcome after first-line bevacizumab-based treatment in metastatic colorectal cancer.” Gut 59, 341-347 (2010)
2. Ladoire, S., et al. “Visceral fat area as a new independent predictive factor of survival in patients with metastatic renal cell carcinoma treated with antiangiogenic agents.”Oncologist 16, 71-81 (2011).
3. Incio, J. & Soares, R. “Obesity, diabetes and metabolic syndrome impact on tumor angiogenesis” in Tumor Angiogenesis Regulators (ed. Rueda, R.R.G.-P.B.R.) (CRC, 2013).
4. Costa, C., Incio, J. & Soares, R. “Angiogenesis and chronic inflammation: cause or consequence?”Angiogensis 10, 149-166 (2007).
5. Jain, R. K. “Normalizing tumor microenvironment to treat cancer: bench to bedside to biomarkers.”Journal of Clinical Oncology 31, 2205-2218 (2013).

### Example 4

### Role of Stromal VEGFR-1 Signaling in Obesity-Induced Tumor Progression

Background: With the current obesity epidemic, the majority of patients diagnosed with pancreatic ductal adenocarcinoma (PDAC) and breast cancer (BC) are overweight or obese [1-2]. This excess body weight is associated with accelerated tumor progression and poor prognosis, but the mechanisms are poorly understood [1-3]. Obesity associates with angiogenesis and increased macrophage infiltration in adipose tissues during weight gain [4]. Whether these effects also occur in cancer to promote tumor progression in obese condition remains unclear.

We have shown that the vascular endothelial growth factor receptor-1 (VEGFR-1) pathway can modulate tumor angiogenesis and recruitment of tumor-associated macrophages (TAMs) [5-6]. Here, we tested the emerging hypothesis that obesity enhances tumor progression and metastasis by augmenting angiogenesis and TAM recruitment via activation of the VEGFR-1 pathway.

Methods: We used a high-fat diet-induced obesity model in either wild type (WT) or VEGFR-1 tyrosine kinase null (F1t1TK−/−) C57BL/6 mice. Then, we implanted orthotopically syngeneic pancreatic (PAN02) or breast (E0771) carcinomas. We evaluated the role of VEGFR-1 activity on systemic metabolism, tumor angiogenesis and immune environment, and tumor growth and metastasis. Results: Obesity increased p38- [symbol] MAPK activation and TAM infiltration, tumor growth (p=0.001) and metastasis (p=0.035) in PAN02 tumors. VEGFR-1 inhibition reduced tumor growth (p=0.007) and metastasis (p=0.017) in obese but not lean mice. This was associated with a decreased p38-MAPK activity and a shift in TAM polarization towards the M1 phenotype with reduced secretion of pro-tumor cytokines, but no change in vascular density or number of TAMs. In the E0771 model, VEGFR-1 inhibition reduced MMP-9 expression and decreased lung metastatic burden (p=0.026) in obese mice. In addition to these tumor effects, VEGFR-1 inhibition reduced weight gain, but caused metabolic disorder-hyperinsulinemia- [symbol] during obesity. Combining metformin with VEGFR-1 inhibition not only prevented this metabolic alteration, but also by recruiting cytotoxic cells further decreased tumor growth in the PAN02 model (p=0.047).

Conclusion: Inactivation of VEGFR-1 signaling prevents weight gain and obesity-induced acceleration of tumor progression in pancreatic and breast cancer models. Targeting VEGFR-1 signaling axis in combination with an anti-diabetic drug such as metformin might be a considerable cancer therapeutic option in the obese setting.

### REFERENCES FOR EXAMPLE 4

1. Sparano J A, Strickler H D. Breast cancer patients who are obese at diagnosis: alea iacta est? or “is the die cast”? Oncology (Williston Park) 2011; 25(11):1002, 1004, 1007
2. McWilliams R R, Matsumoto M E, Burch P A, et al. Obesity adversely affects survival in pancreatic cancer patients. Cancer 2010; 116(21):5054-62.
3. Hursting S D, Dunlap S M. Obesity, metabolic dysregulation, and cancer: a growing concern and an inflammatory (and microenvironmental) issue. Ann N Y Acad Sci 2012; 1271:82-7.
4. Incio J, Soares R. Obesity, Diabetes and Metabolic Syndrome Impact on TUMOR ANGIOGENESIS. In: Ruben R. Gonzalez-Perez B R R, (ed). Tumor Angiogenesis Regulators: CRC Press; 2013.
5. Shibuya M. Involvement of Flt-1 (VEGF receptor-1) in cancer and preeclampsia. Proc Jpn Acad Ser B Phys Biol Sci 2011; 87(4):167-78.
6. Hiratsuka S, Duda D G, Huang Y, et al. C—X—C receptor type 4 promotes metastasis by activating p38 mitogen-activated protein kinase in myeloid differentiation antigen (Gr-1)-positive cells. Proc Natl Acad Sci USA 2011; 108(1):302-7.

### Example 5

### Role of VEGFR-1 Signaling in Obesity-Induced Tumor Progression

### SUMMARY

Obesity associates with angiogenesis and increased macrophage infiltration in adipose tissues during weight gain. Whether these effects also occur in cancer to promote tumor progression in obese condition remains unclear. The inventors have shown that the vascular endothelial growth factor receptor-1 (VEGFR-1) pathway can modulate tumor angiogenesis and recruitment of tumor-associated macrophages (TAMs). The inventors investigated whether the obesity enhances tumor progression and metastasis by augmenting angiogenesis and TAM recruitment via activation of the VEGFR-1 pathway. To do this, a high-fat diet-induced obesity model in either wild type (WT) or VEGFR-1 tyrosine kinase null (Flt1^{TK/}) C57BL/6 mice were used. Then, orthotopically syngeneic pancreatic (PAN02) or breast (E0771) carcinomas were implanted. The role of VEGFR-1 activity on systemic metabolism, tumor angiogenesis and immune environment, and tumor growth and metastasis were evaluated.

It is shown that obesity increased p38-MAPK activation and TAM infiltration, tumor growth (p=0.001) and metastasis (p=0.035) in PAN02 tumors. VEGFR-1 inhibition reduced tumor growth (p=0.007) and metastasis (p=0.017) in obese but not lean mice. This was associated with a decreased p38-MAPK activity and a shift in TAM polarization towards the M1 phenotype with reduced secretion of pro-tumor cytokines, but no change in vascular density or number of TAMs. In the E0771 model, VEGFR-1 inhibition reduced MMP-9 expression and decreased lung metastatic burden (p=0.026) in obese mice. In addition to these tumor effects, VEGFR-1 inhibition reduced weight gain, but caused metabolic disorder—hyperinsulinemia—during obesity. Combining metformin with VEGFR-1 inhibition not only prevented this metabolic alteration, but also by recruiting cytotoxic cells further decreased tumor growth in the PAN02 model (p=0.047). Taken together, inactivation of VEGFR-1 signaling prevents weight gain and obesity-induced acceleration of tumor progression in pancreatic and breast cancer models. Targeting VEGFR-1 signaling axis in combination with an anti-diabetic drug such as metformin can be a cancer therapeutic option in the obese setting.

### Introduction

With the current obesity epidemic, the majority of patients diagnosed with pancreatic ductal adenocarcinoma (PDAC) and breast cancer (BC) are overweight or obese [1-3]. This excess body weight appears to be associated with accelerated tumor progression and poor prognosis, but the mechanisms are poorly understood [2-15].

It has been previously shown that vascular endothelial growth factor receptor 1 (VEGFR-1) is expressed in endothelial cells and macrophages, binds to VEGF and P1GF, and promotes tumor angiogenesis and p38-MAPK-dependent recruitment and activation (e.g., cytokine production) of tumor-associated macrophages (TAMs) [16-21]. In addition, mice genetically deficient for P1GF have been shown previously to gain less weight on a high-fat diet than wild-type animals [22], but presented with insulin resistance and hyperinsulinemia [23]. However, it is not clear whether targeting the P1GF/VEGFR-1 pathway can cause metabolically adverse effects that require treatment with an anti-diabetic drug such as metformin [24].

Accordingly, the relevance of VEGFR-1 activity in both the tumor microenvironment and adipose tissue (AT) as well as in the obesity-cancer interaction was evaluated. To this end, the role of VEGFR-1 activity in three major mechanisms during obesity-induced tumor progression: (1) angiogenesis, (2) immune cell recruitment and cytokine profile, and (3) AT expansion was assessed.

### Methods

### Animal Models

All experimental use of animals was approved by the institutional animal care and use committee at Massachusetts General Hospital (MGH). All animal procedures abided by Public Health Service Policy on Humane Care of Laboratory Animals guidelines. All mice were bred and maintained MGH animal facility. Wild-type (WT) C57BL/6 mice were originally obtained from the Jackson Laboratory (The Jackson Laboratory, Bar Harbor, Me.) but were bred and maintained in a defined-flora colony. VEGFR1 tyrosine kinase-knockout mice (Flt1^{TK-/-}) of mixed C57BL/6 and SV129 strain background were originally developed at the University of Tokyo, Japan (Hiratsuka et al. Proc Natl Acad Sci USA (1998)95(16): 9349-54), and subsequently backcrossed to 99.9% C57BL/6 strain background (N10 equivalent) at Massachusetts General Hospital. Strain background was verified by the Jackson Laboratory's Speed Congenic Development Service. Genotyping was performed using polymerase chain reaction (PCR) analysis, as previously described (Hiratsuka et al.). Age-matched animals were used for all experiments. Mice were maintained on a 12-h light-dark cycle in a temperature-controlled barrier facility, with ad libitum access to food and acidified water, unless otherwise specified.

### Body Composition

Mice (7-week old) were given either 10% or a 60% fat diet (D12450J and D12492, Research Diets, New Brunswick, N.J.) for 10 weeks, as described in Surwit et al. Metabolism (1995) 44(5):645-51. Body weight was monitored weekly and mice on a high-fat diet were defined as obese if their body weight was greater than 3 SD above the weight of the low fat diet group. At the end of the experiment, animals were weighed and euthanized, and the inguinal, perigonadal, and perineal fat pads were excised and weighed.

### Glucose Homeostasis

Glucose (GTT) and insulin tolerance tests (ITT) were performed after 8 and 9 weeks on the diet, respectively. Male mice were fasted for 16 hours (5pm-9am) for GTT, or 3 hours (9am-12pm) for ITT. Glucose (0.75 mg/g body weight) or insulin (1U/kg body weight) was administered by intraperitoneal (i.p.) injection, and tail blood was sampled at 0, 15, 30, 60, 90, and 120 minutes after administration of glucose/insulin. Blood glucose levels were measured using a Precision Xtra glucose meter (Abbott Diagnostics, Bedford, MA, USA). Glucose tolerance was evaluated calculating the area under the curve (AUC) for each mouse according to the following equation:

$$AUC = {\sum }_{n = 1}^{5} ( ( {T}_{n + 1} - {T}_{n} ) \times ( \frac{\vert {Glc}_{Tn} - {Glc}_{Tn + 1} \vert}{2} + min ( {Glc}_{Tn} , {Glc}_{Tn + 1} ) - {Glc}_{T 0} ) )$$

where Glc is glucose concentration, T is time, n is the time point number, and min(GlcTn,GlcTn+1) is the lower of two consecutive glucose concentrations (Heikkinen et al. Curr Protoc Mol Biol 2007; Chapter 29:Unit 29B 3). This equation adjusts for differences in fasting glucose levels, so that the evaluation is based on incremental area above the fasting baseline. For plasma insulin measurements, animals were fasted overnight before plasma was collected by tail bleed at 0 and 30 minutes after intraperitoneal (i.p.) injection of 0.75 mg/g body weight glucose. Plasma samples were stored at −80° C. until used. Plasma insulin concentration was determined using the Ultra Sensitive Mouse Insulin ELISA kit (Crystal Chem, Downers Grove, Ill., USA).

### Tumor Models

C57BL/6 VEGFR1(TK)−/− or WT obese and lean mice (after 10 weeks of diets) were used for tumor implantation studies. To induce pancreatic tumors, small (˜1 mm in diameter) chunks from a PAN02 pancreatic tumor source were grafted in the pancreata of obese and lean male mice. To induce breast tumors, 1×10⁶ E0771 breast cancer cells were implanted in the mammary fat pad of lean and obese female mice. About 3 weeks after implantation, tumors were removed and tissue was assessed for immune cell infiltration, gene and protein expression of inflammatory, metabolic and major signaling pathway markers. Plasma was also collected and assessed for insulin, IGF-1, glucose and inflammatory markers (see below). Metastatic burden was assessed by counting lung metastasis in the E0771 breast cancer model, and evaluating abdominal wall invasion as well as mesentery metastasis in the PAN02 pancreatic cancer model. For abdominal wall invasion, animals were scored from 0 to 3 based on the extent of invasion: 0=no invasion, 1=less than 3 metastasis, 2=less than 6 metastasis, 3=more than 6 metastasis.

For tumor implantation, mice were anesthetized for 30 minutes using intramuscular injections of ketamine/xylazine (90 mg/9 mg per kg BW). After surgery, Buprenorphine 0.1 mg/kg was administered every 12 hours for 72 hours. Criteria used for the decision to administer analgesics included ruffled fur, inability to self ambulate, hyper- or hypo-activity and the appearance of dehydration. If the aforementioned symptoms persisted, despite the use of analgesics for 3 days, the animal harboring them was removed from the study and euthanized. For tumor bearing animals, body weight was monitored at least twice a week to ensure that the mice did not experience greater than a 15% decrease in body weight. For tumor collection, mice were anesthetized for 30 minutes using intramuscular injections of ketamine/xylazine (90 mg/9 mg per kg BW). All animals were monitored on a daily basis.

E0771 cells were cultured in DMEM with 10% Serum Supreme (Lonza) and 1% HEPES buffer (Invitrogen). PAN02 tumor chunks and E0771 cells were authenticated in 2013 by IDEXX laboratories (PAN02: IDEXX RADIL Case # 22366-2013). The sample was confirmed to be of mouse origin and no mammalian interspecies contamination was detected. A genetic profile was generated for the sample by using a panel of microsatellite markers for genotyping. The sample is consistent with a C57BL/6 mouse strain from which this cell line originated. A comparison profile is provided and the reference profile for the C57BL/6NCr mouse strain is identical to the cell line except for an allele size difference at marker 39. The genetic profile generated for this sample can be used for comparison of samples. E0771: IDEXX RADIL Case # 14116-2013. The sample was confirmed to be of mouse origin and no mammalian interspecies contamination was detected. The genetic profile for this cell line contains extra alleles at 12 markers and different allele sizes at 5 markers. In addition, there are more than 2 alleles at 3 markers. The genetic profile for this cell line is not consistent with that of the C57BL/6 inbred mouse strain and is instead more consistent with having been derived from a mouse with a mixed/stock genetic background. Re-testing of this cell line after another 5-10 passages (IDEXX RADIL Case # 15518-2013) clarified that the genetic profile is intrinsic to this cell line but not likely cross-contaminated with another mouse cell line.)

### Gene Expression

Immediately following excision, tumor tissue was snap frozen and stored in liquid nitrogen until used. Total RNA was extracted and relative gene expression was determined using RT2 Profiler PCR Arrays system (Qiagen) on a Stratagene Mx3000P QPCR System. The pre-made pathway-focused arrays used (mouse genes, Qiagen) were “Inflammatory Cytokines and Receptors” (Cat. Number: PAMM011Z), “Common Cytokines” (Cat. Number: PAMM021Z), “T-cell and B-cell activation” (Cat. Number: PAMM053Z), and “Glucose Metabolism” (Cat. Number: PAMM006Z).

Western blot analysis

Each tumor sample was homogenated directly in lysis buffer for protein extraction. 20 ug of denatured protein per sample was loaded on 7%, 10% and 12% SDS-polyacrylamide gels. Membranes were blotted with antibodies against: phospho-AKT^{Ser473} and AKT; phospho-p38 mApK^{T180/Y182} and p38; phospho-pS6^{Ser235/236} and S6; phospho-stat3^{Tyr705} and Stat3; phospho-ERK(p44/42 MAPK)^{T202/Y204} and ERK; MMP-9; phospho-4EBP1 and 4EBP1, cleaved caspase 3; phospho-ACC^{Ser79} and ACC; phospho-Ampkβ^{Ser108} and Ampk; phospho-IRS-1^{Ser612}; phospho-IGF-1Rβ^{Tyr1135}; phospho-IR^{Y972}; LC-3; GAPDH and β-actin. Antibodies that listed above were obtained from Cell Signaling (Beverly, Mass.), and diluted 1:100 with the exception of phospho-IRS-1 (1:200); phospho-IGF-1receptor β (1:200) and phospho-IR (1:500); MMP-9 (1:500, EMD Millipore-Billerica, Mass.), GAPDH (1:2000, Ambion, N.Y.) and β-actin (1:5000, Sigma, Mo.).

### ELISA/Multiplex Array

Each tumor sample was homogenated directly in lysis buffer for protein extraction. 2 ug/ul of sample was used. Plasma was obtained from centrifugation of peripheral blood. The pre-made inflammatory multiple cytokines protein array was used in tumor homogenates and plasma (V-PLEX Proinflammatory Panell mouse kit, Cat. Number K15048D). Insulin and Glucose were measured as described above. IGF-1 was measured using a Mouse Immunoassay (Quantikine MG-100, RD Systems).

### Immunohistochemistry/Immunofluorescence

Freshly excised pancreata and adipose tissues were fixed in 4% (vol/vol) paraformaldehyde overnight. 10 μm sections were stained with hematoxylin and eosin (H&E) then examined for gross morphology. Insulin-producing cells were identified by staining with an anti-insulin primary antibody (R&D systems, Minneapolis, Minn., USA) and a fluorescent secondary antibody. Islet size and pancreas surface area were quantified using a custom-written computer script. Pancreatic macrophages were identified by staining with a primary antibody against ER-MP23 (Geutskens et al. J Leukoc Biol (2005) 78(4):845-52) (AbD Serotec, Raleigh, N.C., USA) and a fluorescent secondary antibody. The number of macrophages (from at least 5 randomly selected 10× fields) was normalized by the pancreas surface area. Adipose tissues were evaluated for vessel density using a biotinylated rat anti-CD31 (Dianova) antibody, leukocytes using an anti-CD45 antibody (Serotec), and macrophages using F4/80 (Serotec). The expression of each marker (from at least 5 randomly selected 10× fields) was normalized by the total tissue area. Tissue sections stained with non-specific IgG, instead of specific primary antibodies, were used as negative controls. Images were taken using a brightfield microscope with camera attached. For analysis of tumor tissues, primary tumor tissues were snap frozen in liquid nitrogen, and later embedded in OCT compound. Staining was carried out on frozen sections (10-μm thick) using rabbit antibodies against Ki67 (Abcam), VEGFR1, F4/80 (Serotec), CD31 (Millipore), IL-1β (Abcam), and phospho-p38 MAPK^{T180/Y182} (Cell Signaling). Cy3-, Cy5- or FITC-conjugated secondary antibodies were used for the detection of signals by confocal microscopy. Slides were counterstained with DAPI for nuclear staining. Mosaic images of tumors were collected using an Olympus FV1000 confocal laser-scanning microscope. A 10× air objective acquired 1260-μm square tiles, and an automated stage scanned throughout the entire cross-section of tumor tissue. The imaged tiles were stitched into a final mosaic image using Olympus software. Antigen expression was quantified by measuring the area occupied by the stain of interest normalized by the area of DAPI-stained nuclei (i.e., unitless), and analyzed using a custom algorithm in MATLAB.

Flow cytometry

Tumor-bearing mice were perfused via intracardiac injection of PBS and killed. Pancreatic and breast tumor tissues were harvested, minced, and digested at 37° C. for 1 h with DMEM containing collagenase type 1A (1.5 mg/mL), hyaluronidase (1.5 mg/mL), and DNase (2 mg/mL). The digestion mixtures were filtered through 70-μm cell strainers. Single-cell suspensions were incubated with rat anti-mouse CD16/CD32 mAb and then were stained, washed, and resuspended in cold buffer (1% BSA, 0.1% NaN3 in PBS). 7-Amino-actinomycin D (7AAD) reagent (eBioscience) was added to the stained tubes (5 μL per tube) just before running the flow analysis. The doublet/aggregated events were gated out using forward scatter area (FSC-A) vs. forward scatter width (FSC-W) and side scatter area (SSC-A) vs. side scatter width (SSC-W). Flow cytometry data were acquired on an LSRII flow cytometer (Becton Dickinson) and were analyzed with FACSDiva software. FSC-A vs. FSC-W and SSC-A vs. SSC-W were applied to discriminate the doublet/aggregated events. The appropriate fluorochrome-conjugated, isotype- matched control IgGs were used in all experiments. The following monoclonal anti-mouse antibodies were used: 7AAD (PerCP-Cy5.5, eBioscience), CD45 (PE-Cy7, Biolegend), CD45 (PerCP-Cy5.5eBioscience), CD4 (FITC, BD Bioscience), CD8 (PE, BD Bioscience), CD25 (APC-Cy7, BD Bioscience), NK (APC, BD Bioscience), F4/80 (PE-Cy7, Biolegend), CD11b (APC-Cy7, BD Bioscience), CD206 (FITC, Biolegend), Ly6C (APC, BD Bioscience), CD86 (PE, BD Biosciences).

### Statistical Analysis

The normal distribution of samples was determined by D'Agostino & Pearson omnibus normality test. Statistical differences were assessed by two-tailed student t test or Mann-Whitney test for comparisons between lean and obese, or obese and obese Flt1^{TK/} mice; one-way analysis of variance followed by post-hoc Turkey's multiple comparisons test or Kruskal-Wallis followed by Dunn's multiple comparisons test when analyzing multiple pairs of interest without a priori selection within the obese mice population; and two-way analysis of variance when comparing the effect of Flt1^{TK-/-} versus WT in lean and obese mice. Incidence of metastasis and wall invasion was analyzed using Fisher's exact test. All statistical tests were two-sided, and were performed using Prism Graphpad software. All results were considered statistically significant when P was less than 0.05 when calculated with the appropriate statistical test. Results are presented as the mean±standard error.

### Results

### Effect of VEGFR-1 Signaling Inhibition on Obesity-Induced Tumor Progression

High-fat diet led to a significant increase in body weight (BW) by the time of tumor implantation (FIG. 51A). In obese mice, PAN02 tumor growth rate and metastatic burden were approximately twice as high as in lean mice (FIGS. 51B-51C). This was associated with increased cell proliferation and reduced apoptosis (FIGS. 51D-51E). Tumors grown in Flt1^{TK-/-} lean mice were similar in size to lean WT. However, tumors grown in Flt1^{TK-/-} obese mice were significantly smaller compared to those grafted in obese WT mice (FIG. 51F). Similarly, in obese but not lean mice, VEGFR1 inhibition reduced the number of mesenteric metastases per animal (FIG. 51G) as well as the number of mice affected with metastasis from 60 to 20%, in addition to reducing the extent of retroperitoneal abdominal wall invasion (FIGS. 51H-51I). The loss of BW from the time of implantation until tumor extraction (a measure of health and systemic disease burden - in an extreme case, cachexia) was also significantly improved (˜4 times less body weight loss) in obese Flt1^{TK/} mice (FIG. 51J). The effect of VEGFR-1 inhibition on tumor progression in obese mice associated at the tumor level with decreased tumor cell proliferation, despite no apparent change in apoptosis (FIGS. 51K-51L).

### Effects of VEGFR-1 Signaling Inhibition on Tumor Angiogenesis and Immune Environment in Obesity

Tumor vessel density was not affected by obesity (FIG. 58A). However, the number of TAMs and the expression of pro-tumor cytokines IL-1β, IL-2, IL-4 and IL-5 were all increased in obese compared to lean mice (FIGS. 52A-52B). VEGFR-1 expression in tumors was predominantly associated with TAMs (FIG. 52C), approximately half of which were positive for this receptor. Surprisingly, VEGFR-1 inhibition in obese mice did not affect tumor vessel density (FIG. 58B) or the number of CD45+ leukocytes, TAMs, NK cells, or CD4+, CD8+ and Tregulatory lymphocytes (FIG. 52D). Instead, a decrease in protein levels was observed in pro-tumor M2 cytokines IL-10, IL-4, IL-5 and IL-1β (M1/M2a cytokine) in tumor tissue, and also in IL-1β and IL-2 (trend for IL-10) in plasma (FIG. 52E, and FIG. 66). IL-1β in particular, which showed the most dramatic change (50% decrease in obese Flt1^{TK-/-} VS. obese WT mice), was robustly expressed by TAMs (FIG. 59). Gene arrays were used to show that VEGFR-1 inhibition associated with the upregulation of M1-associated genes and concomitant downregulation of M2-associated genes, including IL-10 and T-cell function inhibitors CTLA-4 and PD-L2[30] (FIG. 52F). Furthermore, TAMs from obese Flt1^{TK-/-} mice were enriched with M1 surface markers (fraction of F4/80+ cells expressing LY6C+ or CD86+) compared to those from obese WT mice (FIG. 52G). The absolute number of M1 TAMs (F4/80+ LY6C+ double positive cells among the total viable cells) also increased by ˜70% (not shown). Collectively, these findings show that VEGFR-1 signaling in obese mice shifts TAM polarization and immune response towards an M2 (pro-tumor) phenotype in the PAN02 tumor model.

### Effect of VEGFR-1 Signaling Inhibition on Obesity-Induced Activation of Inflammatory Signaling Pathways

It was next sought to identify the signaling pathways that mediate the effects of VEGFR-1 on the tumor immune environment during obesity. It was found that VEGFR-1 inhibition decreased phosphorylated-p38-MAPK expression in tumors from obese mice, without affecting the phosphorylation of AKT, ERK, STAT3, S6, or 4EBP1 (FIG. 53A). Paralleling the findings above for IL-1β, IL-4, IL-5, IL-10 and tumor growth, it was shown that phospho-p38-MAPK was increased in obesity and subsequently normalized after VEGFR-1 inhibition to the levels in lean animals (FIG. 53B). Consistently, TAMs abundantly express phospho-p38-MAPK, particularly at the invading edge (FIG. 53C). Collectively, these data indicate that the obesity-induced changes in TAMs and the immune environment in PAN02 were mediated, at least in part, by VEGFR-1/p38-MAPK pathway activation.

### Effect of VEGFR-1 Signaling Inhibition on Obesity-Induced Breast Tumor Progression

Next, the effects of VEGFR-1 signaling blockade was determined in the E0771 BC model. Similar to PAN02, the average number of lung metastases was significantly decreased by VEGFR-1 inhibition in obese animals (FIG. 54A), and the incidence of metastasis tended to decrease (FIG. 54B). Tumor-induced cachexia was also significantly reduced in obese Flt1^{TK-/-} mice (FIG. 54C). Moreover, P38-MAPK activity was reduced after VEGFR-1 inhibition, as in PAN02 (FIG. 60). Consistent with the previous finding that VEGFR-1 regulates the production of matrix metalloproteinase 9 (MMP-9) by TAMs to promote lung metastasis [31], it was determined that obesity promoted MMP-9 expression in tumors (FIG. 61A), and that VEGFR-1 inhibition was able to decrease MMP-9 expression in obese mice (FIG. 54D). Of note, VEGFR-1 inhibition also tended to decrease MMP-9 expression in obese PAN02 tumors (FIG. 61B). In primary BC, similar to PAN02, immune cell recruitment and vessel density were unaltered with VEGFR-1 inhibition. However, unlike PAN02, VEGFR-1 inhibition did not affect primary BC growth (FIGS. 62A-62C). It was also found that compared to E0771, PAN02 tumors have ˜4 times higher protein levels of P1GF (FIG. 62D), which may prime them to respond to VEGFR-1 inhibition. In addition, unlike PAN02 tumors, the cytokine profile in E0771 tumors was mostly unaltered by VEGFR-1 inhibition (FIG. 62E and FIG. 66), indicating that the immune microenvironment from different tumors differs in its response/sensitivity to VEGFR-1 inhibition.

### Effects of VEGFR-1 Activity on Adipose Tissue, Systemic and Tumor Metabolism During Obesity

In addition to the direct effects on tumors, it was next sought to determine if VEGFR-1 signaling inhibition could indirectly be affecting obesity-induced tumor progression by attenuating BW gain. Indeed, it was found that Flt1^{TK-/-} mice on a high-fat diet (HFD) (but not on a low-fat diet) gained significantly less BW than WT mice [BW at tumor (PAN02 or E0771) implantation in FIGS. 55A-55B, BW gain kinetics in FIG. 63A)]. The reduction in weight gain by VEGFR-1 inhibition was associated with reduced visceral perigonadal fat and adipocyte size (FIGS. 63B-63C). Remarkably similar to the tumor setting, these effects in AT occurred without a reduction in the number of CD45+ leucocytes, F4/80+ macrophages, macrophage-rich crown-like structures or vessel density (FIGS. 63D-63J). However, despite prevention of weight gain by VEGFR-1 inhibition in mice on HFD, it was found that these mice showed elevated glucose levels after fasting and during a glucose tolerance test (GTT) (FIG. 55C). There was no significant difference in area under the curve, indicating that the elevated baseline levels could account for the higher glucose levels during GTT. Suprisingly, insulin tolerance remained similar between the two genotypes (FIG. 64A). Again, the differences in fasting glucose only occurred in obese but not lean mice (data not shown). Importantly, the above findings on glucose metabolism were not due to macrophage infiltration, altered pancreatic β-cell, insulin production or an overall change in pancreatic tissue mass (FIGS. 64B-64F). It was next investigated if the altered systemic metabolism observed in obese Flt1^{TK-/-} mice also affects tumor metabolism upon tumor inoculation. It was observed in both tumor models that compared to WT mice, Flt1^{TK/} mice presented with increased plasma insulin (FIG. 55D), but not insulin growth factor 1 (IGF-1) or glucose (not shown). Elevated insulin associated with increased phosphorylation of insulin receptor (p-IR) and IGF-1 receptor (pIGF-1R) in PAN02 tumors, with a trend for increased phosphorylated insulin receptor substrate 1 (pIRS-1) (FIG. 55E). These findings were associated with decreased expression of gluconeogenic genes and autophagy in tumors (FIGS. 65A-65B), but no major changes in other metabolic pathways (not shown). In BC, VEGFR-1 inhibition in obese mice was also associated with significantly increased p-IGF1R (FIG. 55E). Collectively, these results indicate that in addition to modulating immune cell function, VEGFR-1 activity inhibition affects systemic and tumor metabolism in obese mice.

Effects of VEGFR-1 Inhibition in Combination with Metformin on Systemic Metabolism and Tumor Environment During Obesity

The effects of the anti-diabetic drug metformin to ameliorate the metabolic aberrations of VEGFR-1 inhibition in obesity, particularly in PAN02-bearing male mice (>2-fold higher insulin levels in PDAC/male compared to BC/female in obese Flt1^{TK-/-} mice, FIG. 55D) [32] were assessed. Metformin prevented the increased insulin observed in obese Flt1^{TK-/-} mice (FIG. 56A). Furthermore, PAN02 tumor weight in obese mice was at its lowest with VEGFR-1 inhibition and metformin combined (FIG. 56B). This combination increased pericyte coverage in tumors despite unaltered vessel density in either tumor (FIG. 56C) or in adipose tissue (FIG. 65C). Consistent with increased vessel maturation, the fraction of perfused vessels was significantly higher in the combination group compared to either control or anti-VEGFR-1 alone (FIG. 56C). The increased vessel perfusion associated with increased recruitment of cytotoxic CD8+ T cells (p=0.05) and NK cells (p=0.06) (FIG. 56D). In addition, the inhibitory effect of VEGFR-1 inhibition on CTLA-4 and PD-L2 expression was maintained in the combination group (FIG. 56E). This indicates a synergism of enhanced T cell function by VEGFR-1 inhibition and T/NK cell number by metformin. This consequently associated with increased tumor cell apoptosis and decreased proliferation (FIGS. 56F-56G). Surprisingly, metformin restored expression of major gluconeogenic genes downregulated by VEGFR-1 inhibition in obese tumors (FIG. 65A), but no changes were seen in IR/IGF-1 signaling or AMPK/ACC activation (FIG. 65D). These findings indicate that metformin normalizes abnormal tumor vasculature, immunity and metabolism in obese Flt1TK^{-/-} mice thereby inhibiting tumor progression.

### Discussion

As presented herein, diet-induced weight gain and obesity-induced tumor growth and metastasis can be dependent on VEGFR-1 signaling (FIG. 57). In PDAC, obesity associated with increased TAM infiltration, and VEGFR-1 inhibition shifted TAM polarization and cytokine profile from a pro-tumor to an anti-tumor phenotype, and reduced markers of T cell dysfunction. This is the first study to demonstrate a modulation of immune cell activity by VEGFR-1. Ronly et al reported an M1 shift in macrophage polarization after inhibition of P1GF in a non-tumor/non-obese setting [33]. Although inhibition of P1GF/VEGFR1 signaling in this and previous studies did not alter immune cell infiltration in tumors [34-36], this effect has been shown to be highly tumor and context dependent [21, 37-39]. In addition to normalizing the obesity-augmented production of M2 cytokines and the M1/M2 cytokine IL-1β (produced in ˜50% by TAMs), VEGFR-1 inhibition reduced the obesity-induced overexpression of p38-MAPK, also abundantly expressed in TAMs. These findings are consistent with previous studies reporting that P1GF/VEGFR-1 signaling promotes activation of p38-MAPK and secretion of IL-1β by monocytes/macrophages [21, 40], and that p38-MAPK promotes M2 polarization and production of the cytokines above [41-43]. Consistently, similarly to VEGFR1 inhibition, it was found that IL-1B inhibition reduced PAN02 growth in obese but not in lean mice (data not shown). Collectively, these findings indicate that VEGFR-1 activity is, at least in part, important for obesity-accelerated tumor progression: VEGFR-1 activity in TAMs promotes p38-MAPK activity, polarization towards the tumor-promoting M2 phenotype, reduced T cell function, and upregulation of IL-1β and other cytokines to facilitate tumor growth in obesity (FIG. 57).

In the E0771 breast cancer model, it was found that VEGFR-1 inhibition normalizes obesity-amplified MMP-9 expression and lung metastasis. This is consistent with a previous report on VEGFR-1 promoting the expression of MMP-9 in lung macrophages, which facilitated metastasis in an experimental model [31]. In lean mice, no effect of VEGFR-1 genetic or pharmacological inhibition was previously shown on spontaneous metastasis formation [35, 36, 44]. It was discovered herein that in obese mice, but not in lean, spontaneous metastasis was reduced after VEGFR-1 inhibition in both BC and PDAC models. On the other hand, VEGFR-1 inhibition affected cytokine profile and primary tumor growth in obese mice only in the PDAC. Without wishing to be bound by theory, this could be explained, in part, by the increased P1GF levels in PAN02 compared to E0771 tumors (˜4 times higher). Previous studies have reported that VEGFR-1 inhibition affects primary tumor growth only in the presence of P1GF overexpression [37, 39]. Importantly, it was discovered that P1GF overexpression was tumor and not gender specific as systemic (plasma) P1GF levels were similar in both males and females (FIG. 62D). Another relevant factor could be the effect of obesity on TAM infiltration and cytokine profile. Obesity alone did not promote TAM infiltration (FIG. 62F) or increased M2 pro-tumor cytokines in E0771 tumors compared to PAN02 tumors. Hence, the TAM-mediated effect of VEGFR-1 inhibition may not be present in E0771 tumors. The obesity-induced TAM-rich environment and P1GF overexpression may thus “prime” PAN02 tumors to respond to VEGFR-1 inhibition.

In addition to the effect on immune cells, it was previously reported that VEGFR-1 can regulate tumor angiogenesis in a context dependent manner [21, 45, 46]. However, it was found herein that VEGFR-1 inhibition did not modify vessel density in adipose tissue (AT) or either tumor model. Carmeliet et al have previously reported that the angiogenic role of VEGFR-1 is in part due to the recruitment of TAMs that secrete pro-angiogenic factors in the tumor microenvironment [18, 47]. Hence, the lack of the effect on TAM recruitment observed in the study described herein may explain at least in part the lack of the effect on angiogenesis.

Besides its direct effects on cancer, it was discovered herein that VEGFR-1 deficiency also reduced weight gain in mice under HFD. The reduction of body weight gain with VEGFR-1 inhibition was not associated with reduced vasculature or immune cell infiltration. However, the M1 macrophages and M1 cytokine profile observed are known to be associated with insulin resistance [49] and promote adipocyte cell death [50], which could explain, in part, the impaired glucose metabolism and prevention of weight gain in obese mice. Hemmeryckx et al previously reported that P1GF deficiency in mice fed a high-fat diet promoted insulin resistance and hyperinsulinemia, presumably via reduced fraction of brown adipocytes and stimulation of white adipocyte hypertrophy [23]. It was discovered herein, however, that VEGFR-1 inhibition actually reduced adipocyte size. Moreover, various pancreatic morphological parameters including 13-cells and insulin production, as well as macrophage infiltration in the pancreas, which can also influence insulin production [51, 52], were unaltered with VEGFR-1 inhibition. This indicates that the lack of VEGFR-1 signaling did not alter pancreatic insulin production, likely affecting peripheral resistance to this hormone. Inhibition of Pi3K-eNOS signaling by VEGFR-1 inhibition may have been involved, as previously observed in a model of diabetic nephropathy [53].

The aggravated diabetes-like systemic metabolism in obese Flt1^{TK-/-} compared to obese WT mice was associated with increased plasma insulin and activation of insulin/IGF-1 signaling in tumors. The anti-diabetic drug metformin [24, 54-58] normalized plasma insulin levels in obese Flt1^{TK-/-} mice. Remarkably, metformin also normalized pancreatic tumor vasculature and immune microenvironment - by increasing perfusion and recruitment of CD8+ T cells and NK cells - that were associated with increased cell death and reduced tumor growth. This indicated a synergism between the enhanced Tcell function by VEGFR-1 inhibition (hence acting as an immune checkpoint inhibitor) and the increased infiltration of T/NK cells by metformin. Contrary to previous studies [54, 57, 59, 60], a significant reduction in the major metabolic pathways—including IGF-1 and AMPK—was not found after metformin treatment.

Collectively, these findings indicate that VEGFR-1 mediates obesity-promoted tumor growth and metastasis both indirectly by interfering with adipose tissue (AT) expansion and directly by altering tumor microenvironment. VEGFR-1 inhibition was effective in obese setting, and consistent with our results, plasma levels of P1GF are increased in obese PDAC and BC patients compared to lean (unpublished results). Obesity associates with worse prognosis in these cancers, and with the current obesity epidemics, PDAC and BC patients that have excess body weight at diagnosis are the majority [1-6]. Stratifying these patients by body weight for treatment can enhance the efficacy of anti-VEGFR-1 agents such as multi-kinase inhibitors, which have failed to show efficacy in unselected populations [62-64]. In addition, this study indicate that VEGFR-1 can be a valid target in obese patients particularly in combination with a drug that controls systemic metabolism such as metformin.

### REFERENCES FOR EXAMPLE 5

1. Sparano J A, Strickler H D. Breast cancer patients who are obese at diagnosis: alea iacta est? or “is the die cast”? Oncology (Williston Park) 2011; 25(11):1002, 1004, 1007.
2. McWilliams R R, Matsumoto M E, Burch P A, et al. Obesity adversely affects survival in pancreatic cancer patients. Cancer 2010; 116(21):5054-62.
3. Li D, Morris J S, Liu J, et al. Body mass index and risk, age of onset, and survival in patients with pancreatic cancer. JAMA 2009; 301(24):2553-62.
4. Hursting S D, Dunlap S M. Obesity, metabolic dysregulation, and cancer: a growing concern and an inflammatory (and microenvironmental) issue. Ann N Y Acad Sci 2012; 1271:82-7.
5. Pischon T, Nothlings U, Boeing H. Obesity and cancer. Proc Nutr Soc 2008; 67(2):128-45.
6. Calle E E, Thun M J. Obesity and cancer. Oncogene 2004; 23(38):6365-78.
7. Incio J, Soares R. Obesity, Diabetes and Metabolic Syndrome Impact on TUMOR ANGIOGENESIS. In: Ruben R. Gonzalez-Perez B R R, (ed). Tumor Angiogenesis Regulators: CRC Press; 2013.
8. Hosogai N, Fukuhara A, Oshima K, et al. Adipose tissue hypoxia in obesity and its impact on adipocytokine dysregulation. Diabetes 2007; 56(4):901-11.
9. Costa C, Incio J, Soares R. Angiogenesis and chronic inflammation: cause or consequence? Angiogenesis 2007; 10(3):149-66.
10. Weisberg S P, McCann D, Desai M, et al. Obesity is associated with macrophage accumulation in adipose tissue. J Clin Invest 2003; 112(12):1796-808.
11. Silverman K J, Lund D P, Zetter B R, et al. Angiogenic activity of adipose tissue. Biochem Biophys Res Commun 1988; 153(1):347-52.
12. Murano I, Barbatelli G, Parisani V, et al. Dead adipocytes, detected as crown-like structures, are prevalent in visceral fat depots of genetically obese mice. J Lipid Res 2008; 49(7):1562-8.
13. Xu H, Barnes G T, Yang Q, et al. Chronic inflammation in fat plays a crucial role in the development of obesity-related insulin resistance. J Clin Invest 2003; 112(12):1821-30.
14. Chen C T, Du Y, Yamaguchi H, et al. Targeting the IKKbeta/mTOR/VEGF signaling pathway as a potential therapeutic strategy for obesity-related breast cancer. Mol Cancer Ther 2012; 11(10):2212-21.
15. Okwan-Duodu D, Umpierrez G E, Brawley O W, et al. Obesity-driven inflammation and cancer risk: role of myeloid derived suppressor cells and alternately activated macrophages. Am J Cancer Res 2013; 3(1):21-33.
16. Murakami M, Zheng Y, Hirashima M, et al. VEGFR1 tyrosine kinase signaling promotes lymphangiogenesis as well as angiogenesis indirectly via macrophage recruitment. Arterioscler Thromb Vasc Biol 2008; 28(4):658-64.
17. Li B, Sharpe E E, Maupin A B, et al. VEGF and P1GF promote adult vasculogenesis by enhancing EPC recruitment and vessel formation at the site of tumor neovascularization. Faseb J 2006; 20(9):1495-7.
18. Fischer C, Mazzone M, Jonckx B, et al. FLT1 and its ligands VEGFB and P1GF: drug targets for anti-angiogenic therapy? Nat Rev Cancer 2008; 8(12):942-56.
19. Murakami M, Iwai S, Hiratsuka S, et al. Signaling of vascular endothelial growth factor receptor-1 tyrosine kinase promotes rheumatoid arthritis through activation of monocytes/macrophages. Blood 2006; 108(6):1849-56.
20. Shibuya M. Involvement of Flt-1 (VEGF receptor-1) in cancer and preeclampsia. Proc Jpn Acad Ser B Phys Biol Sci 2011; 87(4):167-78.
21. Hiratsuka S, Duda DG, Huang Y, et al. C—X—C receptor type 4 promotes metastasis by activating p38 mitogen-activated protein kinase in myeloid differentiation antigen (Gr-1)-positive cells. Proc Natl Acad Sci USA 2011; 108(1):302-7.
22. Lijnen H R, Christiaens V, Scroyen I, et al. Impaired adipose tissue development in mice with inactivation of placental growth factor function. Diabetes 2006; 55(10):2698-704.
23. Hemmeryckx B, van Bree R, Van Hoef B, et al. Adverse adipose phenotype and hyperinsulinemia in gravid mice deficient in placental growth factor. Endocrinology 2008; 149(5):2176-83.
24. Muti P, Berrino F, Krogh V, et al. Metformin, diet and breast cancer: an avenue for chemoprevention. Cell Cycle 2009; 8(16):2661.
25. Hiratsuka S, Minowa O, Kuno J, et al. Flt-1 lacking the tyrosine kinase domain is sufficient for normal development and angiogenesis in mice. Proc Natl Acad Sci USA 1998; 95(16):9349-54.
26. Heikkinen S, Argmann C A, Champy M F, et al. Evaluation of glucose homeostasis. Curr Protoc Mol Biol 2007;Chapter 29:Unit 29B 3.
27. Kodack D P, Chung E, Yamashita H, et al. Combined targeting of HER2 and VEGFR2 for effective treatment of HER2-amplified breast cancer brain metastases. Proc Natl Acad Sci USA 2012; 109(45):E3119-27.
28. Chauhan V P, Martin J D, Liu H, et al. Angiotensin inhibition enhances drug delivery and potentiates chemotherapy by decompressing tumour blood vessels. Nature communications 2013; 4.
29. Huang Y, Yuan J, Righi E, et al. Vascular normalizing doses of antiangiogenic treatment reprogram the immunosuppressive tumor microenvironment and enhance immunotherapy. Proc Natl Acad Sci USA 2012; 109(43):17561-6.
30. Zamarin D, Postow M A. Immune checkpoint modulation: Rational design of combination strategies. Pharmacol Ther 2015; 10.1016/j.pharmthera.2015.01.003.
31. Hiratsuka S, Nakamura K, Iwai S, et al. MMP9 induction by vascular endothelial growth factor receptor-1 is involved in lung-specific metastasis. Cancer Cell 2002; 2(4):289-300.
32. Sadeghi N, Abbruzzese J L, Yeung S C, et al. Metformin use is associated with better survival of diabetic patients with pancreatic cancer. Clin Cancer Res 2012; 18(10):2905-12.
33. Rolny C, Mazzone M, Tugues S, et al. HRG inhibits tumor growth and metastasis by inducing macrophage polarization and vessel normalization through downregulation of P1GF. Cancer Cell 2011; 19(1):31-44.
34. Casanovas O, Hicklin D J, Bergers G, et al. Drug resistance by evasion of antiangiogenic targeting of VEGF signaling in late-stage pancreatic islet tumors. Cancer Cell 2005; 8(4):299-309.
35. Dawson M R, Duda D G, Fukumura D, et al. VEGFR1-activity-independent metastasis formation. Nature 2009; 461(7262):E4; discussion E5.
36. Dawson M R, Duda D G, Chae S S, et al. VEGFR1 activity modulates myeloid cell infiltration in growing lung metastases but is not required for spontaneous metastasis formation. PLoS One 2009; 4(9):e6525.
37. Muramatsu M, Yamamoto S, Osawa T, et al. Vascular endothelial growth factor receptor-1 signaling promotes mobilization of macrophage lineage cells from bone marrow and stimulates solid tumor growth. Cancer Res 2010; 70(20):8211-21.
38. Kerber M, Reiss Y, Wickersheim A, et al. Flt-1 signaling in macrophages promotes glioma growth in vivo. Cancer Res 2008; 68(18):7342-51.
39. Hiratsuka S, Maru Y, Okada A, et al. Involvement of Flt-1 tyrosine kinase (vascular endothelial growth factor receptor-1) in pathological angiogenesis. Cancer Res 2001; 61(3):1207-13.
40. Selvaraj S K, Giri R K, Perelman N, et al. Mechanism of monocyte activation and expression of proinflammatory cytochemokines by placenta growth factor. Blood 2003; 102(4):1515-24.
41. Liu J, Liu L, Cui Y, et al. p38 MAPK regulates Th2 cytokines release in PBMCs in allergic rhinitis rats. J Huazhong Univ Sci Technolog Med Sci 2010; 30(2):222-5.
42. Parhar K, Ray A, Steinbrecher U, et al. The p38 mitogen-activated protein kinase regulates interleukin-1beta-induced IL-8 expression via an effect on the IL-8 promoter in intestinal epithelial cells. Immunology 2003; 108(4):502-12.
43. Perdiguero E, Sousa-Victor P, Ruiz-Bonilla V, et al. p38/MKP-1-regulated AKT coordinates macrophage transitions and resolution of inflammation during tissue repair. J Cell Biol 2011; 195(2):307-22.
44. Duda D G, Jain R K. Premetastatic lung “niche”: is vascular endothelial growth factor receptor 1 activation required? Cancer Res 2010; 70(14):5670-3.
45. Luttun A, Tjwa M, Moons L, et al. Revascularization of ischemic tissues by P1GF treatment, and inhibition of tumor angiogenesis, arthritis and atherosclerosis by anti-Flt1. Nat Med 2002; 8(8):831-40.
46. Shibuya M. Vascular endothelial growth factor and its receptor system: physiological functions in angiogenesis and pathological roles in various diseases. J Biochem 2013; 153(1):13-9.
47. Dewerchin M, Carmeliet P. Placental growth factor in cancer. Expert Opin Ther Targets 2014; 18(11):1339-54.
48. Tam J, Duda D G, Perentes J Y, et al. Blockade of VEGFR2 and not VEGFR1 can limit diet-induced fat tissue expansion: role of local versus bone marrow-derived endothelial cells. PLoS One 2009; 4(3):e4974.
49. Hong E G, Ko H J, Cho Y R, et al. Interleukin-10 prevents diet-induced insulin resistance by attenuating macrophage and cytokine response in skeletal muscle. Diabetes 2009; 58(11):2525-35.
50. Mauer J, Chaurasia B, Goldau J, et al. Signaling by IL-6 promotes alternative activation of macrophages to limit endotoxemia and obesity-associated resistance to insulin. Nat Immuno! 2014; 15(5):423-30.
51. Geutskens S B, Otonkoski T, Pulkkinen M A, et al. Macrophages in the murine pancreas and their involvement in fetal endocrine development in vitro. J Leukoc Biol 2005; 78(4):845-52.
52. Tessem J S, Jensen J N, Pelli H, et al. Critical roles for macrophages in islet angiogenesis and maintenance during pancreatic degeneration. Diabetes 2008; 57(6):1605-17.
53. Yang K S, Lim J H, Kim T W, et al. Vascular endothelial growth factor-receptor 1 inhibition aggravates diabetic nephropathy through eNOS signaling pathway in db/db mice. PLoS One 2014; 9(4):e94540.
54. Pollak M. Insulin, insulin-like growth factors and neoplasia. Best Pract Res Clin Endocrinol Metab 2008; 22(4):625-38.
55. Deng X S, Wang S, Deng A, et al. Metformin targets Stat3 to inhibit cell growth and induce apoptosis in triple-negative breast cancers. Cell Cycle 2012; 11(2):367-76.
56. Zakikhani M, Dowling R, Fantus I G, et al. Metformin is an AMP kinase-dependent growth inhibitor for breast cancer cells. Cancer Res 2006; 66(21):10269-73.
57. Goodwin P J, Stambolic V. Obesity and insulin resistance in breast cancer-Chemoprevention strategies with a focus on metformin. Breast;20 Suppl 3:S31-5.
58. Bao B, Wang Z, Ali S, et al. Metformin inhibits cell proliferation, migration and invasion by attenuating CSC function mediated by deregulating miRNAs in pancreatic cancer cells. Cancer Prey Res (Phila) 2012; 5(3):355-64.
59. Algire C, Zakikhani M, Blouin M J, et al. Metformin attenuates the stimulatory effect of a high-energy diet on in vivo LLC1 carcinoma growth. Endocr Relat Cancer 2008; 15(3):833-9.
60. Mohammed A, Janakiram N B, Brewer M, et al. Antidiabetic Drug Metformin Prevents Progression of Pancreatic Cancer by Targeting in Part Cancer Stem Cells and mTOR Signaling. Trans1 Oncol 2013; 6(6):649-59.
61. Yuan C, Rubinson D A, Qian Z R, et al. Survival Among Patients With Pancreatic Cancer and Long-Standing or Recent-Onset Diabetes Mellitus. J Clin Oncol 2014; 10.1200/JCO.2014.57.5688.
62. Tan Q X, Qin Q H, Lian B, et al. Sorafenib-based therapy in HER2-negative advanced breast cancer: Results from a retrospective pooled analysis of randomized controlled trials. Exp Ther Med 2014; 7(5): 1420-1426.
63. Cardin D B, Goff L, Li C I, et al. Phase II trial of sorafenib and erlotinib in advanced pancreatic cancer. Cancer Med 2014; 3(3): 572-9.
64. Kindler H L, Wroblewski K, Wallace J A, et al. Gemcitabine plus sorafenib in patients with advanced pancreatic cancer: a phase II trial of the University of Chicago Phase II Consortium. Invest New Drugs 2012; 30(1): 382-6.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. (canceled)

2. A method of improving the delivery and/or efficacy of a cancer therapy or an anti-fibrotic therapy, or treating or preventing a cancer or a fibrotic disorder, in a subject, comprising administering a metformin agent to the subject in combination with one, two, three or more of:
(i) an anti-hypertensive and/or a collagen modifying agent (AHCM) (e.g., an angiotensin receptor blocker (ARB));
(ii) a microenvironment modulator (e.g., an anti-angiogenic inhibitor e.g., a low-dose anti-angiogenic inhibitor), and/or other stromal modulators;
(iii) an anti-inflammatory agent (e.g., a cytokine inhibitor); or
(iv) an inhibitor of an immune checkpoint molecule, and
optionally, administering the cancer or the anti-fibrotic therapy, under conditions sufficient to treat or prevent the cancer therapy or the fibrotic disorder, in the subject, or to improve the delivery and/or efficacy of the cancer therapy or the anti-fibrotic therapy provided to the subject.

3-5. (canceled)

6. The method of claim 2, further comprising a step of identifying and/or stratifying, responsiveness of a subject, e.g., a cancer patient, for a metformin therapy and/or an AHCM therapy, said step comprising:
acquiring a value for, a weight/metabolic-related parameter (e.g., BMI) for the subject; and, responsive to a determination of a weight/metabolic-related parameter indicative of overweight or obesity (e.g., BMI value greater than 25), performing one, two, three or more of:
(i) identifying the subject as being likely to respond to the metformin therapy and/or the AHCM therapy;
(ii) stratifying the subject, or a patient populations (e.g., stratifying the subject) as being likely to respond (e.g., responders vs. non-responders) to the metformin therapy and/or the AHCM therapy;
(iii) more effectively monitor the metformin therapy and/or the AHCM therapy; or
(iv) administering the metformin agent, alone or in combination with, one, two, three or more of: (i) an AHCM (e.g., an ARB); (ii) a microenvironment modulator (e.g., an anti-angiogenic inhibitor), and/or other stromal modulators; (iii) an anti-inflammatory agent (e.g., a cytokine inhibitor); or (iv) an inhibitor of an immune checkpoint molecule.

7. The method of claim 2, further comprising identifying the subject as having a desmoplastic disorder (e.g., a cancer or a fibrotic disorder).

8-10. (canceled)

11. The method of claim 2, wherein the metformin agent is a biguanide or any functional analog, derivative, or salt thereof.

12. The method of claim 2, wherein the metformin agent comprises two linked guanidine moieties.

13. The method of claim 2, wherein the metformin agent is chosen from metformin, phenformin, buformin, and biguanide, or any functional analog, derivative, or salt of any of the aforesaid compounds.

14. The method of claim 2, wherein the metformin agent is described by a compound of Formula (I): 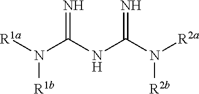
or a pharmaceutically acceptable salt thereof, wherein each of R1a, R1b, R2a, and R2b is independently hydrogen, C1-C6 alkyl, cycloalkylalkyl, or arylalkyl.

15. The method of claim 14, wherein each of R1a and R1b is hydrogen; each of R2a and R2b is independently hydrogen or C1-C6 alkyl; each of R2a and R2b is independently C1-C4 alkyl; each of R2a and R2b is independently C1-C2 alkyl. In some embodiments, each of R2a and R2b is independently methyl; each of R1a and R1b is hydrogen, and each of R2a and R2b is methyl.

16. The method of claim 2, wherein the metformin agent is metformin, e.g., 3-(diaminomethylidene)-1,1-dimethylguanidine.

17-34. (canceled)

35. The method of claim 2, wherein the metformin agent, the AHCM, the microenvironment modulator and/or the other stromal modulator is administered prior to, after cessation, and/or in combination with the cancer or the fibrotic therapy.

36. (canceled)

37. (canceled)

38. The method of claim 2, wherein the metformin, the AHCM, the microenvironment modulator, or the other stromal modulator is administered as a particle or in free form in a dosage sufficient to improve the delivery or effectiveness of the cancer or fibrotic therapy.

39. (canceled)

40. The method of claim 2, wherein the subject has a pre-neoplastic condition or a pre-disposition to cancer.

41. The method of claim 2, wherein the subject is at risk of having, or has a solid, fibrotic tumor.

42. (canceled)

43. (canceled)

44. The method of claim 2, wherein when the subject is treated with an AHCM, the method further comprising administering to the subject an anti-angiogenic agent, e.g., a VEGF/VEGFR inhibitor.

45-62. (canceled)

63. A method for treating or preventing a liver disorder or condition in a subject., comprising administering to the subject an AHCM and a vascular/stromal normalizing dose (e.g., a sub-anti-angiogenic dose) of a second agent chosen from one or more of: anti-angiogenic agent, sorafenib or an inhibitor of the angiopoietin-Tie-2 pathway (e.g., an Ang-1 or an Ang-2 inhibitor), thereby treating or preventing the liver disorder or condition.

64. The method of claim 63, wherein the second agent is administered at a sub-anti-angiogenic dose.

65-69. (canceled)

70. A combination or composition (e.g., one or more compositions or dosage forms), that includes a metformin agent in combination with one, two, three or more of:
(i) an AHCM (e.g., an ARB);
(ii) a microenvironment modulator (e.g., an anti-angiogenic inhibitor, e.g., a low-dose anti-angiogenic inhibitor) and/or other stromal modulators;
(iii) an anti-inflammatory agent (e.g., a cytokine inhibitor); or
(iv) an inhibitor of an immune checkpoint molecule; and
optionally, a cancer or an anti-fibrotic therapy.

71-94. (canceled)

95. The combination or composition of claim 70, wherein the metformin agent, the AHCM, the microenvironment modulator and/or the other stromal modulator is administered prior to and/or in combination with the cancer or the fibrotic therapy.

96-104. (canceled)

105. The combination or composition of claim 70, wherein the AHCM, the microenvironment modulator, the other stromal modulator, or the cancer therapy is administered to the subject by a systemic administration chosen from oral, parenteral, subcutaneous, intravenous, rectal, intramuscular, intraperitoneal, intranasal, transdermal, or by inhalation or intracavitary installation.

106-109. (canceled)

110. The method of claim 2, further comprising a step of identifying the subject as being in need of improved delivery and/or efficacy of a cancer therapy or an anti-fibrotic therapy; responsive to said identification, administering a metformin agent to the subject in combination with one, two, three or more of:
(i) an anti-hypertensive and/or a collagen modifying agent (AHCM) (e.g., an angiotensin receptor blocker (ARB));
(ii) a microenvironment modulator (e.g., an anti-angiogenic inhibitor e.g., a low-dose anti-angiogenic inhibitor), and/or other stromal modulators;
(iii) an anti-inflammatory agent (e.g., a cytokine inhibitor); or
(iv) an inhibitor of an immune checkpoint molecule, and
optionally, administering the cancer or the anti-fibrotic therapy, under conditions sufficient to treat or prevent the cancer therapy or the fibrotic disorder, in the subject, or to improve the delivery and/or efficacy of the cancer therapy or the anti-fibrotic therapy provided to the subject.
